(19) United States
(12) Patent Application Publication
(10) Pub. No.: US 2024/0327888 A1
(43) Pub. Date: Oct. 3, 2024

(54) LACTO-N-BIOSE CONVERTING FUCOSYLTRANSFERASES

(71) Applicant: INBIOSE N.V., Zwijnaarde (BE)

(72) Inventors: Sofie Aesaert, Zwijnaarde (BE); Joeri Beauprez, Zwijnaarde (BE); Gert Peters, Zwijnaarde (BE); Kristof Vandewalle, Zwijnaarde (BE); Annelies Vercauteren, Zwijnaarde (BE)

(21) Appl. No.: 18/579,664

(22) PCT Filed: Jul. 15, 2022
(86) PCT No.: PCT/EP2022/069849

§ 371 Date: Jan. 16, 2024

(30) Foreign Application Priority Data
Jul. 16, 2021 (EP) 21186204.0
Jul. 16, 2021 (LU) LU500444

(51) Int. Cl. C12P 19/26; C12N 9/10; C12N 15/63

(52) U.S. Cl. CPC C12P 19/26; C12N 9/1051; C12N 15/63; C12Y 204/01069

(57) ABSTRACT

The present invention is in the technical field of synthetic biology and metabolic engineering. More particularly, the present invention is in the technical field of metabolically engineered cells and use of said cells in a cultivation, preferably a fermentation. The present invention describes a cell and a method for production of a compound. The cell expresses an alpha-1,2-fucosyltransferase that has galactoside alpha-1,2-fucosyltransferase activity on the galactose residue of Gal-b1,3-GlcNAc (LNB, lacto-N-biose). Furthermore, the present invention provides for purification of said compound from the cultivation.

### FIELD OF THE INVENTION

The present invention is in the technical field of synthetic biology and metabolic engineering. More particularly, the present invention is in the technical field of metabolically engineered cells and use of said cells in a cultivation, preferably a fermentation. The present invention describes a cell and a method for production of a compound. The cell expresses an alpha-1,2-fucosyltransferase that has galactoside alpha-1,2-fucosyltransferase activity on the galactose residue of Gal-b1,3-GlcNAc (LNB, lacto-N-biose). Furthermore, the present invention provides for purification of said compound from the cultivation.

### BACKGROUND

Oligosaccharides, often present as glyco-conjugated forms to proteins and lipids, are involved in many vital phenomena such as differentiation, development and biological recognition processes related to the development and progress of fertilization, embryogenesis, inflammation, metastasis, and host pathogen adhesion. Oligosaccharides can also be present as unconjugated glycans in body fluids and human milk wherein they also modulate important developmental and immunological processes (Bode, Early Hum. Dev. 1-4 (2015); Reily et al., Nat. Rev. Nephrol. 15, 346-366 (2019); Varki, Glycobiology 27, 3-49 (2017)). Fucose-alpha1,2-galactose-beta1,3-N-acetylglucosamine (Fuc-a1,2-Gal-b1,3-GlcNAc) or 2′-fucosyllacto-N-biose (2′FLNB), also known as the H type-1 antigen (H1) is a structure within the Type I Lewis antigens that has been reported to be involved, amongst others, in inflammation reactions, rotavirus infections and cancer pathogenesis (Blanas et al. 2018, Front. Oncol., https://doi.org/10.3389/fonc.2018.00039). The Fuc-a1,2-Gal-b1,3-GlcNAc group is also present in lacto-N-fucopentaose I (LNFP-I, Fuc-a1,2-Gal-b1,3-GlcNAc-b1,3-Gal-b1,4-Glc) and lacto-N-difucohexaose I (LNDFH I, Fuc-a1,2-Gal-b1,3-[Fuc-a1,4]lcNAc-b1,3-Gal-b1,4-Glc) which are abundant oligosaccharides present in human milk. Human milk oligosaccharides (HMOs) have multiple function, including prebiotic, immune, gut and cognition benefits (Reverri et al., Nutrients 10 (10), 1346 (2018)). LNDFH I has been reported to have immunomodulating capacities being involved in viral infections (Triantis et al. Front. Pediatr. 2018). LNFP-I represents an important immunomodulator to prevent nursing infants from severe infectious diarrhoea by inhibition of the adhesion of pathogenic bacteria like Escherichia coli (EPEC, UPEC) and viruses. LNFP-I has also been linked to binding of pathogen toxins, growth inhibition of Group B Streptococci and selective stimulation of bifidobacterial communities (Derya et al., J. Biotechnol. 318, 31-38 (2020); Gotoh et al., Sci. Rep. 8, 13958 (2018); Lin et al. J. Biol. Chem. 292, 11243-11249 (2017); Sotgiu et al., Int. J. Biomed. Sci. 2 (2), 114-120 (2006)).

There is large scientific and commercial interest in these structures or compounds, yet the availability is limited as production relies on chemical or chemo-enzymatic synthesis or on purification from natural sources such as e.g. animal milk. Chemical synthesis methods are laborious and time-consuming and because of the large number of steps involved they are difficult to scale-up. Enzymatic approaches offer advantages above chemical synthesis, but stereospecificity and regioselectivity of the necessary enzymes are still a formidable challenge.

It is an object of the present invention to provide for tools and methods by means of which these structures can be produced by a cell and preferably in an efficient, time and cost-effective way and which yields high amounts of the desired compound. According to the invention, this and other objects are achieved by providing a cell and a method for the production of a compound comprising a structure of Formula I, II or III as described in the present invention, wherein the cell is genetically modified for the production of said compound.

### DESCRIPTION

### Summary of the Invention

Surprisingly, it has now been found that it is possible to produce a compound comprising a structure of Formula I, II or III:

Fuc-α1,2-Gal-β1,3-GlcNAc Formula I

Fuc-α1,2-Gal-β1,3-GlcNAc-R¹ Formula II

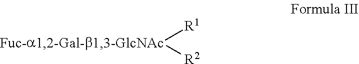

wherein:

R¹ is a monosaccharide, disaccharide, oligosaccharide, protein, glycoprotein, peptide, glycopeptide, lipid or glycolipid; R² is a monosaccharide, disaccharide or oligosaccharide,
by a single cell. The present invention provides a cell and a method for the production of said compound comprising a structure of Formula I, II or III. The method comprises the steps of providing a cell which expresses an alpha-1,2-fucosyltransferase that has a galactoside alpha-1,2-fucosyltransferase activity on the galactose residue of Gal-b1,3-GlcNAc (LNB, lacto-N-biose) and which is capable to produce said compound comprising a structure of Formula I, II or III, and cultivating and/or incubating said cell under conditions permissive for producing said compound comprising a structure of Formula I, II or III. The present invention also provides methods to separate and purify said compound comprising a structure of Formula I, II or III. Furthermore, the present invention provides a cell genetically engineered for production of compound comprising a structure of Formula I, II or III.

### Definitions

The words used in this specification to describe the invention and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various aspects and embodiments of aspects of the invention disclosed herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, purification steps are performed according to the manufacturer's specifications.

In the specification, there have been disclosed embodiments of the invention, and although specific terms are employed, the terms are used in a descriptive sense only and not for purposes of limitation, the scope of the invention being set forth in the following claims. It must be understood that the illustrated embodiments have been set forth only for the purposes of example and that it should not be taken as limiting the invention. It will be apparent to those skilled in the art that alterations, other embodiments, improvements, details and uses can be made consistent with the letter and spirit of the disclosure herein and within the scope of this invention, which is limited only by the claims, construed in accordance with the patent law, including the doctrine of equivalents. In the claims which follow, reference characters used to designate claim steps are provided for convenience of description only, and are not intended to imply any particular order for performing the steps.

In this document and in its claims, the verbs “to comprise”, “to have” and “to contain”, and their conjugations are used in their non-limiting sense to mean that items following the word are included, but items not specifically mentioned are not excluded. The verb “to consist essentially of” means that additional component(s) may be present than the ones specifically identified, said additional component(s) not altering the unique characteristic of the invention. Throughout the application and claims, unless specifically stated otherwise, the verbs “to comprise”, “to have” and “to contain”, and their conjugations, may be preferably replaced by “to consist” (and its conjugations) or “to consist essentially of” (and its conjugations) and vice versa. In addition, reference to an element by the indefinite article “a” or “an” does not exclude the possibility that more than one of the element is present, unless the context clearly requires that there be one and only one of the elements. The indefinite article “a” or “an” thus usually means “at least one”.

Throughout the application, unless explicitly stated otherwise, the articles “a” and “an” are preferably replaced by “at least two”, more preferably by “at least three”, even more preferably by “at least four”, even more preferably by “at least five”, even more preferably by “at least six”, most preferably by “at least two”.

Throughout the application, unless explicitly stated otherwise, the features “synthesize”, “synthesized” and “synthesis” are interchangeably used with the features “produce”, “produced” and “production”, respectively.

Each embodiment as identified herein may be combined together unless otherwise indicated. All publications, patents, and patent applications mentioned in this specification are herein incorporated by reference to the same extent as if each individual publication, patent, or patent application was specifically and individually indicated to be incorporated by reference.

According to the present invention, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to the present invention. Moreover, DNAs or RNAs comprising unusual bases, such as inosine, or modified bases, such as tritylated bases, are to be understood to be covered by the term “polynucleotides”. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid sidechains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulphide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

The term “polynucleotide encoding a polypeptide” as used herein encompasses polynucleotides that include a sequence encoding a polypeptide of the invention. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Similarly, a “synthetic” sequence, as the term is used herein, means any sequence that has been generated synthetically and not directly isolated from a natural source. “Synthesized”, as the term is used herein, means any synthetically generated sequence and not directly isolated from a natural source.

Throughout the application, unless explicitly stated otherwise, the features “synthesize”, “synthesized” and “synthesis” are interchangeably used with the features “produce”, “produced” and “production”, respectively.

The terms “recombinant” or “transgenic” or “metabolically engineered” or “genetically modified”, as used herein with reference to a cell or host cell are used interchangeably and indicates that the cell replicates a heterologous nucleic acid, or expresses a peptide or protein encoded by a heterologous nucleic acid (i.e., a sequence “foreign to said cell” or a sequence “foreign to said location or environment in said cell”). Such cells are described to be transformed with at least one heterologous or exogenous gene, or are described to be transformed by the introduction of at least one heterologous or exogenous gene. Metabolically engineered or recombinant or transgenic cells can contain genes that are not found within the native (non-recombinant) form of the cell. Recombinant cells can also contain genes found in the native form of the cell wherein the genes are modified and re-introduced into the cell by artificial means. The terms also encompass cells that contain a nucleic acid endogenous to the cell that has been modified or its expression or activity has been modified without removing the nucleic acid from the cell; such modifications include those obtained by gene replacement, replacement of a promoter; site-specific mutation; and related techniques. Accordingly, a “recombinant polypeptide” is one which has been produced by a recombinant cell. A “heterologous sequence” or a “heterologous nucleic acid”, as used herein, is one that originates from a source foreign to the particular cell (e.g. from a different species), or, if from the same source, is modified from its original form or place in the genome. Thus, a heterologous nucleic acid operably linked to a promoter is from a source different from that from which the promoter was derived, or, if from the same source, is modified from its original form or place in the genome. The heterologous sequence may be stably introduced, e.g. by transfection, transformation, conjugation or transduction, into the genome of the host microorganism cell, wherein techniques may be applied which will depend on the cell and the sequence that is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989). The term “mutant” cell or microorganism as used within the context of the present invention refers to a cell or microorganism which is genetically modified.

The term “endogenous,” within the context of the present invention refers to any polynucleotide, polypeptide or protein sequence which is a natural part of a cell and is occurring at its natural location in the cell chromosome and of which the control of expression has not been altered compared to the natural control mechanism acting on its expression. The term “exogenous” refers to any polynucleotide, polypeptide or protein sequence which originates from outside the cell under study and not a natural part of the cell or which is not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g. a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e. in the genome of a non-genetically engineered organism) is referred to herein as a “heterologous promoter,” even though the promoter may be derived from the same species (or, in some cases, the same organism) as the gene to which it is linked.

The term “modified activity” of a protein or an enzyme relates to a change in activity of the protein or the enzyme compared to the wild type, i.e. natural, activity of said protein or enzyme. Said modified activity can either be an abolished, impaired, reduced or delayed activity of said protein or enzyme compared to the wild type activity of the protein or the enzyme but can also be an accelerated or an enhanced activity of said protein or the enzyme compared to the wild type activity of the protein or the enzyme. A modified activity of a protein or an enzyme is obtained by modified expression of said protein or enzyme or is obtained by expression of a modified, i.e. mutant form of the protein or enzyme. A modified activity of an enzyme further relates to a modification in the apparent Michaelis constant Km and/or the apparent maximal velocity (Vmax) of the enzyme.

The term “modified expression” of a gene relates to a change in expression compared to the wild type expression of said gene in any phase of the production process of the desired di- and/or oligosaccharide. Said modified expression is either a lower or higher expression compared to the wild type, wherein the term “higher expression” is also defined as “overexpression” of said gene in the case of an endogenous gene or “expression” in the case of a heterologous gene that is not present in the wild type strain. Lower expression is obtained by means of common well-known technologies for a skilled person (such as the usage of siRNA, CrispR, CrispRi, riboswitch, recombineering, homologous recombination, ssDNA mutagenesis, RNAi, miRNA, asRNA, mutating genes, knocking-out genes, transposon mutagenesis, . . . ) which are used to change the genes in such a way that they are less-able (i.e. statistically significantly ‘less-able’ compared to a functional wild-type gene) or completely unable (such as knocked-out genes) to produce functional final products. The term “riboswitch” as used herein is defined to be part of the messenger RNA that folds into intricate structures that block expression by interfering with translation. Binding of an effector molecule induces conformational change(s) permitting regulated expression post-transcriptionally. Next to changing the gene of interest in such a way that lower expression is obtained as described above, lower expression can also be obtained by changing the transcription unit, the promoter, an untranslated region, the ribosome binding site, the Shine Dalgarno sequence or the transcription terminator. Lower expression or reduced expression can for instance be obtained by mutating one or more base pairs in the promoter sequence or changing the promoter sequence fully to a constitutive promoter with a lower expression strength compared to the wild type or an inducible promoter which result in regulated expression or a repressible promoter which results in regulated expression.

Overexpression or expression is obtained by means of common well-known technologies for a skilled person (such as the usage of artificial transcription factors, de novo design of a promoter sequence, ribosome engineering, introduction or re-introduction of an expression module at euchromatin, usage of high-copy-number plasmids), wherein said gene is part of an “expression cassette” which relates to any sequence in which a promoter sequence, untranslated region sequence (containing either a ribosome binding sequence, Shine Dalgarno or Kozak sequence), a coding sequence and optionally a transcription terminator is present, and leading to the expression of a functional active protein. Said expression is either constitutive or regulated.

The term “constitutive expression” is defined as expression that is not regulated by transcription factors other than the subunits of RNA polymerase (e.g. bacterial sigma factors like s⁷⁰, s⁵⁴, or related s-factors and the yeast mitochondrial RNA polymerase specificity factor MTF1 that co-associate with the RNA polymerase core enzyme) under certain growth conditions. Non-limiting examples of such transcription factors are CRP, Lacl, ArcA, Cra, IclR in E. coli, or, Aft2p, Crz1p, Skn7 in Saccharomyces cerevisiae, or, DeoR, GntR, Fur in B. subtilis. The RNA polymerase is the catalytic machinery for the synthesis of RNA from a DNA template. RNA polymerase binds a specific DNA sequence to initiate transcription, for instance via a sigma factor in prokaryotic hosts or via MTF1 in yeasts. Constitutive expression offers a constant level of expression with no need for induction or repression.

The term “regulated expression” is defined as a facultative or regulatory or tuneable expression of a gene that is only expressed upon a certain natural condition of the host (e.g. mating phase of budding yeast, stationary phase of bacteria), as a response to an inducer or repressor such as but not limited to glucose, allo-lactose, lactose, galactose, glycerol, arabinose, rhamnose, fucose, IPTG, methanol, ethanol, acetate, formate, aluminium, copper, zinc, nitrogen, phosphates, xylene, carbon or nitrogen depletion, or substrates or the produced product or chemical repression, as a response to an environmental change (e.g. anaerobic or aerobic growth, oxidative stress, pH shifts, temperature changes like e.g. heat-shock or cold-shock, osmolarity, light conditions, starvation) or dependent on the position of the developmental stage or the cell cycle of said host cell including but not limited to apoptosis and autophagy. Regulated expression allows for control as to when a gene is expressed. The term “inducible expression by a natural inducer” is defined as a facultative or regulatory expression of a gene that is only expressed upon a certain natural condition of the host (e.g. organism being in labour, or during lactation), as a response to an environmental change (e.g. including but not limited to hormone, heat, cold, pH shifts, light, oxidative or osmotic stress/signalling), or dependent on the position of the developmental stage or the cell cycle of said host cell including but not limited to apoptosis and autophagy. The term “inducible expression upon chemical treatment” is defined as a facultative or regulatory expression of a gene that is only expressed upon treatment with a chemical inducer or repressor, wherein said inducer and repressor comprise but are not limited to an alcohol (e.g. ethanol, methanol), a carbohydrate (e.g. glucose, galactose, glycerol, lactose, arabinose, rhamnose, fucose, allo-lactose), metal ions (e.g. aluminium, copper, zinc), nitrogen, phosphates, IPTG, acetate, formate, xylene.

The term “control sequences” refers to sequences recognized by the cells transcriptional and translational systems, allowing transcription and translation of a polynucleotide sequence to a polypeptide. Such DNA sequences are thus necessary for the expression of an operably linked coding sequence in a particular cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. Said control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of said polynucleotide to a polypeptide.

Generally, “operably linked” means that the DNA sequences being linked are contiguous, and, in the case of a secretory leader, contiguous and in reading phase. However, enhancers do not have to be contiguous.

The term “wild type” refers to the commonly known genetic or phenotypical situation as it occurs in nature.

The term “modified expression of a protein” as used herein refers to i) higher expression or overexpression of an endogenous protein, ii) expression of a heterologous protein or iii) expression and/or overexpression of a variant protein that has a higher activity compared to the wild-type (i.e. native) protein.

As used herein, the term “mammary cell(s)” generally refers to mammary epithelial cell(s), mammary-epithelial luminal cell(s), or mammalian epithelial alveolar cell(s), or any combination thereof. As used herein, the term “mammary-like cell(s)” generally refers to cell(s) having a phenotype/genotype similar (or substantially similar) to natural mammary cell(s) but is/are derived from non-mammary cell source(s). Such mammary-like cell(s) may be engineered to remove at least one undesired genetic component and/or to include at least one predetermined genetic construct that is typical of a mammary cell. Non-limiting examples of mammary-like cell(s) may include mammary epithelial-like cell(s), mammary epithelial luminal-like cell(s), non-mammary cell(s) that exhibits one or more characteristics of a cell of a mammary cell lineage, or any combination thereof. Further non-limiting examples of mammary-like cell(s) may include cell(s) having a phenotype similar (or substantially similar) to natural mammary cell(s), or more particularly a phenotype similar (or substantially similar) to natural mammary epithelial cell(s). A cell with a phenotype or that exhibits at least one characteristic similar to (or substantially similar to) a natural mammary cell or a mammary epithelial cell may comprise a cell (e.g., derived from a mammary cell lineage or a non-mammary cell lineage) that exhibits either naturally, or has been engineered to, be capable of expressing at least one milk component.

As used herein, the term “non-mammary cell(s)” may generally include any cell of non-mammary lineage. In the context of the invention, a non-mammary cell can be any mammalian cell capable of being engineered to express at least one milk component. Non-limiting examples of such non-mammary cell(s) include hepatocyte(s), blood cell(s), kidney cell(s), cord blood cell(s), epithelial cell(s), epidermal cell(s), myocyte(s), fibroblast(s), mesenchymal cell(s), or any combination thereof. In some instances, molecular biology and genome editing techniques can be engineered to eliminate, silence, or attenuate myriad genes simultaneously.

Throughout the application, unless explicitly stated otherwise, the expressions “capable of . . . <verb>” and “capable to . . . <verb>” are preferably replaced with the active voice of said verb and vice versa. For example, the expression “capable of expressing” is preferably replaced with “expresses” and vice versa, i.e. “expresses” is preferably replaced with “capable of expressing”.

“Variant(s)” as the term is used herein, is a polynucleotide or polypeptide that differs from a reference polynucleotide or polypeptide respectively but retains essential properties. In the context of the present invention, a variant of an alpha-1,2-fucosyltransferase as disclosed herein (i.e. reference polypeptide) is a polypeptide that differs from said reference polypeptide but retains its enzymatic activities as described herein, e.g. galactoside alpha-1,2-fucosyltransferase activity on the galactose residue of Gal-b1,3-GlcNAc (LNB, lacto-N-biose) or e.g. galactoside alpha-1,2-fucosyltransferase activity on the galactose residue of Gal-b1,3-GlcNAc and additional galactoside alpha-1,2-fucosyltransferase activity on the galactose residue at the non-reducing end of LNT, depending on the identity of the alpha-1,2-fucosyltransferase as described herein. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally.

Non-naturally occurring variants of polynucleotides and polypeptides may be made by mutagenesis techniques, by direct synthesis, and by other recombinant methods known to the persons skilled in the art. In the context of the present invention, a “variant” of a reference polypeptide is preferably a polypeptide having an amino acid sequence having at least 80% sequence identity to the full-length sequence of the reference polypeptide.

The term “derivative” of a polypeptide, as used herein, is a polypeptide which may contain deletions, additions or substitutions of amino acid residues within the amino acid sequence of the polypeptide, but which result in a silent change, thus producing a functionally equivalent polypeptide (i.e. retaining the enzymatic activity of the polypeptide as described herein). Amino acid substitutions may be made based on similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; planar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. Within the context of this invention, a derivative polypeptide as used herein, refers to a polypeptide capable of exhibiting a substantially similar in vitro and/or in vivo activity as the original polypeptide as judged by any of a number of criteria, including but not limited to enzymatic activity, and which may be differentially modified during or after translation. Furthermore, non-classical amino acids or chemical amino acid analogues can be introduced as a substitution or addition into the original polypeptide sequence. In the context of the present invention, a “derivative” of a reference polypeptide is preferably a polypeptide having an amino acid sequence having at least 80% sequence identity to the full-length sequence of the reference polypeptide.

In some embodiments, the present invention contemplates making functional variants by modifying the structure of an enzyme as used in the present invention. Variants can be produced by amino acid substitution, deletion, addition, or combinations thereof. For instance, it is reasonable to expect that an isolated replacement of a leucine with an isoleucine or valine, an aspartate with a glutamate, a threonine with a serine, an asparagine with a glutamine, a lysine with an arginine, a cysteine with a methionine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the invention results in a functional homolog can be readily determined by assessing the ability of the variant polypeptide to produce a response in cells in a fashion similar to the wild-type polypeptide.

“Fragment”, with respect to a polynucleotide, refers to a clone or any part of a polynucleotide molecule, particularly a part of a polynucleotide that retains a usable, functional characteristic of the full-length polynucleotide molecule. Useful fragments include oligonucleotides and polynucleotides that may be used in hybridization or amplification technologies or in the regulation of replication, transcription or translation. A “polynucleotide fragment” refers to any subsequence of a polynucleotide SEQ ID NO (or Genbank NO.), typically, comprising or consisting of at least about 9, 10, 11, 12 consecutive nucleotides from said polynucleotide SEQ ID NO (or Genbank NO.), for example at least about 30 nucleotides or at least about 50 nucleotides of any of the polynucleotide sequences provided herein. Exemplary fragments can additionally or alternatively include fragments that comprise, consist essentially of, or consist of a region that encodes a conserved family domain of a polypeptide. Exemplary fragments can additionally or alternatively include fragments that comprise a conserved domain of a polypeptide. As such, a fragment of a polynucleotide SEQ ID NO (or Genbank NO.) preferably means a nucleotide sequence which comprises or consists of said polynucleotide SEQ ID NO (or Genbank NO.) wherein no more than 200, 150, 100, 50 or 25 consecutive nucleotides are missing, preferably no more than 50 consecutive nucleotides are missing, and which retains a usable, functional characteristic (e.g. activity) of the full-length polynucleotide molecule which can be assessed by the skilled person through routine experimentation. Alternatively, a fragment of a polynucleotide SEQ ID NO (or Genbank NO.) preferably means a nucleotide sequence which comprises or consists of an amount of consecutive nucleotides from said polynucleotide SEQ ID NO (or Genbank NO.) and wherein said amount of consecutive nucleotides is at least 50.0%, 60.0%, 70.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 95.5%, 96.0%, 96.5%, 97.0%, 97.5%, 98.0%, 98.5%, 99.0%, 99.5%, 100%, preferably at least 80.0%, more preferably at least 87.0%, even more preferably at least 90.0%, even more preferably at least 95.0%, most preferably at least 97.0%, of the full-length of said polynucleotide SEQ ID NO (or Genbank NO.) and retains a usable, functional characteristic (e.g. activity) of the full-length polynucleotide molecule. As such, a fragment of a polynucleotide SEQ ID NO (or Genbank NO.) preferably means a nucleotide sequence which comprises or consists of said polynucleotide SEQ ID NO (or Genbank NO.), wherein an amount of consecutive nucleotides is missing and wherein said amount is no more than 50.0%, 40.0%, 30.0% of the full-length of said polynucleotide SEQ ID NO (or Genbank NO.), preferably no more than 20.0%, 15.0%, 10.0%, 9.0%, 8.0%, 7.0%, 6.0%, 5.0%, 4.5%, 4.0%, 3.5%, 3.0%, 2.5%, 2.0%, 1.5%, 1.0%, 0.5%, more preferably no more than 15.0%, even more preferably no more than 10.0%, even more preferably no more than 5.0%, most preferably no more than 2.5%, of the full-length of said polynucleotide SEQ ID NO (or Genbank NO.) and wherein said fragment retains a usable, functional characteristic (e.g. activity) of the full-length polynucleotide molecule which can be routinely assessed by the skilled person.

“Fragment”, with respect to a polypeptide, refers to a subsequence of the polypeptide which performs at least one biological function of the intact polypeptide in substantially the same manner, or to a similar extent, as does the intact polypeptide. A “subsequence of the polypeptide” as defined herein refers to a sequence of contiguous amino acid residues derived from the polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, for example at least about 20 amino acid residues in length, for example at least about 30 amino acid residues in length. As such, a fragment of a polypeptide SEQ ID NO (or UniProt ID or Genbank NO.) preferably means a polypeptide sequence which comprises or consists of said polypeptide SEQ ID NO (or UniProt ID or Genbank NO.) wherein no more than 80, 60, 50, 40, 30, 20 or 15 consecutive amino acid residues are missing, preferably no more than 40 consecutive amino acid residues are missing, and performs at least one biological function of the intact polypeptide in substantially the same manner, preferably to a similar or greater extent, as does the intact polypeptide which can be routinely assessed by the skilled person. Alternatively, a fragment of a polypeptide SEQ ID NO (or UniProt ID or Genbank NO.) preferably means a polypeptide sequence which comprises or consists of an amount of consecutive amino acid residues from said polypeptide SEQ ID NO (or UniProt ID or Genbank NO.) and wherein said amount of consecutive amino acid residues is at least 50.0%, 60.0%, 70.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 95.5%, 96.0%, 96.5%, 97.0%, 97.5%, 98.0%, 98.5%, 99.0%, 99.5%, 100%, preferably at least 80.0%, more preferably at least 87.0%, even more preferably at least 90.0%, even more preferably at least 95.0%, most preferably at least 97.0% of the full-length of said polypeptide SEQ ID NO (or UniProt ID or Genbank NO.) and which performs at least one biological function of the intact polypeptide in substantially the same manner, preferably to a similar or greater extent, as does the intact polypeptide which can be routinely assessed by the skilled person. As such, a fragment of a polypeptide SEQ ID NO (or UniProt ID or Genbank NO.) preferably means a polypeptide sequence which comprises or consists of said polypeptide SEQ ID NO (or UniProt ID or Genbank NO.), wherein an amount of consecutive amino acid residues is missing and wherein said amount is no more than 50.0%, 40.0%, 30.0% of the full-length of said polypeptide SEQ ID NO (or UniProt ID or Genbank NO.), preferably no more than 20.0%, 15.0%, 10.0%, 9.0%, 8.0%, 7.0%, 6.0%, 5.0%, 4.5%, 4.0%, 3.5%, 3.0%, 2.5%, 2.0%, 1.5%, 1.0%, 0.5%, more preferably no more than 15.0%, even more preferably no more than 10.0%, even more preferably no more than 5.0%, most preferably no more than 2.5%, of the full-length of said polypeptide SEQ ID NO (or UniProt ID or Genbank NO.) and which performs at least one biological function of the intact polypeptide in substantially the same manner, preferably to a similar or greater extent, as does the intact polypeptide which can be routinely assessed by the skilled person (e.g. by assessing the ability of the polypeptide fragment to produce a response in cells in a fashion similar to the wild-type, i.e. full-length polypeptide). Throughout the application and claims, the terms “at least one biological function”, “at least one property” and “at least one activity” preferably refers to the alpha-1,2-fucosyltransferase activity as described herein.

Throughout the application, the sequence of a polypeptide can be represented by a SEQ ID NO or alternatively by an UniProt ID or GenBank NO. Therefore, the terms “polypeptide SEQ ID NO” and “polypeptide UniProt ID” and “polypeptide GenBank NO.” can be interchangeably used, unless explicitly stated otherwise.

A “functional fragment” of a polypeptide has at least one property or activity of the polypeptide from which it is derived, preferably to a similar or greater extent. A functional fragment can, for example, include a functional domain or conserved domain of a polypeptide. It is understood that a polypeptide or a fragment thereof may have conservative amino acid substitutions which have substantially no effect on the polypeptide's activity. By conservative substitutions is intended substitutions of one hydrophobic amino acid for another or substitution of one polar amino acid for another or substitution of one acidic amino acid for another or substitution of one basic amino acid for another etc. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa.

Homologous sequences as used herein describes those nucleotide sequences that have sequence similarity and encode polypeptides that share at least one functional characteristic such as a biochemical activity. More specifically, the term “functional homolog” as used herein describes those polypeptides that have sequence similarity (in other words, homology) and at the same time have at least one functional similarity such as a biochemical activity (Altenhoff et al., PLOS Comput. Biol. 8 (2012) e1002514). In the context of the present invention, a functional homolog of a reference polypeptide is preferably a polypeptide having an amino acid sequence having at least 80% sequence identity to the full-length sequence of the reference polypeptide.

Functional homologs are sometimes referred to as orthologs, where “ortholog” refers to a homologous gene or protein that is the functional equivalent of the referenced gene or protein in another species. Orthologous sequences are homologous sequences in different species that originate by vertical descent from a single sequence of the last common ancestor, wherein the sequence and its main function are conserved. A homologous sequence is a sequence inherited in two species by a common ancestor. The term “ortholog” when used in reference to an amino acid or nucleotide/nucleic acid sequence from a given species refers to the same amino acid or nucleotide/nucleic acid sequence from a different species. It should be understood that two sequences are orthologs of each other when they are derived from a common ancestor sequence via linear descent and/or are otherwise closely related in terms of both their sequence and their biological function. Orthologs will usually have a high degree of sequence identity but may not (and often will not) share 100% sequence identity. Paralogous sequences are homologous sequences that originate by a sequence duplication event. Paralogous sequences often belong to the same species, but this is not necessary. Paralogs can be split into in-paralogs (paralogous pairs that arose after a speciation event) and out-paralogs (paralogous pairs that arose before a speciation event). Between species out-paralogs are pairs of paralogs that exist between two organisms due to duplication before speciation. Within species out-paralogs are pairs of paralogs that exist in the same organism, but whose duplication event happened after speciation. Paralogs typically have the same or similar function. Functional homologs will typically give rise to the same characteristics to a similar, but not necessarily the same, degree. Functionally homologous polypeptides give the same characteristics where the quantitative measurement produced by one homolog is at least 10 percent of the other; more typically, at least 20 percent, between about 30 percent and about 40 percent; for example, between about 50 percent and about 60 percent; between about 70 percent and about 80 percent; or between about 90 percent and about 95 percent; between about 98 percent and about 100 percent, or greater than 100 percent of that produced by the original molecule. Thus, where the molecule has enzymatic activity the functional homolog will have the above-recited percent enzymatic activities compared to the original enzyme. Where the molecule is a DNA-binding molecule (e.g., a polypeptide) the homolog will have the above-recited percentage of binding affinity as measured by weight of bound molecule compared to the original molecule.

A functional homolog and the reference polypeptide may be naturally occurring polypeptides, and the sequence similarity may be due to convergent or divergent evolutionary events.

Functional homologs can be identified by analysis of nucleotide and polypeptide sequence alignments.

For example, performing a query on a database of nucleotide or polypeptide sequences can identify homologs of the polypeptide of interest like e.g. a biomass-modulating polypeptide, a glycosyltransferase, a protein involved in nucleotide-activated sugar synthesis or a membrane transporter protein. Sequence analysis can involve BLAST, Reciprocal BLAST, or PSI-BLAST analysis of non-redundant databases using amino acid sequence of a biomass-modulating polypeptide, a glycosyltransferase, a protein involved in nucleotide-activated sugar synthesis or a membrane transporter protein, respectively, as the reference sequence. Amino acid sequence is, in some instances, deduced from the nucleotide sequence. Typically, those polypeptides in the database that have greater than 40 percent sequence identity are candidates for further evaluation for suitability as a biomass-modulating polypeptide, a glycosyltransferase, a protein involved in nucleotide-activated sugar synthesis or a membrane transporter protein, respectively. Amino acid sequence similarity allows for conservative amino acid substitutions, such as substitution of one hydrophobic residue for another or substitution of one polar residue for another or substitution of one acidic amino acid for another or substitution of one basic amino acid for another etc. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa. If desired, manual inspection of such candidates can be carried out to narrow the number of candidates to be further evaluated. Manual inspection can be performed by selecting those candidates that appear to have domains present in productivity-modulating polypeptides, e.g., conserved functional domains.

A domain can be characterized, for example, by a Pfam (El-Gebali et al., Nucleic Acids Res. 47 (2019) D427-D432), an IPR (InterPro domain) (Mitchell et al., Nucleic Acids Res. 47 (2019) D351-D360), a protein fingerprint domain (PRINTS) (Attwood et al., Nucleic Acids Res. 31 (2003) 400-402), a SUBFAM domain (Gough et al., J. Mol. Biol. 313 (2001) 903-919), a TIGRFAM domain (Selengut et al., Nucleic Acids Res. 35 (2007) D260-D264), a Conserved Domain Database (CDD) designation (https://www.ncbi.nlm.nih.gov/cdd) (Lu et al., Nucleic Acids Res. 48 (2020) D265-D268), a PTHR domain (http://www.pantherdb.org) (Mi et al., Nucleic Acids. Res. 41 (2013) D377-D386; Thomas et al., Genome Research 13 (2003) 2129-2141) or a PATRIC identifier or PATRIC DB global family domain (https://www.patricbrc.org/) (Davis et al., Nucleic Acids Res. 48 (D1) (2020) D606-D612). It should be understood for those skilled in the art that for the databases used herein, comprising Pfam 32.0 (released September 2018), CDD v3.17 (released 3rd April 2019), eggnogdb 4.5.1 (released September 2016), InterPro 75.0 (released 4th July 2019), TCDB (released 17th June 2019) and PATRIC 3.6.9 (released March 2020), the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

Protein or polypeptide sequence information and functional information can be provided by a comprehensive resource for protein sequence and annotation data like e.g. the Universal Protein Resource (UniProt) (www.uniprot.org) (Nucleic Acids Res. 2021, 49 (D1), D480-D489). UniProt comprises the expertly and richly curated protein database called the UniProt Knowledgebase (UniProtKB), together with the UniProt Reference Clusters (UniRef) and the UniProt Archive (UniParc). The UniProt identifiers (UniProt ID) are unique for each protein present in the database. UniProt IDs as used herein are the UniProt IDs in the UniProt database version of 5 May 2021. Proteins that do not have an UniProt ID are referred herein using the respective GenBank Accession number (GenBank No.) as present in the NIH genetic sequence database (https://www.ncbi.nlm.nih.gov/genbank/) (Nucleic Acids Res. 2013, 41 (D1), D36-D42) version of 5 May 2021.

The terms “identical” or “percent identity” or “% identity” in the context of two or more nucleic acid or polypeptide sequences, refer to two or more sequences or subsequences that are the same or have a specified percentage of amino acid residues or nucleotides that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Percent identity may be calculated globally over the full-length sequence of the reference sequence, resulting in a global percent identity score. Alternatively, percent identity may be calculated over a partial sequence of the reference sequence, resulting in a local percent identity score. Using the full-length of the reference sequence in a local sequence alignment results in a global percent identity score between the test and the reference sequence.

Percent identity can be determined using different algorithms like for example BLAST and PSI-BLAST (Altschul et al., 1990, J Mol Biol 215:3, 403-410; Altschul et al., 1997, Nucleic Acids Res 25:17, 3389-402), the Clustal Omega method (Sievers et al., 2011, Mol. Syst. Biol. 7:539), the MatGAT method (Campanella et al., 2003, BMC Bioinformatics, 4:29) or EMBOSS Needle.

The BLAST (Basic Local Alignment Search Tool)) method of alignment is an algorithm provided by the National Center for Biotechnology Information (NCBI) to compare sequences using default parameters. The program compares nucleotide or protein sequences to sequence databases and calculates the statistical significance. PSI-BLAST (Position-Specific Iterative Basic Local Alignment Search Tool) derives a position-specific scoring matrix (PSSM) or profile from the multiple sequence alignment of sequences detected above a given score threshold using protein-protein BLAST (BLASTp). The BLAST method can be used for pairwise or multiple sequence alignments. Pairwise Sequence Alignment is used to identify regions of similarity that may indicate functional, structural and/or evolutionary relationships between two biological sequences (protein or nucleic acid). The web interface for BLAST is available at: https://blast.ncbi.nlm.nih.gov/Blast.cgi.

Clustal Omega (Clustal W) is a multiple sequence alignment program that uses seeded guide trees and HMM profile-profile techniques to generate alignments between three or more sequences. It produces biologically meaningful multiple sequence alignments of divergent sequences. The web interface for Clustal W is available at https://www.ebi.ac.uk/Tools/msa/clustalo/. Default parameters for multiple sequence alignments and calculation of percent identity of protein sequences using the Clustal W method are: enabling de-alignment of input sequences: FALSE; enabling mbed-like clustering guide-tree: TRUE; enabling mbed-like clustering iteration: TRUE; Number of (combined guide-tree/HMM) iterations: default (0); Max Guide Tree Iterations: default [−1]; Max HMM Iterations: default [−1]; order: aligned.

MatGAT (Matrix Global Alignment Tool) is a computer application that generates similarity/identity matrices for DNA or protein sequences without needing pre-alignment of the data. The program performs a series of pairwise alignments using the Myers and Miller global alignment algorithm, calculates similarity and identity, and then places the results in a distance matrix. The user may specify which type of alignment matrix (e.g. BLOSUM50, BLOSUM62, and PAM250) to employ with their protein sequence examination. EMBOSS Needle (https://galaxy-iuc.github.io/emboss-5.0-docs/needle.html) uses the Needleman-Wunsch global alignment algorithm to find the optimal alignment (including gaps) of two sequences when considering their entire length. The optimal alignment is ensured by dynamic programming methods by exploring all possible alignments and choosing the best. The Needleman-Wunsch algorithm is a member of the class of algorithms that can calculate the best score and alignment in the order of mn steps, (where ‘n’ and ‘m’ are the lengths of the two sequences). The gap open penalty (default 10.0) is the score taken away when a gap is created. The default value assumes you are using the EBLOSUM62 matrix for protein sequences. The gap extension (default 0.5) penalty is added to the standard gap penalty for each base or residue in the gap. This is how long gaps are penalized.

As used herein, a polypeptide having an amino acid sequence (or polypeptide sequence) having at least 80% sequence identity to the full-length sequence of a reference polypeptide sequence is to be understood as that the sequence has 80%, 81%, 82%, 83%, 84%, 85%, 86%, 87%, 88%, 89%, 90%, 91%, 91.50%, 92.00%, 92.50%, 93.00%, 93.50%, 94.00%, 94.50%, 95.00%, 95,50%, 96.00%, 96,50%, 97.00%, 97,50%, 98.00%, 98,50%, 99.00%, 99,50%, 99,60%, 99,70%, 99,80%, 99,90%, 100% sequence identity to the full-length of the amino acid sequence of the reference polypeptide sequence. Throughout the application, unless explicitly specified otherwise, a polypeptide comprising, consisting or having an amino acid sequence having at least 80% sequence identity to the full-length amino acid sequence of a reference polypeptide, usually indicated with a SEQ ID NO, UniProt ID or Genbank NO., preferably has at least 85.0%, 90.0%, 91.0%, 92.0%, 93.0%, 94.0%, 95.0%, 96.0%, 97.0%, 98.0% or 99.0%, more preferably has at least 85.0%, even more preferably has at least 90.0%, most preferably has at least 95.0%, sequence identity to the full length reference sequence. Additionally, unless explicitly specified otherwise, a polynucleotide sequence comprising/consisting/having a nucleotide sequence having at least 80.0% sequence identity to the full-length nucleotide sequence of a reference polynucleotide sequence, usually indicated with a SEQ ID NO or Genbank No., preferably has at least 85.0%, 90.0%, 91.0%, 92.0%, 93.0%, 94.0%, 95.0%, 96.0%, 97.0%, 98.0% or 99.0%, more preferably has at least 85.0%, even more preferably has at least 90.0%, most preferably has at least 95.0%, sequence identity to the full length reference sequence.

For the purposes of this invention, percent identity is determined using MatGAT2.01 (Campanella et al., 2003, BMC Bioinformatics 4:29). The following default parameters for protein are employed: (1) Gap cost Existence: 12 and Extension: 2; (2) The Matrix employed was BLOSUM65. In a preferred embodiment, sequence identity is calculated based on the full-length sequence of a given SEQ ID NO, i.e. the reference sequence, or a part thereof. Part thereof preferably means at least 50%, 60%, 70%, 80%, 90% or 95% of the complete reference sequence.

The terms “mannose-6-phosphate isomerase”, “phosphomannose isomerase”, “mannose phosphate isomerase”, “phosphohexoisomerase”, “phosphomannoisomerase”, “phosphomannose-isomerase”, “phosphohexomutase”, “D-mannose-6-phosphate ketol-isomerase” and “manA” are used interchangeably and refer to an enzyme that catalyses the reversible conversion of D-fructose 6-phosphate to D-mannose 6-phosphate.

The terms “phosphomannomutase”, “mannose phosphomutase”, “phosphomannose mutase”, “D-mannose 1,6-phosphomutase” and “manB” are used interchangeably and refer to an enzyme that catalyses the reversible conversion of D-mannose 6-phosphate to D-mannose 1-phosphate.

The terms “mannose-1-phosphate guanylyltransferase”, “GTP-mannose-1-phosphate guanylyltransferase”, “PIM-GMP (phosphomannose isomerase-guanosine 5′-diphospho-D-mannose pyrophosphorylase)”, “GDP-mannose pyrophosphorylase”, “guanosine 5′-diphospho-D-mannose pyrophosphorylase”, “guanosine diphosphomannose pyrophosphorylase”, “guanosine triphosphate-mannose 1-phosphate guanylyltransferase”, “mannose 1-phosphate guanylyltransferase (guanosine triphosphate)” and “manC” are used interchangeably and refer to an enzyme that converts D-mannose-1-phosphate using GTP into GDP-mannose and diphosphate.

The terms “GDP-mannose 4,6-dehydratase”, “guanosine 5′-diphosphate-D-mannose oxidoreductase”, “guanosine diphosphomannose oxidoreductase”, “guanosine diphosphomannose 4,6-dehydratase”, “GDP-D-mannose dehydratase”, “GDP-D-mannose 4,6-dehydratase”, “GDP-mannose 4,6-hydro-lyase”, “GDP-mannose 4,6-hydro-lyase (GDP-4-dehydro-6-deoxy-D-mannose-forming)” and “gmd” are used interchangeably and refer to an enzyme that forms the first step in the biosynthesis of GDP-rhamnose and GDP-fucose.

The terms “GDP-L-fucose synthase”, “GDP-4-keto-6-deoxy-D-mannose-3,5-epimerase-4-reductase”, “GDP-L-fucose: NADP+4-oxidoreductase (3,5-epimerizing)” and “fcl” are used interchangeably and refer to an enzyme that forms the second step in the biosynthesis of GDP-fucose.

The terms “L-fucokinase/GDP-fucose pyrophosphorylase”, “L-fucokinase/L-fucose-1-P guanylyltransferase”, “GDP-fucose pyrophosphorylase”, “GDP-L-fucose pyrophosphorylase”, and “fkp” are used interchangeably and refer to an enzyme that catalyses the conversion of L-fucose-1-phosphate into GDP-fucose using GTP.

The terms “L-glutamine-D-fructose-6-phosphate aminotransferase”, “glutamine—fructose-6-phosphate transaminase (isomerizing)”, “hexosephosphate aminotransferase”, “glucosamine-6-phosphate isomerase (glutamine-forming)”, “glutamine-fructose-6-phosphate transaminase (isomerizing)”, “D-fructose-6-phosphate amidotransferase”, “glucosaminephosphate isomerase”, “glucosamine 6-phosphate synthase”, “GlcN6P synthase”, “GFA” and “glmS” are used interchangeably and refer to an enzyme that catalyses the conversion of D-fructose-6-phosphate into D-glucosamine-6-phosphate using L-glutamine.

The terms “glucosamine-6-P deaminase”, “glucosamine-6-phosphate deaminase”, “GlcN6P deaminase”, “glucosamine-6-phosphate isomerase”, “glmD” and “nagB” are used interchangeably and refer to an enzyme that catalyses the reversible isomerization-deamination of glucosamine-6-phosphate (GlcN6P) to form fructose-6-phosphate and an ammonium ion.

The terms “phosphoglucosamine mutase” and “glmM” are used interchangeably and refer to an enzyme that catalyses the conversion glucosamine-6-phosphate to glucosamine-1-phosphate. Phosphoglucosamine mutase can also catalyse the formation of glucose-6-P from glucose-1-P, although at a 1400-fold lower rate.

The terms “N-acetylglucosamine-6-P deacetylase”, “N-acetylglucosamine-6-phosphate deacetylase” and “nagA” are used interchangeably and refer to an enzyme that catalyses the hydrolysis of the N-acetyl group of N-acetylglucosamine-6-phosphate (GlcNAc-6-P) to yield glucosamine-6-phosphate (GlcN6P) and acetate.

An N-acylglucosamine 2-epimerase is an enzyme that catalyses the reaction N-acyl-D-glucosamine=N-acyl-D-mannosamine. Alternative names for this enzyme comprise N-acetylglucosamine 2-epimerase, N-acetyl-D-glucosamine 2-epimerase, GlcNAc 2-epimerase, N-acyl-D-glucosamine 2-epimerase and N-acetylglucosamine epimerase.

An UDP-N-acetylglucosamine 2-epimerase is an enzyme that catalyses the reaction N-acetyl-D-glucosamine=N-acetylmannosamine. Alternative names for this enzyme comprise UDP-N-acylglucosamine 2-epimerase, UDP-GlcNAc-2-epimerase and UDP-N-acetyl-D-glucosamine 2-epimerase. An N-acetylmannosamine-6-phosphate 2-epimerase is an enzyme that catalyses the reaction N-acetyl-D-glucosamine 6-phosphate=N-acetyl-D-mannosamine 6-phosphate.

A bifunctional UDP-GlcNAc 2-epimerase/kinase is a bifunctional enzyme that catalyses the reaction UDP-N-acetyl-D-glucosamine=N-acetyl-D-mannosamine and the reaction N-acetyl-D-mannosamine+ATP=ADP+N-acetyl-D-mannosamine 6-phosphate.

A glucosamine 6-phosphate N-acetyltransferase is an enzyme that catalyses the transfer of an acetyl group from acetyl-CoA to D-glucosamine-6-phosphate thereby generating a free CoA and N-acetyl-D-glucosamine 6-phosphate. Alternative names comprise aminodeoxyglucosephosphate acetyltransferase, D-glucosamine-6-P N-acetyltransferase, glucosamine 6-phosphate acetylase, glucosamine 6-phosphate N-acetyltransferase, glucosamine-phosphate N-acetyltransferase, glucosamine-6-phosphate acetylase, N-acetylglucosamine-6-phosphate synthase, phosphoglucosamine acetylase, phosphoglucosamine N-acetylase phosphoglucosamine N-acetylase, phosphoglucosamine transacetylase, GNA and GNA1. The term “N-acetylglucosamine-6-phosphate phosphatase” refers to an enzyme that dephosphorylates N-acetylglucosamine-6-phosphate (GlcNAc-6-P) hereby synthesizing N-acetylglucosamine (GlcNAc)., The term “N-acetylmannosamine-6-phosphate phosphatase” refers to an enzyme that dephosphorylates N-acetylmannosamine-6-phosphate (ManNAc-6P) to N-acetylmannosamine (ManNAc).

The terms “N-acetylmannosamine-6-phosphate 2-epimerase”, “ManNAc-6-P isomerase”, “ManNAc-6-P 2-epimerase”, N-acetylglucosamine-6P 2-epimerase and “nanE” are used interchangeably and refer to an enzyme that converts ManNAc-6-P to N-acetylglucosamine-6-phosphate (GlcNAc-6-P).

The terms “phosphoacetylglucosamine mutase”, “acetylglucosamine phosphomutase”, “acetylaminodeoxyglucose phosphomutase”, “phospho-N-acetylglucosamine mutase” and “N-acetyl-D-glucosamine 1,6-phosphomutase” are used interchangeably and refer to an enzyme that catalyses the conversion of N-acetyl-glucosamine 1-phosphate into N-acetylglucosamine 6-phosphate.

The terms “N-acetylglucosamine 1-phosphate uridylyltransferase”, “N-acetylglucosamine-1-phosphate uridyltransferase”, “UDP-N-acetylglucosamine diphosphorylase”, “UDP-N-acetylglucosamine pyrophosphorylase”, “uridine diphosphoacetylglucosamine pyrophosphorylase”, “UTP: 2-acetamido-2-deoxy-alpha-D-glucose-1-phosphate uridylyltransferase”, “UDP-GlcNAc pyrophosphorylase”, “GlmU uridylyltransferase”, “Acetylglucosamine 1-phosphate uridylyltransferase”, “UDP-acetylglucosamine pyrophosphorylase”, “uridine diphosphate-N-acetylglucosamine pyrophosphorylase”, “uridine diphosphoacetylglucosamine phosphorylase”, and “acetylglucosamine 1-phosphate uridylyltransferase” are used interchangeably and refer to an enzyme that catalyses the conversion of N-acetylglucosamine 1-phosphate (GlcNAc-1-P) into UDP-N-acetylglucosamine (UDP-GlcNAc) by the transfer of uridine 5-monophosphate (from uridine 5-triphosphate (UTP)).

The term glucosamine-1-phosphate acetyltransferase refers to an enzyme that catalyses the transfer of the acetyl group from acetyl coenzyme A to glucosamine-1-phosphate (GlcN-1-P) to produce N-acetylglucosamine-1-phosphate (GlcNAc-1-P).

The term “glmU” refers to a bifunctional enzyme that has both N-acetylglucosamine-1-phosphate uridyltransferase and glucosamine-1-phosphate acetyltransferase activity and that catalyses two sequential reactions in the de novo biosynthetic pathway for UDP-GlcNAc. The C-terminal domain catalyses the transfer of acetyl group from acetyl coenzyme A to GlcN-1-P to produce GlcNAc-1-P, which is converted into UDP-GlcNAc by the transfer of uridine 5-monophosphate, a reaction catalysed by the N-terminal domain.

The terms “NeunAc synthase”, “N-acetylneuraminic acid synthase”, “N-acetylneuraminate synthase”, “sialic acid synthase”, “NeuAc synthase”, “NeuB”, “NeuB1”, “NeuNAc synthase”, “NANA condensing enzyme”, “N-acetylneuraminate lyase synthase”, “N-acetylneuraminic acid condensing enzyme” as used herein are used interchangeably and refer to an enzyme capable to synthesize sialic acid from N-acetylmannosamine (ManNAc) in a reaction using phosphoenolpyruvate (PEP).

The terms “N-acetylneuraminate lyase”, “Neu5Ac lyase”, “N-acetylneuraminate pyruvate-lyase”, “N-acetylneuraminic acid aldolase”, “NALase”, “sialate lyase”, “sialic acid aldolase”, “sialic acid lyase” and “nanA” are used interchangeably and refer to an enzyme that degrades N-acetylneuraminate into N-acetylmannosamine (ManNAc) and pyruvate.

The terms “N-acylneuraminate-9-phosphate synthase”, “N-acylneuraminate-9-phosphate synthetase”, “NANA synthase”, “NANAS”, “NANS”, “NmeNANAS”, “N-acetylneuraminate pyruvate-lyase (pyruvate-phosphorylating)” as used herein are used interchangeably and refer to an enzyme capable to synthesize N-acylneuraminate-9-phosphate from N-acetylmannosamine-6-phosphate (ManNAc-6-phosphate) in a reaction using phosphoenolpyruvate (PEP).

The term “N-acylneuraminate-9-phosphatase” refers to an enzyme capable to dephosphorylate N-acylneuraminate-9-phosphate to synthesise N-acylneuraminate.

The terms “CMP-sialic acid synthase”, “N-acylneuraminate cytidylyltransferase”, “CMP-sialate synthase”, “CMP-NeuAc synthase”, “NeuA” and “CMP-N-acetylneuraminic acid synthase” as used herein are used interchangeably and refer to an enzyme capable to synthesize CMP-N-acetylneuraminate from N-acetylneuraminate using CTP in the reaction.

The terms “galactose-1-epimerase”, “aldose 1-epimerase”, “mutarotase”, “aldose mutarotase”, “galactose mutarotase”, “galactose 1-epimerase” and “D-galactose 1-epimerase” are used interchangeably and refer to an enzyme that catalyses the conversion of beta-D-galactose into alpha-D-galactose.

The terms “galactokinase”, “galactokinase (phosphorylating)” and “ATP: D-galactose-1-phosphotransferase” are used interchangeably and refer to an enzyme that catalyses the conversion of alpha-D-galactose into alpha-D-galactose 1-phosphate using ATP.

The terms glucokinase, and “glucokinase (phosphorylating)” are used interchangeably and refer to an enzyme that catalyses the conversion of D-glucose into D-glucose 6-phosphate using ATP.

The terms “galactose-1-phosphate uridylyltransferase”, “Gal-1-P uridylyltransferase”, “UDP-glucose—hexose-1-phosphate uridylyltransferase”, “uridyl transferase”, “hexose-1-phosphate uridylyltransferase”, “uridyltransferase”; “hexose 1-phosphate uridyltransferase”, “UDP-glucose: alpha-D-galactose-1-phosphate uridylyltransferase”, “galB” and “galT” are used interchangeably and refer to an enzyme that catalyses the reaction D-galactose 1-phosphate+UDP-D-glucose=D-glucose 1-phosphate+UDP-D-galactose.

The terms “UDP-glucose 4-epimerase”, “UDP-galactose 4-epimerase”, “uridine diphosphoglucose epimerase”, “galactowaldenase”, “UDPG-4-epimerase”, “uridine diphosphate galactose 4-epimerase”, “uridine diphospho-galactose-4-epimerase”, “UDP-glucose epimerase”, “4-epimerase”, “uridine diphosphoglucose 4-epimerase”, “uridine diphosphate glucose 4-epimerase” and “UDP-D-galactose 4-epimerase” are used interchangeably and refer to an enzyme that catalyses the conversion of UDP-D-glucose into UDP-galactose.

The terms “glucose-1-phosphate uridylyltransferase”, “UTP—glucose-1-phosphate uridylyltransferase”, “UDP glucose pyrophosphorylase”, “UDPG phosphorylase”, “UDPG pyrophosphorylase”, “uridine 5′-diphosphoglucose pyrophosphorylase”, “uridine diphosphoglucose pyrophosphorylase”, “uridine diphosphate-D-glucose pyrophosphorylase”, “uridine-diphosphate glucose pyrophosphorylase” and “galU” are used interchangeably and refer to an enzyme that catalyses the conversion of D-glucose-1-phosphate into UDP-glucose using UTP.

The terms “phosphoglucomutase (alpha-D-glucose-1,6-bisphosphate-dependent)”, “glucose phosphomutase (ambiguous)” and “phosphoglucose mutase (ambiguous)” are used interchangeably and refer to an enzyme that catalyses the conversion of D-glucose 1-phosphate into D-glucose 6-phosphate. The terms “UDP-N-acetylglucosamine 4-epimerase”, “UDP acetylglucosamine epimerase”, “uridine diphosphoacetylglucosamine epimerase”, “uridine diphosphate N-acetylglucosamine-4-epimerase”, “uridine 5′-diphospho-N-acetylglucosamine-4-epimerase” and “UDP-N-acetyl-D-glucosamine 4-epimerase” are used interchangeably and refer to an enzyme that catalyses the epimerization of UDP-N-acetylglucosamine (UDP-GlcNAc) to UDP-N-acetylgalactosamine (UDP-GalNAc).

The terms “N-acetylgalactosamine kinase”, “GALK2”, “GK2”, “GalNAc kinase”, “N-acetylgalactosamine (GalNAc)-1-phosphate kinase” and “ATP: N-acetyl-D-galactosamine 1-phosphotransferase” are used interchangeably and refer to an enzyme that catalyses the synthesis of N-acetylgalactosamine 1-phosphate (GalNAc-1-P) from N-acetylgalactosamine (GalNAc) using ATP.

The terms “UDP-N-acetylgalactosamine pyrophosphorylase” and “UDP-GalNAc pyrophosphorylase” are used interchangeably and refer to an enzyme that catalyses the conversion of N-acetylgalactosamine 1-phosphate (GalNAc-1-P) into UDP-N-acetylgalactosamine (UDP-GalNAc) using UTP.

The terms “N-acetylneuraminate kinase”, “ManNAc kinase”, “N-acetyl-D-mannosamine kinase” and “nanK” are used interchangeably and refer to an enzyme that phosphorylates ManNAc to synthesize N-acetylmannosamine-phosphate (ManNAc-6-P).

The term “glycosyltransferase” as used herein refers to an enzyme capable to catalyse the transfer of sugar moieties from activated donor molecules to specific acceptor molecules, forming glycosidic bonds.

A classification of glycosyltransferases using nucleotide diphospho-sugar, nucleotide monophospho-sugar and sugar phosphates and related proteins into distinct sequence-based families has been described (Campbell et al., Biochem. J. 326, 929-939 (1997)) and is available on the CAZy (CArbohydrate-Active EnZymes) website (www.cazy.org).

As used herein the glycosyltransferase can be selected from the list comprising but not limited to: fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

Fucosyltransferases are glycosyltransferases that transfer a fucose residue (Fuc) from a GDP-fucose (GDP-Fuc) donor onto an acceptor. Fucosyltransferases comprise alpha-1,2-fucosyltransferases, alpha-1,3-fucosyltransferases, alpha-1,4-fucosyltransferases and alpha-1,6-fucosyltransferases that catalyse the transfer of a Fuc residue from GDP-Fuc onto an acceptor via alpha-glycosidic bonds. Fucosyltransferases can be found but are not limited to the GT10, GT11, GT23, GT65, GT68 and GT74 CAZy families.

The terms “alpha-1,2-fucosyltranferase”, “alpha 1,2 fucosyltransferase”, “2-fucosyltransferase, “α-1,2-fucosyltransferase”, “a 1,2 fucosyltransferase”, “2 fucosyltransferase, “2-FT” or “2 FT” as used in the present invention, are used interchangeably and refer to a glycosyltransferase that catalyses the transfer of fucose from the donor GDP-L-fucose to the acceptor molecule in an alpha-1,2-linkage.

The wording “alpha-1,2-fucosyltransferase that has galactoside alpha-1,2-fucosyltransferase activity on the galactose residue of Gal-b1,3-GlcNAc (LNB, lacto-N-biose)” refers to an alpha-1,2-fucosyltransferase that catalyses the transfer of fucose from the donor GDP-L-fucose to the galactose residue of Gal-b1,3-GlcNAc in an alpha-1,2-linkage producing Fuc-α1,2-Gal-b1,3-GlcNAc (2′-fucosyllacto-N-biose, 2′FLNB).

In the present invention, polypeptide sequence stretches are being used to refer to fragments of the alpha-1,2-fucosyltransferases used in the present invention which are common to those alpha-1,2-fucosyltransferase. Such polypeptide stretches are written in the form of a sequence of amino acids in one-letter code. In case an amino acid at a specific place in such polypeptide stretch can be several amino acids, that specific place will have amino acid code X. Unless otherwise mentioned herein, the letter “X” refers to any amino acid possible. The term “X (no M)” refers to any amino acid possible except methionine (Met, M). The term “X (no F)” refers to any amino acid possible except phenylalanine (Phe, F). The term “X (no N)” refers to any amino acid possible except asparagine (Asn, N). The term [ILMV] in a sequence refers to an isoleucine (IIe, I), leucine (Leu, L), methionine (Met, M) or valine (Val, V) as possible amino acid at that specific place. The term “X (no E,S)” refers to any amino acid possible except glutamic acid (Glu, E) and serine (Ser, S). The term “X (no E)” refers to any amino acid possible except glutamic acid (Glu, E). The term “X (no F, S)” refers to any amino acid possible except phenylalanine (Phe, F) and serine (Ser, S). The term “X (no Y)” refers to any amino acid possible except tyrosine (Tyr, Y). The term “X (no H, S, Y)” refers to any amino acid possible except histidine (His, H), serine (Ser, S) and tyrosine (Tyr, Y). The term [DE] in a sequence refers to an aspartic acid (Asp, D) or a glutamic acid (Glu, E) as possible amino acid at that specific place. The term [FWY] in a sequence refers to a phenylalanine (Phe, F), tryptophan (W) or tyrosine (Y) as possible amino acid at that specific place. The term “X (no D,E)” refers to any amino acid possible except aspartic acid (Asp, D) and glutamic acid (Glu, E). The term “(Xn)” with n being 10 to 40 refers to a polypeptide stretch of 10 to 40 amino acid residues X, wherein X refers to any amino acid possible.

Sialyltransferases are glycosyltransferases that transfer a sialic acid (like Neu5Ac or Neu5Gc) from a donor (like CMP-Neu5Ac or CMP-Neu5Gc) onto an acceptor. Sialyltransferases comprise alpha-2,3-sialyltransferases, alpha-2,6-sialyltransferases and alpha-2,8-sialyltransferases that catalyse the transfer of a sialic acid onto an acceptor via alpha-glycosidic bonds. Sialyltransferases can be found but are not limited to the GT29, GT42, GT80 and GT97 CAZy families. Galactosyltransferases are glycosyltransferases that transfer a galactosyl group (Gal) from an UDP-galactose (UDP-Gal) donor onto an acceptor. Galactosyltransferases comprise beta-1,3-galactosyltransferases, N-acetylglucosamine beta-1,3-galactosyltransferases, beta-1,4-galactosyltransferases, N-acetylglucosamine beta-1,4-galactosyltransferases, alpha-1,3-galactosyltransferases and alpha-1,4-galactosyltransferases that transfer a Gal residue from UDP-Gal onto an acceptor via alpha- or beta-glycosidic bonds. Galactosyltransferases can be found but are not limited to the GT2, GT6, GT8, GT25 and GT92 CAZy families. Glucosyltransferases are glycosyltransferases that transfer a glucosyl group (Glc) from an UDP-glucose (UDP-Glc) donor onto an acceptor. Glucosyltransferases comprise alpha-glucosyltransferases, beta-1,2-glucosyltransferases, beta-1,3-glucosyltransferases and beta-1,4-glucosyltransferases that transfer a Glc residue from UDP-Glc onto an acceptor via alpha- or beta-glycosidic bonds. Glucosyltransferases can be found but are not limited to the GT1, GT4 and GT25 CAZy families. Mannosyltransferases are glycosyltransferases that transfer a mannose group (Man) from a GDP-mannose (GDP-Man) donor onto an acceptor. Mannosyltransferases comprise alpha-1,2-mannosyltransferases, alpha-1,3-mannosyltransferases and alpha-1,6-mannosyltransferases that transfer a Man residue from GDP-Man onto an acceptor via alpha-glycosidic bonds. Mannosyltransferases can be found but are not limited to the GT22, GT39, GT62 and GT69 CAZy families. N-acetylglucosaminyltransferases are glycosyltransferases that transfer an N-acetylglucosamine group (GlcNAc) from an UDP-N-acetylglucosamine (UDP-GlcNAc) donor onto an acceptor. N-acetylglucosaminyltransferases can be found but are not limited to GT2 and GT4 CAZy families. Galactoside beta-1,3-N-acetylglucosaminyltransferases are part of N-acetylglucosaminyltransferases and transfer GlcNAc from an UDP-GlcNAc donor onto a terminal galactose unit present in an acceptor via a beta-1,3-linkage. Beta-1,6-N-acetylglucosaminyltransferases are N-acetylglucosaminyltransferases that transfer GlcNAc from an UDP-GlcNAc donor onto an acceptor via a beta-1,6-linkage. N-acetylgalactosaminyltransferases are glycosyltransferases that transfer an N-acetylgalactosamine group (GalNAc) from an UDP-N-acetylgalactosamine (UDP-GalNAc) donor onto an acceptor. N-acetylgalactosaminyltransferases can be found but are not limited to GT7, GT12 and GT27 CAZy families. Alpha-1,3-N-acetylgalactosaminyltransferases are part of the N-acetylgalactosaminyltransferases and transfer GalNAc from an UDP-GalNAc donor to an acceptor via an alpha-1,3-linkage. N-acetylmannosaminyltransferases are glycosyltransferases that transfer an N-acetylmannosamine group (ManNAc) from an UDP-N-acetylmannosamine (UDP-ManNAc) donor onto an acceptor. Xylosyltransferases are glycosyltransferases that transfer a xylose residue (Xyl) from an UDP-xylose (UDP-Xyl) donor onto an acceptor. Xylosyltransferases can be found but are not limited to GT14, GT61 and GT77 CAZy families. Glucuronyltransferases are glycosyltransferases that transfer a glucuronate from an UDP-glucuronate donor onto an acceptor via alpha- or beta-glycosidic bonds. Glucuronyltransferases can be found but are not limited to GT4, GT43 and GT93 CAZy families. Galacturonyltransferases are glycosyltransferases that transfer a galacturonate from an UDP-galacturonate donor onto an acceptor. N-glycolylneuraminyltransferases are glycosyltransferases that transfer an N-glycolylneuraminic acid group (Neu5Gc) from a CMP-Neu5Gc donor onto an acceptor. Rhamnosyltransferases are glycosyltransferases that transfer a rhamnose residue from a GDP-rhamnose donor onto an acceptor. Rhamnosyltransferases can be found but are not limited to the GT1, GT2 and GT102 CAZy families. N-acetylrhamnosyltransferases are glycosyltransferases that transfer an N-acetylrhamnosamine residue from an UDP-N-acetyl-L-rhamnosamine donor onto an acceptor. UDP-4-amino-4,6-dideoxy-N-acetyl-beta-L-altrosamine transaminases are glycosyltransferases that use an UDP-2-acetamido-2,6-dideoxy—L-arabino-4-hexulose in the biosynthesis of pseudaminic acid, which is a sialic acid-like sugar that is used to modify flagellin. UDP-N-acetylglucosamine enolpyruvyl transferases (murA) are glycosyltransferases that transfer an enolpyruvyl group from phosphoenolpyruvate (PEP) to UDP-N-acetylglucosamine (UDPAG) to form UDP-N-acetylglucosamine enolpyruvate. Fucosaminyltransferases are glycosyltransferases that transfer an N-acetylfucosamine residue from a dTDP-N-acetylfucosamine or an UDP-N-acetylfucosamine donor onto an acceptor.

The terms “activated monosaccharide”, “nucleotide-activated sugar”, “nucleotide-sugar”, “activated sugar”, “nucleoside” or “nucleotide donor” are used herein interchangeably and refer to activated forms of monosaccharides. Examples of activated monosaccharides comprise UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy—L-arabino-4-hexulose, UDP-2-acetamido-2,6-dideoxy—L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N3, CMP-Neu4,5Ac2, CMP-Neu5,7Ac2, CMP-Neu5,9Ac2, CMP-Neu5,7 (8,9) Ac2, CMP-N-glycolylneuraminic acid (CMP-Neu5Gc), GDP-rhamnose or UDP-xylose. Nucleotide-sugars act as glycosyl donors in glycosylation reactions. Those reactions are catalysed by glycosyltransferases.

The term “monosaccharide” as used herein refers to a sugar that is not decomposable into simpler sugars by hydrolysis, is classed either an aldose or ketose, and contains one or more hydroxyl groups per molecule. Monosaccharides are saccharides containing only one simple sugar. Examples of monosaccharides comprise Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LDmanHep), D-glycero-D-manno-Heptopyranose (DDmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyranose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Gulopyranuronic acid, L-Gulopyranuronic acid, L-Idopyranuronic acid, D-Talopyranuronic acid, sialic acid, 5-Amino-3,5-dideoxy-D-glycero-D-galacto-non-2-ulosonic acid, 5-Acetamido-3,5-dideoxy-D-glycero-D-galacto-non-2-ulosonic acid, 5-Glycolylamido-3,5-dideoxy-D-glycero-D-galacto-non-2-ulosonic acid, Erythritol, Arabinitol, Xylitol, Ribitol, Glucitol, Galactitol, Mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-lyxo-Hex-2-ulopyranose, D-threo-Pent-2-ulopyranose, D-altro-Hept-2-ulopyranose, 3-C-(Hydroxymethyl)-D-erythofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O—[(R)-1-carboxyethyl]-2-deoxy-D-glucopyranose, 2-Acetamido-3-O—[(R)-carboxyethyl]-2-deoxy-D-glucopyranose, 2-Glycolylamido-3-O—[(R)-1-carboxyethyl]-2-deoxy-D-glucopyranose, 3-Deoxy-D-lyxo-hept-2-ulopyranosaric acid, 3-Deoxy-D-manno-oct-2-ulopyranosonic acid, 3-Deoxy-D-glycero-D-galacto-non-2-ulopyranosonic acid, 5,7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5,7-Diamino-3,5,7,9-tetradeoxy-D-glycero-D-galacto-non-2-ulopyranosonic acid, 5,7-Diamino-3,5,7,9-tetradeoxy-D-glycero-D-talo-non-2-ulopyranosonic acid, 2-acetamido-2,6-dideoxy—L-arabino-4-hexulose, 2-acetamido-2,6-dideoxy—L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, glucose (Glc), galactose (Gal), N-acetylglucosamine (GlcNAc), glucosamine (Glcn), mannose (Man), xylose (Xyl), N-acetylmannosamine (ManNAc), N-glycolylneuraminic acid, N-acetylgalactosamine (GalNAc), galactosamine (Galn), fucose (Fuc), rhamnose (Rha), glucuronic acid, gluconic acid, fructose (Fru) and polyols.

With the term polyol is meant an alcohol containing multiple hydroxyl groups. For example, glycerol, sorbitol, or mannitol.

The terms “sialic acid”, “N-acetylneuraminate”, “N-acylneuraminate”, “N-acetylneuraminic acid” are used interchangeably and refer to an acidic sugar with a nine-carbon backbone comprising but not limited to Neu4Ac; Neu5Ac; Neu4,5Ac2; Neu5,7Ac2; Neu5,8Ac2; Neu5,9Ac2; Neu4,5,9Ac3; Neu5,7,9Ac3; Neu5,8,9Ac3; Neu4,5,7,9Ac4; Neu5,7,8,9Ac4, Neu4,5,7,8,9Ac5 and Neu5Gc.

Neu4Ac is also known as 4-O-acetyl-5-amino-3,5-dideoxy-D-glycero-D-galacto-non-2-ulopyranosonic acid or 4-O-acetyl neuraminic acid and has C11H19NO9 as molecular formula. Neu5Ac is also known as 5-acetamido-3,5-dideoxy-D-glycero-D-galacto-non-2-ulopyranosonic acid, D-glycero-5-acetamido-3,5-dideoxy-D-galacto-non-2-ulo-pyranosonic acid, 5-(acetylamino)-3,5-dideoxy-D-glycero-D-galacto-2-nonulopyranosonic acid, 5-(acetylamino)-3,5-dideoxy-D-glycero-D-galacto-2-nonulosonic acid, 5-(acetylamino)-3,5-dideoxy-D-glycero-D-galacto-non-2-nonulosonic acid or 5-(acetylamino)-3,5-dideoxy-D-glycero-D-galacto-non-2-ulopyranosonic acid and has C11H19NO9 as molecular formula. Neu4,5Ac2 is also known as N-acetyl-4-O-acetylneuraminic acid, 4-O-acetyl-N-acetylneuraminic acid, 4-O-acetyl-N-acetylneuraminate, 4-acetate 5-acetamido-3,5-dideoxy-D-glycero-D-galacto-nonulosonate, 4-acetate 5-(acetylamino)-3,5-dideoxy-D-glycero-D-galacto-2-nonulosonate, 4-acetate 5-acetamido-3,5-dideoxy-D-glycero-D-galacto-nonulosonic acid or 4-acetate 5-(acetylamino)-3,5-dideoxy-D-glycero-D-galacto-2-nonulosonic acid and has C13H21NO10 as molecular formula. Neu5,7Ac2 is also known as 7-O-acetyl-N-acetylneuraminic acid, N-acetyl-7-O-acetylneuraminic acid, 7-O-acetyl-N-acetylneuraminate, 7-acetate 5-acetamido-3,5-dideoxy-D-glycero-D-galacto-nonulosonate, 7-acetate 5-(acetylamino)-3,5-dideoxy-D-glycero-D-galacto-2-nonulosonate, 7-acetate 5-acetamido-3,5-dideoxy-D-glycero-D-galacto-nonulosonic acid or 7-acetate 5-(acetylamino)-3,5-dideoxy-D-glycero-D-galacto-2-nonulosonic acid and has C13H21NO10 as molecular formula. Neu5,8Ac2 is also known as 5-n-acetyl-8-O-acetyl neuraminic acid and has C13H21NO10 as molecular formula. Neu5,9Ac2 is also known as N-acetyl-9-O-acetylneuraminic acid, 9-anana, 9-O-acetylsialic acid, 9-O-acetyl-N-acetylneuraminic acid, 5-n-acetyl-9-O-acetyl neuraminic acid, N,9-O-diacetylneuraminate or N,9-O-diacetylneuraminate and has C13H21NO10 as molecular formula. Neu4,5,9Ac3 is also known as 5-N-acetyl-4,9-di-O-acetylneuraminic acid. Neu5,7,9Ac3 is also known as 5-N-acetyl-7,9-di-O-acetylneuraminic acid. Neu5,8,9Ac3 s also known as 5-N-acetyl-8,9-di-O-acetylneuraminic acid. Neu4,5,7,9Ac4 is also known as 5-N-acetyl-4,7,9-tri-O-acetylneuraminic acid.

Neu5,7,8,9Ac4 is also known as 5-N-acetyl-7,8,9-tri-O-acetylneuraminic acid. Neu4,5,7,8,9Ac5 is also known as 5-N-acetyl-4,7,8,9-tetra-O-acetylneuraminic acid. Neu5Gc is also known as N-glycolylneuraminic acid, N-glycolylneuraminic acid, N-glycolylneuraminate, N-glycoloyl-neuraminate, N-glycoloylneuraminic acid, N-glycoloylneuraminic acid, 3,5-dideoxy-5-((hydroxyacetyl)amino)-D-glycero-D-galacto-2-nonulosonic acid, 3,5-dideoxy-5-(glycoloylamino)-D-glycero-D-galacto-2-nonulopyranosonic acid, 3,5-dideoxy-5-(glycoloylamino)-D-glycero-D-galacto-non-2-ulopyranosonic acid, 3,5-dideoxy-5-[(hydroxyacetyl)amino]-D-glycero-D-galacto-non-2-ulopyranosonic acid, D-glycero-5-glycolylamido-3,5-dideoxy-D-galacto-non-2-ulo-pyranosonic acid and has C11H19NO10 as molecular formula.

The term “disaccharide” as used herein refers to a saccharide polymer containing two simple sugars, i.e. monosaccharides. Such disaccharides contain monosaccharides preferably selected from the list of monosaccharides as used herein. Examples of disaccharides comprise lactose (Gal-b1,4-Glc), lacto-N-biose (Gal-b1,3-GlcNAc), N-acetyllactosamine (Gal-b1,4-GlcNAc), LacDiNAc (GaINAc-b1,4-GlcNAc), N-acetylgalactosaminylglucose (GalNAc-b1,4-Glc), Neu5Ac-a2,3-Gal, Neu5Ac-a2,6-Gal and fucopyranosyl-(1-4)—N-glycolylneuraminic acid (Fuc-(1-4)-Neu5Gc).

“Oligosaccharide” as the term is used herein and as generally understood in the state of the art, refers to a saccharide polymer containing a small number, typically three to twenty, of simple sugars, i.e. monosaccharides. Preferably the oligosaccharide as described herein contains monosaccharides selected from the list as used herein. The oligosaccharide as used in the present invention can be a linear structure or can include branches. The linkage (e.g. glycosidic linkage, galactosidic linkage, glucosidic linkage, etc.) between two sugar units can be expressed, for example, as 1,4, 1->4, or (1-4), used interchangeably herein. For example, the terms “Gal-b1,4-Glc”, “b-Gal-(1->4)-Glc”, “Galbeta1-4-Glc” and “Gal-b (1-4)-Glc” have the same meaning, i.e. a beta-glycosidic bond links carbon-1 of galactose (Gal) with the carbon-4 of glucose (Glc). Each monosaccharide can be in the cyclic form (e.g. pyranose or furanose form). Linkages between the individual monosaccharide units may include alpha 1->2, alpha 1->3, alpha 1->4, alpha 1->6, alpha 2->1, alpha 2->3, alpha 2->4, alpha 2->6, beta 1->2, beta 1->3, beta 1->4, beta 1->6, beta 2->1, beta 2->3, beta 2->4, and beta 2->6. An oligosaccharide can contain both alpha- and beta-glycosidic bonds or can contain only alpha-glycosidic or only beta-glycosidic bonds. The term “polysaccharide” refers to a compound consisting of a large number, typically more than twenty, of monosaccharides linked glycosidically.

Examples of oligosaccharides include but are not limited to Lewis-type antigen oligosaccharides, mammalian (including human) milk oligosaccharides, O-antigen, enterobacterial common antigen (ECA), the glycan chain present in lipopolysaccharides (LPS), the oligosaccharide repeats present in capsular polysaccharides, peptidoglycan (PG), amino-sugars and antigens of the human ABO blood group system. As used herein, “mammalian milk oligosaccharide” refers to oligosaccharides such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 8,3-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharide and/or sialylated oligosaccharides.

A ‘fucosylated oligosaccharide’ as used herein and as generally understood in the state of the art is an oligosaccharide that is carrying a fucose-residue. Examples comprise 2′-fucosyllactose (2′FL), 3-fucosyllactose (3FL), 4-fucosyllactose (4FL), 6-fucosyllactose (6FL), difucosyllactose (diFL), lactodifucotetraose (LDFT), Lacto-N-fucopentaose I (LNF I), Lacto-N-fucopentaose II (LNF II), Lacto-N-fucopentaose III (LNF III), lacto-N-fucopentaose V (LNF V), lacto-N-fucopentaose VI (LNF VI), lacto-N-neofucopentaose I, lacto-N-difucohexaose I (LDFH I), lacto-N-difucohexaose II (LDFH II), Monofucosyllacto-N-hexaose III (MFLNH III), Difucosyllacto-N-hexaose (DFLNHa), difucosyl-lacto-N-neohexaose.

As used herein, a ‘sialylated oligosaccharide’ is to be understood as a charged sialic acid containing oligosaccharide, i.e. an oligosaccharide having a sialic acid residue. It has an acidic nature. Some examples are 3-SL (3′-sialyllactose or 3′-SL or Neu5Ac-a2,3-Gal-b1,4-Glc), 3′-sialyllactosamine, 6-SL (6′-sialyllactose or 6′-SL or Neu5Ac-a2,6-Gal-b1,4-Glc), 3,6-disialyllactose (Neu5Ac-a2,3-(Neu5Ac-a2,6)-Gal-b1,4-Glc), 6,6′-disialyllactose (Neu5Ac-a2,6-Gal-b1,4-(Neu5Ac-a2,6)-Glc), 8,3-disialyllactose (Neu5Ac-a2,8-Neu5Ac-a2,3-Gal-b1,4-Glc), 6′-sialyllactosamine, oligosaccharides comprising 6′-sialyllactose, SGG hexasaccharide (Neu5Acα-2,3Galβ-1,3GalNacβ-1,3Gala-1,4Galβ-1,4Gal), sialylated tetrasaccharide (Neu5Acα-2,3Galβ-1,4GlcNacβ-14GlcNAc), pentasaccharide LSTD (Neu5Acα-2,3Galβ-1,4GlcNacβ-1,3Galβ-1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residue(s), including but not limited to: oligosaccharide moieties of the gangliosides selected from GM3 (3′sialyllactose, Neu5Acα-2,3GalB-4Glc) and oligosaccharides comprising the GM3 motif, GD3 Neu5Acα-2,8Neu5Acα-2,3GalB-1,4Glc GT3 (Neu5Acα-2,8Neu5Acα-2,8Neu5Acα-2,3Galβ-1,4Glc); GM2 GalNAcβ-1,4 (Neu5Acα-2,3) Galβ-1,4Glc, GM1 Galβ-1,3GalNAcβ-1,4 (Neu5Acα-2,3) Galβ-1,4Glc, GD1a Neu5Acα-2,3Galβ-1,3GalNAcβ-1,4 (Neu5Acα-2,3) Galβ-1,4Glc, GT1a Neu5Acα-2,8Neu5Acα-2,3Galβ-1,3GalNAcβ-1,4 (Neu5Acα-2,3) Galβ-1,4Glc, GD2 GalNAcβ-1,4 (Neu5Acα-2,8Neu5Acα2,3) Galβ-1,4Glc, GT2 GalNAcβ-1,4 (Neu5Acα-2,8Neu5Acα-2,8Neu5Acα2,3) Galβ-1,4Glc, GD1b, Galβ-1,3GalNAcβ-1,4 (Neu5Acα-2,8Neu5Acα2,3) Galβ-1,4Glc, GT1b Neu5Acα-2,3Galβ-1,3GalNAcβ-1,4 (Neu5Acα-2,8Neu5Acα2,3) Galβ-1,4Glc, GQ1b Neu5Acα-2,8Neu5Acα-2,3Galβ-1,3GalNAc β-1,4 (Neu5Acα-2,8Neu5Acα2,3) Galβ-1,4Glc, GT1c Galβ-1,3GalNAcβ-1,4 (Neu5Acα-2,8Neu5Acα-2,8Neu5Acα2,3) Galβ-1,4Glc, GQ1c Neu5Acα-2,3Galβ-1,3GalNAc β-1,4 (Neu5Acα-2,8Neu5Acα-2,8Neu5Acα2,3) Galβ-1,4Glc, GP1c Neu5Acα-2,8Neu5Acα-2,3Galβ-1,3GalNAc β-1,4 (Neu5Acα-2,8Neu5Acα-2,8Neu5Acα2,3) Galβ-1,4Glc, GD1a Neu5Acα-2,3Galβ-1,3 (Neu5Acα-2,6) GalNAcβ-1,4Galβ-1,4Glc, Fucosyl-GM1 Fuca-1,2Galβ-1,3GalNAcβ-1,4 (Neu5Acα-2,3) Gal β-1,4Glc; all of which may be extended to the production of the corresponding gangliosides by reacting the above oligosaccharide moieties with ceramide or synthetizing the above oligosaccharides on a ceramide.

A ‘neutral oligosaccharide’ as used herein and as generally understood in the state of the art is an oligosaccharide that has no negative charge originating from a carboxylic acid group. Examples of such neutral oligosaccharide are 2′-fucosyllactose (2′FL), 3-fucosyllactose (3FL), 2′,3-difucosyllactose (diFL), lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose.

Mammalian milk oligosaccharides or MMOs comprise oligosaccharides present in milk found in any phase during lactation including colostrum milk from humans (i.e. human milk oligosaccharides or HMOs) and mammals including but not limited to cows (Bos Taurus), sheep (Ovis aries), goats (Capra aegagrus hircus), bactrian camels (Camelus bactrianus), horses (Equus ferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus vulpecula), koalas (Phascolarctos cinereus), eastern quolls (Dasyurus viverrinus), platypus (Ornithorhynchus anatinus). Human milk oligosaccharides are also known as human identical milk oligosaccharides which are chemically identical to the human milk oligosaccharides found in human breast milk but which are biotechnologically-produced (e.g. using cell free systems or cells and organisms comprising a bacterium, a fungus, a yeast, a plant, animal, or protozoan cell, preferably genetically engineered cells and organisms). Human identical milk oligosaccharides are marketed under the name HiMO.

As used herein the term “Lewis-type antigens” comprise the following oligosaccharides: H1 antigen, which is Fucα1-2Galβ1-3GlcNAc, or in short 2′FLNB; Lewisa, which is the trisaccharide Galβ1-3 [Fucα1-4]GlcNAc, or in short 4-FLNB; Lewisb, which is the tetrasaccharide Fucα1-2Galβ1-3 [Fucα1-4]GlcNAc, or in short DiF-LNB; sialyl Lewisa which is 5-acetylneuraminyl-(2-3)-galactosyl-(1-3)-(fucopyranosyl-(1-4))—N-acetylglucosamine, or written in short Neu5Acα2-3Galβ1-3 [Fucα1-4]GlcNAc; H2 antigen, which is Fucα1-2Galβ1-4GlcNAc, or otherwise stated 2′fucosyl-N-acetyl-lactosamine, in short 2′FLacNAc; Lewisx, which is the trisaccharide Galβ1-4 [Fucα1-3]GlcNAc, or otherwise known as 3-Fucosyl-N-acetyl-lactosamine, in short 3-FLacNAc, Lewisy, which is the tetrasaccharide Fucα1-2Galβ1-4 [Fucα1-3]GlcNAc and sialyl Lewisx which is 5-acetylneuraminyl-(2-3)-galactosyl-(1-4)-(fucopyranosyl-(1-3))—N-acetylglucosamine, or written in short Neu5Acα2-3Galβ1-4 [Fucα1-3]GlcNAc.

As used herein, the term “O-antigen” refers to the repetitive glycan component of the surface lipopolysaccharide (LPS) of Gram-negative bacteria. The term “lipopolysaccharide” or “LPS” refers to glycolipids found in the outer membrane of Gram-negative bacteria which are composed of a lipid A, a core oligosaccharide and the O-antigen. The term “enterobacterial common antigen” or “ECA” refers to a specific carbohydrate antigen built of repeating units of three amino sugars, i.e. N-acetylglucosamine, N-acetyl-d-mannosaminuronic acid and 4-acetamido-4,6-dideoxy-d-galactose, which is shared by all members of the Enterobacteriaceae and which is located in the outer leaflet of the outer membrane and in the periplasm. The term “capsular polysaccharides” refers to long-chain polysaccharides with oligosaccharide repeat structures that are present in bacterial capsules, the latter being a polysaccharide layer that lies outside the cell envelope. The terms “peptidoglycan” or “murein” refers to an essential structural element in the cell wall of most bacteria, being composed of sugars and amino acids, wherein the sugar components consist of alternating residues of beta-1,4 linked GlcNAc and N-acetylmuramic acid. The term “amino-sugar” as used herein refers to a sugar molecule in which a hydroxyl group has been replaced with an amine group. As used herein, an antigen of the human ABO blood group system is an oligosaccharide. Such antigens of the human ABO blood group system are not restricted to human structures. Said structures involve the A determinant GalNAc-alpha1,3 (Fuc-alpha1,2)-Gal-, the B determinant Gal-alpha1,3 (Fuc-alpha1,2)-Gal- and the H determinant Fuc-alpha1,2-Gal that are present on disaccharide core structures comprising Gal-beta1,3-GlcNAc, Gal-beta1,4-GlcNAc, Gal-beta1,3-GalNAc and Gal-beta1,4-Glc.

The terms “LNT II”, “LNT-II”, “LN3”, “lacto-N-triose II”, “lacto-N-triose II”, “lacto-N-triose”, “lacto-N-triose” or “GlcNAcβ1-3Galβ1-4Glc” as used in the present invention, are used interchangeably.

The terms “LNT”, “lacto-N-tetraose”, “lacto-N-tetraose” or “Galβ1-3GlcNAcβ1-3Galβ1-4Glc” as used in the present invention, are used interchangeably.

The terms “LNnT”, “lacto-N-neotetraose”, “lacto-N-neotetraose”, “neo-LNT” or “Galβ1-4GlcNAcβ1-3Galβ1-4Glc” as used in the present invention, are used interchangeably.

The terms “LSTa”, “LS-Tetrasaccharide a”, “Sialyl-lacto-N-tetraose a”, “sialyllacto-N-tetraose a” or “Neu5Ac-a2,3-Gal-b1,3-GlcNAc-b1,3-Gal-b1,4-Glc” as used in the present invention, are used interchangeably.

The terms “LSTb”, “LS-Tetrasaccharide b”, “Sialyl-lacto-N-tetraose b”, “sialyllacto-N-tetraose b” or “Gal-b1,3-(Neu5Ac-a2,6)-GlcNAc-b1,3-Gal-b1,4-Glc” as used in the present invention, are used interchangeably.

The terms “LSTc”, “LS-Tetrasaccharide c”, “Sialyl-lacto-N-tetraose c”, “sialyllacto-N-tetraose c”, “sialyllacto-N-neotetraose c” or “Neu5Ac-a2,6-Gal-b1,4-GlcNAc-b1,3-Gal-b1,4-Glc” as used in the present invention, are used interchangeably.

The terms “LSTd”, “LS-Tetrasaccharide d”, “Sialyl-lacto-N-tetraose d”, “sialyllacto-N-tetraose d”, “sialyllacto-N-neotetraose d” or “Neu5Ac-a2,3-Gal-b1,4-GlcNAc-b1,3-Gal-b1,4-Glc” as used in the present invention, are used interchangeably.

The terms “DSLNnT” and “Disialyllacto-N-neotetraose” are used interchangeably and refer to Neu5Ac-a2,6-[Neu5Ac-a2,6-Gal-b1,4-GlcNAc-b1,3]-Gal-b1,4-Glc.

The terms “DSLNT” and “Disialyllacto-N-tetraose” are used interchangeably and refer to Neu5Ac-a2,6-[Neu5Ac-a2,3-Gal-b1,3-GlcNAc-b1,3]-Gal-b1,4-Glc. The terms “LNFP-I”, “lacto-N-fucopentaose I”, “LNFP I”, “LNF I OH type I determinant”, “LNF 1”, “LNF1”, “LNF 1” and “Blood group H antigen pentaose type 1” are used interchangeably and refer to Fuc-α1,2-Gal-b1,3-GlcNAc-b1,3-Gal-b1,4-Glc.

The terms “GalNAc-LNFP-I” and “blood group A antigen hexaose type I” are used interchangeably and refer to GalNAc-a1,3-(Fuc-α1,2)-Gal-b1,3-GlcNAc-b1,3-Gal-b1,4-Glc.

The terms “LNFP-II” and “lacto-N-fucopentaose II” are used interchangeably and refer to Gal-b1,3-(Fuc-a1,4)-GlcNAc-b1,3-Gal-b1,4-Glc.

The terms “LNFP-III” and “lacto-N-fucopentaose III” are used interchangeably and refer to Gal-b1,4-(Fuc-a1,3)-GlcNAc-b1,3-Gal-b1,4-Glc.

The terms “LNFP-V” and “lacto-N-fucopentaose V” are used interchangeably and refer to Gal-b1,3-GlcNAc-b1,3-Gal-b1,4-(Fuc-α1,3)-Glc.

The terms “LNFP-VI”, “LNnFP V” and “lacto-N-neofucopentaose V” are used interchangeably and refer to Gal-b1,4-GlcNAc-b1,3-Gal-b1,4-(Fuc-α1,3)-Glc.

The terms “LNnFP I” and “Lacto-N-neofucopentaose I” are used interchangeably and refer to Fuc-α1,2-Gal-b1,4-GlcNAc-b1,3-Gal-b1,4-Glc.

The terms “LNDFH I”, “Lacto-N-difucohexaose I”, “LNDFH-I”, “LDFH I”, “Leb-lactose” and “Lewis-b hexasaccharide” are used interchangeably and refer to Fuc-α1,2-Gal-b1,3-[Fuc-α1,4]-GlcNAc-b1,3-Gal-b1,4-Glc.

The terms “LNDFH II”, “Lacto-N-difucohexaose II”, “Lewis a-Lewis x” and “LDFH II” are used interchangeably and refer to Fuc-α1,4-(Gal-b1,3)-GlcNAc-b1,3-Gal-b1,4-(Fuc-α1,3)-Glc.

The terms “LNnDFH”, “Lacto-N-neoDiFucohexaose” and “Lewis x hexaose” are used interchangeably and refer to Gal-b1,4-(Fuc-α1,3)-GlcNAc-b1,3-Gal-b1,4-(Fuc-α1,3)-Glc.

The terms “alpha-tetrasaccharide” and “A-tetrasaccharide” are used interchangeably and refer to GalNAc-a1,3-(Fuc-α1,2)-Gal-b1,4-Glc.

The terms “Fuc-α1,2-Gal-b1,3-GlcNAc”, “2-fucosyllacto-N-biose”, “2FLNB”, “2 FLNB”, “2-FLNB” and “2′FLNB” are used interchangeably and refer to a trisaccharide wherein a fucose residue is linked to the galactose residue of lacto-N-biose (LNB, Gal-b1,3-GlcNAc) in an alpha-1,2 linkage.

The term “glycopeptide” as used herein refers to a peptide that contains one or more saccharide groups, being mono-, di-, oligo-, polysaccharides and/or glycans, that is/are covalently attached to the side chains of the amino acid residues of the peptide. Glycopeptides comprise natural glycopeptide antibiotics such as e.g. the glycosylated non-ribosomal peptides produced by a diverse group of soil actinomycetes that target Gram-positive bacteria by binding to the acyl-D-alanyl-D-alanine (D-Ala-D-Ala) terminus of the growing peptidoglycan on the outer surface of the cytoplasmatic membrane, and synthetic glycopeptide antibiotics. The common core of natural glycopeptides is made of a cyclic peptide consisting in 7 amino acids, to which are bound 2 sugars. Examples of glycopeptides comprise vancomycin, teicoplanin, oritavancin, chloroeremomycin, telavancin and dalbavancin.

The terms “glycoprotein” and “glycopolypeptide” are used interchangeably and refer to a polypeptide that contains one or more saccharide groups, being mono-, di-, oligo-, polysaccharides and/or glycans, that is/are covalently attached to the side chains of the amino acid residues of the polypeptide. As used herein, the term “glycolipid” refers to any of the glycolipids which are generally known in the art. Glycolipids (GLs) can be subclassified into Simple (SGLs) and Complex (CGLs)glycolipids. Simple GLs, sometimes called saccharolipids, are two-component (glycosyl and lipid moieties) GLs in which the glycosyl and lipid moieties are directly linked to each other. Examples of SGLs include glycosylated fatty acids, fatty alcohols, carotenoids, hopanoids, sterols or paraconic acids. Bacterially produced SGLs can be classified into rhamnolipids, glucolipids, trehalolipids, other glycosylated (non-trehalose containing) mycolates, trehalose-containing oligosaccharide lipids, glycosylated fatty alcohols, glycosylated macro-lactones and macro-lactams, glycomacrodiolides (glycosylated macrocyclic dilactones), glyco-carotenoids and glyco-terpenoids, and glycosylated hopanoids/sterols. Complex glycolipids (CGLs) are, however, structurally more heterogeneous, as they contain, in addition to the glycosyl and lipid moieties, other residues like for example glycerol (glycoglycerolipids), peptide (glycopeptidolipids), acylated-sphingosine (glycosphingolipids), or other residues (lipopolysaccharides, phenolic glycolipids, nucleoside lipids).

The term “membrane transporter proteins” as used herein refers to proteins that are part of or interact with the cell membrane and control the flow of molecules and information across the cell. The membrane proteins are thus involved in transport, be it import into or export out of the cell.

Such membrane transporter proteins can be porters, P-P-bond-hydrolysis-driven transporters, B-Barrel Porins, auxiliary transport proteins, putative transport proteins and phosphotransfer-driven group translocators as defined by the Transporter Classification Database that is operated and curated by the Saier Lab Bioinformatics Group available via www.tcdb.org and providing a functional and phylogenetic classification of membrane transport proteins This Transporter Classification Database details a comprehensive IUBMB approved classification system for membrane transporter proteins known as the Transporter Classification (TC) system. The TCDB classification searches as described here are defined based on TCDB.org as released on 17th June 2019.

Porters is the collective name of uniporters, symporters, and antiporters that utilize a carrier-mediated process (Saier et al., Nucleic Acids Res. 44 (2016) D372-D379). They belong to the electrochemical potential-driven transporters and are also known as secondary carrier-type facilitators. Membrane transporter proteins are included in this class when they utilize a carrier-mediated process to catalyse uniport when a single species is transported either by facilitated diffusion or in a membrane potential-dependent process if the solute is charged; antiport when two or more species are transported in opposite directions in a tightly coupled process, not coupled to a direct form of energy other than chemiosmotic energy; and/or symport when two or more species are transported together in the same direction in a tightly coupled process, not coupled to a direct form of energy other than chemiosmotic energy, of secondary carriers (Forrest et al., Biochim. Biophys. Acta 1807 (2011) 167-188). These systems are usually stereospecific. Solute:solute countertransport is a characteristic feature of secondary carriers. The dynamic association of porters and enzymes creates functional membrane transport metabolons that channel substrates typically obtained from the extracellular compartment directly into their cellular metabolism (Moraes and Reithmeier, Biochim. Biophys. Acta 1818 (2012), 2687-2706). Solutes that are transported via this porter system include but are not limited to cations, organic anions, inorganic anions, nucleosides, amino acids, polyols, phosphorylated glycolytic intermediates, osmolytes, siderophores. Membrane transporter proteins are included in the class of P-P-bond hydrolysis-driven transporters if they hydrolyse the diphosphate bond of inorganic pyrophosphate, ATP, or another nucleoside triphosphate, to drive the active uptake and/or extrusion of a solute or solutes (Saier et al., Nucleic Acids Res. 44 (2016) D372-D379). The membrane transporter protein may or may not be transiently phosphorylated, but the substrate is not phosphorylated. Substrates that are transported via the class of P-P-bond hydrolysis-driven transporters include but are not limited to cations, heavy metals, beta-glucan, UDP-glucose, lipopolysaccharides, teichoic acid.

The B-Barrel porins membrane transporter proteins form transmembrane pores that usually allow the energy independent passage of solutes across a membrane. The transmembrane portions of these proteins consist exclusively of β-strands which form a β-barrel (Saier et al., Nucleic Acids Res. 44 (2016) D372-D379). These porin-type proteins are found in the outer membranes of Gram-negative bacteria, mitochondria, plastids, and possibly acid-fast Gram-positive bacteria. Solutes that are transported via these β-Barrel porins include but are not limited to nucleosides, raffinose, glucose, beta-glucosides, oligosaccharides.

The auxiliary transport proteins are defined to be proteins that facilitate transport across one or more biological membranes but do not themselves participate directly in transport. These membrane transporter proteins always function in conjunction with one or more established transport systems such as but not limited to outer membrane factors (OMFs), polysaccharide (PST) porters, the ATP-binding cassette (ABC)-type transporters. They may provide a function connected with energy coupling to transport, play a structural role in complex formation, serve a biogenic or stability function or function in regulation (Saier et al., Nucleic Acids Res. 44 (2016) D372-D379). Examples of auxiliary transport proteins include but are not limited to the polysaccharide copolymerase family involved in polysaccharide transport, the membrane fusion protein family involved in bacteriocin and chemical toxin transport. Putative transport protein comprises families which will either be classified elsewhere when the transport function of a member becomes established or will be eliminated from the Transporter Classification system if the proposed transport function is disproven. These families include a member or members for which a transport function has been suggested, but evidence for such a function is not yet compelling (Saier et al., Nucleic Acids Res. 44 (2016) D372-D379). Examples of putative transporters classified in this group under the TCDB system as released on 17th June 2019 include but are not limited to copper transporters.

The phosphotransfer-driven group translocators are also known as the PEP-dependent phosphoryl transfer-driven group translocators of the bacterial phosphoenolpyruvate: sugar phosphotransferase system (PTS). The product of the reaction, derived from extracellular sugar, is a cytoplasmic sugar-phosphate. The enzymatic constituents, catalysing sugar phosphorylation, are superimposed on the transport process in a tightly coupled process. The PTS system is involved in many different aspects comprising in regulation and chemotaxis, biofilm formation, and pathogenesis (Lengeler, J. Mol. Microbiol. Biotechnol. 25 (2015) 79-93; Saier, J. Mol. Microbiol. Biotechnol. 25 (2015) 73-78). Membrane transporter protein families classified within the phosphotransfer-driven group translocators under the TCDB system as released on 17th June 2019 include PTS systems linked to transport of glucose-glucosides, fructose-mannitol, lactose-N,N′-diacetylchitobiose-beta-glucoside, glucitol, galactitol, mannose-fructose-sorbose and ascorbate.

The major facilitator superfamily (MFS) is a superfamily of membrane transporter proteins catalysing uniport, solute: cation (H+, but seldom Na+) symport and/or solute: H+ or solute: solute antiport. Most are of 400-600 amino acyl residues in length and possess either 12, 14, or occasionally, 24 transmembrane a-helical spanners (TMSs) as defined by the Transporter Classification Database operated by the Saier Lab Bioinformatics Group (www.tcdb.org).

“SET” or “Sugar Efflux Transporter” as used herein refers to membrane proteins of the SET family which are proteins with InterPRO domain IPR004750 and/or are proteins that belong to the eggNOGv4.5 family ENOG410XTE9. Identification of the InterPro domain can be done by using the online tool on https://www.ebi.ac.uk/interpro/or a standalone version of InterProScan (https://www.ebi.ac.uk/interpro/download.html) using the default values. Identification of the orthology family in eggNOGv4.5 can be done using the online version or a standalone version of eggNOG-mapperv1 (http://eggnogdb.embl.de/#/app/home).

The term “Siderophore” as used herein is referring to the secondary metabolite of various microorganisms which are mainly ferric ion specific chelators. These molecules have been classified as catecholate, hydroxamate, carboxylate and mixed types. Siderophores are in general synthesized by a nonribosomal peptide synthetase (NRPS) dependent pathway or an NRPS independent pathway (NIS). The most important precursor in NRPS-dependent siderophore biosynthetic pathway is chorismate. 2, 3-DHBA could be formed from chorismate by a three-step reaction catalysed by isochorismate synthase, isochorismatase, and 2, 3-dihydroxybenzoate-2, 3-dehydrogenase. Siderophores can also be formed from salicylate which is formed from isochorismate by isochorismate pyruvate lyase. When ornithine is used as precursor for siderophores, biosynthesis depends on the hydroxylation of ornithine catalysed by L-ornithine N5-monooxygenase. In the NIS pathway, an important step in siderophore biosynthesis is N (6)-hydroxylysine synthase.

A transporter is needed to export the siderophore outside the cell. Four superfamilies of membrane proteins are identified so far in this process: the major facilitator superfamily (MFS); the Multidrug/Oligosaccharidyl-lipid/Polysaccharide Flippase Superfamily (MOP); the resistance, nodulation and cell division superfamily (RND); and the ABC superfamily. In general, the genes involved in siderophore export are clustered together with the siderophore biosynthesis genes. The term “siderophore exporter” as used herein refers to such transporters needed to export the siderophore outside of the cell.

The ATP-binding cassette (ABC) superfamily contains both uptake and efflux transport systems, and the members of these two groups generally cluster loosely together. ATP hydrolysis without protein phosphorylation energizes transport. There are dozens of families within the ABC superfamily, and family generally correlates with substrate specificity. Members are classified according to class 3.A.1 as defined by the Transporter Classification Database operated by the Saier Lab Bioinformatics Group available via www.tcdb.org and providing a functional and phylogenetic classification of membrane transporter proteins.

It should be understood for those skilled in the art that for the databases used herein, comprising eggnogdb 4.5.1 (released September 2016) and InterPro 75.0 (released 4th July 2019), the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

A ‘fucosylation pathway’ as used herein is a biochemical pathway comprising at least one of the enzymes and their respective genes chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase combined with a fucosyltransferase leading to a 1,2; a 1,3; a 1,4 and/or a 1,6 fucosylated oligosaccharides.

A ‘sialylation pathway’ is a biochemical pathway comprising at least one of the enzymes and their respective genes chosen from the list comprising N-acylglucosamine 2-epimerase, UDP-N-acetylglucosamine 2-epimerase, N-acetylmannosamine-6-phosphate 2-epimerase, UDP-GlcNAc 2-epimerase/kinase hydrolyzing, N-acylneuraminate-9-phosphate synthase, phosphatase, N-acetylneuraminate synthase, N-acylneuraminate cytidylyltransferase and sialic acid transporter combined with a sialyltransferase leading to a 2,3; a 2,6 and/or a 2,8 sialylated oligosaccharides.

A ‘galactosylation pathway’ as used herein is a biochemical pathway comprising at least one of the enzymes and their respective genes chosen from the list comprising galactose-1-epimerase, galactokinase, glucokinase, galactose-1-phosphate uridylyltransferase, UDP-glucose 4-epimerase, glucose-1-phosphate uridylyltransferase, phosphoglucomutase combined with a galactosyltransferase leading to a galactosylated compound comprising a mono-, di-, or oligosaccharide having an alpha or beta bound galactose on any one or more of the 2, 3, 4 and 6 hydroxyl group of said mono-, di-, or oligosaccharide.

An ‘N-acetylglucosaminylation pathway’ as used herein is a biochemical pathway comprising at least one of the enzymes and their respective genes chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase, glucosamine-1-phosphate acetyltransferase combined with a glycosyltransferase leading to a GlcNAc-modified compound comprising a mono-, di-, or oligosaccharide having an alpha or beta bound N-acetylglucosamine (GlcNAc) on any one or more of the 3, 4 and 6 hydroxyl group of said mono-, di- or oligosaccharide.

An ‘N-acetylgalactosaminylation pathway’ as used herein is a biochemical pathway comprising at least one of the enzymes and their respective genes chosen from the list comprising L-glutamine-D-fructose-6-phosphate aminotransferase, phosphoglucosamine mutase, N-acetylglucosamine 1-phosphate uridylyltransferase, glucosamine-1-phosphate acetyltransferase, UDP-N-acetylglucosamine 4-epimerase, UDP-glucose 4-epimerase, N-acetylgalactosamine kinase and/or UDP-N-acetylgalactosamine pyrophosphorylase combined with a glycosyltransferase leading to a GalNAc-modified compound comprising a mono-, di- or oligosaccharide having an alpha or beta bound N-acetylgalactosamine on said mono-, di- or oligosaccharide.

A ‘mannosylation pathway’ as used herein is a biochemical pathway comprising at least one of the enzymes and their respective genes chosen from the list comprising mannose-6-phosphate isomerase, phosphomannomutase and/or mannose-1-phosphate guanylyltransferase combined with a glycosyltransferase leading to a mannosylated compound comprising a mono-, di- or oligosaccharide having an alpha or beta bound mannose on said mono-, di- or oligosaccharide.

An ‘N-acetylmannosaminylation pathway’ as used herein is a biochemical pathway comprising at least one of the enzymes and their respective genes chosen from the list comprising L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, glucosamine-1-phosphate acetyltransferase, UDP-GlcNAc 2-epimerase and/or ManNAc kinase combined with a glycosyltransferase leading to a ManNAc-modified compound comprising a mono-, di- or oligosaccharide having an alpha or beta bound N-acetylmannosamine on said mono-, di- or oligosaccharide.

The term “enabled efflux” means to introduce the activity of transport of a solute over the cytoplasm membrane and/or the cell wall. Said transport may be enabled by introducing and/or increasing the expression of a membrane transporter protein as described in the present invention. The term “enhanced efflux” means to improve the activity of transport of a solute over the cytoplasm membrane and/or the cell wall. Transport of a solute over the cytoplasm membrane and/or cell wall may be enhanced by introducing and/or increasing the expression of a membrane transporter protein as described in the present invention. “Expression” of a membrane transporter protein is defined as “overexpression” of the gene encoding said membrane transporter protein in the case said gene is an endogenous gene or “expression” in the case the gene encoding said membrane transporter protein is a heterologous gene that is not present in the wild type strain or cell.

The terms “acetyl-coenzyme A synthetase”, “acs”, “acetyl-CoA synthetase”, “AcCoA synthetase”, “acetate—CoA ligase”, “acyl-activating enzyme” and “yfaC” are used interchangeably and refer to an enzyme that catalyses the conversion of acetate into acetyl-coezyme A (AcCoA) in an ATP-dependent reaction.

The terms “pyruvate dehydrogenase”, “pyruvate oxidase”, “POX”, “poxB” and “pyruvate: ubiquinone-8 oxidoreductase” are used interchangeably and refer to an enzyme that catalyses the oxidative decarboxylation of pyruvate to produce acetate and CO2.

The terms “lactate dehydrogenase”, “D-lactate dehydrogenase”, “IdhA”, “hsll”, “htpH”, “D-LDH”, “fermentative lactate dehydrogenase” and “D-specific 2-hydroxyacid dehydrogenase” are used interchangeably and refer to an enzyme that catalyses the conversion of lactate into pyruvate hereby generating NADH.

As used herein, the term “cell productivity index (CPI)” refers to the mass of the product produced by the cells divided by the mass of the cells produced in the culture.

The term “purified” refers to material that is substantially or essentially free from components which interfere with the activity of the biological molecule. For cells, saccharides, nucleic acids, polypeptides, peptides, glycoproteins, glycopeptides, lipids and glycolipids the term “purified” refers to material that is substantially or essentially free from components which normally accompany the material as found in its native state. Typically, purified saccharides, oligosaccharides, peptides, glycopeptides, proteins, glycoproteins, lipids, glycolipids or nucleic acids of the invention are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For di- and oligosaccharides, purity can be determined using methods such as but not limited to thin layer chromatography, gas chromatography, NMR, HPLC, capillary electrophoresis or mass spectroscopy.

The term “cultivation” refers to the culture medium wherein the cell is cultivated or fermented, the cell itself, and the Fuc-a1,2-Gal-b1,3-GlcNAc-(Rn) that is produced by the cell in whole broth, i.e. inside (intracellularly) as well as outside (extracellularly) of the cell.

The term “precursor” as used herein refers to substances which are taken up and/or synthetized by the cell for the specific production of Fuc-a1,2-Gal-b1,3-GlcNAc-(Rn) according to the present invention. In this sense a precursor can be an acceptor as defined herein, but can also be another substance, metabolite, which is first modified within the cell as part of the biochemical synthesis route of Fuc-a1,2-Gal-b1,3-GlcNAc-(Rn). Examples of such precursors comprise the acceptors as defined herein, and/or glucose, galactose, fructose, glycerol, sialic acid, fucose, mannose, maltose, sucrose, lactose, dihydroxyacetone, glucosamine, N-acetyl-glucosamine, mannosamine, N-acetyl-mannosamine, galactosamine, N-acetylgalactosamine, phosphorylated sugars like e.g. but not limited to glucose-1-phosphate, galactose-1-phosphate, glucose-6-phosphate, fructose-6-phosphate, fructose-1,6-bisphosphate, mannose-6-phosphate, mannose-1-phosphate, glycerol-3-phosphate, glyceraldehyde-3-phosphate, dihydroxyacetone-phosphate, glucosamine-6-phosphate, N-acetyl-glucosamine-6-phosphate, N-acetylmannosamine-6-phosphate, N-acetylglucosamine-1-phosphate, N-acetyl-neuraminic acid-9-phosphate and/or nucleotide-activated sugars as defined herein like e.g. UDP-glucose, UDP-galactose, UDP-N-acetylglucosamine, CMP-sialic acid, GDP-mannose, GDP-4-dehydro-6-deoxy-a-D-mannose, GDP-fucose.

Optionally, the cell is transformed to comprise and to express at least one nucleic acid sequence encoding a protein selected from the group consisting of lactose transporter, fucose transporter, transporter for a nucleotide-activated sugar wherein said transporter internalizes a to the medium added precursor for the production of Fuc-a1,2-Gal-b1,3-GlcNAc-(Rn) of present invention.

The term “acceptor” as used herein refers to a mono-, di- or oligosaccharide, a protein, a glycoprotein, a peptide, a glycopeptide, a lipid or glycolipid which can be modified by a glycosyltransferase. Examples of such acceptors comprise glucose, galactose, fructose, glycerol, sialic acid, fucose, mannose, maltose, sucrose, lactose, lacto-N-biose (LNB), lacto-N-triose, lacto-N-tetraose (LNT), lacto-N-neotetraose (LNnT), N-acetyl-lactosamine (LacNAc), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, galactosyllactose, and oligosaccharide containing 1 or more N-acetyllactosamine units and/or 1 or more lacto-N-biose units or an intermediate into oligosaccharide, fucosylated and sialylated versions thereof, peptides, polypeptides, lipids, sphingolipids, cerebrosides, ceramide lipids, phosphatidylinositol lipids and glycosylated versions of peptides, polypeptides, lipids, sphingolipids, cerebrosides, ceramide lipids, phosphatidylinositol lipids.

### DETAILED DESCRIPTION OF THE INVENTION

According to a first aspect, the present invention provides a method for the production of a compound comprising a structure of Formula I, II or III:

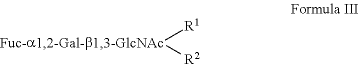

Fuc-α1,2-Gal-β1,3-GlcNAc Formula I

Fuc-α1,2-Gal-β1,3-GlcNAc-R¹ Formula II

wherein:
R¹ is a monosaccharide, disaccharide, oligosaccharide, protein, glycoprotein, peptide, glycopeptide, lipid or glycolipid; and, when present, R² is a monosaccharide, disaccharide or oligosaccharide. The method comprises the steps of:
i. providing a cell, preferably a single cell, that expresses an alpha-1,2-fucosyltransferase having galactoside alpha-1,2-fucosyltransferase activity on the galactose residue of Gal-b1,3-GlcNAc (LNB, Lacto-N-biose) as described herein, and
ii. cultivating and/or incubating said cell under conditions permissive to express said alpha-1,2-fucosyltransferase and to produce said compound comprising a structure of Formula I, II or III,
iii. preferably, separating said compound comprising a structure of Formula I, II or III from the cultivation.

In a second aspect, the present invention provides a cell genetically engineered for the production of said compound comprising a structure of Formula I, II or III as described herein. In the context of the invention, said compound comprising a structure of Formula I, II or III as described herein preferably does not occur in the wild type progenitor of said cell.

A genetically engineered cell, preferably a single cell, is provided which expresses an alpha-1,2-fucosyltransferase that has galactoside alpha-1,2-fucosyltransferase activity on the galactose residue of Gal-b1,3-GlcNAc (LNB, lacto-N-biose).

According to the invention, said method for the production of said compound comprising a structure of Formula I, II or III can make use of a non-genetically engineered cell or can make use of a genetically engineered cell as disclosed herein.

In the context of the invention, it should be understood that said compound comprising a structure of Formula I, II or III is preferably produced intracellularly. The skilled person will further understand that a fraction or substantially all of said produced compound comprising a structure of Formula I, II or III remains intracellularly and/or is excreted outside the cell either passively or through active transport. Throughout the application, unless explicitly stated otherwise, a “genetically modified cell” or “genetically engineered cell” preferably means a cell which is genetically modified or genetically engineered, respectively, for the production of said compound comprising a structure of Formula I, II or III according to the invention.

In the context of the invention, said term “compound comprising a structure of Formula I, II or III” refers to the trisaccharide Fuc-a1,2-Gal-b1,3-GlcNAc as well as to a compound wherein the N-acetylglucosamine (GlcNAc) residue of said trisaccharide Fuc-a1,2-Gal-b1,3-GlcNAc is (a) linked to one R group wherein said R is a monosaccharide, disaccharide, oligosaccharide, protein, glycoprotein, peptide, glycopeptide, lipid or glycolipid or (b) linked to two R groups, wherein one of said two R groups is a monosaccharide, disaccharide, oligosaccharide, protein, glycoprotein, peptide, glycopeptide, lipid or glycolipid and the other one of said two R groups is a monosaccharide, a disaccharide or an oligosaccharide. Preferably, each of said two R groups is a monosaccharide, a disaccharide or an oligosaccharide.

In a preferred embodiment of the method and/or cell of the present invention, said compound comprising a structure of Formula I, II or III is an oligosaccharide, preferably said oligosaccharide is a mammalian milk oligosaccharide (MMO) as defined herein, more preferably said oligosaccharide is a human milk oligosaccharide (HMO).

In another preferred embodiment of the method and/or cell of the present invention, said compound comprising a structure of Formula I, II or III is a charged oligosaccharide. In a more preferred embodiment of the method and/or cell of the present invention, said compound comprising a structure of Formula I, II or III is a sialylated oligosaccharide. In an alternative preferred embodiment of the method and/or cell of the present invention, said compound comprising a structure of Formula I, II or III is a neutral oligosaccharide.

In another preferred embodiment of the method and/or cell of the present invention, said compound comprising a structure of Formula I, II or III is the trisaccharide Fuc-a1,2-Gal-b1,3-GlcNAc, also known as 2′-fucosyllacto-N-biose or 2′FLNB.

In another preferred embodiment of the method and/or cell of the present invention, said compound comprising a structure of Formula I, II or III is a compound comprising one R group chosen from the list comprising a monosaccharide, disaccharide, oligosaccharide, protein, glycoprotein, peptide, glycopeptide, lipid or glycolipid wherein the GlcNAc residue of said Fuc-a1,2-Gal-b1,3-GlcNAc is linked to said R group via an alpha- or a beta-glycosidic linkage.

In another preferred embodiment of the method and/or cell of the present invention, said compound comprising a structure of Formula I, II or III is a compound comprising two R groups, wherein one of said two R groups is a monosaccharide, disaccharide, oligosaccharide, protein, glycoprotein, peptide, glycopeptide, lipid or glycolipid and the other one of said two R groups is a monosaccharide, a disaccharide or an oligosaccharide wherein the GlcNAc residue of said Fuc-a1,2-Gal-b1,3-GlcNAc is bound via a glycosidic linkage with each of said R groups. Herein, said GlcNAc residue can be bound to a first R group with an alpha-glycosidic linkage and to the second R group with a beta-glycosidic linkage. Alternatively, said GlcNAc residue can be bound to both R groups with beta-glycosidic linkages.

In another preferred embodiment of the method and/or cell of the present invention, said compound comprising a structure of Formula I, II or III is an oligosaccharide comprising two R groups wherein each of said R groups is a monosaccharide, a disaccharide or an oligosaccharide wherein the GlcNAc residue of said Fuc-a1,2-Gal-b1,3-GlcNAc is bound via a glycosidic linkage with each of said R groups. Herein, said GlcNAc residue can be bound to a first R group with an alpha-glycosidic linkage and to the second R group with a beta-glycosidic linkage. Alternatively, said GlcNAc residue can be bound to both R groups with beta-glycosidic linkages.

In another preferred embodiment of the method and/or cell of the present invention, said compound comprising a structure of Formula I, II or III is Fuc-a1,2-Gal-b1,3-GlcNAc-b1,3-R comprising one R group wherein the N-acetylglucosamine (GlcNAc) residue of the trisaccharide Fuc-a1,2-Gal-b1,3-GlcNAc is bound to said R group via a beta-1,3 glycosidic linkage wherein said R group is chosen from the list comprising a monosaccharide, a disaccharide or an oligosaccharide. In a more preferred embodiment, said compound comprising a structure of Formula I, II or III is Fuc-a1, 2-Gal-b1,3-GlcNAc-b1,3-Gal-R comprising one R group chosen from the list comprising a monosaccharide, a disaccharide or an oligosaccharide. In an even more preferred embodiment, said compound comprising a structure of Formula I, II or III is lacto-N-fucopentaose I (LNFP-I, Fuc-a1,2-Gal-b1,3-GlcNAc-b1,3-Gal-b1,4-Glc) wherein the trisaccharide Fuc-a 1,2-Gal-b1,3-GlcNAc is linked via its GlcNAc residue in a beta-1,3 linkage to the galactose residue of lactose (Gal-b1,4-Glc).

In another preferred embodiment of the method and/or cell of the present invention, said compound comprising a structure of Formula I, II or III is Fuc-a1,2-Gal-b1,3-GlcNAc-b1,6-R comprising one R group wherein the GlcNAc residue of the trisaccharide Fuc-a 1,2-Gal-b1,3-GlcNAc is bound to said R group via a beta-1,6 glycosidic linkage wherein said R group is chosen from the list comprising a monosaccharide, a disaccharide or an oligosaccharide.

In another preferred embodiment of the method and/or cell of the present invention, said compound comprising a structure of Formula I, II or III is LNDFH I (Fuc-a1,2-Gal-b1,3-[Fuc-a1,4]-GlcNAc-b1,3-Gal-b1,4-Glc).

In another preferred embodiment of the method and/or cell of the present invention, said compound comprising a structure of Formula I, II or III is an oligosaccharide chosen from the list comprising Fuc-a1,2-Gal-b1,3-GlcNAc-b1,3-Gal; Fuc-a1,2-Gal-b1,3-GlcNAc-b1,3-Gal-b1,4-GalNAc; Fuc-a1,2-Gal-b1,3-GlcNAc-b1,3-Gal-b1,4-GlcNAc-b1,6-[Gal-b1,4-GlcNAc-b1,3]-GalNAc; Fuc-a1,2-Gal-b1,3-GlcNAc-b1,3-Gal-b1,3-GalNAc; Fuc-a1,2-Gal-b1,3-GlcNAc-b1,3-GalNAc; Fuc-a1,2-Gal-b1,3-GlcNAc-b1,3-[NeuAc-a2,6]-GalNAc; NeuAc-a2,3-Gal-b1,4-[Fuc-a1,3]-GlcNAc-b1,6-[Fuc-a1,2-Gal-b1,3-GlcNAc-b1,3]-GalNAc; Fuc-α1,2-Gal-b1,3-GlcNAc-b1,3-[Fuca1,2]-Gal-b1,3-[Fuc-a1,2-Gal-b1,4-GlcNAc-b1,6]-GalNAc; Fuc-a1,3-[Gal-b1,4]-GlcNAc-b1,3-Gal-b1,4-GlcNAc-b1,6-[Fuc-a1,2-Gal-b1,3-GlcNAc-b1,3]-GalNAc; Fuc-a1,2-Gal-b1,3-GlcNAc-b1,3-[NeuAc-a2,3-Gal-b1,4-GlcNAc-b1,6]-Gal-b1,3-GalNAc; Fuca1,2-Gal-b1,4-[Fuca1,3]-GlcNAc-b1,6-[Fuc-a1, 2-Gal-b1,3-GlcNAc-b1,3]-GalNAc; Fuc-a1,2-Gal-b1,3-GlcNAc-b1,3-[Fuca1,2-Gal-b1,4-GlcNAc-b1,6]-GalNAc; Fuc-a1,2-Gal-b1,3-GlcNAc-b1,3-Gal-b1,3-[Fuc-a1,2-[GalNAc-a1,3]-Gal-b1,4-GlcNAc-b1,6]-GalNAc; Gal-b1,4-GlcNAc-b1,3-Gal-b1,4-GlcNAc-b1,6-[Fuc-a1,2-Gal1,3-GlcNAc-b1,3]-GalNAc; Fuc-a1,2-Gal-b1,3-GlcNAc-b1,3-[Fuc-a1,3-[Gal-b1,4]-GlcNAc-b1,6]-Gal-b1,4-Glc; Fuc-a1,2-Gal-b1,3-GlcNAc-b1,3-[Fuc-a1,2-Gal-b1,4-GlcNAc-b1,6]-Gal-b1,4-GlcNAc-b1,3-[NeuAc-a2,6]-Gal-b1,3-[Fuc-a1,2-Gal-b1,4-GlcNAc-b1,6]-GalNAc; Fuc-a1, 2-Gal-b1,3-GlcNAc-b1,3-Gal-b1,3-[Gal-b1,4-GlcNAc-b1,6]-GalNAc; Fuc-a1,2-Gal-b1,3-GlcNAc-b1,3-[Gal-b1,4-[HSO3 (-6)]GlcNAc-b1,6]-GalNAc; Fuc-a1,2-Gal-b1,3-GlcNAc-b1,3-Gal-b1,4-GlcNAc-b1,6-[Gal-b1,3]-GalNAc; Fuc-a1,4-[HSO3 (-3) Gal-b1,3]-GlcNAc-b1,3-Gal-b1,4-GlcNAc-b1,6-[Fuc-a1,2-Gal-b1,3-GlcNAc-b1,3]-GalNAc; Fuc-a1,2-Gal-b1,3-GlcNAc-b1,3-[GlcNAc-b1,6]-Gal-b1,4-GlcNAc-b1,3-[NeuAc-a2,6]-Gal-b1,3-[Fuc-a1,2-Gal-b1,4-GlcNAc-b1,6]-GalNAc; Fuc-a1,2-Gal-b1,3-GlcNAc-b1,3-[NeuAc-a2,3-Gal-b1,4-GlcNAc-b1,6]-GalNAc; Fuc-a1,3-[HSO3 (-3) Gal-b1,4]-GlcNAc-b1,6-[Fuc-a1,2-Gal-b1,3-GlcNAc-b1,3]-GalNAc; Fuc-a1,2-Gal-b1,3-GlcNAc-b1,3-Gal-b1,4-GlcNAc-b1,6-[NeuAc-a2,3-Gal-b1,3]-GalNAc; Fuc-a1,2-Gal-b1,3-GlcNAc-b1,3-[Fuc-a1,2-Gal-b1,4-GlcNAc-b1,6]-Gal-b1,3-GalNAc; Fuc-a1, 2-Gal-b1,3-GlcNAc-b1,3-[Gal-b1,4-GlcNAc-b1,6]-GalNAc; Fuc-a1,2-Gal-b1,3-GlcNAc-b1,3-[HSO3 (-3) Gal-b1,4-GlcNAc-b1,6]-GalNAc; Fuc-a1,2-Gal-b1,3-GlcNAc-b1,6-[Fuc-a1,2-Gal-b1,3]-GalNAc; Fuc-a1,2-Gal-b1,3-GlcNAc-b1,3-[Gal-b1,4-GlcNAc-b1,6]-Gal-b1,3-[Gal-b1,4-GlcNAc-b1,6]-GalNAc; Fuc-a1,2-Gal-b1,3-[Fuc-a1,4]-GlcNAc-b1,3-Gal-b1,3-GalNAc; Fuc-a1,2-Gal-b1,3-[Fuc-a1,4]-GlcNAc-b1,3-Gal-b1,4-GlcNAc-b1,3-Gal-b1,4-GlcNAc-b1,6-[NeuAc-a2,3-Gal-b1,3]-GalNAc; Fuc-a1,2-Gal-b1,3-[Fuc-a1,4]-GlcNAc-b1,3-Gal-b1,3-[Gal-b1,4-GlcNAc-b1,6]-GalNAc; Fuc-a1,2-Gal-b1,3-[Fuc-a1,4]-GlcNAc-b1,6-[Gal-b1,3]-GalNAc; Fuc-a1, 2-Gal-b1,3-[Fuc-a1,4]-GlcNAc-b1,3-[Fuc-a 1,2]-Gal-b1,4-GlcNAc-b1,6-[GlcNAc-b1,3]-GalNAc; Fuc-a 1,2-Gal-b1,3-[Fuc-a1,4]-GlcNAc-b1,3-Gal-b1,4-GlcNAc-b1,6-[Fuc-a1,2-Gal-b1,3]-GalNAc; Fuc-a1, 2-Gal-b1,3-[Fuc-a1,4]-GlcNAc-b1,3-[Gal-b1,4-GlcNAc-b1,6]-Gal-b1,4-[Fuc-a1,3]-GlcNAc-b1,6-[NeuAc-a2,3-Gal-b1,3]-GalNAc; Fuc-a1,2-Gal-b1,3-[NeuAc-a2,6]-GlcNAc-b1,3-Gal-b1,3-Glc; Fuc-a1,2-Gal-b1,3-[Fuc-a1,4]-GlcNAc-b1,3-Gal-b1,4-GlcNAc-b1,6-[GlcNAc-b1,3]-GalNAc; Fuc-a1,2-Gal-b1,3-[Fuc-a1,4]-GlcNAc-b1,3-[Fuc-a1,3-[Gal-b1,4]-GlcNAc-b1,6]-Gal-b1,4-GlcNAc-b1,6-[NeuAc-a2,3-Gal-b1,3]-GalNAc and Fuc-α1,2-Gal-b1,3-[Fuc-α1,4]-GlcNAc-b1,3-Gal-b1,4-GlcNAc-b1,6-[Gal-b1,4-GlcNAc-b1,3]-GalNAc.

In the scope of the present invention, the wording “permissive conditions to produce said compound comprising a structure of Formula I, II or III” is to be understood to be conditions relating to physical or chemical parameters including but not limited to temperature, pH, pressure, osmotic pressure and product/precursor/acceptor concentration.

In a particular embodiment, such conditions may include a temperature-range of 30+/−20 degrees centigrade, a pH-range of 7+/−3.

In a preferred embodiment of the method, the permissive conditions comprise use of a culture medium comprising at least one precursor and/or acceptor as defined herein for the production of said compound comprising a structure of Formula I, II or III. In an alternative and/or additional preferred embodiment of the method, the permissive conditions comprise adding to the culture medium at least one precursor and/or acceptor feed for the production of said compound comprising a structure of Formula I, II or III.

According to a preferred embodiment of the present invention, the cell is modified with one or more expression modules. Said expression modules are also known as transcriptional units and comprise polynucleotides for expression of recombinant genes including coding gene sequences and appropriate transcriptional and/or translational control signals that are operably linked to the coding genes. Said control signals comprise promoter sequences, untranslated regions, ribosome binding sites, terminator sequences. Said expression modules can contain elements for expression of one single recombinant gene but can also contain elements for expression of more recombinant genes or can be organized in an operon structure for integrated expression of two or more recombinant genes. Said polynucleotides may be produced by recombinant DNA technology using techniques well-known in the art. Methods which are well known to those skilled in the art to construct expression modules include, for example, in vitro recombinant DNA techniques, synthetic techniques, and in vivo genetic recombination. See, for example, the techniques described in Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates).

The expression of each of said expression modules can be constitutive or is created by a natural or chemical inducer. As used herein, constitutive expression should be understood as expression of a gene that is transcribed continuously in an organism. Expression that is created by a natural inducer should be understood as a facultative or regulatory expression of a gene that is only expressed upon a certain natural condition of the host (e.g. organism being in labour, or during lactation), as a response to an environmental change (e.g. including but not limited to hormone, heat, cold, pH shifts, light, oxidative or osmotic stress/signalling), or dependent on the position of the developmental stage or the cell cycle of said host cell including but not limited to apoptosis and autophagy. Expression that is created by a chemical inducer should be understood as a facultative or regulatory expression of a gene that is only expressed upon sensing of external chemicals (e.g. IPTG, arabinose, lactose, allo-lactose, rhamnose or fucose) via an inducible promoter or via a genetic circuit that either induces or represses the transcription or translation of said polynucleotide to a polypeptide.

The expression modules can be integrated in the genome of said cell or can be presented to said cell on a vector. Said vector can be present in the form of a plasmid, cosmid, phage, liposome, or virus, which is to be stably transformed/transfected into said metabolically engineered cell. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. These vectors may contain selection markers such as but not limited to antibiotic markers, auxotrophic markers, toxin-antitoxin markers, RNA sense/antisense markers. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., see above. For recombinant production, cells can be genetically engineered to incorporate expression systems or portions thereof or polynucleotides of the invention. Introduction of a polynucleotide into the cell can be effected by methods described in many standard laboratory manuals, such as Davis et al., Basic Methods in Molecular Biology, (1986), and Sambrook et al., 1989, supra.

As used herein an expression module comprises polynucleotides for expression of at least one recombinant gene. Said recombinant gene is involved in the expression of a polypeptide acting in the production of a compound comprising a structure of Formula I, II or III; or said recombinant gene is linked to other pathways in said host cell that are not involved in the production of a compound comprising a structure of Formula I, II or III. Said recombinant genes encode endogenous proteins with a modified expression or activity, preferably said endogenous proteins are overexpressed; or said recombinant genes encode heterologous proteins that are heterogeneously introduced and expressed in said modified cell, preferably overexpressed. The endogenous proteins can have a modified expression in the cell which also expresses a heterologous protein.

In a preferred embodiment of the method and/or cell according to the invention, the cell is capable to produce a compound comprising a structure of Formula IV, V or VI:

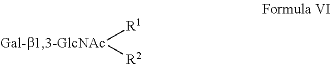

Gal-β1,3-GlcNAc Formula IV

Gal-β1,3-GlcNAc-R¹ Formula V
wherein:
R¹ is a monosaccharide, disaccharide, oligosaccharide, protein, glycoprotein, peptide, glycopeptide, lipid or glycolipid; and, when present, R² is a monosaccharide, disaccharide or oligosaccharide.

In a more preferred embodiment of the method and/or cell according to the invention, the cell is capable to produce Gal-b1,3-GlcNAc or lacto-N-biose (LNB). LNB production in a cell can be obtained by expression and/or over-expression of an N-acetylglucosamine beta-1,3-galactosyltransferase gene which transfers a galactose (Gal) residue from UDP-Gal to an N-acetylglucosamine (GlcNAc) moiety in a beta-1,3-linkage. The GlcNAc and UDP-Gal that are needed in said reaction can be fed to the cultivation and/or can be produced by the metabolism of the cell and/or can be provided by enzymes expressed in the cell.

In an alternative and/or additional preferred embodiment of the method and/or cell according to the invention, the cell is capable to produce GlcNAc-b1,3-Gal-b1,4-Glc or lacto-N-triose (LN3). LN3 production in a cell can be obtained by over-expression of a galactoside beta-1,3-N-acetylglucosaminyltransferase gene which transfers a GlcNAc residue from UDP-GlcNAc to lactose to form LN3. The UDP-GlcNAc and lactose that are needed in said reaction can be fed to the cultivation and/or can be produced by the metabolism of the cell and/or can be provided by enzymes expressed in the cell.

In an alternative and/or additional preferred embodiment of the method and/or cell according to the invention, the cell is capable to produce Gal-b1,3-GlcNAc-b1,3-Gal-b1,4-Glc or lacto-N-tetraose (LNT). LNT production in a cell can be obtained by over-expression of a galactoside beta-1,3-N-acetylglucosaminyltransferase gene and an N-acetylglucosamine beta-1,3-galactosyltransferase gene which respectively transfers a GlcNAc residue from UDP-GlcNAc to lactose to form LN3 and that transfers a Gal residue from UDP-Gal to LN3 to form LNT. The UDP-GlcNAc, UDP-Gal and lactose that is/are needed in said reaction can be fed to the cultivation and/or can be produced by the metabolism of the cell and/or can be provided by enzymes expressed in the cell.

A cell producing GlcNAc can express a phosphatase as e.g. chosen from the list comprising any one or more of the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonas putida, DOG1 from S. cerevisiae and AraL from Bacillus subtilis as described in WO18122225, which dephosphorylates GlcNAc-6-phosphate (GlcNAc-6P) to GlcNAc. Preferably, the cell is modified to produce GlcNAc. More preferably, the cell is modified for enhanced GlcNAc production. Said modification can be any one or more chosen from the group comprising knock-out of N-acetylglucosamine-6-phosphate deacetylase, knock-out of glucosamine-6-phosphate deaminase and over-expression of any one or more of the genes comprising L-glutamine-D-fructose-6-phosphate aminotransferase, glucosamine 6-phosphate N-acetyltransferase and a phosphatase as described in WO18122225.

A cell producing UDP-Gal can express an enzyme converting, e.g. UDP-glucose, to UDP-Gal. This enzyme may be, e.g., the UDP-glucose 4-epimerase GalE like as known from several species including Homo sapiens, Escherichia coli, and Rattus norvegicus. Preferably, the cell is modified to produce UDP-Gal. More preferably, the cell is modified for enhanced UDP-Gal production. Said modification can be any one or more chosen from the group comprising knock-out of an bifunctional 5′-nucleotidase/UDP-sugar hydrolase encoding gene, knock-out of a galactose-1-phosphate uridylyltransferase encoding gene and over-expression of an UDP-glucose 4-epimerase encoding gene.

A cell producing UDP-GlcNAc can express enzymes converting, e.g. GlcNAc, which is to be added to the cell, to UDP-GlcNAc. These enzymes may be an N-acetyl-D-glucosamine kinase, an N-acetylglucosamine-6-phosphate deacetylase, a phosphoglucosamine mutase, and an N-acetylglucosamine-1-phosphate uridylyltransferase/glucosamine-1-phosphate acetyltransferase from several species including Homo sapiens, and Escherichia coli. Preferably, the cell is modified to produce UDP-GlcNAc. More preferably, the cell is modified for enhanced UDP-GlcNAc production. Said modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridylyltransferase/glucosamine-1-phosphate acetyltransferase.

A cell producing lactose can express an beta-1,4-galactosyltransferase which transfers a Gal residue from UDP-Gal to glucose in a beta-1,4-linkage, wherein said glucose can be fed to the cultivation and/or can be produced by the metabolism of the cell and/or can be provided by enzymes expressed in the cell like e.g. an UDP-glucose 4-epimerase. Preferably, the cell using lactose for LN3, LNT and/or derivatives thereof does not have an active galactosidase like e.g. lacZ that degrades lactose into glucose and galactose.

In a preferred embodiment of the method and/or cell according to the invention, the cell resists the phenomenon of lactose killing when grown in an environment in which lactose is combined with one or more other carbon source(s). With the term “lactose killing” is meant the hampered growth of the cell in medium in which lactose is present together with another carbon source. In a preferred embodiment, the cell is genetically modified such that it retains at least 50% of the lactose influx without undergoing lactose killing, even at high lactose concentrations, as is described in WO 2016/075243. Said genetic modification comprises expression and/or over-expression of an exogenous and/or an endogenous lactose transporter gene by a heterologous promoter that does not lead to a lactose killing phenotype and/or modification of the codon usage of the lactose transporter to create an altered expression of said lactose transporter that does not lead to a lactose killing phenotype. The content of WO 2016/075243 in this regard is incorporated by reference. In the context of the present invention, lactose is preferably taken up by a cell as disclosed herein, wherein said lactose is further glycosylated by a glycosyltransferase as disclosed herein to synthesize an MMO, preferably an HMO.

GDP-fucose can be provided by an enzyme expressed in the cell or by the metabolism of the cell. Such cell producing GDP-fucose can express an enzyme converting, e.g., fucose, which is to be added to the cell, to GDP-fucose. This enzyme may be, e.g., a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus.

Preferably, the cell is modified to produce GDP-fucose. More preferably, the cell is modified for enhanced GDP-fucose production. Said modification can be any one or more chosen from the group comprising knock-out of an UDP-glucose: undecaprenyl-phosphate glucose-1-phosphate transferase encoding gene, over-expression of a GDP-L-fucose synthase encoding gene, over-expression of a GDP-mannose 4,6-dehydratase encoding gene, over-expression of a mannose-1-phosphate guanylyltransferase encoding gene, over-expression of a phosphomannomutase encoding gene and over-expression of a mannose-6-phosphate isomerase encoding gene.

In the context of the invention, the cell expresses an alpha-1,2-fucosyltransferase that has galactoside alpha-1,2-fucosyltransferase activity on the galactose residue of Gal-b1,3-GlcNAc (LNB, lacto-N-biose). More specifically, said alpha-1,2-fucosyltransferase transfers a fucose residue from GDP-fucose to the galactose residue in the disaccharide Gal-b1,3-GlcNAc (LNB) of said compound comprising a structure of Formula I, II or III.

According to an embodiment of the method and/or cell of the invention, the alpha-1,2-fucosyltransferase is a polypeptide belonging to the gt11 fucosyltransferase family and comprises the motif X (no M) X (no F) XXXGNX (no N) [ILMV]X (no E,S) X (no E) XXXX (no F, S) X (no Y) XXXXX (no H, S, Y) with SEQ ID NO 38 wherein X can be any amino acid residue. In an alternative embodiment, the alpha-1,2-fucosyltransferase is a polypeptide belonging to the gt74 fucosyltransferase family.

In an additional and/or alternative embodiment, the alpha-1,2-fucosyltransferase comprises a polypeptide sequence according to any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 or 37, preferably any one of SEQ ID NO 01, 02, 03, 04, 05, 06, 07, 08 or 09, more preferably any one of SEQ ID NO 01, 02, 03, 04, 05, 06, 07 or 08, even more preferably any one of SEQ ID NO 05, 06, 07 or 08, most preferably any one of SEQ ID NO 01, 02, 03 or 04.

In an additional and/or alternative embodiment, the alpha-1,2-fucosyltransferase is a functional homolog, variant or derivative of SEQ ID NO 03 having at least 15.0% overall sequence identity to the full-length of said polypeptide with SEQ ID NO 03, preferably said alpha-1,2-fucosyltransferase comprises an amino acid sequence having at least 15.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of said polypeptide with SEQ ID NO 03.

In an additional and/or alternative embodiment, the alpha-1,2-fucosyltransferase is a functional homolog, variant or derivative of any one of SEQ ID NO 15, 34, 35, 36 or 37 having at least 22.0% overall sequence identity to the full-length of any one of said polypeptide with SEQ ID NO 15, 34, 35, 36 or 37, preferably said alpha-1,2-fucosyltransferase comprises an amino acid sequence having at least 22.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 15, 34, 35, 36 or 37.

In an additional and/or alternative embodiment, the alpha-1,2-fucosyltransferase is a functional homolog, variant or derivative of any one of SEQ ID NO 05, 08, 11, 21, 30 or 31 having at least 30.0% overall sequence identity to the full-length of any one of said polypeptide with SEQ ID NO 05, 08, 11, 21, 30 or 31, preferably said alpha-1,2-fucosyltransferase comprises an amino acid sequence having at least 30.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 05, 08, 11, 21, 30 or 31, preferably SEQ ID NO 05 or 08.

In an additional and/or alternative embodiment, the alpha-1,2-fucosyltransferase is a functional homolog, variant or derivative of any one of SEQ ID NO 06, 07, 09, 19, 25, 27, 32 or 33 having at least 35.0% overall sequence identity to the full-length of any one of said polypeptide with SEQ ID NO 06, 07, 09, 19, 25, 27, 32 or 33, preferably said alpha-1,2-fucosyltransferase comprises an amino acid sequence having at least 35.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 06, 07, 09, 19, 25, 27, 32 or 33, preferably SEQ ID NO 06, 07 or 09, more preferably SEQ ID NO 06 or 07.

In an additional and/or alternative embodiment, the alpha-1,2-fucosyltransferase is a functional homolog, variant or derivative of any one of SEQ ID NO 02, 04, 14, 16, 17 or 28 having at least 40.0% overall sequence identity to the full-length of any one of said polypeptide with SEQ ID NO 02, 04, 14, 16, 17 or 28, preferably said alpha-1,2-fucosyltransferase comprises an amino acid sequence having at least 40.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 02, 04, 14, 16, 17 or 28, preferably SEQ ID NO 02 or 04.

In an additional and/or alternative embodiment, the alpha-1,2-fucosyltransferase is a functional homolog, variant or derivative of any one of SEQ ID NO 01, 10, 12, 13, 18, 20, 22, 24 or 26 having at least 45.0% overall sequence identity to the full-length of any one of said polypeptide with SEQ ID NO 01, 10, 12, 13, 18, 20, 22, 24 or 26, preferably said alpha-1,2-fucosyltransferase comprises an amino acid sequence having at least 45.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 01, 10, 12, 13, 18, 20, 22, 24 or 26, preferably SEQ ID NO 01. In an additional and/or alternative embodiment, the alpha-1,2-fucosyltransferase is a functional homolog, variant or derivative of SEQ ID NO 23 having at least 50.0% overall sequence identity to the full-length of said polypeptide with SEQ ID NO 23, preferably said alpha-1,2-fucosyltransferase comprises an amino acid sequence having at least 50.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of said polypeptide with SEQ ID NO 23.

In an additional and/or alternative embodiment, the alpha-1,2-fucosyltransferase is a functional homolog, variant or derivative of SEQ ID NO 29 having at least 70.0% overall sequence identity to the full-length of said polypeptide with SEQ ID NO 29, preferably said alpha-1,2-fucosyltransferase comprises an amino acid sequence having at least 70.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of said polypeptide with SEQ ID NO 29.

In an alternative embodiment, the alpha-1,2-fucosyltransferase is a functional fragment comprising an oligopeptide sequence of at least 10 consecutive amino acid residues from any one of SEQ ID NO 03, 05, 11, 15, 21, 31, 34, 35, 36 or 37, preferably SEQ ID NO 03 or 05.

In an additional and/or alternative embodiment, the alpha-1,2-fucosyltransferase is a functional fragment comprising an oligopeptide sequence of at least 13 consecutive amino acid residues from any one of SEQ ID NO 06, 08, 13, 17, 19, 20, 25, 28 or 30, preferably SEQ ID NO 06 or 08.

In an additional and/or alternative embodiment, the alpha-1,2-fucosyltransferase is a functional fragment comprising an oligopeptide sequence of at least 15 consecutive amino acid residues from any one of SEQ ID NO 04, 07, 09, 10, 16, 26, 27, 32 or 33, preferably SEQ ID NO 04, 07 or 09, more preferably SEQ ID NO 04.

In an additional and/or alternative embodiment, the alpha-1,2-fucosyltransferase is a functional fragment comprising an oligopeptide sequence of at least 18 consecutive amino acid residues from any one of SEQ ID NO 01, 02, 14, 18, 22 or 24, preferably SEQ ID NO 01 or 02.

In an additional and/or alternative embodiment, the alpha-1,2-fucosyltransferase is a functional fragment comprising an oligopeptide sequence of at least 22 consecutive amino acid residues from any one of SEQ ID NO 12, 23 or 29.

Said alpha-1,2-fucosyltransferase can also use other acceptors in addition to the galactose residue within LNB for fucosylation. Said additional acceptor can include but is not limited to a mono-, di- and oligosaccharide like e.g. galactose, glucose, N-acetylglucosamine (GlcNAc), lactose, lactulose, N-acetyllactosamine (LacNAc), 3′-fucosyllactose (3′FL), lacto-N-triose (LN3), lacto-N-tetraose (LNT) and lacto-N-neotetraose (LNnT). Alternatively, said alpha-1,2-fucosyltransferase solely uses the galactose residue within LNB for fucosylation. With the term “solely” is meant only. In other words, said alpha-1,2-fucosyltransferase only accepts the galactose residue within LNB as acceptor for fucosylation in an alpha-1,2 linkage and no other acceptors.

According to another embodiment of the method and/or cell of the invention, the alpha-1,2-fucosyltransferase having galactoside alpha-1,2-fucosyltransferase activity on the galactose residue of Gal-b1,3-GlcNAc has additional galactoside alpha-1,2-fucosyltransferase activity on the galactose residue at the non-reducing end of Gal-b1,3-GlcNAc-b1,3-Gal-b1,4-Glc (LNT, lacto-N-tetraose).

In an embodiment, the alpha-1,2-fucosyltransferase is a polypeptide belonging to the gt74 fucosyltransferase family and comprises the motif [DE]CC [FWY]XXX (no D,E) (Xn) [FWY]X [ILMV][DE][DE] with SEQ ID NO 39 wherein X can be any amino acid residue and wherein n is 10 to 40.

In an additional and/or alternative embodiment, the alpha-1,2-fucosyltransferase comprises a polypeptide sequence according to any one of SEQ ID NO 01, 02, 03, 04, 05, 06, 07, 08, 09, 10, 11, 12, 13, 14, 15, 16, 17 or 18, preferably any one of SEQ ID NO 01, 02, 03, 04, 05, 06, 07, 08 or 09, more preferably any one of SEQ ID NO 01, 02, 03, 04, 05, 06, 07 or 08, even more preferably any one of SEQ ID NO 05, 06, 07 or 08, most preferably any one of SEQ ID NO 01, 02, 03 or 04.

In an additional and/or alternative embodiment, the alpha-1,2-fucosyltransferase is a functional homolog, variant or derivative of any one of SEQ ID NO 03 or 15 having at least 20.0% overall sequence identity to the full-length of any one of said polypeptide with SEQ ID NO 03 or 15, preferably said alpha-1,2-fucosyltransferase comprises an amino acid sequence having at least 20.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 03 or 15, preferably SEQ ID NO 03.

In an additional and/or alternative embodiment, the alpha-1,2-fucosyltransferase is a functional homolog, variant or derivative of any one of SEQ ID NO 05, 08 or 11 having at least 30.0% overall sequence identity to the full-length of any one of said polypeptide with SEQ ID NO 05, 08 or 11, preferably said alpha-1,2-fucosyltransferase comprises an amino acid sequence having at least 30.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 05, 08 or 11.

In an additional and/or alternative embodiment, the alpha-1,2-fucosyltransferase is a functional homolog, variant or derivative of any one of SEQ ID NO 02, 04, 06, 07, 09 or 17 having at least 37.50% overall sequence identity to the full-length of any one of said polypeptide with SEQ ID NO 02, 04, 06, 07, 09 or 17, preferably said alpha-1,2-fucosyltransferase comprises an amino acid sequence having at least 37.50%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 02, 04, 06, 07, 09 or 17.

In an additional and/or alternative embodiment, the alpha-1,2-fucosyltransferase is a functional homolog, variant or derivative of any one of SEQ ID NO 01, 10, 12, 13, 14, 16 or 18 having at least 45.0% overall sequence identity to the full-length of any one of said polypeptide with SEQ ID NO 01, 10, 12, 13, 14, 16 or 18, preferably said alpha-1,2-fucosyltransferase comprises an amino acid sequence having at least 45.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 01, 10, 12, 13, 14, 16 or 18.

In an additional and/or alternative embodiment, the alpha-1,2-fucosyltransferase is a functional fragment comprising an oligopeptide sequence of at least 10 consecutive amino acid residues from any one of SEQ ID NO 03, 05, 11 or 15, preferably SEQ ID NO 03 or 05.

In an additional and/or alternative embodiment, the alpha-1,2-fucosyltransferase is a functional fragment comprising an oligopeptide sequence of at least 13 consecutive amino acid residues from any one of SEQ ID NO 06, 08, 13 or 17, preferably SEQ ID NO 06 or 08.

In an additional and/or alternative embodiment, the alpha-1,2-fucosyltransferase is a functional fragment comprising an oligopeptide sequence of at least 15 consecutive amino acid residues from any one of SEQ ID NO 04, 07, 09, 10, 16, preferably SEQ ID NO 04, 07 or 09, more preferably SEQ ID NO 04 or 07, most preferably SEQ ID NO 04.

In an additional and/or alternative embodiment, the alpha-1,2-fucosyltransferase is a functional fragment comprising an oligopeptide sequence of at least 18 consecutive amino acid residues from any one of SEQ ID NO 01, 02, 14 or 18, preferably SEQ ID NO 01 or 02.

In an additional and/or alternative embodiment, the alpha-1,2-fucosyltransferase is a functional fragment comprising an oligopeptide sequence of at least 20 consecutive amino acid residues from SEQ ID NO 12. According to a further embodiment of the method and/or cell of the present invention, the alpha-1,2-fucosyltransferase having galactoside alpha-1,2-fucosyltransferase activity on the galactose residue of Gal-b1,3-GlcNAc and additional galactoside alpha-1,2-fucosyltransferase activity on the galactose residue at the non-reducing end of LNT has no additional galactoside alpha-1,2-fucosyltransferase activity on lactose. According to an alternative further embodiment of the method and/or cell of the present invention, said alpha-1,2-fucosyltransferase having galactoside alpha-1,2-fucosyltransferase activity on the galactose residue of Gal-b1,3-GlcNAc and additional galactoside alpha-1,2-fucosyltransferase activity on the galactose residue at the non-reducing end of Gal-b1,3-GlcNAc-b1,3-Gal-b1,4-Glc (LNT, lacto-N-tetraose) has an additional galactoside alpha-1,2-fucosyltransferase activity on lactose which is lower than its additional galactoside alpha-1,2-fucosyltransferase activity on the galactose residue at the non-reducing end of LNT.

In an embodiment, the alpha-1,2-fucosyltransferase is a polypeptide belonging to the gt74 fucosyltransferase family and comprises the motif [DE]CC [FWY]XXX (no D,E) (Xn) [FWY]X [ILMV][DE][DE] with SEQ ID NO 39 wherein X can be any amino acid residue and wherein n is 10 to 40.

In an additional and/or alternative embodiment, the alpha-1,2-fucosyltransferase comprises a polypeptide sequence according to any one of SEQ ID NO 01, 02, 03, 04, 07, 09, 10, 12, 13, 14, 15, 16, 17 or 18, preferably any one of SEQ ID NO 01, 02, 03, 04, 07 or 09, more preferably any one of SEQ ID NO 07 or 09, most preferably any one of SEQ ID NO 01, 02, 03 or 04.

In an additional and/or alternative embodiment, the alpha-1,2-fucosyltransferase is a functional homolog, variant or derivative of any one of SEQ ID NO 03 or 15 having at least 20.0% overall sequence identity to the full-length of any one of said polypeptide with SEQ ID NO 03 or 15, preferably said alpha-1,2-fucosyltransferase comprises an amino acid sequence having at least 20.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 03 or 15, preferably SEQ ID NO 03.

In an additional and/or alternative embodiment, the alpha-1,2-fucosyltransferase is a functional homolog, variant or derivative of any one of SEQ ID NO 02, 04, 07, 09 or 17 having at least 37.50% overall sequence identity to the full-length of any one of said polypeptide with SEQ ID NO 02, 04, 06, 07, 09 or 17, preferably said alpha-1,2-fucosyltransferase comprises an amino acid sequence having at least 37.50%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 02, 04, 06, 07, 09 or 17, preferably SEQ ID NO 02, 04, 06, 07 or 09, more preferably SEQ ID NO 02, 04, 06 or 07, even more preferably SEQ ID NO 02 or 04.

In an additional and/or alternative embodiment, the alpha-1,2-fucosyltransferase is a functional homolog, variant or derivative of any one of SEQ ID NO 01, 10, 12, 13, 14, 16 or 18 having at least 45.0% overall sequence identity to the full-length of any one of said polypeptide with SEQ ID NO 01, 10, 12, 13, 14, 16 or 18, preferably said alpha-1,2-fucosyltransferase comprises an amino acid sequence having at least 45.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 01, 10, 12, 13, 14, 16 or 18, preferably SEQ ID NO 01.

In an additional and/or alternative embodiment, the alpha-1,2-fucosyltransferase is a functional fragment comprising an oligopeptide sequence of at least 10 consecutive amino acid residues from any one of SEQ ID NO 03 or 15, preferably SEQ ID NO 03.

In an additional and/or alternative embodiment, the alpha-1,2-fucosyltransferase is a functional fragment comprising an oligopeptide sequence of at least 13 consecutive amino acid residues from any one of SEQ ID NO 13 or 17.

In an additional and/or alternative embodiment, the alpha-1,2-fucosyltransferase is a functional fragment comprising an oligopeptide sequence of at least 15 consecutive amino acid residues from any one of SEQ ID NO 04, 07, 09, 10 or 16, preferably SEQ ID NO 04, 07 or 09, more preferably SEQ ID NO 04 or 07, most preferably SEQ ID NO 04.

In an additional and/or alternative embodiment, the alpha-1,2-fucosyltransferase is a functional fragment comprising an oligopeptide sequence of at least 18 consecutive amino acid residues from any one of SEQ ID NO 01, 02, 14 or 18, preferably SEQ ID NO 01 or 02.

In an additional and/or alternative embodiment, the alpha-1,2-fucosyltransferase is a functional fragment comprising an oligopeptide sequence of at least 20 consecutive amino acid residues from SEQ ID NO 12. According to another further embodiment of the method and/or cell of the present invention, the alpha-1,2-fucosyltransferase having galactoside alpha-1,2-fucosyltransferase activity on the galactose residue of Gal-b1,3-GlcNAc and additional galactoside alpha-1,2-fucosyltransferase activity on the galactose residue at the non-reducing end of LNT has additional galactoside alpha-1,2-fucosyltransferase activity on lactose that is higher than its additional galactoside alpha-1,2-fucosyltransferase activity on the galactose residue at the non-reducing end of LNT.

In an embodiment, the alpha-1,2-fucosyltransferase comprises a polypeptide sequence according to any one of SEQ ID NO 05, 06, 08 or 11, preferably SEQ ID NO 05, 06 or 08.

In an additional and/or alternative embodiment, the alpha-1,2-fucosyltransferase is a functional homolog, variant or derivative of any one of SEQ ID NO 05, 06, 08 or 11 having at least 35.0% overall sequence identity to the full-length of any one of said polypeptide with SEQ ID NO 05, 06, 08 or 11, preferably said alpha-1,2-fucosyltransferase comprises an amino acid sequence having at least 35.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 05, 06, 08 or 11, preferably SEQ ID NO 05, 06 or 08.

In an additional and/or alternative embodiment, the alpha-1,2-fucosyltransferase is a functional fragment comprising an oligopeptide sequence of at least 13 consecutive amino acid residues from SEQ ID NO 05, 06, 08 or 11, preferably SEQ ID NO 05, 06 or 08.

According to another embodiment of the method and/or cell of the present invention, the alpha-1,2-fucosyltransferase having galactoside alpha-1,2-fucosyltransferase activity on the galactose residue of Gal-b1,3-GlcNAc has no galactoside alpha-1,2-fucosyltransferase activity on the galactose residue at the non-reducing end of LNT.

In an embodiment, the alpha-1,2-fucosyltransferase comprises a polypeptide sequence according to any one of SEQ ID NO 19, 20, 21, 22, 23, 24, 25, 26, 27, 28, 29, 30, 31, 32, 33, 34, 35, 36 or 37.

In an additional and/or alternative embodiment, the alpha-1,2-fucosyltransferase is a functional homolog, variant or derivative of any one of SEQ ID NO 34, 35, 36 or 37 having at least 22.0% overall sequence identity to the full-length of any one of said polypeptide with SEQ ID NO 34, 35, 36 or 37, preferably said alpha-1,2-fucosyltransferase comprises an amino acid sequence having at least 22.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 34, 35, 36 or 37.

In an additional and/or alternative embodiment, the alpha-1,2-fucosyltransferase is a functional homolog, variant or derivative of any one of SEQ ID NO 21, 30 or 31 having at least 30.0% overall sequence identity to the full-length of any one of said polypeptide with SEQ ID NO 21, 30 or 31, preferably said alpha-1,2-fucosyltransferase comprises an amino acid sequence having at least 35.0%, preferably at least 30.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 21, 30 or 31.

In an additional and/or alternative embodiment, the alpha-1,2-fucosyltransferase is a functional homolog, variant or derivative of any one of SEQ ID NO 19, 25, 27, 32 or 33 having at least 35.0% overall sequence identity to the full-length of any one of said polypeptide with SEQ ID NO 19, 25, 27, 32 or 33, preferably said alpha-1,2-fucosyltransferase comprises an amino acid sequence having at least 35.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 19, 25, 27, 32 or 33.

In an additional and/or alternative embodiment, the alpha-1,2-fucosyltransferase is a functional homolog, variant or derivative of any one of SEQ ID NO 20, 22, 24, 26 or 28 having at least 45.0% overall sequence identity to the full-length of any one of said polypeptide with SEQ ID NO 20, 22, 24, 26 or 28, preferably said alpha-1,2-fucosyltransferase comprises an amino acid sequence having at least 45.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 20, 22, 24, 26 or 28.

In an additional and/or alternative embodiment, the alpha-1,2-fucosyltransferase is a functional homolog, variant or derivative of SEQ ID NO 23 having at least 50.0% overall sequence identity to the full-length of said polypeptide with SEQ ID NO 23, preferably said alpha-1,2-fucosyltransferase comprises an amino acid sequence having at least 50.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of said polypeptide with SEQ ID NO 23.

In an additional and/or alternative embodiment, the alpha-1,2-fucosyltransferase is a functional homolog, variant or derivative of SEQ ID NO 29 having at least 70.0% overall sequence identity to the full-length of said polypeptide with SEQ ID NO 29, preferably said alpha-1,2-fucosyltransferase comprises an amino acid sequence having at least 70.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of said polypeptide with SEQ ID NO 29.

In an additional and/or alternative embodiment, the alpha-1,2-fucosyltransferase is a functional fragment comprising an oligopeptide sequence of at least 10 consecutive amino acid residues from any one of SEQ ID NO 21, 31, 34, 35, 36 or 37.

In an additional and/or alternative embodiment, the alpha-1,2-fucosyltransferase is a functional fragment comprising an oligopeptide sequence of at least 13 consecutive amino acid residues from any one of SEQ ID NO 19, 20, 25, 28 or 30.

In an additional and/or alternative embodiment, the alpha-1,2-fucosyltransferase is a functional fragment comprising an oligopeptide sequence of at least 15 consecutive amino acid residues from any one of SEQ ID NO 26, 27, 32 or 33.

In an additional and/or alternative embodiment, the alpha-1,2-fucosyltransferase is a functional fragment comprising an oligopeptide sequence of at least 17 consecutive amino acid residues from any one of SEQ ID NO 22 or 24.

In an additional and/or alternative embodiment, the alpha-1,2-fucosyltransferase is a functional fragment comprising an oligopeptide sequence of at least 22 consecutive amino acid residues from any one of SEQ ID NO 23 or 29.

According to a further embodiment of the method and/or cell of the present invention, the alpha-1,2-fucosyltransferase having galactoside alpha-1,2-fucosyltransferase activity on the galactose residue of Gal-b1,3-GlcNAc and no galactoside alpha-1,2-fucosyltransferase activity on the galactose residue at the non-reducing end of LNT has no galactoside alpha-1,2-fucosyltransferase activity on lactose. According to an alternative further embodiment of the method and/or cell of the present invention, the alpha-1,2-fucosyltransferase having galactoside alpha-1,2-fucosyltransferase activity on the galactose residue of Gal-b1,3-GlcNAc and no galactoside alpha-1,2-fucosyltransferase activity on the galactose residue at the non-reducing end of LNT has additional galactoside alpha-1,2-fucosyltransferase activity on lactose which is lower than 3.0% of the galactoside alpha-1,2-fucosyltransferase activity on lactose of the alpha-1,2-fucosyltransferase with SEQ ID NO 06.

In an embodiment, the alpha-1,2-fucosyltransferase comprises a polypeptide sequence according to any one of SEQ ID NO 19, 20, 21, 22, 23, 24, 25, 26, 27, 30, 33, 34, 35, 36 or 37.

In an additional and/or alternative embodiment, the alpha-1,2-fucosyltransferase is a functional homolog, variant or derivative of any one of SEQ ID NO 34, 35, 36 or 37 having at least 22.0% overall sequence identity to the full-length of any one of said polypeptide with SEQ ID NO 34, 35, 36 or 37, preferably said alpha-1,2-fucosyltransferase comprises an amino acid sequence having at least 22.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 34, 35, 36 or 37.

In an additional and/or alternative embodiment, the alpha-1,2-fucosyltransferase is a functional homolog, variant or derivative of any one of SEQ ID NO 21 or 30 having at least 30.0% overall sequence identity to the full-length of any one of said polypeptide with SEQ ID NO 21 or 30, preferably said alpha-1,2-fucosyltransferase comprises an amino acid sequence having at least 30.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 21 or 30.

In an additional and/or alternative embodiment, the alpha-1,2-fucosyltransferase is a functional homolog, variant or derivative of any one of SEQ ID NO 19, 25, 27 or 33 having at least 35.0% overall sequence identity to the full-length of any one of said polypeptide with SEQ ID NO 19, 25, 27 or 33, preferably said alpha-1,2-fucosyltransferase comprises an amino acid sequence having at least 35.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 19, 25, 27 or 33.

In an additional and/or alternative embodiment, the alpha-1,2-fucosyltransferase is a functional homolog, variant or derivative of any one of SEQ ID NO 20, 22, 24 or 26 having at least 45.0% overall sequence identity to the full-length of any one of said polypeptide with SEQ ID NO 20, 22, 24 or 26, preferably said alpha-1,2-fucosyltransferase comprises an amino acid sequence having at least 45.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 20, 22, 24 or 26.

In an additional and/or alternative embodiment, the alpha-1,2-fucosyltransferase is a functional homolog, variant or derivative of SEQ ID NO 23 having at least 50.0% overall sequence identity to the full-length of said polypeptide with SEQ ID NO 23, preferably said alpha-1,2-fucosyltransferase comprises an amino acid sequence having at least 50.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of said polypeptide with SEQ ID NO 23.

In an additional and/or alternative embodiment, the alpha-1,2-fucosyltransferase is a functional fragment comprising an oligopeptide sequence of at least 10 consecutive amino acid residues from any one of SEQ ID NO 21, 34, 35, 36 or 37.

In an additional and/or alternative embodiment, the alpha-1,2-fucosyltransferase is a functional fragment comprising an oligopeptide sequence of at least 13 consecutive amino acid residues from any one of SEQ ID NO 19, 20, 25 or 30.

In an additional and/or alternative embodiment, the alpha-1,2-fucosyltransferase is a functional fragment comprising an oligopeptide sequence of at least 15 consecutive amino acid residues from any one of SEQ ID NO 26, 27 or 33.

In an additional and/or alternative embodiment, the alpha-1,2-fucosyltransferase is a functional fragment comprising an oligopeptide sequence of at least 17 consecutive amino acid residues from any one of SEQ ID NO 22 or 24.

In an additional and/or alternative embodiment, the alpha-1,2-fucosyltransferase is a functional fragment comprising an oligopeptide sequence of at least 20 consecutive amino acid residues from SEQ ID NO 23. According to another further embodiment of the method and/or cell of the present invention, the alpha-1,2-fucosyltransferase having galactoside alpha-1,2-fucosyltransferase activity on the galactose residue of Gal-b1,3-GlcNAc and no additional galactoside alpha-1,2-fucosyltransferase activity on the galactose residue at the non-reducing end of LNT has additional galactoside alpha-1,2-fucosyltransferase activity on lactose that is between 4.0 and 20.0% of the galactoside alpha-1,2-fucosyltransferase activity on lactose of the alpha-1,2-fucosyltransferase with SEQ ID NO 06.

In an embodiment, the alpha-1,2-fucosyltransferase comprises a polypeptide sequence according to any one of SEQ ID NO 28, 29, 31 or 32.

In an additional and/or alternative embodiment, the alpha-1,2-fucosyltransferase is a functional homolog, variant or derivative of any one of SEQ ID NO 31 or 32 having at least 35.0% overall sequence identity to the full-length of any one of said polypeptide with SEQ ID NO 31 or 32, preferably said alpha-1,2-fucosyltransferase comprises an amino acid sequence having at least 35.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of any one of said polypeptide with SEQ ID NO 31 or 32.

In an additional and/or alternative embodiment, the alpha-1,2-fucosyltransferase is a functional homolog, variant or derivative of SEQ ID NO 28 having at least 40.0% overall sequence identity to the full-length of said polypeptide with SEQ ID NO 28, preferably said alpha-1,2-fucosyltransferase comprises an amino acid sequence having at least 40.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of said polypeptide with SEQ ID NO 28.

In an additional and/or alternative embodiment, the alpha-1,2-fucosyltransferase is a functional homolog, variant or derivative of SEQ ID NO 29 having at least 70.0% overall sequence identity to the full-length of said polypeptide with SEQ ID NO 29, preferably said alpha-1,2-fucosyltransferase comprises an amino acid sequence having at least 70.0%, preferably at least 80.0%, more preferably at least 85.0%, 90.0%, 91.0%, 92.0%, 93.0%, 94.0%, 95.0%, 96.0%, 97.0%, 98.0% or 99.0%, even more preferably at least 85.0%, even more preferably at least 90.0%, most preferably at least 95.0%, overall sequence identity to the full-length of said polypeptide with SEQ ID NO 29.

In an additional and/or alternative embodiment, the alpha-1,2-fucosyltransferase is a functional fragment comprising an oligopeptide sequence of at least 10 consecutive amino acid residues from SEQ ID NO 31.

In an additional and/or alternative embodiment, the alpha-1,2-fucosyltransferase is a functional fragment comprising an oligopeptide sequence of at least 14 consecutive amino acid residues from any one of SEQ ID NO 28 or 32.

In an additional and/or alternative embodiment, the alpha-1,2-fucosyltransferase is a functional fragment comprising an oligopeptide sequence of at least 22 consecutive amino acid residues from SEQ ID NO 29.

The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides). Compared to overall sequence identity, the sequence identity will generally be higher when only conserved domains or motifs are considered.

A functional fragment comprising an oligopeptide sequence of at least 10 consecutive amino acid residues from any one of SEQ ID NO 03, 05, 11, 15, 21, 31, 34, 35, 36 or 37 should be understood as any functional fragment comprising an oligopeptide sequence of at least 10, 11, 12, 13, 14, 15 up to the total number of consecutive amino acid residues from any one of the polypeptides with SEQ ID NO 03, 05, 11, 15, 21, 31, 34, 35, 36 or 37 as given herein and having alpha-1,2-fucosyltransferase activity on the Gal residue within LNB.

A functional fragment comprising an oligopeptide sequence of at least 13 consecutive amino acid residues from any one of SEQ ID NO 06, 08, 13, 17, 19, 20, 25, 28 or 30 should be understood as any functional fragment comprising an oligopeptide sequence of at least 13, 14, 15, 16, 17 up to the total number of consecutive amino acid residues from any one of the polypeptides with SEQ ID NO 06, 08, 13, 17, 19, 20, 25, 28 or 30 as given herein and having alpha-1,2-fucosyltransferase activity on the Gal residue within LNB.

A functional fragment comprising an oligopeptide sequence of at least 15 consecutive amino acid residues from any one of SEQ ID NO 04, 07, 09, 10, 16, 26, 27, 32 or 33 should be understood as any functional fragment comprising an oligopeptide sequence of at least 15, 16, 17, 18, 19 up to the total number of consecutive amino acid residues from any one of the polypeptides with SEQ ID NO 04, 07, 09, 10, 16, 26, 27, 32 or 33 as given herein and having alpha-1,2-fucosyltransferase activity on the Gal residue within LNB.

A functional fragment comprising an oligopeptide sequence of at least 18 consecutive amino acid residues from any one of SEQ ID NO 01, 02, 14, 18, 22 or 24 should be understood as any functional fragment comprising an oligopeptide sequence of at least 18, 19, 20, 21, 22 up to the total number of consecutive amino acid residues from any one of the polypeptides with SEQ ID NO 01, 02, 14, 18, 22 or 24 as given herein and having alpha-1,2-fucosyltransferase activity on the Gal residue within LNB.

A functional fragment comprising an oligopeptide sequence of at least 22 consecutive amino acid residues from any one of SEQ ID NO 12, 23 or 29 should be understood as any functional fragment comprising an oligopeptide sequence of at least 22, 23, 24, 25, 26 up to the total number of consecutive amino acid residues from any one of the polypeptides with SEQ ID NO 12, 23 or 29 as given herein and having alpha-1,2-fucosyltransferase activity on the Gal residue within LNB.

A functional fragment comprising an oligopeptide sequence of at least 10 consecutive amino acid residues from any one of SEQ ID NO 03, 05, 11 or 15 should be understood as any functional fragment comprising an oligopeptide sequence of at least 10, 11, 12, 13, 14 up to the total number of consecutive amino acid residues from any one of the polypeptides with SEQ ID NO 03, 05, 11 or 15 as given herein and having alpha-1,2-fucosyltransferase activity on the Gal residue within LNB and additional galactoside alpha-1,2-fucosyltransferase activity on the galactose residue at the non-reducing end of LNT.

A functional fragment comprising an oligopeptide sequence of at least 13 consecutive amino acid residues from any one of SEQ ID NO 06, 08, 13 or 17 should be understood as any functional fragment comprising an oligopeptide sequence of at least 13, 14, 15, 16, 17 up to the total number of consecutive amino acid residues from any one of the polypeptides with SEQ ID NO 06, 08, 13 or 17 as given herein and having alpha-1,2-fucosyltransferase activity on the Gal residue within LNB and additional galactoside alpha-1,2-fucosyltransferase activity on the galactose residue at the non-reducing end of LNT.

A functional fragment comprising an oligopeptide sequence of at least 15 consecutive amino acid residues from any one of SEQ ID NO 04, 07, 09, 10 or 16 should be understood as any functional fragment comprising an oligopeptide sequence of at least 15, 16, 17, 18, 19 up to the total number of consecutive amino acid residues from any one of the polypeptides with SEQ ID NO 04, 07, 09, 10 or 16 as given herein and having alpha-1,2-fucosyltransferase activity on the Gal residue within LNB and additional galactoside alpha-1,2-fucosyltransferase activity on the galactose residue at the non-reducing end of LNT.

A functional fragment comprising an oligopeptide sequence of at least 18 consecutive amino acid residues from any one of SEQ ID NO 01, 02, 14 or 18 should be understood as any functional fragment comprising an oligopeptide sequence of at least 18, 19, 20, 21, 22 up to the total number of consecutive amino acid residues from any one of the polypeptides with SEQ ID NO 01, 02, 14 or 18 as given herein and having alpha-1,2-fucosyltransferase activity on the Gal residue within LNB and additional galactoside alpha-1,2-fucosyltransferase activity on the galactose residue at the non-reducing end of LNT.

A functional fragment comprising an oligopeptide sequence of at least 20 consecutive amino acid residues from SEQ ID NO 12 should be understood as any functional fragment comprising an oligopeptide sequence of at least 20, 21, 22, 23, 24 up to the total number of consecutive amino acid residues from the polypeptide with SEQ ID NO 12 as given herein and having alpha-1,2-fucosyltransferase activity on the Gal residue within LNB and additional galactoside alpha-1,2-fucosyltransferase activity on the galactose residue at the non-reducing end of LNT.

A functional fragment comprising an oligopeptide sequence of at least 10 consecutive amino acid residues from any one of SEQ ID NO 03 or 15 should be understood as any functional fragment comprising an oligopeptide sequence of at least 10, 11, 12, 13, 14 up to the total number of consecutive amino acid residues from any one of the polypeptides with SEQ ID NO 03 or 15 as given herein and having alpha-1,2-fucosyltransferase activity on the Gal residue within LNB and additional galactoside alpha-1,2-fucosyltransferase activity on the galactose residue at the non-reducing end of LNT and either no additional galactoside alpha-1,2-fucosyltransferase activity on lactose or additional galactoside alpha-1,2-fucosyltransferase activity on lactose which is lower than its additional galactoside alpha-1,2-fucosyltransferase activity on the galactose residue at the non-reducing end of LNT.

A functional fragment comprising an oligopeptide sequence of at least 13 consecutive amino acid residues from any one of SEQ ID NO 13 or 17 should be understood as any functional fragment comprising an oligopeptide sequence of at least 13, 14, 15, 16, 17 up to the total number of consecutive amino acid residues from any one of the polypeptides with SEQ ID NO 13 or 17 as given herein and having alpha-1,2-fucosyltransferase activity on the Gal residue within LNB and additional galactoside alpha-1,2-fucosyltransferase activity on the galactose residue at the non-reducing end of LNT and either no additional galactoside alpha-1,2-fucosyltransferase activity on lactose or additional galactoside alpha-1,2-fucosyltransferase activity on lactose which is lower than its additional galactoside alpha-1,2-fucosyltransferase activity on the galactose residue at the non-reducing end of LNT.

A functional fragment comprising an oligopeptide sequence of at least 15 consecutive amino acid residues from any one of SEQ ID NO 04, 07, 09, 10 or 16 should be understood as any functional fragment comprising an oligopeptide sequence of at least 15, 16, 17, 18, 19 up to the total number of consecutive amino acid residues from any one of the polypeptides with SEQ ID NO 04, 07, 09, 10 or 16 as given herein and having alpha-1,2-fucosyltransferase activity on the Gal residue within LNB and additional galactoside alpha-1,2-fucosyltransferase activity on the galactose residue at the non-reducing end of LNT and either no additional galactoside alpha-1,2-fucosyltransferase activity on lactose or additional galactoside alpha-1,2-fucosyltransferase activity on lactose which is lower than its additional galactoside alpha-1,2-fucosyltransferase activity on the galactose residue at the non-reducing end of LNT.

A functional fragment comprising an oligopeptide sequence of at least 18 consecutive amino acid residues from any one of SEQ ID NO 01, 02, 14 or 18 should be understood as any functional fragment comprising an oligopeptide sequence of at least 18, 19, 20, 21, 22 up to the total number of consecutive amino acid residues from any one of the polypeptides with SEQ ID NO 01, 02, 14 or 18 as given herein and having alpha-1,2-fucosyltransferase activity on the Gal residue within LNB and additional galactoside alpha-1,2-fucosyltransferase activity on the galactose residue at the non-reducing end of LNT and either no additional galactoside alpha-1,2-fucosyltransferase activity on lactose or additional galactoside alpha-1,2-fucosyltransferase activity on lactose which is lower than its additional galactoside alpha-1,2-fucosyltransferase activity on the galactose residue at the non-reducing end of LNT.

A functional fragment comprising an oligopeptide sequence of at least 20 consecutive amino acid residues from SEQ ID NO 12 should be understood as any functional fragment comprising an oligopeptide sequence of at least 20, 21, 22, 23, 24 up to the total number of consecutive amino acid residues from the polypeptide with SEQ ID NO 12 as given herein and having alpha-1,2-fucosyltransferase activity on the Gal residue within LNB and additional galactoside alpha-1,2-fucosyltransferase activity on the galactose residue at the non-reducing end of LNT and either no additional galactoside alpha-1,2-fucosyltransferase activity on lactose or additional galactoside alpha-1,2-fucosyltransferase activity on lactose which is lower than its additional galactoside alpha-1,2-fucosyltransferase activity on the galactose residue at the non-reducing end of LNT.

A functional fragment comprising an oligopeptide sequence of at least 13 consecutive amino acid residues from SEQ ID NO 05, 06, 08 or 11 should be understood as any functional fragment comprising an oligopeptide sequence of at least 13, 14, 15, 16, 17 up to the total number of consecutive amino acid residues from any one of the polypeptides with SEQ ID NO 05, 06, 08 or 11 and having alpha-1,2-fucosyltransferase activity on the Gal residue within LNB, additional galactoside alpha-1,2-fucosyltransferase activity on the galactose residue at the non-reducing end of LNT and additional galactoside alpha-1,2-fucosyltransferase activity on lactose that is higher than its additional galactoside alpha-1,2-fucosyltransferase activity on the galactose residue at the non-reducing end of LNT.

A functional fragment comprising an oligopeptide sequence of at least 10 consecutive amino acid residues from any one of SEQ ID NO 21, 31, 34, 35, 36 or 37 should be understood as any functional fragment comprising an oligopeptide sequence of at least 10, 11, 12, 13, 14 up to the total number of consecutive amino acid residues from any one of the polypeptides with SEQ ID NO 21, 31, 34, 35, 36 or 37 and having alpha-1,2-fucosyltransferase activity on the Gal residue within LNB and no additional galactoside alpha-1,2-fucosyltransferase activity on the galactose residue at the non-reducing end of LNT.

A functional fragment comprising an oligopeptide sequence of at least 13 consecutive amino acid residues from any one of SEQ ID NO 19, 20, 25, 28 or 30 should be understood as any functional fragment comprising an oligopeptide sequence of at least 13, 14, 15, 16, 17 up to the total number of consecutive amino acid residues from any one of the polypeptides with SEQ ID NO 19, 20, 25, 28 or 30 and having alpha-1,2-fucosyltransferase activity on the Gal residue within LNB and no additional galactoside alpha-1,2-fucosyltransferase activity on the galactose residue at the non-reducing end of LNT.

A functional fragment comprising an oligopeptide sequence of at least 15 consecutive amino acid residues from any one of SEQ ID NO 26, 27, 32 or 33 should be understood as any functional fragment comprising an oligopeptide sequence of at least 15, 16, 17, 18, 19 up to the total number of consecutive amino acid residues from any one of the polypeptides with SEQ ID NO 26, 27, 32 or 33 and having alpha-1,2-fucosyltransferase activity on the Gal residue within LNB and no additional galactoside alpha-1,2-fucosyltransferase activity on the galactose residue at the non-reducing end of LNT.

A functional fragment comprising an oligopeptide sequence of at least 17 consecutive amino acid residues from any one of SEQ ID NO 22 or 24 should be understood as any functional fragment comprising an oligopeptide sequence of at least 17, 18, 19, 20, 21 up to the total number of consecutive amino acid residues from any one of the polypeptides with SEQ ID NO 22 or 24 and having alpha-1,2-fucosyltransferase activity on the Gal residue within LNB and no additional galactoside alpha-1,2-fucosyltransferase activity on the galactose residue at the non-reducing end of LNT.

A functional fragment comprising an oligopeptide sequence of at least 22 consecutive amino acid residues from any one of SEQ ID NO 23 or 29 should be understood as any functional fragment comprising an oligopeptide sequence of at least 22, 23, 24, 25, 26 up to the total number of consecutive amino acid residues from any one of the polypeptides with SEQ ID NO 23 or 29 and having alpha-1,2-fucosyltransferase activity on the Gal residue within LNB and no additional galactoside alpha-1,2-fucosyltransferase activity on the galactose residue at the non-reducing end of LNT.

A functional fragment comprising an oligopeptide sequence of at least 10 consecutive amino acid residues from any one of SEQ ID NO 21, 34, 35, 36 or 37 should be understood as any functional fragment comprising an oligopeptide sequence of at least 10, 11, 12, 13, 14 up to the total number of consecutive amino acid residues from any one of the polypeptides with SEQ ID NO 21, 34, 35, 36 or 37 and having alpha-1,2-fucosyltransferase activity on the Gal residue within LNB, no additional galactoside alpha-1,2-fucosyltransferase activity on the galactose residue at the non-reducing end of LNT and either no additional galactoside alpha-1,2-fucosyltransferase activity on lactose or an additional galactoside alpha-1,2-fucosyltransferase activity on lactose which is lower than 3.0% of the galactoside alpha-1,2-fucosyltransferase activity on lactose of the alpha-1,2-fucosyltransferase with SEQ ID NO 06.

A functional fragment comprising an oligopeptide sequence of at least 13 consecutive amino acid residues from any one of SEQ ID NO 19, 20, 25 or 30 should be understood as any functional fragment comprising an oligopeptide sequence of at least 13, 14, 15, 16, 17 up to the total number of consecutive amino acid residues from any one of the polypeptides with SEQ ID NO 19, 20, 25 or 30 and having alpha-1,2-fucosyltransferase activity on the Gal residue within LNB, no additional galactoside alpha-1,2-fucosyltransferase activity on the galactose residue at the non-reducing end of LNT and either no additional galactoside alpha-1,2-fucosyltransferase activity on lactose or an additional galactoside alpha-1,2-fucosyltransferase activity on lactose which is lower than 3.0% of the galactoside alpha-1,2-fucosyltransferase activity on lactose of the alpha-1,2-fucosyltransferase with SEQ ID NO 06.

A functional fragment comprising an oligopeptide sequence of at least 15 consecutive amino acid residues from any one of SEQ ID NO 26, 27 or 33 should be understood as any functional fragment comprising an oligopeptide sequence of at least 15, 16, 17, 18, 19 up to the total number of consecutive amino acid residues from any one of the polypeptides with SEQ ID NO 26, 27 or 33 and having alpha-1,2-fucosyltransferase activity on the Gal residue within LNB, no additional galactoside alpha-1,2-fucosyltransferase activity on the galactose residue at the non-reducing end of LNT and either no additional galactoside alpha-1,2-fucosyltransferase activity on lactose or an additional galactoside alpha-1,2-fucosyltransferase activity on lactose which is lower than 3.0% of the galactoside alpha-1,2-fucosyltransferase activity on lactose of the alpha-1,2-fucosyltransferase with SEQ ID NO 06.

A functional fragment comprising an oligopeptide sequence of at least 17 consecutive amino acid residues from any one of SEQ ID NO 22 or 24 should be understood as any functional fragment comprising an oligopeptide sequence of at least 17, 18, 19, 20, 21 up to the total number of consecutive amino acid residues from any one of the polypeptides with SEQ ID NO NO 22 or 24 and having alpha-1,2-fucosyltransferase activity on the Gal residue within LNB, no additional galactoside alpha-1,2-fucosyltransferase activity on the galactose residue at the non-reducing end of LNT and either no additional galactoside alpha-1,2-fucosyltransferase activity on lactose or an additional galactoside alpha-1,2-fucosyltransferase activity on lactose which is lower than 3.0% of the galactoside alpha-1,2-fucosyltransferase activity on lactose of the alpha-1,2-fucosyltransferase with SEQ ID NO 06.

A functional fragment comprising an oligopeptide sequence of at least 20 consecutive amino acid residues from SEQ ID NO 23 should be understood as any functional fragment comprising an oligopeptide sequence of at least 20, 21, 22, 23, 24 up to the total number of consecutive amino acid residues from the polypeptide with SEQ ID NO 23 and having alpha-1,2-fucosyltransferase activity on the Gal residue within LNB, no additional galactoside alpha-1,2-fucosyltransferase activity on the galactose residue at the non-reducing end of LNT and either no additional galactoside alpha-1,2-fucosyltransferase activity on lactose or an additional galactoside alpha-1,2-fucosyltransferase activity on lactose which is lower than 3.0% of the galactoside alpha-1,2-fucosyltransferase activity on lactose of the alpha-1,2-fucosyltransferase with SEQ ID NO 06.

A functional fragment comprising an oligopeptide sequence of at least 10 consecutive amino acid residues from SEQ ID NO 31 should be understood as any functional fragment comprising an oligopeptide sequence of at least 10, 11, 12, 13, 14 up to the total number of consecutive amino acid residues from the polypeptide with SEQ ID NO 31 and having alpha-1,2-fucosyltransferase activity on the Gal residue within LNB, no additional galactoside alpha-1,2-fucosyltransferase activity on the galactose residue at the non-reducing end of LNT and additional galactoside alpha-1,2-fucosyltransferase activity on lactose that is between 4.0 and 20.0% of the galactoside alpha-1,2-fucosyltransferase activity on lactose of the alpha-1,2-fucosyltransferase with SEQ ID NO 06.

A functional fragment comprising an oligopeptide sequence of at least 14 consecutive amino acid residues from any one of SEQ ID NO 28 or 32 should be understood as any functional fragment comprising an oligopeptide sequence of at least 14, 15, 16, 17, 18 up to the total number of consecutive amino acid residues from the polypeptides with SEQ ID NO 28 or 32 and having alpha-1,2-fucosyltransferase activity on the Gal residue within LNB, no additional galactoside alpha-1,2-fucosyltransferase activity on the galactose residue at the non-reducing end of LNT and additional galactoside alpha-1,2-fucosyltransferase activity on lactose that is between 4.0 and 20.0% of the galactoside alpha-1,2-fucosyltransferase activity on lactose of the alpha-1,2-fucosyltransferase with SEQ ID NO 06.

A functional fragment comprising an oligopeptide sequence of at least 22 consecutive amino acid residues from SEQ ID NO 29 should be understood as any functional fragment comprising an oligopeptide sequence of at least 22, 23, 24, 25, 26 up to the total number of consecutive amino acid residues from the polypeptide with SEQ ID NO 29 and having alpha-1,2-fucosyltransferase activity on the Gal residue within LNB, no additional galactoside alpha-1,2-fucosyltransferase activity on the galactose residue at the non-reducing end of LNT and additional galactoside alpha-1,2-fucosyltransferase activity on lactose that is between 4.0 and 20.0% of the galactoside alpha-1,2-fucosyltransferase activity on lactose of the alpha-1,2-fucosyltransferase with SEQ ID NO 06.

In a preferred embodiment of the method and/or cell of the present invention, the cell expresses an alpha-1,2-fucosyltransferase that preferably uses LNB as acceptor for alpha-1,2-fucosylation of the galactose residue within said LNB over other acceptors like e.g. galactose, glucose, GlcNAc, lactose, lactulose, LacNAc, 3′FL, LN3, LNT and LNnT. In a more preferred embodiment, at least 50% of the fucosylated compound obtained in a mixture by the alpha-1,2-fucosyltransferase expressed in the cell is derived from alpha-1,2-fucosylation of the galactose residue of LNB. In other words, at least 50% of the fucosylated compound obtained in a mixture by the alpha-1,2-fucosyltransferase expressed in the cell is alpha-1,2 fucosylated LNB or 2′FLNB. At least 50% of the fucosylated compound in a mixture should be understood as at least 50%, 55%, 60%, 65%, 70%, 75%, 80%, 81%, 82%, 83%, 84%, 85%, 86%, 87%, 88%, 89%, 90%, 91%, 91.50%, 92.00%, 92.50%, 93.00%, 93.50%, 94.00%, 94.50%, 95.00%, 95,50%, 96.00%, 96,50%, 97.00%, 97,50%, 98.00%, 98,50%, 99.00%, 99,50%, 99,60%, 99,70%, 99,80%, 99,90%, 100% of the fucosylated compound in a mixture is alpha-1,2 fucosylated LNB or 2′FLNB.

Preferably, at least 60%, more preferably at least 70%, even more preferably at least 75%, even more preferably at least 80%, even more preferably at least 85%, even more preferably at least 90%, most preferably at least 95% of the fucosylated compound obtained in a mixture by the alpha-1,2-fucosyltransferase expressed in the cell is alpha-1,2 fucosylated LNB or 2′FLNB.

In another preferred embodiment of the method and/or cell of the present invention, the cell expresses an alpha-1,2-fucosyltransferase as described herein that is capable to modify the intracellularly produced compound comprising a structure of Formula IV, V or VI:

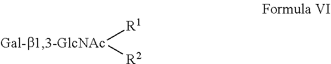

Gal-β1,3-GlcNAc Formula IV

Gal-β1,3-GlcNAc-R¹ Formula V
wherein:
R¹ is a monosaccharide, disaccharide, oligosaccharide, protein, glycoprotein, peptide, glycopeptide, lipid or glycolipid; and, when present, R² is a monosaccharide, disaccharide or oligosaccharide.

Preferably, said cell is capable to produce GDP-fucose which is donor for said alpha-1,2-fucosyltransferase.

In a further embodiment of the method and/or cell of the invention, the cell is modified in the expression or activity of any one of said alpha-1,2-fucosyltransferases.

In an embodiment of the method and/or cell of the present invention, the cell is capable to produce one or more nucleotide-activated sugars chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy—L-arabino-4-hexulose, UDP-2-acetamido-2,6-dideoxy--L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7 (8,9) Ac₂, CMP-N-glycolylneuraminic acid (CMP-Neu5Gc), GDP-rhamnose and UDP-xylose.

In a preferred embodiment of the method and/or cell of the invention, the cell expresses one or more polypeptides chosen from the list comprising mannose-6-phosphate isomerase, phosphomannomutase, mannose-1-phosphate guanylyltransferase, GDP-mannose 4,6-dehydratase, GDP-L-fucose synthase, pyrophosphorylase, fucose-1-phosphate fucose permease, fucose kinase, GDP-fucose guanylyltransferase, L-glutamine-D-fructose-6-phosphate aminotransferase, glucosamine-6-phosphate deaminase, phosphoglucosamine mutase, N-acetylglucosamine-6-phosphate deacetylase, N-acylglucosamine 2-epimerase, UDP-N-acetylglucosamine 2-epimerase, N-acetylmannosamine-6-phosphate 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridylyltransferase, glucosamine-1-phosphate acetyltransferase, N-acetylneuraminate synthase, N-acetylneuraminate lyase, N-acylneuraminate-9-phosphate synthase, N-acylneuraminate-9-phosphate phosphatase, N acylneuraminate cytidylyltransferase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase.

In a more preferred embodiment of the method and/or cell, the cell is modified in the expression or activity of any one of said polypeptides. Any one of said polypeptides is an endogenous protein of the cell with a modified expression or activity, preferably said endogenous polypeptide is overexpressed; alternatively any one of said polypeptides is a heterologous protein that is heterogeneously introduced and expressed in said cell, preferably overexpressed. Said endogenous polypeptide can have a modified expression in the cell which also expresses a heterologous polypeptide of said list.

GDP-fucose can be provided by an enzyme expressed in the cell or by the metabolism of the cell. Such cell producing GDP-fucose can express an enzyme converting, e.g., fucose, which is to be added to the cell, to GDP-fucose. This enzyme may be, e.g., a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus. Preferably, the cell is modified to produce GDP-fucose. More preferably, the cell is modified for enhanced GDP-fucose production. Said modification can be any one or more chosen from the group comprising knock-out of an UDP-glucose: undecaprenyl-phosphate glucose-1-phosphate transferase encoding gene, over-expression of a GDP-L-fucose synthase encoding gene, over-expression of a GDP-mannose 4,6-dehydratase encoding gene, over-expression of a mannose-1-phosphate guanylyltransferase encoding gene, over-expression of a phosphomannomutase encoding gene and over-expression of a mannose-6-phosphate isomerase encoding gene.

CMP-Neu5Ac can be provided by an enzyme expressed in the cell or by the metabolism of the cell. Such cell producing CMP-Neu5Ac can express an enzyme converting, e.g., sialic acid, which is to be added to the cell, to CMP-Neu5Ac. This enzyme may be a CMP-sialic acid synthetase, like the N-acylneuraminate cytidylyltransferase from several species including Homo sapiens, Neisseria meningitidis, and Pasteurella multocida. Preferably, the cell is modified to produce CMP-Neu5Ac. More preferably, the cell is modified for enhanced CMP-Neu5Ac production. Said modification can be any one or more chosen from the group comprising knock-out of an N-acetylglucosamine-6-phosphate deacetylase, knock-out of an glucosamine-6-phosphate deaminase, over-expression of a sialate synthase encoding gene, and over-expression of an N-acetyl-D-glucosamine-2-epimerase encoding gene.

UDP-GalNAc can be synthesized from UDP-GlcNAc by the action of a single-step reaction using an UDP-N-acetylglucosamine 4-epimerase like e.g. wbgU from Plesiomonas shigelloides, gne from Yersinia enterocolitica or wbpP from Pseudomonas aeruginosa serotype 06. Preferably, the cell is modified to produce UDP-GalNAc. More preferably, the cell is modified for enhanced UDP-GalNAc production.

UDP-ManNAc can be synthesized directly from UDP-GlcNAc via an epimerization reaction performed by an UDP-GlcNAc 2-epimerase (like e.g. cap5P from Staphylococcus aureus, RffE from E. coli, Cps19fK from S. pneumoniae, and RfbC from S. enterica). Preferably, the cell is modified to produce UDP-ManNAc. More preferably, the cell is modified for enhanced UDP-ManNAc production.

CMP-Neu5Gc can be synthesized directly from CMP-Neu5Ac via a hydroxylation reaction performed by a vertebrate CMP-Neu5Ac hydroxylase (CMAH) enzyme. Preferably, the cell is modified to produce CMP-Neu5Gc. More preferably, the cell is modified for enhanced CMP-Neu5Gc production.

According to a preferred embodiment of the method and/or cell of the present invention, the cell expresses one or more glycosyltransferases chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

In a more preferred embodiment of the method and/or cell, the fucosyltransferase is chosen from the list comprising alpha-1,2-fucosyltransferase, alpha-1,3-fucosyltransferase, alpha-1,4-fucosyltransferase and alpha-1,6-fucosyltransferase.

In another more preferred embodiment of the method and/or cell, the sialyltransferase is chosen from the list comprising alpha-2,3-sialyltransferase, alpha-2,6-sialyltransferase and alpha-2,8-sialyltransferase. In another more preferred embodiment of the method and/or cell, the galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase. In another more preferred embodiment of the method and/or cell, the glucosyltransferase is chosen from the list comprising alpha-glucosyltransferase, beta-1,2-glucosyltransferase, beta-1,3-glucosyltransferase and beta-1,4-glucosyltransferase.

In another more preferred embodiment of the method and/or cell, the mannosyltransferase is chosen from the list comprising alpha-1,2-mannosyltransferase, alpha-1,3-mannosyltransferase and alpha-1,6-mannosyltransferase.

In another more preferred embodiment of the method and/or cell, the N-acetylglucosaminyltransferase is chosen from the list comprising galactoside beta-1,3-N-acetylglucosaminyltransferase and beta-1,6-N-acetylglucosaminyltransferase.

In another more preferred embodiment of the method and/or cell, the N-acetylgalactosaminyltransferase is an alpha-1,3-N-acetylgalactosaminyltransferase.

In another more preferred embodiment of the method and/or cell of the invention, the cell is modified in the expression or activity of at least one of said glycosyltransferases. Said glycosyltransferase is an endogenous protein of the cell with a modified expression or activity, preferably said endogenous glycosyltransferase is overexpressed; alternatively said glycosyltransferase is a heterologous protein that is heterogeneously introduced and expressed in said cell, preferably overexpressed. Said endogenous glycosyltransferase can have a modified expression in the cell which also expresses a heterologous glycosyltransferase.

According to another preferred embodiment of the method and/or cell of the invention, the cell is using a precursor for the production of said compound comprising a structure of Formula I, II or III, preferably said precursor being fed to the cell from the cultivation medium. According to a more preferred embodiment of the method and/or cell, the cell is using at least two precursors for the production of said compound comprising a structure of Formula I, II or III, preferably said precursors being fed to the cell from the cultivation medium. According to another preferred embodiment of the method and/or cell of the invention, the cell is producing at least one precursor, preferably at least two precursors, for the production of said compound comprising a structure of Formula I, II or III. In a preferred embodiment of the method and/or cell, the precursor that is used by the cell for the production of said compound comprising a structure of Formula I, II or III is completely converted into said compound comprising a structure of Formula I, II or III.

In another preferred embodiment of the method and/or cell of the invention, the cell expresses a membrane transporter protein or a polypeptide having transport activity hereby transporting compounds across the outer membrane of the cell wall. In another preferred embodiment of the method and/or cell of the invention, the cell expresses more than one membrane transporter protein or polypeptide having transport activity hereby transporting compounds across the outer membrane of the cell wall. In a more preferred embodiment of the method and/or cell of the invention, the cell is modified in the expression or activity of said membrane transporter protein or polypeptide having transport activity. Said membrane transporter protein or polypeptide having transport activity is an endogenous protein of the cell with a modified expression or activity, preferably said endogenous membrane transporter protein or polypeptide having transport activity is overexpressed; alternatively said membrane transporter protein or polypeptide having transport activity is a heterologous protein that is heterogeneously introduced and expressed in said cell, preferably overexpressed. Said endogenous membrane transporter protein or polypeptide having transport activity can have a modified expression in the cell which also expresses a heterologous membrane transporter protein or polypeptide having transport activity.

In a more preferred embodiment of the method and/or cell of the invention, the membrane transporter protein or polypeptide having transport activity is chosen from the list comprising porters, P-P-bond-hydrolysis-driven transporters, b-barrel porins, auxiliary transport proteins, putative transport proteins and phosphotransfer-driven group translocators. In an even more preferred embodiment of the method and/or cell of the invention, the porters comprise MFS transporters, sugar efflux transporters and siderophore exporters. In another more preferred embodiment of the method and/or cell of the invention, the P-P-bond-hydrolysis-driven transporters comprise ABC transporters and siderophore exporters.

In another preferred embodiment of the method and/or cell of the invention, the membrane transporter protein or polypeptide having transport activity controls the flow over the outer membrane of the cell wall of said compound comprising a structure of Formula I, II or III. In an alternative and/or additional preferred embodiment of the method and/or cell of the invention, the membrane transporter protein or polypeptide having transport activity controls the flow over the outer membrane of the cell wall of one or more precursor(s) to be used in said production of said compound comprising a structure of Formula I, II or III. In an alternative and/or additional preferred embodiment of the method and/or cell of the invention, the membrane transporter protein or polypeptide having transport activity controls the flow over the outer membrane of the cell wall of one or more acceptor(s) to be used in said production of said compound comprising a structure of Formula I, II or III.

In another preferred embodiment of the method and/or cell of the invention, the membrane transporter protein or polypeptide having transport activity provides improved production of said compound comprising a structure of Formula I, II or III. In an alternative and/or additional preferred embodiment of the method and/or cell of the invention, the membrane transporter protein or polypeptide having transport activity provides enabled efflux of said compound comprising a structure of Formula I, II or III.

In an alternative and/or additional preferred embodiment of the method and/or cell of the invention, the membrane transporter protein or polypeptide having transport activity provides enhanced efflux of said compound comprising a structure of Formula I, II or III.

In another preferred embodiment of the method and/or cell of the invention, the cell expresses a polypeptide selected from the group comprising a lactose transporter like e.g. the LacY or lac12 permease, a fucose transporter, a glucose transporter, a galactose transporter, a transporter for a nucleotide-activated sugar like for example a transporter for UDP-GlcNAc, UDP-Gal and/or GDP-Fuc.

In another preferred embodiment of the method and/or cell of the present invention, the cell expresses a membrane transporter protein belonging to the family of MFS transporters like e.g. an MdfA polypeptide of the multidrug transporter MdfA family from species comprising E. coli (UniProt ID POAEY8, sequence version 1), Cronobacter muytjensii (UniProt ID A0A2T7ANQ9, sequence version 1), Citrobacter youngae (UniProt ID D4BC23, sequence version 1) and Yokenella regensburgei (UniProt ID G9Z5F4, sequence version 1). In another preferred embodiment of the method and/or cell of the present invention, the cell expresses a membrane transporter protein belonging to the family of sugar efflux transporters like e.g. a SetA polypeptide of the SetA family from species comprising E. coli (UniProt ID P31675, sequence version 3), Citrobacter koseri (UniProt ID A0A078 LM16, sequence version 1) and Klebsiella pneumoniae (UniProt ID A0A0C4MGS7, sequence version 1). In another preferred embodiment of the method and/or cell of the present invention, the cell expresses a membrane transporter protein belonging to the family of siderophore exporters like e.g. the E. coli entS (UniProt ID P24077, sequence version 2) and the E. coli iceT (UniProt ID A0A024L207, sequence version 1). In another preferred embodiment of the method and/or cell of the present invention, the cell expresses a membrane transporter protein belonging to the family of ABC transporters like e.g. oppF from E. coli (UniProt ID P77737, sequence version 1), ImrA from Lactococcus lactis subsp. lactis bv. diacetylactis (UniProt ID A0A1VONEL4, sequence version 1) and Blon_2475 from Bifidobacterium longum subsp. infantis (UniProt ID B7GPD4, sequence version 1). In a more preferred embodiment of the method and/or cell of the present invention, the cell expresses more than one membrane transporter protein chosen from the list comprising a lactose transporter like e.g. the LacY or lac12 permease, a fucose transporter, a glucose transporter, a galactose transporter, a transporter for a nucleotide-activated sugar like for example a transporter for UDP-GlcNAc, UDP-Gal and/or GDP-Fuc, the MdfA protein from E. coli (UniProt ID POAEY8, sequence version 1), the MdfA protein from Cronobacter muytjensii (UniProt ID A0A2T7ANQ9, sequence version 1), the MdfA protein from Citrobacter youngae (UniProt ID D4BC23, sequence version 1), the MdfA protein from Yokenella regensburgei (UniProt ID G9Z5F4, sequence version 1), the SetA protein from E. coli (UniProt ID P31675, sequence version 3), the SetA protein from Citrobacter koseri (UniProt ID A0A078 LM16, sequence version 1), the SetA protein from Klebsiella pneumoniae (UniProt ID A0A0C4MGS7, sequence version 1), the entS protein from E. coli (UniProt ID P24077, sequence version 2), the iceT protein from E. coli (UniProt ID A0A024L207, sequence version 1), the oppF protein from E. coli (UniProt ID P77737, sequence version 1), the ImrA protein from Lactococcus lactis subsp. lactis bv. diacetylactis (UniProt ID AOA1VONEL4, sequence version 1) and Blon_2475 from Bifidobacterium longum subsp. infantis (UniProt ID B7GPD4, sequence version 1).

In a preferred embodiment of the method and/or cell of the invention, the cell comprises multiple copies of the same coding DNA sequence encoding for one protein. In the context of the present invention, said protein can be a glycosyltransferase, a membrane transporter protein or any other protein as disclosed herein. Throughout the application, the feature “multiple” means at least 2, preferably at least 3, more preferably at least 4, even more preferably at least 5.

According to another preferred embodiment of the method and/or cell of the invention, the cell comprises a modification for reduced production of acetate. Said modification can be any one or more chosen from the group comprising overexpression of an acetyl-coenzyme A synthetase, a full or partial knock-out or rendered less functional pyruvate dehydrogenase and a full or partial knock-out or rendered less functional lactate dehydrogenase.

In a further embodiment of the method and/or cell of the invention, the cell is modified in the expression or activity of at least one acetyl-coenzyme A synthetase like e.g. acs from E. coli, S. cerevisiae, H. sapiens, M. musculus. In a preferred embodiment, said acetyl-coenzyme A synthetase is an endogenous protein of the cell with a modified expression or activity, preferably said endogenous acetyl-coenzyme A synthetase is overexpressed; alternatively, said acetyl-coenzyme A synthetase is a heterologous protein that is heterogeneously introduced and expressed in said cell, preferably overexpressed. Said endogenous acetyl-coenzyme A synthetase can have a modified expression in the cell which also expresses a heterologous acetyl-coenzyme A synthetase. In a more preferred embodiment, the cell is modified in the expression or activity of the acetyl-coenzyme A synthetase acs from E. coli (UniProt ID P27550, sequence version 2). In another and/or additional preferred embodiment, the cell is modified in the expression or activity of a functional homolog, variant or derivative of acs from E. coli (UniProt ID P27550, sequence version 2) having at least 80% overall sequence identity to the full-length of said polypeptide from E. coli (UniProt ID P27550, sequence version 2) and having acetyl-coenzyme A synthetase activity. In an alternative and/or additional further embodiment of the method and/or cell of the invention, the cell is modified in the expression or activity of at least one pyruvate dehydrogenase like e.g. from E. coli, S. cerevisiae, H. sapiens and R. norvegicus. In a preferred embodiment, the cell has been modified to have at least one partially or fully knocked out or mutated pyruvate dehydrogenase encoding gene by means generally known by the person skilled in the art resulting in at least one protein with less functional or being disabled for pyruvate dehydrogenase activity. In a more preferred embodiment, the cell has a full knock-out in the poxB encoding gene resulting in a cell lacking pyruvate dehydrogenase activity. In an alternative and/or additional further embodiment of the method and/or cell of the invention, the cell is modified in the expression or activity of at least one lactate dehydrogenase like e.g. from E. coli, S. cerevisiae, H. sapiens and R. norvegicus. In a preferred embodiment, the cell has been modified to have at least one partially or fully knocked out or mutated lactate dehydrogenase encoding gene by means generally known by the person skilled in the art resulting in at least one protein with less functional or being disabled for lactate dehydrogenase activity. In a more preferred embodiment, the cell has a full knock-out in the IdhA encoding gene resulting in a cell lacking lactate dehydrogenase activity.

According to another preferred embodiment of the method and/or cell of the invention, the cell comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose: undecaprenyl-phosphate glucose-1-phosphate transferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1 ATP-dependent 6-phosphofructokinase isozyme 2, glucose-6-phosphate isomerase, aerobic respiration control protein, transcriptional repressor IclR, Ion protease, glucose-specific translocating phosphotransferase enzyme IIBC component ptsG, glucose-specific translocating phosphotransferase (PTS) enzyme IIBC component maIX, enzyme IIA^{Glc}, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, pyruvate decarboxylase.

According to another preferred embodiment of the method and/or cell of the present invention, the cell is capable to produce phosphoenolpyruvate (PEP). In another preferred embodiment of the method and/or cell of the present invention, the cell is modified for enhanced production and/or supply of phosphoenolpyruvate (PEP).

In a preferred embodiment and as a means for enhanced production and/or supply of PEP, one or more PEP-dependent, sugar-transporting phosphotransferase system(s) is/are disrupted such as but not limited to: 1) the N-acetyl-D-glucosamine Npi-phosphotransferase (EC 2.7.1.193), which is for instance encoded by the nagE gene (or the cluster nagABCD) in E. coli or Bacillus species, 2) ManXYZ which encodes the Enzyme II Man complex (mannose PTS permease, protein-Npi-phosphohistidine-D-mannose phosphotransferase) that imports exogenous hexoses (mannose, glucose, glucosamine, fructose, 2-deoxyglucose, mannosamine, N-acetylglucosamine, etc.) and releases the phosphate esters into the cell cytoplasm, 3) the glucose-specific PTS transporter (for instance encoded by PtsG/Crr) which takes up glucose and forms glucose-6-phosphate in the cytoplasm, 4) the sucrose-specific PTS transporter which takes up sucrose and forms sucrose-6-phosphate in the cytoplasm, 5) the fructose-specific PTS transporter (for instance encoded by the genes fruA and fruB and the kinase fruk which takes up fructose and forms in a first step fructose-1-phosphate and in a second step fructose1,6 bisphosphate, 6) the lactose PTS transporter (for instance encoded by lacE in Lactococcus casei) which takes up lactose and forms lactose-6-phosphate, 7) the galactitol-specific PTS enzyme which takes up galactitol and/or sorbitol and forms galactitol-1-phosphate or sorbitol-6-phosphate respectively, 8) the mannitol-specific PTS enzyme which takes up mannitol and/or sorbitol and forms mannitol-1-phosphate or sorbitol-6-phosphate respectively, and 9) the trehalose-specific PTS enzyme which takes up trehalose and forms trehalose-6-phosphate. In another and/or additional preferred embodiment and as a means for enhanced production and/or supply of PEP, the full PTS system is disrupted by disrupting the PtsIH/Crr gene cluster. PtsI (Enzyme I) is a cytoplasmic protein that serves as the gateway for the phosphoenolpyruvate: sugar phosphotransferase system (PTS^{sugar}) of E. coli K-12. PtsI is one of two (PtsI and PtsH) sugar non-specific protein constituents of the PTS^{sugar} which along with a sugar-specific inner membrane permease effects a phosphotransfer cascade that results in the coupled phosphorylation and transport of a variety of carbohydrate substrates. HPr (histidine containing protein) is one of two sugar-non-specific protein constituents of the PTS^{sugar}. It accepts a phosphoryl group from phosphorylated Enzyme I (PtsI-P) and then transfers it to the EIIA domain of any one of the many sugar-specific enzymes (collectively known as Enzymes II) of the PTS^{sugar}. Crr or EIIA^{Glc} is phosphorylated by PEP in a reaction requiring PtsH and PtsI.

In another and/or additional preferred embodiment, the cell is further modified to compensate for the deletion of a PTS system of a carbon source by the introduction and/or overexpression of the corresponding permease. These are e.g. permeases or ABC transporters that comprise but are not limited to transporters that specifically import lactose such as e.g. the transporter encoded by the LacY gene from E. coli, sucrose such as e.g. the transporter encoded by the cscB gene from E. coli, glucose such as e.g. the transporter encoded by the galP gene from E. coli, fructose such as e.g. the transporter encoded by the frul gene from Streptococcus mutans, or the Sorbitol/mannitol ABC transporter such as the transporter encoded by the cluster SmoEFGK of Rhodobacter sphaeroides, the trehalose/sucrose/maltose transporter such as the transporter encoded by the gene cluster ThuEFGK of Sinorhizobium meliloti and the N-acetylglucosamine/galactose/glucose transporter such as the transporter encoded by NagP of Shewanella oneidensis. Examples of combinations of PTS deletions with overexpression of alternative transporters are: 1) the deletion of the glucose PTS system, e.g. ptsG gene, combined with the introduction and/or overexpression of a glucose permease (e.g. galP of glcP), 2) the deletion of the fructose PTS system, e.g. one or more of the fruB, fruA, fruk genes, combined with the introduction and/or overexpression of fructose permease, e.g. frul, 3) the deletion of the lactose PTS system, combined with the introduction and/or overexpression of lactose permease, e.g. LacY, and/or 4) the deletion of the sucrose PTS system, combined with the introduction and/or overexpression of a sucrose permease, e.g. cscB.

In a further preferred embodiment, the cell is modified to compensate for the deletion of a PTS system of a carbon source by the introduction of carbohydrate kinases, such as glucokinase (EC 2.7.1.1, EC 2.7.1.2, EC 2.7.1.63), galactokinase (EC 2.7.1.6), and/or fructokinase (EC 2.7.1.3, EC 2.7.1.4). Examples of combinations of PTS deletions with overexpression of alternative transporters and a kinase are: 1) the deletion of the glucose PTS system, e.g. ptsG gene, combined with the introduction and/or overexpression of a glucose permease (e.g. galP of glcP), combined with the introduction and/or overexpression of a glucokinase (e.g. glk), and/or 2) the deletion of the fructose PTS system, e.g. one or more of the fruB, fruA, fruk genes, combined with the introduction and/or overexpression of fructose permease, e.g. frul, combined with the introduction and/or overexpression of a fructokinase (e.g. frk or mak).

In another and/or additional preferred embodiment and as a means for enhanced production and/or supply of PEP, the cell is modified by the introduction of or modification in any one or more of the list comprising phosphoenolpyruvate synthase activity (EC: 2.7.9.2 encoded for instance in E. coli by ppsA), phosphoenolpyruvate carboxykinase activity (EC 4.1.1.32 or EC 4.1.1.49 encoded for instance in Corynebacterium glutamicum by PCK or in E. coli by pckA, resp.), phosphoenolpyruvate carboxylase activity (EC 4.1.1.31 encoded for instance in E. coli by ppc), oxaloacetate decarboxylase activity (EC 4.1.1.112 encoded for instance in E. coli by eda), pyruvate kinase activity (EC 2.7.1.40 encoded for instance in E. coli by pykA and pykF), pyruvate carboxylase activity (EC 6.4.1.1 encoded for instance in B. subtilis by pyc) and malate dehydrogenase activity (EC 1.1.1.38 or EC 1.1.1.40 encoded for instance in E. coli by maeA or maeB, resp.).

In a more preferred embodiment, the cell is modified to overexpress any one or more of the polypeptides comprising ppsA from E. coli (UniProt ID P23538, sequence version 5), PCK from C. glutamicum (UniProt ID Q6F5A5, sequence version 1), pcka from E. coli (UniProt ID P22259, sequence version 2), eda from E. coli (UniProt ID POA955, sequence version 1), maeA from E. coli (UniProt ID P26616, sequence version 4) and maeB from E. coli (UniProt ID P76558, sequence version 1).

In another and/or additional preferred embodiment, the cell is modified to express any one or more polypeptide having phosphoenolpyruvate synthase activity, phosphoenolpyruvate carboxykinase activity, oxaloacetate decarboxylase activity, or malate dehydrogenase activity.

In another and/or additional preferred embodiment and as a means for enhanced production and/or supply of PEP, the cell is modified by a reduced activity of phosphoenolpyruvate carboxylase activity, and/or pyruvate kinase activity, preferably a deletion of the genes encoding for phosphoenolpyruvate carboxylase, the pyruvate carboxylase activity and/or pyruvate kinase.

In an exemplary embodiment, the cell is genetically modified by different adaptations such as the overexpression of phosphoenolpyruvate synthase combined with the deletion of a pyruvate kinase gene, the overexpression of phosphoenolpyruvate synthase combined with the deletion of a phosphoenolpyruvate carboxylase gene, the overexpression of phosphoenolpyruvate synthase combined with the deletion of a pyruvate carboxylase gene, the overexpression of phosphoenolpyruvate carboxykinase combined with the deletion of a pyruvate kinase gene, the overexpression of phosphoenolpyruvate carboxykinase combined with the deletion of a phosphoenolpyruvate carboxylase gene, the overexpression of phosphoenolpyruvate carboxykinase combined with the deletion of a pyruvate carboxylase gene, the overexpression of oxaloacetate decarboxylase combined with the deletion of a pyruvate kinase gene, the overexpression of oxaloacetate decarboxylase combined with the deletion of a phosphoenolpyruvate carboxylase gene, the overexpression of oxaloacetate decarboxylase combined with the deletion of a pyruvate carboxylase gene, the overexpression of malate dehydrogenase combined with the deletion of a pyruvate kinase gene, the overexpression of malate dehydrogenase combined with the deletion of a phosphoenolpyruvate carboxylase gene and/or the overexpression of malate dehydrogenase combined with the deletion of a pyruvate carboxylase gene.

In another exemplary embodiment, the cell is genetically modified by different adaptations such as the overexpression of phosphoenolpyruvate synthase combined with the overexpression of a phosphoenolpyruvate carboxykinase, the overexpression of phosphoenolpyruvate synthase combined with the overexpression of an oxaloacetate decarboxylase, the overexpression of phosphoenolpyruvate synthase combined with the overexpression of a malate dehydrogenase, the overexpression of phosphoenolpyruvate carboxykinase combined with the overexpression of an oxaloacetate decarboxylase, the overexpression of phosphoenolpyruvate carboxykinase combined with the overexpression of a malate dehydrogenase, the overexpression of oxaloacetate decarboxylase combined with the overexpression of a malate dehydrogenase, the overexpression of phosphoenolpyruvate synthase combined with the overexpression of a phosphoenolpyruvate carboxykinase and the overexpression of an oxaloacetate decarboxylase, the overexpression of phosphoenolpyruvate synthase combined with the overexpression of a phosphoenolpyruvate carboxykinase and the overexpression of a malate dehydrogenase, the overexpression of phosphoenolpyruvate synthase combined with the overexpression of a phosphoenolpyruvate carboxykinase and the overexpression of an oxaloacetate decarboxylase and the overexpression of a malate dehydrogenase, the overexpression of phosphoenolpyruvate carboxykinase combined with the overexpression of an oxaloacetate decarboxylase and the overexpression of a malate dehydrogenase and/or the overexpression of phosphoenolpyruvate synthase combined with the overexpression of an oxaloacetate decarboxylase and the overexpression of a malate dehydrogenase.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate kinase gene, the overexpression of phosphoenolpyruvate synthase combined with the overexpression of an oxaloacetate decarboxylase combined with the deletion of a pyruvate kinase gene, the overexpression of phosphoenolpyruvate synthase combined with the overexpression of a malate dehydrogenase combined with the deletion of a pyruvate kinase gene, the overexpression of phosphoenolpyruvate carboxykinase combined with the overexpression of an oxaloacetate decarboxylase combined with the deletion of a pyruvate kinase gene, the overexpression of phosphoenolpyruvate carboxykinase combined with the overexpression of a malate dehydrogenase combined with the deletion of a pyruvate kinase gene, the overexpression of an oxaloacetate decarboxylase combined with the overexpression of a malate dehydrogenase combined with the deletion of a pyruvate kinase gene, the overexpression of phosphoenolpyruvate synthase combined with the overexpression of a phosphoenolpyruvate carboxykinase and the overexpression of an oxaloacetate decarboxylase combined with the deletion of a pyruvate kinase gene, the overexpression of phosphoenolpyruvate synthase combined with the overexpression of a phosphoenolpyruvate carboxykinase and the overexpression of a malate dehydrogenase combined with the deletion of a pyruvate kinase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate kinase gene, the overexpression of a phosphoenolpyruvate carboxykinase combined with the overexpression of an oxaloacetate decarboxylase and the overexpression of a malate dehydrogenase combined with the deletion of a pyruvate kinase gene, the overexpression of phosphoenolpyruvate synthase combined the overexpression of oxaloacetate decarboxylase and the overexpression of a malate dehydrogenase combined with the deletion of a pyruvate kinase gene.

In another exemplary embodiment, the cell is genetically modified by different adaptations such as the overexpression of phosphoenolpyruvate synthase combined with the overexpression of a phosphoenolpyruvate carboxykinase combined with the deletion of a phosphoenolpyruvate carboxylase gene, the overexpression of phosphoenolpyruvate synthase combined with the overexpression of an oxaloacetate decarboxylase combined with the deletion of a phosphoenolpyruvate carboxylase gene, the overexpression of phosphoenolpyruvate synthase combined with the overexpression of a malate dehydrogenase combined with the deletion of a phosphoenolpyruvate carboxylase gene, the overexpression of a phosphoenolpyruvate carboxykinase combined with the overexpression of an oxaloacetate decarboxylase combined with the deletion of a phosphoenolpyruvate carboxylase gene, the overexpression of a phosphoenolpyruvate carboxykinase combined with the overexpression of a malate dehydrogenase combined with the deletion of a phosphoenolpyruvate carboxylase gene, the overexpression of an oxaloacetate decarboxylase combined with the overexpression of a malate dehydrogenase combined with the deletion of a phosphoenolpyruvate carboxylase gene, the overexpression of phosphoenolpyruvate synthase combined with the overexpression of a phosphoenolpyruvate carboxykinase and the overexpression of an oxaloacetate decarboxylase combined with the deletion of a phosphoenolpyruvate carboxylase gene, the overexpression of phosphoenolpyruvate synthase combined with the overexpression of a phosphoenolpyruvate carboxykinase and the overexpression of a malate dehydrogenase combined with the deletion of a phosphoenolpyruvate carboxylase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a phosphoenolpyruvate carboxylase gene, the overexpression of a phosphoenolpyruvate carboxykinase combined with the overexpression of an oxaloacetate decarboxylase and the overexpression of a malate dehydrogenase combined with the deletion of a phosphoenolpyruvate carboxylase gene, the overexpression of phosphoenolpyruvate synthase combined the overexpression of an oxaloacetate decarboxylase and the overexpression of a malate dehydrogenase combined with the deletion of a phosphoenolpyruvate carboxylase gene.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate carboxylase gene, the overexpression of phosphoenolpyruvate synthase combined with the overexpression of an oxaloacetate decarboxylase combined with the deletion of a pyruvate carboxylase gene, the overexpression of phosphoenolpyruvate synthase combined with the overexpression of a malate dehydrogenase combined with the deletion of a pyruvate carboxylase gene, the overexpression of a phosphoenolpyruvate carboxykinase combined with the overexpression of an oxaloacetate decarboxylase combined with the deletion of a pyruvate carboxylase gene, the overexpression of a phosphoenolpyruvate carboxykinase combined with the overexpression of a malate dehydrogenase combined with the deletion of a pyruvate carboxylase gene, the overexpression of an oxaloacetate decarboxylase combined with the overexpression of a malate dehydrogenase combined with the deletion of a pyruvate carboxylase gene, the overexpression of phosphoenolpyruvate synthase combined with the overexpression of a phosphoenolpyruvate carboxykinase and the overexpression of an oxaloacetate decarboxylase combined with the deletion of a pyruvate carboxylase gene, the overexpression of phosphoenolpyruvate synthase combined with the overexpression of a phosphoenolpyruvate carboxykinase and the overexpression of a malate dehydrogenase combined with the deletion of a pyruvate carboxylase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate carboxylase gene, the overexpression of a phosphoenolpyruvate carboxykinase combined with the overexpression of an oxaloacetate decarboxylase and the overexpression of a malate dehydrogenase combined with the deletion of a pyruvate carboxylase gene, the overexpression of phosphoenolpyruvate synthase combined with the overexpression of an oxaloacetate decarboxylase and the overexpression of a malate dehydrogenase combined with the deletion of a pyruvate carboxylase gene.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate kinase gene and a phosphoenolpyruvate carboxylase gene, the overexpression of phosphoenolpyruvate synthase combined with the overexpression of an oxaloacetate decarboxylase combined with the deletion of a pyruvate kinase gene and a phosphoenolpyruvate carboxylase gene, the overexpression of phosphoenolpyruvate synthase combined with the overexpression of a malate dehydrogenase combined with the deletion of a pyruvate kinase gene and a phosphoenolpyruvate carboxylase gene, the overexpression of a phosphoenolpyruvate carboxykinase combined with the overexpression of an oxaloacetate decarboxylase combined with the deletion of a pyruvate kinase gene and a phosphoenolpyruvate carboxylase gene, the overexpression of a phosphoenolpyruvate carboxykinase combined with the overexpression of a malate dehydrogenase combined with the deletion of a pyruvate kinase gene and a phosphoenolpyruvate carboxylase gene, the overexpression of an oxaloacetate decarboxylase combined with the overexpression of a malate dehydrogenase combined with the deletion of a pyruvate kinase gene and a phosphoenolpyruvate carboxylase gene, the overexpression of a phosphoenolpyruvate synthase combined with the overexpression of a phosphoenolpyruvate carboxykinase and the overexpression of an oxaloacetate decarboxylase combined with the deletion of a pyruvate kinase gene and a phosphoenolpyruvate carboxylase gene, the overexpression of a phosphoenolpyruvate synthase combined with the overexpression of a phosphoenolpyruvate carboxykinase and the overexpression of a malate dehydrogenase combined with the deletion of a pyruvate kinase gene and a phosphoenolpyruvate carboxylase gene, the overexpression of a phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate kinase gene and a phosphoenolpyruvate carboxylase gene, the overexpression of a phosphoenolpyruvate carboxykinase combined with the overexpression of an oxaloacetate decarboxylase and the overexpression of a malate dehydrogenase combined with the deletion of a pyruvate kinase gene and a phosphoenolpyruvate carboxylase gene, the overexpression of a phosphoenolpyruvate synthase combined with the overexpression of an oxaloacetate decarboxylase and the overexpression of a malate dehydrogenase combined with the deletion of a pyruvate kinase gene and a phosphoenolpyruvate carboxylase gene.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate kinase gene and a pyruvate carboxylase gene and a phosphoenolpyruvate carboxylase gene, the overexpression of phosphoenolpyruvate synthase combined with the overexpression of an oxaloacetate decarboxylase combined with the deletion of a pyruvate kinase gene and a pyruvate carboxylase gene and a phosphoenolpyruvate carboxylase gene, the overexpression of phosphoenolpyruvate synthase combined with the overexpression of a malate dehydrogenase combined with the deletion of a pyruvate kinase gene and a pyruvate carboxylase gene and a phosphoenolpyruvate carboxylase gene, the overexpression of a phosphoenolpyruvate carboxykinase combined with the overexpression of an oxaloacetate decarboxylase combined with the deletion of a pyruvate kinase gene and a pyruvate carboxylase gene and a phosphoenolpyruvate carboxylase gene, the overexpression of a phosphoenolpyruvate carboxykinase combined with the overexpression of a malate dehydrogenase combined with the deletion of a pyruvate kinase gene and a pyruvate carboxylase gene and a phosphoenolpyruvate carboxylase gene, the overexpression of an oxaloacetate decarboxylase combined with the overexpression of a malate dehydrogenase combined with the deletion of a pyruvate kinase gene and a pyruvate carboxylase gene and a phosphoenolpyruvate carboxylase gene, the overexpression of phosphoenolpyruvate synthase combined with the overexpression of a phosphoenolpyruvate carboxykinase and the overexpression of an oxaloacetate decarboxylase combined with the deletion of a pyruvate kinase gene and a pyruvate carboxylase gene and a phosphoenolpyruvate carboxylase gene, the overexpression of phosphoenolpyruvate synthase combined with the overexpression of a phosphoenolpyruvate carboxykinase and the overexpression of a malate dehydrogenase combined with the deletion of a pyruvate kinase gene and a pyruvate carboxylase gene and a phosphoenolpyruvate carboxylase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate kinase gene and pyruvate carboxylase gene and a phosphoenolpyruvate carboxylase gene, the overexpression of a phosphoenolpyruvate carboxykinase combined with the overexpression of an oxaloacetate decarboxylase and the overexpression of a malate dehydrogenase combined with the deletion of a pyruvate kinase gene and a pyruvate carboxylase gene and a phosphoenolpyruvate carboxylase gene, the overexpression of phosphoenolpyruvate synthase combined with the overexpression of an oxaloacetate decarboxylase and the overexpression of a malate dehydrogenase combined with the deletion of a pyruvate kinase gene and a pyruvate carboxylase gene and a phosphoenolpyruvate carboxylase gene. According to another preferred embodiment of the method and/or cell of the invention, the cell comprises a catabolic pathway for selected mono-, di- or oligosaccharides which is at least partially inactivated, the mono-, di-, or oligosaccharides being involved in and/or required for the production of said compound comprising a structure of Formula I, II or III.

According to another preferred embodiment of the method and/or cell of the invention, the cell produces 90 g/L or more of said compound comprising a structure of Formula I, II or III in the whole broth and/or supernatant and/or wherein said compound comprising a structure of Formula I, II or III in the whole broth and/or supernatant has a purity of at least 80% measured on the total amount of said compound comprising a structure of Formula I, II or III and its precursor(s) in the whole broth and/or supernatant, respectively.

Another embodiment of the invention provides for a method and a cell wherein said compound comprising a structure of Formula I, II or III is produced in and/or by a fungal, yeast, bacterial, insect, plant, animal or protozoan cell as described herein. The cell is chosen from the list comprising a bacterium, a yeast, or a fungus, or, refers to a plant, animal, or protozoan cell. The latter bacterium preferably belongs to the phylum of the Proteobacteria or the phylum of the Firmicutes or the phylum of the Cyanobacteria or the phylum Deinococcus-Thermus. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains-designated as E. coli K12 strains-which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, the present invention specifically relates to a mutated and/or transformed Escherichia coli cell or strain as indicated above wherein said E. coli strain is a K12 strain. More preferably, the Escherichia coli K12 strain is E. coli MG1655. The latter bacterium belonging to the phylum Firmicutes belongs preferably to the Bacilli, preferably Lactobacilliales, with members such as Lactobacillus lactis, Leuconostoc mesenteroides, or Bacillales with members such as from the genus Bacillus, such as Bacillus subtilis or, B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces (with members like e.g. Saccharomyces cerevisiae, S. bayanus, S. boulardii), Zygosaccharomyces, Pichia (with members like e.g. Pichia pastoris, P. anomala, P. kluyveri), Komagataella, Hansenula, Kluyveromyces (with members like e.g. Kluyveromyces lactis, K. marxianus, K. thermotolerans), Debaromyces, Yarrowia (like e.g. Yarrowia lipolytica) or Starmerella (like e.g. Starmerella bombicola). The latter yeast is preferably selected from Pichia pastoris, Yarrowia lipolitica, Saccharomyces cerevisiae and Kluyveromyces lactis. The latter fungus belongs preferably to the genus Rhizopus, Dictyostelium, Penicillium, Mucor or Aspergillus. Plant cells include cells of flowering and non-flowering plants, as well as algal cells, for example Chlamydomonas, Chlorella, etc. Preferably, said plant is a tobacco, alfalfa, rice, tomato, cotton, rapeseed, soy, maize or corn plant. The latter animal cell is preferably derived from non-human mammals (e.g. cattle, buffalo, pig, sheep, mouse, rat), birds (e.g. chicken, duck, ostrich, turkey, pheasant), fish (e.g. swordfish, salmon, tuna, sea bass, trout, catfish), invertebrates (e.g. lobster, crab, shrimp, clams, oyster, mussel, sea urchin), reptiles (e.g. snake, alligator, turtle), amphibians (e.g. frogs) or insects (e.g. fly, nematode) or is a genetically modified cell line derived from human cells excluding embryonic stem cells. Both human and non-human mammalian cells are preferably chosen from the list comprising an epithelial cell like e.g. a mammary epithelial cell, an embryonic kidney cell (e.g. HEK293 or HEK 293T cell), a fibroblast cell, a COS cell, a Chinese hamster ovary (CHO) cell, a murine myeloma cell like e.g. an N20, SP2/0 or YB2/0 cell, an NIH-3T3 cell, a non-mammary adult stem cell or derivatives thereof such as described in WO21067641. The latter insect cell is preferably derived from Spodoptera frugiperda like e.g. Sf9 or Sf21 cells, Bombyx mori, Mamestra brassicae, Trichoplusia ni like e.g. BTI-TN-5B1-4 cells or Drosophila melanogaster like e.g. Drosophila S2 cells. The latter protozoan cell preferably is a Leishmania tarentolae cell.

According to a preferred embodiment of the method and/or cell of the invention, said compound comprising a structure of Formula I, II or III is produced in and/or by a cell which is a viable Gram-negative bacterium that comprises a reduced or abolished synthesis of poly-N-acetyl-glucosamine (PNAG), Enterobacterial Common Antigen (ECA), cellulose, colanic acid, core oligosaccharides, Osmoregulated Periplasmic Glucans (OPG), Glucosylglycerol, glycan, and/or trehalose.

In a more preferred embodiment of the method and/or cell, said reduced or abolished synthesis of poly-N-acetyl-glucosamine (PNAG), Enterobacterial Common Antigen (ECA), cellulose, colanic acid, core oligosaccharides, Osmoregulated Periplasmic Glucans (OPG), Glucosylglycerol, glycan, and/or trehalose is provided by a mutation in any one or more glycosyltransferases involved in the synthesis of any one of said poly-N-acetyl-glucosamine (PNAG), Enterobacterial Common Antigen (ECA), cellulose, colanic acid, core oligosaccharides, Osmoregulated Periplasmic Glucans (OPG), Glucosylglycerol, glycan, and/or trehalose, wherein said mutation provides for a deletion or lower expression of any one of said glycosyltransferases. Said glycosyltransferases comprise glycosyltransferase genes encoding poly-N-acetyl-D-glucosamine synthase subunits, UDP-N-acetylglucosamine-undecaprenyl-phosphate N-acetylglucosaminephosphotransferase, Fuc4NAc (4-acetamido-4,6-dideoxy-D-galactose) transferase, UDP-N-acetyl-D-mannosaminuronic acid transferase, the glycosyltransferase genes encoding the cellulose synthase catalytic subunits, the cellulose biosynthesis protein, colanic acid biosynthesis glucuronosyltransferase, colanic acid biosynthesis galactosyltransferase, colanic acid biosynthesis fucosyltransferase, UDP-glucose: undecaprenyl-phosphate glucose-1-phosphate transferase, putative colanic biosynthesis glycosyl transferase, UDP-glucuronate: LPS (HepIII)glycosyltransferase, ADP-heptose-LPS heptosyltransferase 2, ADP-heptose: LPS heptosyltransferase 1, putative ADP-heptose: LPS heptosyltransferase 4, lipopolysaccharide core biosynthesis protein, UDP-glucose: (glucosyl) LPS α-1,2-glucosyltransferase, UDP-D-glucose: (glucosyl) LPS α-1,3-glucosyltransferase, UDP-D-galactose: (glucosyl) lipopolysaccharide-1,6-D-galactosyltransferase, lipopolysaccharide glucosyltransferase I, lipopolysaccharide core heptosyltransferase 3, β-1,6-galactofuranosyltransferase, undecaprenyl-phosphate 4-deoxy-4-formamido-L-arabinose transferase, lipid IVA 4-amino-4-deoxy-L-arabinosyltransferase, bactoprenol glucosyl transferase, putative family 2 glycosyltransferase, the osmoregulated periplasmic glucans (OPG) biosynthesis protein G, OPG biosynthesis protein H, glucosylglycerate phosphorylase, glycogen synthase, 1,4-a-glucan branching enzyme, 4-a-glucanotransferase and trehalose-6-phosphate synthase. In an exemplary embodiment, the cell is mutated in any one or more of the glycosyltransferases comprising pgaC, pgaD, rfe, rffT, rffM, bcsA, bcsB, bcsC, wcaA, wcaC, wcaE, wcaI, wcaJ, wcaL, waaH, waaF, waaC, waaU, waaZ, waaJ, waaO, waaB, waas, waaG, waaQ, wbbI, arnC, arnT, yfdH, wbbK, opgG, opgH, ycjM, glgA, glgB, malQ, otsA and yaiP, wherein said mutation provides for a deletion or lower expression of any one of said glycosyltransferases.

In an alternative and/or additional preferred embodiment of the method and/or cell, said reduced or abolished synthesis of poly-N-acetyl-glucosamine (PNAG) is provided by over-expression of a carbon storage regulator encoding gene, deletion of a Na+/H+ antiporter regulator encoding gene and/or deletion of the sensor histidine kinase encoding gene.

Another embodiment provides for a cell to be stably cultured in a medium, wherein said medium can be any type of growth medium as well-known to the skilled person comprising minimal medium, complex medium or growth medium enriched in certain compounds like for example but not limited to vitamins, trace elements, amino acids and/or, precursors and/or acceptors as defined herein.

The cell as used herein is capable to grow on a monosaccharide, disaccharide, oligosaccharide, polysaccharide, polyol, glycerol, a complex medium including molasses, corn steep liquor, peptone, tryptone, yeast extract or a mixture thereof like e.g. a mixed feedstock, preferably a mixed monosaccharide feedstock like e.g. hydrolysed sucrose as the main carbon source. With the term “complex medium” is meant a medium for which the exact constitution is not determined. With the term “main” is meant the most important carbon source for the cell for the production of the di- and/or oligosaccharide of interest, biomass formation, carbon dioxide and/or by-products formation (such as acids and/or alcohols, such as acetate, lactate, and/or ethanol), i.e. 20, 30, 40, 50, 60, 70, 75, 80, 85, 90, 95, 98, 99% of all the required carbon is derived from the above-indicated carbon source. In one embodiment of the invention, said carbon source is the sole carbon source for said organism, i.e. 100% of all the required carbon is derived from the above-indicated carbon source. Common main carbon sources comprise but are not limited to glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate. As used herein, a precursor as defined herein cannot be used as a carbon source for the production of said compound comprising a structure of Formula I, II or III.

According to another embodiment of the method of the invention, the conditions permissive to produce said compound comprising a structure of Formula I, II or III comprise the use of a culture medium comprising at least one precursor and/or acceptor for the production of said compound comprising a structure of Formula I, II or III. Preferably, the culture medium contains at least one precursor selected from the group comprising lactose, galactose, fucose, sialic acid, GlcNAc, GalNAc, lacto-N-biose (LNB), N-acetyllactosamine (LacNAc).

According to an alternative and/or additional embodiment of the method of the invention, the conditions permissive to produce said compound comprising a structure of Formula I, II or III comprise adding to the culture medium at least one precursor and/or acceptor feed for the production of compound comprising a structure of Formula I, II or III.

According to an alternative embodiment of the method of the invention, the conditions permissive to produce said compound comprising a structure of Formula I, II or III comprise the use of a culture medium to cultivate a cell of present invention for the production of compound comprising a structure of Formula I, II or III wherein said culture medium lacks any precursor and/or acceptor for the production of said compound comprising a structure of Formula I, II or III and is combined with a further addition to said culture medium of at least one precursor and/or acceptor feed for the production of said compound comprising a structure of Formula I, II or III.

In a preferred embodiment, the method for the production of said compound comprising a structure of Formula I, II or III as described herein comprises at least one of the following steps:
i) Use of a culture medium comprising at least one precursor and/or acceptor;
ii) Adding to the culture medium in a reactor at least one precursor and/or acceptor feed wherein the total reactor volume ranges from 250 ml (millilitre) to 10.000 m³ (cubic meter), preferably in a continuous manner, and preferably so that the final volume of the culture medium is not more than three-fold, preferably not more than two-fold, more preferably less than two-fold of the volume of the culture medium before the addition of said precursor and/or acceptor feed;
iii) Adding to the culture medium in a reactor at least one precursor and/or acceptor feed wherein the total reactor volume ranges from 250 ml (millilitre) to 10.000 m³ (cubic meter), preferably in a continuous manner, and preferably so that the final volume of the culture medium is not more than three-fold, preferably not more than two-fold, more preferably less than two-fold of the volume of the culture medium before the addition of said precursor and/or acceptor feed and wherein preferably, the pH of said precursor and/or acceptor feed is set between 3 and 7 and wherein preferably, the temperature of said precursor and/or acceptor feed is kept between 20° C. and 80° C.;
iv) Adding at least one precursor and/or acceptor feed in a continuous manner to the culture medium over the course of 1 day, 2 days, 3 days, 4 days, 5 days by means of a feeding solution;
v) Adding at least one precursor and/or acceptor feed in a continuous manner to the culture medium over the course of 1 day, 2 days, 3 days, 4 days, 5 days by means of a feeding solution and wherein preferably, the pH of said feeding solution is set between 3 and 7 and wherein preferably, the temperature of said feeding solution is kept between 20° C. and 80° C.;
said method resulting in said compound comprising a structure of Formula I, II or III with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final cultivation.

In another and/or additional preferred embodiment, the method for the production of said compound comprising a structure of Formula I, II or III as described herein comprises at least one of the following steps:
i) Use of a culture medium comprising at least 50, more preferably at least 75, more preferably at least 100, more preferably at least 120, more preferably at least 150 gram of lactose per litre of initial reactor volume wherein the reactor volume ranges from 250 ml to 10.000 m³ (cubic meter);
ii) Adding to the culture medium at least one precursor and/or acceptor in one pulse or in a discontinuous (pulsed) manner wherein the total reactor volume ranges from 250 ml (millilitre) to 10.000 m³ (cubic meter), preferably so that the final volume of the culture medium is not more than three-fold, preferably not more than two-fold, more preferably less than two-fold of the volume of the culture medium before the addition of said precursor and/or acceptor feed pulse(s);
iii) Adding to the culture medium in a reactor at least one precursor and/or acceptor feed in one pulse or in a discontinuous (pulsed) manner wherein the total reactor volume ranges from 250 ml (millilitre) to 10.000 m³ (cubic meter), preferably so that the final volume of the culture medium is not more than three-fold, preferably not more than two-fold, more preferably less than two-fold of the volume of the culture medium before the addition of said precursor and/or acceptor feed pulse(s) and wherein preferably, the pH of said precursor and/or acceptor feed pulse(s) is set between 3 and 7 and wherein preferably, the temperature of said precursor and/or acceptor feed pulse(s) is kept between 20° C. and 80° C.;
iv) Adding at least one precursor and/or acceptor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution;
v) Adding at least one precursor and/or acceptor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution and wherein preferably, the pH of said feeding solution is set between 3 and 7 and wherein preferably, the temperature of said feeding solution is kept between 20° C. and 80° C.;
said method resulting in said compound comprising a structure of Formula I, II or III with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final cultivation.

In a further, more preferred embodiment, the method for the production of said compound comprising a structure of Formula I, II or III as described herein comprises at least one of the following steps:
i) Use of a culture medium comprising at least 50, more preferably at least 75, more preferably at least 100, more preferably at least 120, more preferably at least 150 gram of lactose per litre of initial reactor volume wherein the reactor volume ranges from 250 ml to 10.000 m³ (cubic meter);
ii) Adding to the culture medium a lactose feed comprising at least 50, more preferably at least 75, more preferably at least 100, more preferably at least 120, more preferably at least 150 gram of lactose per litre of initial reactor volume wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed;
iii) Adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 ml to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed and wherein preferably the pH of said lactose feed is set between 3 and 7 and wherein preferably the temperature of said lactose feed is kept between 20° C. and 80° C.;
iv) Adding a lactose feed in a continuous manner to the culture medium over the course of 1 day, 2 days, 3 days, 4 days, 5 days by means of a feeding solution;
v) Adding a lactose feed in a continuous manner to the culture medium over the course of 1 day, 2 days, 3 days, 4 days, 5 days by means of a feeding solution and wherein the concentration of said lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said feeding solution is set between 3 and 7 and wherein preferably the temperature of said feeding solution is kept between 20° C. and 80° C.;
said method resulting in said compound comprising a structure of Formula I, II or III with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final cultivation.

Preferably the lactose feed is accomplished by adding lactose from the beginning of the cultivation at a concentration of at least 5 mM, preferably in a concentration of 30, 40, 50, 60, 70, 80, 90, 100, 150 mM, more preferably at a concentration >300 mM.

In another embodiment the lactose feed is accomplished by adding lactose to the cultivation in a concentration, such that throughout the production phase of the cultivation a lactose concentration of at least 5 mM, preferably 10 mM or 30 mM is obtained.

In a further embodiment of the methods described herein the cells are cultivated for at least about 60, 80, 100, or about 120 hours or in a continuous manner.

In a preferred embodiment, a carbon source is provided, preferably sucrose, in the culture medium for 3 or more days, preferably up to 7 days; and/or provided, in the culture medium, at least 100, advantageously at least 105, more advantageously at least 110, even more advantageously at least 120 grams of sucrose per litre of initial culture volume in a continuous manner, so that the final volume of the culture medium is not more than three-fold, advantageously not more than two-fold, more advantageously less than two-fold of the volume of the culturing medium before the culturing.

Preferably, when performing the method as described herein, a first phase of exponential cell growth is provided by adding a carbon source, preferably glucose or sucrose, to the culture medium before the lactose is added to the culture medium in a second phase.

In another preferred embodiment of the method of present invention, a first phase of exponential cell growth is provided by adding a carbon-based substrate, preferably glucose or sucrose, to the culture medium comprising a precursor, preferably lactose, followed by a second phase wherein only a carbon-based substrate, preferably glucose or sucrose, is added to the culture medium.

In another preferred embodiment of the method of present invention, a first phase of exponential cell growth is provided by adding a carbon-based substrate, preferably glucose or sucrose, to the culture medium comprising a precursor, preferably lactose, followed by a second phase wherein a carbon-based substrate, preferably glucose or sucrose, and a precursor, preferably lactose, are added to the culture medium.

In an alternative preferable embodiment, in the method as described herein, the lactose is added already in the first phase of exponential growth together with the carbon-based substrate.

According to the present invention, the methods as described herein preferably comprises a step of separating said compound comprising a structure of Formula I, II or III from said cultivation. The terms “separating from said cultivation” means harvesting, collecting, or retrieving said compound comprising a structure of Formula I, II or III from the cell and/or the medium of its growth.

Said compound comprising a structure of Formula I, II or III can be separated in a conventional manner from the aqueous culture medium, in which the cell was grown. In case said compound comprising a structure of Formula I, II or III is still present in the cells producing compound comprising a structure of Formula I, II or III, conventional manners to free or to extract said compound comprising a structure of Formula I, II or III out of the cells can be used, such as cell destruction using high pH, heat shock, sonication, French press, homogenization, enzymatic hydrolysis, chemical hydrolysis, solvent hydrolysis, detergent, hydrolysis, . . . . The culture medium and/or cell extract together and separately can then be further used for separating said compound comprising a structure of Formula I, II or III.

This preferably involves clarifying said compound comprising a structure of Formula I, II or III to remove suspended particulates and contaminants, particularly cells, cell components, insoluble metabolites and debris produced by culturing the genetically modified cell. In this step, said compound comprising a structure of Formula I, II or III can be clarified in a conventional manner. Preferably, said compound comprising a structure of Formula I, II or III is clarified by centrifugation, flocculation, decantation and/or filtration. Another step of separating said compound comprising a structure of Formula I, II or III preferably involves removing substantially all the proteins, peptides, amino acids, RNA and DNA, and any endotoxins and glycolipids that could interfere with the subsequent separation step, from said compound comprising a structure of Formula I, II or III, preferably after it has been clarified. In this step, proteins and related impurities can be removed from said compound comprising a structure of Formula I, II or III in a conventional manner. Preferably, proteins, salts, by-products, colour, endotoxins and other related impurities are removed from said compound comprising a structure of Formula I, II or III by ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis (e.g. using slab-polyacrylamide or sodium dodecyl sulphate-polyacrylamide gel electrophoresis (PAGE)), affinity chromatography (using affinity ligands including e.g. DEAE-Sepharose, poly-L-lysine and polymyxin-B, endotoxin-selective adsorber matrices), ion exchange chromatography (such as but not limited to cation exchange, anion exchange, mixed bed ion exchange, inside-out ligand attachment), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, remaining proteins and related impurities are retained by a chromatography medium or a selected membrane.

In a further preferred embodiment, the methods as described herein also provide for a further purification of said compound comprising a structure of Formula I, II or III as produced according to a method of present invention. A further purification of said compound comprising a structure of Formula I, II or III may be accomplished, for example, by use of (activated) charcoal or carbon, nanofiltration, ultrafiltration, electrophoresis, enzymatic treatment or ion exchange to remove any remaining DNA, protein, LPS, endotoxins, or other impurity. Alcohols, such as ethanol, and aqueous alcohol mixtures can also be used. Another purification step is accomplished by crystallization, evaporation or precipitation of said compound comprising a structure of Formula I, II or III. Another purification step is to dry, e.g. spray dry, lyophilize, spray freeze dry, freeze spray dry, band dry, belt dry, vacuum band dry, vacuum belt dry, drum dry, roller dry, vacuum drum dry or vacuum roller dry the produced compound comprising a structure of Formula I, II or III.

In an exemplary embodiment, the separation and purification of said compound comprising a structure of Formula I, II or III is made in a process, comprising the following steps in any order:
a) contacting the cultivation or a clarified version thereof with a nanofiltration membrane with a molecular weight cut-off (MWCO) of 600-3500 Da ensuring the retention of the produced compound comprising a structure of Formula I, II or III and allowing at least a part of the proteins, salts, by-products, colour and other related impurities to pass,
b) conducting a diafiltration process on the retentate from step a), using said membrane, with an aqueous solution of an inorganic electrolyte, followed by optional diafiltration with pure water to remove excess of the electrolyte,
c) and collecting the retentate enriched in said compound comprising a structure of Formula I, II or III in the form of a salt from the cation of said electrolyte.

In an alternative exemplary embodiment, the separation and purification of said compound comprising a structure of Formula I, II or III is made in a process, comprising the following steps in any order: subjecting the cultivation or a clarified version thereof to two membrane filtration steps using different membranes, wherein
one membrane has a molecular weight cut-off of between about 300 to about 500 Dalton, and
the other membrane as a molecular weight cut-off of between about 600 to about 800 Dalton.

In an alternative exemplary embodiment, the separation and purification of said compound comprising a structure of Formula I, II or III is made in a process, comprising the following steps in any order comprising the step of treating the cultivation or a clarified version thereof with a strong cation exchange resin in H+-form and a weak anion exchange resin in free base form.

In an alternative exemplary embodiment, the separation and purification of said compound comprising a structure of Formula I, II or III is made in the following way. The cultivation comprising the produced compound comprising a structure of Formula I, II or III, biomass, medium components and contaminants is applied to the following purification steps:
i) separation of biomass from the cultivation,
ii) cationic ion exchanger treatment for the removal of positively charged material,
iii) anionic ion exchanger treatment for the removal of negatively charged material,
iv) nanofiltration step and/or electrodialysis step,
wherein a purified solution comprising the produced compound comprising a structure of Formula I, II or III at a purity of greater than or equal to 80 percent is provided. Optionally the purified solution is dried by any one or more drying steps chosen from the list comprising spray drying, lyophilization, spray freeze drying, freeze spray drying, band drying, belt drying, vacuum band drying, vacuum belt drying, drum drying, roller drying, vacuum drum drying and vacuum roller drying.

In an alternative exemplary embodiment, the separation and purification of said compound comprising a structure of Formula I, II or III is made in a process, comprising the following steps in any order: enzymatic treatment of the cultivation; removal of the biomass from the cultivation; ultrafiltration; nanofiltration; and a column chromatography step. Preferably such column chromatography is a single column or a multiple column. Further preferably the column chromatography step is simulated moving bed chromatography. Such simulated moving bed chromatography preferably comprises i) at least 4 columns, wherein at least one column comprises a weak or strong cation exchange resin; and/or ii) four zones I, II, III and IV with different flow rates; and/or iii) an eluent comprising water; and/or iv) an operating temperature of 15 degrees to 60 degrees centigrade.

In a specific embodiment, the present invention provides the produced compound comprising a structure of Formula I, II or III which is dried to powder by any one or more drying steps chosen from the list comprising spray drying, lyophilization, spray freeze drying, freeze spray drying, band drying, belt drying, vacuum band drying, vacuum belt drying, drum drying, roller drying, vacuum drum drying and vacuum roller drying, wherein the dried powder contains <15 percent-wt. of water, preferably <10 percent-wt. of water, more preferably <7 percent-wt. of water, most preferably <5 percent-wt. of water.

Another aspect of the present invention provides the use of a cell as defined herein, in a method for the production of said compound comprising a structure of Formula I, II or III, preferably in a method for the production of said compound comprising a structure of Formula I, II or III according to the invention. An alternative and/or additional aspect of the present invention provides the use of a cell as defined herein, in a method for the production of a mixture of di- and oligosaccharides comprising at least one compound comprising a structure of Formula I, II or III wherein R¹, when present, is a monosaccharide, a disaccharide or an oligosaccharide. An alternative and/or additional aspect of the present invention provides the use of a cell as defined herein, in a method for the production of a mixture of charged and/or neutral di- and oligosaccharides comprising at least one compound comprising a structure of Formula I, II or III wherein R¹, when present, is a monosaccharide, a disaccharide or an oligosaccharide. A preferred aspect of the present invention provides the use of a cell as defined herein, in a method for the production of a mixture of sialylated and/or neutral di- and oligosaccharides comprising at least one compound comprising a structure of Formula I, II or III wherein R¹, when present, is a monosaccharide, a disaccharide or an oligosaccharide. An alternative and/or additional aspect of the present invention provides the use of a cell as defined herein, in a method for the production of a mixture of oligosaccharides comprising at least one compound comprising a structure of Formula I, II or III wherein R¹, when present, is a monosaccharide, a disaccharide or an oligosaccharide. An alternative and/or additional aspect of the present invention provides the use of a cell as defined herein, in a method for the production of a mixture of charged and/or neutral oligosaccharides comprising at least one compound comprising a structure of Formula I, II or III wherein R¹, when present, is a monosaccharide, a disaccharide or an oligosaccharide. A preferred aspect of the present invention provides the use of a cell as defined herein, in a method for the production of a mixture of sialylated and/or neutral oligosaccharides comprising at least one compound comprising a structure of Formula I, II or III wherein R¹, when present, is a monosaccharide, a disaccharide or an oligosaccharide. A preferred aspect provides the use of a cell of present invention in a method for the production of a mixture of mammalian milk oligosaccharides (MMOs) comprising at least one compound comprising a structure of Formula I, II or III wherein R¹, when present, is a monosaccharide, a disaccharide or an oligosaccharide. A further aspect of the present invention provides the use of a method as defined herein for the production of said compound comprising a structure of Formula I, II or III. Furthermore, the invention also relates to said compound comprising a structure of Formula I, II or III obtained by the methods according to the invention, as well as to the use of a polynucleotide, the vector, host cells or the polypeptide as described above for the production of said compound comprising a structure of Formula I, II or III. Said compound comprising a structure of Formula I, II or III may be used as food additive, prebiotic, symbiotic, for the supplementation of baby food, adult food or feed, or as either therapeutically or pharmaceutically active compound or in cosmetic applications. With the novel methods, said compound comprising a structure of Formula I, II or III can easily and effectively be provided, without the need for complicated, time and cost consuming synthetic processes. For identification of said compound comprising a structure of Formula I, II or III produced in the cell as described herein, the monomeric building blocks (e.g. the monosaccharide or glycan unit composition), the anomeric configuration of side chains, the presence and location of substituent groups, degree of polymerization/molecular weight and the linkage pattern can be identified by standard methods known in the art, such as, e.g. methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infrared)/Raman spectroscopy, and NMR (Nuclear magnetic resonance) spectroscopy techniques. The crystal structure can be solved using, e.g., solid-state NMR, FT-IR (Fourier transform infrared spectroscopy), and WAXS (wide-angle X-ray scattering). The degree of polymerization (DP), the DP distribution, and polydispersity can be determined by, e.g., viscosimetry and SEC (SEC-HPLC, high performance size-exclusion chromatography). To identify the monomeric components of the di- and/or oligosaccharide methods such as e.g. acid-catalysed hydrolysis, HPLC (high performance liquid chromatography) or GLC (gas-liquid chromatography) (after conversion to alditol acetates) may be used. To determine the glycosidic linkages, said compound comprising a structure of Formula I, II or III is methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). To determine the glycan sequence, a partial depolymerization is carried out using an acid or enzymes to determine the structures. To identify the anomeric configuration, said compound comprising a structure of Formula I, II or III is subjected to enzymatic analysis, e.g. it is contacted with an enzyme that is specific for a particular type of linkage, e.g., beta-galactosidase, or alpha-glucosidase, etc., and NMR may be used to analyse the products.

The separated and preferably also purified compound comprising a structure of Formula I, II or III as described herein is incorporated into a food (e.g., human food or feed), dietary supplement, pharmaceutical ingredient, cosmetic ingredient or medicine. In some embodiments, said compound comprising a structure of Formula I, II or III is mixed with one or more ingredients suitable for food, feed, dietary supplement, pharmaceutical ingredient, cosmetic ingredient or medicine.

In some embodiments, the dietary supplement comprises at least one prebiotic ingredient and/or at least one probiotic ingredient.

A “prebiotic” is a substance that promotes growth of microorganisms beneficial to the host, particularly microorganisms in the gastrointestinal tract. In some embodiments, a dietary supplement provides multiple prebiotics, including said compound comprising a structure of Formula I, II or III being a prebiotic produced and/or purified by a process disclosed in this specification, to promote growth of one or more beneficial microorganisms. Examples of prebiotic ingredients for dietary supplements include other prebiotic molecules (such as HMOs) and plant polysaccharides (such as inulin, pectin, b-glucan and xylooligosaccharide). A “probiotic” product typically contains live microorganisms that replace or add to gastrointestinal microflora, to the benefit of the recipient. Examples of such microorganisms include Lactobacillus species (for example, L. acidophilus and L. bulgaricus), Bifidobacterium species (for example, B. animalis, B. longum and B. infantis (e.g., Bi-26)), and Saccharomyces boulardii. In some embodiments, said compound comprising a structure of Formula I, II or III produced and/or purified by a process of this specification is orally administered in combination with such microorganism.

Examples of further ingredients for dietary supplements include oligosaccharides (such as 2′-fucosyllactose, 3-fucosyllactose, 3′-sialyllactose, 6′-sialyllactose), disaccharides (such as lactose), monosaccharides (such as glucose, galactose, L-fucose, sialic acid, glucosamine and N-acetylglucosamine), thickeners (such as gum arabic), acidity regulators (such as trisodium citrate), water, skimmed milk, and flavourings.

In some embodiments, said compound comprising a structure of Formula I, II or III is incorporated into a human baby food (e.g., infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to be roughly mimic human breast milk. In some embodiments, said compound comprising a structure of Formula I, II or III produced and/or purified by a process in this specification is included in infant formula to provide nutritional benefits similar to those provided by the oligosaccharides in human breast milk. In some embodiments, said compound comprising a structure of Formula I, II or III is mixed with one or more ingredients of the infant formula. Examples of infant formula ingredients include non-fat milk, carbohydrate sources (e.g., lactose), protein sources (e.g., whey protein concentrate and casein), fat sources (e.g., vegetable oils-such as palm, high oleic safflower oil, rapeseed, coconut and/or sunflower oil; and fish oils), vitamins (such as vitamins A, Bb, Bi2, C and D), minerals (such as potassium citrate, calcium citrate, magnesium chloride, sodium chloride, sodium citrate and calcium phosphate) and possibly human milk oligosaccharides (HMOs). Such HMOs may include, for example, DiFL, lacto-N-triose II, LNT, LNnT, lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.

In some embodiments, the one or more infant formula ingredients comprise non-fat milk, a carbohydrate source, a protein source, a fat source, and/or a vitamin and mineral.

In some embodiments, the one or more infant formula ingredients comprise lactose, whey protein concentrate and/or high oleic safflower oil.

In some embodiments, the concentration of the oligosaccharide in the infant formula is approximately the same concentration as the concentration of the oligosaccharide generally present in human breast milk.

In some embodiments, said compound comprising a structure of Formula I, II or III is incorporated into a feed preparation, wherein said feed is chosen from the list comprising pet food, animal milk replacer, veterinary product, post weaning feed, or creep feed.

As will be shown in the examples herein, the method and the cell of the invention preferably provide at least one of the following surprising advantages:
Higher titres of said compound comprising a structure of Formula I, II or III (g/L),
Higher production rate r (g of said compound comprising a structure of Formula I, II or III/L/h),
Higher cell performance index CPI (g of said compound comprising a structure of Formula I, II or III/g X),
Higher specific productivity Qp (g of said compound comprising a structure of Formula I, II or III/g X/h),
Higher yield on sucrose Ys (g of said compound comprising a structure of Formula I, II or III/g sucrose),
Higher sucrose uptake/conversion rate Qs (g sucrose/g X/h),
Higher lactose conversion/consumption rate rs (g lactose/h),
Higher secretion of said compound comprising a structure of Formula I, II or III, and/or
Higher growth speed of the production host,
when compared to a host for production of said compound comprising a structure of Formula I, II or III lacking an alpha-1,2-fucosyltransferase of present invention having galactoside alpha-1,2-fucosyltransferase activity on the galactose residue of LNB. In the present context, “X” means biomass, “g” means gram, “L” means liter and “h” means hour. Said “g oligosaccharide” can be measured in the whole broth and/or in the supernatant.

Preferably, the method and the cell of the invention preferably provide at least one of the following surprising advantages:
Higher titres of said compound comprising a structure of Formula I, II or III (g/L),
Higher production rate r (g of said compound comprising a structure of Formula I, II or III/L/h),
Higher cell performance index CPI (g of said compound comprising a structure of Formula I, II or III/g X),
Higher specific productivity Qp (g of said compound comprising a structure of Formula I, II or III/g X/h),
Higher yield on sucrose Ys (g of said compound comprising a structure of Formula I, II or III/g sucrose),
Higher sucrose uptake/conversion rate Qs (g sucrose/g X/h),
Higher lactose conversion/consumption rate rs (g lactose/h), and/or
Higher secretion of said compound comprising a structure of Formula I, II or III,
when compared to a host for production of said compound comprising a structure of Formula I, II or III lacking an alpha-1,2-fucosyltransferase of present invention having galactoside alpha-1,2-fucosyltransferase activity on the galactose residue of LNB.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described above and below are those well-known and commonly employed in the art. Standard techniques are used for nucleic acid and peptide synthesis. Generally, purification steps are performed according to the manufacturer's specifications.

Further advantages follow from the specific embodiments and the examples. It goes without saying that the abovementioned features and the features which are still to be explained below can be used not only in the respectively specified combinations, but also in other combinations or on their own, without departing from the scope of the present invention.

Moreover, the present invention relates to the following specific embodiments:

1. A method for production of a compound comprising a structure of Formula I, II or III:

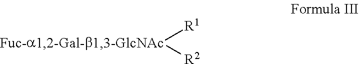

Fuc-α1,2-Gal-β1,3-GlcNAc Formula I

Fuc-α1,2-Gal-β1,3-GlcNAc-R¹ Formula II
wherein:
R¹ is a monosaccharide, disaccharide, oligosaccharide, protein, glycoprotein, peptide, glycopeptide, lipid or glycolipid; R² is a monosaccharide, disaccharide or oligosaccharide;
by a cell, preferably a single cell, wherein said method comprises the steps of:
i. providing a cell expressing an alpha-1,2-fucosyltransferase, and
ii. cultivating and/or incubating said cell under conditions permissive to express said compound comprising a structure of Formula I, II or III,
iii. preferably, separating said compound comprising a structure of Formula I, II or III from said cultivation,
characterized in that said alpha-1,2-fucosyltransferase has galactoside alpha-1,2-fucosyltransferase activity on the galactose residue of Gal-b1,3-GlcNAc (LNB, lacto-N-biose) and:
is a polypeptide belonging to the gt11 fucosyltransferase family and comprising the motif X (no M) X (no F) XXXGNX (no N) [ILMV]X (no E,S) X (no E) XXXX (no F, S) X (no Y) XXXXX (no H, S, Y) with SEQ ID NO 38 wherein X can be any amino acid residue, or
is a polypeptide belonging to the gt74 fucosyltransferase family, or
comprises a polypeptide sequence according to any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 or 37, preferably any one of SEQ ID NO 01, 02, 03, 04, 05, 06, 07, 08 or 09, more preferably any one of SEQ ID NO 01, 02, 03, 04, 05, 06, 07 or 08, even more preferably any one of SEQ ID NO 05, 06, 07 or 08, most preferably any one of SEQ ID NO 01, 02, 03 or 04, or
is a functional homolog, variant or derivative of SEQ ID NO 03 having at least 15.0% overall sequence identity to the full-length of said polypeptide with SEQ ID NO 03, or
is a functional homolog, variant or derivative of any one of SEQ ID NO 15, 34, 35, 36 or 37 having at least 22.0% overall sequence identity to the full-length of any one of said polypeptide with SEQ ID NO 15, 34, 35, 36 or 37, or
is a functional homolog, variant or derivative of any one of SEQ ID NO 05, 08, 11, 21, 30 or 31, preferably SEQ ID NO 05 or 08, having at least 30.0% overall sequence identity to the full-length of any one of said polypeptide with SEQ ID NO 05, 08, 11, 21, 30 or 31, preferably SEQ ID NO 05 or 08, or
is a functional homolog, variant or derivative of any one of SEQ ID NO 06, 07, 09, 19, 25, 27, 32 or 33, preferably SEQ ID NO 06, 07 or 09, more preferably SEQ ID NO 06 or 07, having at least 35.0% overall sequence identity to the full-length of any one of said polypeptide with SEQ ID NO 06, 07, 09, 19, 25, 27, 32 or 33, preferably SEQ ID NO 06, 07 or 09, more preferably SEQ ID NO 06 or 07, or
is a functional homolog, variant or derivative of any one of SEQ ID NO 02, 04, 14, 16, 17 or 28, preferably SEQ ID NO 02 or 04, having at least 40.0% overall sequence identity to the full-length of any one of said polypeptide with SEQ ID NO 02, 04, 14, 16, 17 or 28, preferably SEQ ID NO 02 or 04, or
is a functional homolog, variant or derivative of any one of SEQ ID NO 01, 10, 12, 13, 18, 20, 22, 24 or 26, preferably SEQ ID NO 01, having at least 45.0% overall sequence identity to the full-length of any one of said polypeptide with SEQ ID NO 01, 10, 12, 13, 18, 20, 22, 24 or 26, preferably SEQ ID NO 01, or
is a functional homolog, variant or derivative of SEQ ID NO 23 having at least 50.0% overall sequence identity to the full-length of said polypeptide with SEQ ID NO 23, or
is a functional homolog, variant or derivative of SEQ ID NO 29 having at least 70.0% overall sequence identity to the full-length of said polypeptide with SEQ ID NO 29, or
is a functional fragment comprising an oligopeptide sequence of at least 10 consecutive amino acid residues from any one of SEQ ID NO 03, 05, 11, 15, 21, 31, 34, 35, 36 or 37, preferably SEQ ID NO 03 or 05, more preferably SEQ ID NO 03, or
is a functional fragment comprising an oligopeptide sequence of at least 13 consecutive amino acid residues from any one of SEQ ID NO 06, 08, 13, 17, 19, 20, 25, 28 or 30, preferably SEQ ID NO 06 or 08, or
is a functional fragment comprising an oligopeptide sequence of at least 15 consecutive amino acid residues from any one of SEQ ID NO 04, 07, 09, 10, 16, 26, 27, 32 or 33, preferably SEQ ID NO 04, 07 or 09, more preferably SEQ ID NO 04 or 07, most preferably SEQ ID NO 04, or
is a functional fragment comprising an oligopeptide sequence of at least 18 consecutive amino acid residues from any one of SEQ ID NO 01, 02, 14, 18, 22 or 24, preferably SEQ ID NO 01 or 02, or
is a functional fragment comprising an oligopeptide sequence of at least 22 consecutive amino acid residues from any one of SEQ ID NO 12, 23 or 29.

2. Method according to embodiment 1, wherein said alpha-1,2-fucosyltransferase has additional galactoside alpha-1,2-fucosyltransferase activity on the galactose residue at the non-reducing end of Gal-b1,3-GlcNAc-b1,3-Gal-b1,4-Glc (LNT, lacto-N-tetraose) and wherein said alpha-1,2-fucosyltransferase:
is a polypeptide belonging to the gt74 fucosyltransferase family and comprising the motif [DE]CC [FWY]XXX (no D,E) (Xn) [FWY]X [ILMV][DE][DE] with SEQ ID NO 39 wherein X can be any amino acid residue and wherein n is 10 to 40, or
comprises a polypeptide sequence according to any one of SEQ ID NO 01, 02, 03, 04, 05, 06, 07, 08, 09, 10, 11, 12, 13, 14, 15, 16, 17 or 18, preferably any one of SEQ ID NO 01, 02, 03, 04, 05, 06, 07, 08 or 09, more preferably any one of SEQ ID NO 01, 02, 03, 04, 05, 06, 07 or 08, even more preferably any one of SEQ ID NO 05, 06, 07 or 08, most preferably any one of SEQ ID NO 01, 02, 03 or 04, or
is a functional homolog, variant or derivative of any one of SEQ ID NO 03 or 15, preferably SEQ ID NO 03, having at least 20.0% overall sequence identity to the full-length of any one of said polypeptide with SEQ ID NO 03 or 15, preferably SEQ ID NO 03, or
is a functional homolog, variant or derivative of any one of SEQ ID NO 05, 08 or 11, preferably SEQ ID NO 05 or 08, having at least 30.0% overall sequence identity to the full-length of any one of said polypeptide with SEQ ID NO 05, 08 or 11, preferably SEQ ID NO 05 or 08, or
is a functional homolog, variant or derivative of any one of SEQ ID NO 02, 04, 06, 07, 09 or 17, preferably SEQ ID NO 02, 04, 06, 07 or 09, more preferably SEQ ID NO 02, 04, 06 or 07, even more preferably SEQ ID 02 or 04, having at least 37.50% overall sequence identity to the full-length of any one of said polypeptide with SEQ ID NO 02, 04, 06, 07, 09 or 17, preferably SEQ ID NO 02, 04, 06, 07 or 09, more preferably SEQ ID NO 02, 04, 06 or 07, even more preferably SEQ ID 02 or 04, or
is a functional homolog, variant or derivative of any one of SEQ ID NO 01, 10, 12, 13, 14, 16 or 18, preferably SEQ ID NO 01, having at least 45.0% overall sequence identity to the full-length of any one of said polypeptide with SEQ ID NO 01, 10, 12, 13, 14, 16 or 18, preferably SEQ ID NO 01, or
is a functional fragment comprising an oligopeptide sequence of at least 10 consecutive amino acid residues from any one of SEQ ID NO 03, 05, 11 or 15, preferably SEQ ID NO 03 or 05, or
is a functional fragment comprising an oligopeptide sequence of at least 13 consecutive amino acid residues from any one of SEQ ID NO 06, 08, 13 or 17, preferably SEQ ID NO 06 or 08, or
is a functional fragment comprising an oligopeptide sequence of at least 15 consecutive amino acid residues from any one of SEQ ID NO 04, 07, 09, 10, 16, preferably SEQ ID NO 04, 07 or 09, more preferably SEQ ID NO 04 or 07, even more preferably SEQ ID NO 04, or
is a functional fragment comprising an oligopeptide sequence of at least 18 consecutive amino acid residues from any one of SEQ ID NO 01, 02, 14 or 18, preferably SEQ ID NO 01 or 02, or
is a functional fragment comprising an oligopeptide sequence of at least 20 consecutive amino acid residues from SEQ ID NO 12.

3. Method according to embodiment 2, wherein said alpha-1,2-fucosyltransferase has no additional galactoside alpha-1,2-fucosyltransferase activity on lactose or has additional galactoside alpha-1,2-fucosyltransferase activity on lactose which is lower than its additional galactoside alpha-1,2-fucosyltransferase activity on the galactose residue at the non-reducing end of LNT, and
is a polypeptide belonging to the gt74 fucosyltransferase family and comprising the motif [DE]CC [FWY]XXX (no D,E) (Xn) [FWY]X [ILMV][DE][DE] with SEQ ID NO 39 wherein X can be any amino acid residue and wherein n is 10 to 40, or
comprises a polypeptide sequence according to any one of SEQ ID NO 01, 02, 03, 04, 07, 09, 10, 12, 13, 14, 15, 16, 17 or 18, preferably any one of SEQ ID NO 01, 02, 03, 04, 07 or 09, more preferably any one of SEQ ID NO 01, 02, 03, 04 or 07, even more preferably any one of SEQ ID NO 01, 02, 03 or 04, or
is a functional homolog, variant or derivative of any one of SEQ ID NO 03 or 15, preferably SEQ ID NO 03, having at least 20.0% overall sequence identity to the full-length of any one of said polypeptide with SEQ ID NO 03 or 15, preferably SEQ ID NO 03, or
is a functional homolog, variant or derivative of any one of SEQ ID NO 02, 04, 07, 09 or 17, preferably SEQ ID NO 02, 04, 07 or 09, more preferably SEQ ID NO 02, 04 or 07, even more preferably SEQ ID NO 02 or 04, having at least 37.50% overall sequence identity to the full-length of any one of said polypeptide with SEQ ID NO 02, 04, 06, 07, 09 or 17, preferably SEQ ID NO 02, 04, 07 or 09, more preferably SEQ ID NO 02, 04 or 07, even more preferably SEQ ID NO 02 or 04, or
is a functional homolog, variant or derivative of any one of SEQ ID NO 01, 10, 12, 13, 14, 16 or 18, preferably SEQ ID NO 01, having at least 45.0% overall sequence identity to the full-length of any one of said polypeptide with SEQ ID NO 01, 10, 12, 13, 14, 16 or 18, preferably SEQ ID NO 01, or is a functional fragment comprising an oligopeptide sequence of at least 10 consecutive amino acid residues from any one of SEQ ID NO 03 or 15, preferably SEQ ID NO 03, or
is a functional fragment comprising an oligopeptide sequence of at least 13 consecutive amino acid residues from any one of SEQ ID NO 13 or 17, or
is a functional fragment comprising an oligopeptide sequence of at least 15 consecutive amino acid residues from any one of SEQ ID NO 04, 07, 09, 10 or 16, preferably SEQ ID NO 04, 07 or 09, more preferably SEQ ID NO 04 or 07, even more preferably SEQ ID NO 04, or
is a functional fragment comprising an oligopeptide sequence of at least 18 consecutive amino acid residues from any one of SEQ ID NO 01, 02, 14 or 18, preferably SEQ ID NO 01 or 02, or
is a functional fragment comprising an oligopeptide sequence of at least 20 consecutive amino acid residues from SEQ ID NO 12.

4. Method according to embodiment 2, wherein said alpha-1,2-fucosyltransferase has additional galactoside alpha-1,2-fucosyltransferase activity on lactose that is higher than its additional galactoside alpha-1,2-fucosyltransferase activity on the galactose residue at the non-reducing end of LNT, and
comprises a polypeptide sequence according to any one of SEQ ID NO 05, 06, 08 or 11, preferably SEQ ID NO 05, 06 or 08, or
is a functional homolog, variant or derivative of any one of SEQ ID NO 05, 06, 08 or 11, preferably SEQ ID NO 05, 06 or 08, having at least 35.0% overall sequence identity to the full-length of any one of said polypeptide with SEQ ID NO 05, 06, 08 or 11, preferably SEQ ID NO 05, 06 or 08, or
is a functional fragment comprising an oligopeptide sequence of at least 13 consecutive amino acid residues from SEQ ID NO 05, 06, 08 or 11, preferably SEQ ID NO 05, 06 or 08.

5. Method according to embodiment 1, wherein said alpha-1,2-fucosyltransferase has no galactoside alpha-1,2-fucosyltransferase activity on the galactose residue at the non-reducing end of LNT, and
comprises a polypeptide sequence according to any one of SEQ ID NO 19, 20, 21, 22, 23, 24, 25, 26, 27, 28, 29, 30, 31, 32, 33, 34, 35, 36 or 37, or
is a functional homolog, variant or derivative of any one of SEQ ID NO 34, 35, 36 or 37 having at least 22.0% overall sequence identity to the full-length of any one of said polypeptide with SEQ ID NO 34, 35, 36 or 37, or
is a functional homolog, variant or derivative of any one of SEQ ID NO 21, 30 or 31 having at least 30.0% overall sequence identity to the full-length of any one of said polypeptide with SEQ ID NO 21, 30 or 31, or
is a functional homolog, variant or derivative of any one of SEQ ID NO 19, 25, 27, 32 or 33 having at least 35.0% overall sequence identity to the full-length of any one of said polypeptide with SEQ ID NO 19, 25, 27, 32 or 33, or
is a functional homolog, variant or derivative of any one of SEQ ID NO 20, 22, 24, 26 or 28 having at least 45.0% overall sequence identity to the full-length of any one of said polypeptide with SEQ ID NO 20, 22, 24, 26 or 28, or
is a functional homolog, variant or derivative of SEQ ID NO 23 having at least 50.0% overall sequence identity to the full-length of said polypeptide with SEQ ID NO 23, or
is a functional homolog, variant or derivative of SEQ ID NO 29 having at least 70.0% overall sequence identity to the full-length of said polypeptide with SEQ ID NO 29, or
is a functional fragment comprising an oligopeptide sequence of at least 10 consecutive amino acid residues from any one of SEQ ID NO 21, 31, 34, 35, 36 or 37, or
is a functional fragment comprising an oligopeptide sequence of at least 13 consecutive amino acid residues from any one of SEQ ID NO 19, 20, 25, 28 or 30, or
is a functional fragment comprising an oligopeptide sequence of at least 15 consecutive amino acid residues from any one of SEQ ID NO 26, 27, 32 or 33, or
is a functional fragment comprising an oligopeptide sequence of at least 17 consecutive amino acid residues from any one of SEQ ID NO 22 or 24, or
is a functional fragment comprising an oligopeptide sequence of at least 22 consecutive amino acid residues from any one of SEQ ID NO 23 or 29.

6. Method according to embodiment 5, wherein said alpha-1,2-fucosyltransferase has no galactoside alpha-1,2-fucosyltransferase activity on lactose or has an additional galactoside alpha-1,2-fucosyltransferase activity on lactose which is lower than 3.0% of the galactoside alpha-1,2-fucosyltransferase activity on lactose of the alpha-1,2-fucosyltransferase with SEQ ID NO 06 and
comprises a polypeptide sequence according to any one of SEQ ID NO 19, 20, 21, 22, 23, 24, 25, 26, 27, 30, 33, 34, 35, 36 or 37, or
is a functional homolog, variant or derivative of any one of SEQ ID NO 34, 35, 36 or 37 having at least 22.0% overall sequence identity to the full-length of any one of said polypeptide with SEQ ID NO 34, 35, 36 or 37, or
is a functional homolog, variant or derivative of any one of SEQ ID NO 21 or 30 having at least 30.0% overall sequence identity to the full-length of any one of said polypeptide with SEQ ID NO 21 or 30, or
is a functional homolog, variant or derivative of any one of SEQ ID NO 19, 25, 27 or 33 having at least 35.0% overall sequence identity to the full-length of any one of said polypeptide with SEQ ID NO 19, 25, 27 or 33, or
is a functional homolog, variant or derivative of any one of SEQ ID NO 20, 22, 24 or 26 having at least 45.0% overall sequence identity to the full-length of any one of said polypeptide with SEQ ID NO 20, 22, 24 or 26, or
is a functional homolog, variant or derivative of SEQ ID NO 23 having at least 50.0% overall sequence identity to the full-length of said polypeptide with SEQ ID NO 23, or
is a functional fragment comprising an oligopeptide sequence of at least 10 consecutive amino acid residues from any one of SEQ ID NO 21, 34, 35, 36 or 37, or
is a functional fragment comprising an oligopeptide sequence of at least 13 consecutive amino acid residues from any one of SEQ ID NO 19, 20, 25 or 30, or
is a functional fragment comprising an oligopeptide sequence of at least 15 consecutive amino acid residues from any one of SEQ ID NO 26, 27 or 33, or
is a functional fragment comprising an oligopeptide sequence of at least 17 consecutive amino acid residues from any one of SEQ ID NO 22 or 24, or
is a functional fragment comprising an oligopeptide sequence of at least 20 consecutive amino acid residues from SEQ ID NO 23.

7. Method according to embodiment 5, wherein said alpha-1,2-fucosyltransferase has additional galactoside alpha-1,2-fucosyltransferase activity on lactose that is between 4.0 and 20.0% of the galactoside alpha-1,2-fucosyltransferase activity on lactose of the alpha-1,2-fucosyltransferase with SEQ ID NO 06 and
comprises a polypeptide sequence according to any one of SEQ ID NO 28, 29, 31 or 32, or
is a functional homolog, variant or derivative of any one of SEQ ID NO 31 or 32 having at least 35.0% overall sequence identity to the full-length of any one of said polypeptide with SEQ ID NO 31 or 32, or
is a functional homolog, variant or derivative of SEQ ID NO 28 having at least 40.0% overall sequence identity to the full-length of said polypeptide with SEQ ID NO 28, or
is a functional homolog, variant or derivative of SEQ ID NO 29 having at least 70.0% overall sequence identity to the full-length of said polypeptide with SEQ ID NO 29, or
is a functional fragment comprising an oligopeptide sequence of at least 10 consecutive amino acid residues from SEQ ID NO 31, or
is a functional fragment comprising an oligopeptide sequence of at least 14 consecutive amino acid residues from any one of SEQ ID NO 28 or 32, or
is a functional fragment comprising an oligopeptide sequence of at least 22 consecutive amino acid residues from SEQ ID NO 29.

8. Method according to any one of previous embodiments, wherein said cell is modified in the expression or activity of any one of said alpha-1,2-fucosyltransferases.

9. Method according to any one of previous embodiments, wherein said cell is capable to produce one or more nucleotide-activated sugars chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy--L-arabino-4-hexulose, UDP-2-acetamido-2,6-dideoxy--L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7 (8,9) Ac₂, CMP-N-glycolylneuraminic acid (CMP-Neu5Gc), GDP-rhamnose and UDP-xylose.

10. Method according to any one of previous embodiments, wherein said cell expresses one or more polypeptides chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, GDP-fucose pyrophosphorylase, fucose-1-phosphate guanylyltransferase, L-glutamine-D-fructose-6-phosphate aminotransferase, glucosamine-6-phosphate deaminase, phosphoglucosamine mutase, N-acetylglucosamine-6-phosphate deacetylase, N-acylglucosamine 2-epimerase, UDP-N-acetylglucosamine 2-epimerase, N-acetylmannosamine-6-phosphate 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridylyltransferase, glucosamine-1-phosphate acetyltransferase, N-acetylneuraminate synthase, N-acetylneuraminate lyase, N-acylneuraminate-9-phosphate synthase, N-acylneuraminate-9-phosphate phosphatase, N-acylneuraminate cytidylyltransferase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase, preferably wherein said cell is modified in the expression or activity of any one of said polypeptides.

11. Method according to any one of previous embodiments, wherein said cell expresses one or more glycosyltransferases chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases, preferably, said fucosyltransferase is chosen from the list comprising alpha-1,2-fucosyltransferase, alpha-1,3-fucosyltransferase, alpha-1,4-fucosyltransferase and alpha-1,6-fucosyltransferase,
preferably, said sialyltransferase is chosen from the list comprising alpha-2,3-sialyltransferase, alpha-2,6-sialyltransferase and alpha-2,8-sialyltransferase,
preferably, said galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase, preferably, said glucosyltransferase is chosen from the list comprising alpha-glucosyltransferase, beta-1,2-glucosyltransferase, beta-1,3-glucosyltransferase and beta-1,4-glucosyltransferase,
preferably, said mannosyltransferase is chosen from the list comprising alpha-1,2-mannosyltransferase, alpha-1,3-mannosyltransferase and alpha-1,6-mannosyltransferase, preferably, said N-acetylglucosaminyltransferase is chosen from the list comprising galactoside beta-1,3-N-acetylglucosaminyltransferase and beta-1,6-N-acetylglucosaminyltransferase,
preferably, said N-acetylgalactosaminyltransferase is an alpha-1,3-N-acetylgalactosaminyltransferase,
preferably, said cell is modified in the expression or activity of any one of said glycosyltransferases.

12. Method according to any one of previous embodiments, wherein said compound comprising a structure of Formula I, II or III is an oligosaccharide, preferably said oligosaccharide is a mammalian milk oligosaccharide (MMO), more preferably a human milk oligosaccharide (HMO).

13. Method according to any one of previous embodiments, wherein said compound comprising a structure of Formula I, II or III is a charged, preferably sialylated, compound or a neutral compound, preferably wherein said compound comprising a structure of Formula I, II or III is a charged, preferably sialylated, oligosaccharide or a neutral oligosaccharide.

14. Method according to any one of previous embodiments, wherein said compound comprising a structure of Formula I, II or III is Fuc-α1,2-Gal-b1,3-GlcNAc-b1,3-R comprising one R group chosen from the list comprising a monosaccharide, a disaccharide or an oligosaccharide, preferably wherein said compound comprising a structure of Formula I, II or III is Fuc-a1,2-Gal-b1,3-GlcNAc-b1,3-Gal-R comprising one R group chosen from the list comprising a monosaccharide, a disaccharide or an oligosaccharide, more preferably wherein said compound comprising a structure of Formula I, II or III is lacto-N-fucopentaose I (LNFP-I, Fuc-α1,2-Gal-b1,3-GlcNAc-b1,3-Gal-b1,4-Glc).

15. Method according to any one of previous embodiments, wherein said cell is using one or more precursor(s) for the production of said compound comprising a structure of Formula I, II or III, said precursor(s) being fed to the cell from the cultivation medium.

16. Method according to any one of previous embodiments, wherein said cell is producing one or more precursor(s) for the production of said compound comprising a structure of Formula I, II or III.

17. Method according to any one of embodiments 15 or 16, wherein said precursor for the production of said compound comprising a structure of Formula I, II or III is completely converted into said compound comprising a structure of Formula I, II or III.

18. Method according to any one of previous embodiments, wherein said cell is capable to produce a compound comprising a structure of Formula IV, V or VI:

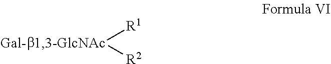

Gal-β1,3-GlcNAc Formula IV

Gal-β1,3-GlcNAc-R¹ Formula V
wherein:
R¹ is a monosaccharide, disaccharide, oligosaccharide, protein, glycoprotein, peptide, glycopeptide, lipid or glycolipid; R² is a monosaccharide, disaccharide or oligosaccharide.

19. Method according to any one of previous embodiments, wherein said cell produces said compound comprising a structure of Formula I, II or III intracellularly and wherein a fraction or substantially all of said produced compound comprising a structure of Formula I, II or III remains intracellularly and/or is excreted outside said cell via passive or active transport.

20. Method according to any one of previous embodiments, wherein said cell expresses a membrane transporter protein or a polypeptide having transport activity hereby transporting compounds across the outer membrane of the cell wall, preferably, said cell is modified in the expression or activity of said membrane transporter protein or polypeptide having transport activity.

21. Method according to embodiment 20, wherein said membrane transporter protein or polypeptide having transport activity is chosen from the list comprising porters, P-P-bond-hydrolysis-driven transporters, b-barrel porins, auxiliary transport proteins, putative transport proteins and phosphotransfer-driven group translocators, preferably, said porters comprise MFS transporters, sugar efflux transporters and siderophore exporters, preferably, said P-P-bond-hydrolysis-driven transporters comprise ABC transporters and siderophore exporters.

22. Method according to any one of embodiments 20 or 21, wherein said membrane transporter protein or polypeptide having transport activity controls the flow over the outer membrane of the cell wall of said compound comprising a structure of Formula I, II or III and/or of one or more precursor(s) and/or acceptor(s) to be used in said production of said compound comprising a structure of Formula I, II or III.

23. Method according to any one of embodiments 20 to 22, wherein said membrane transporter protein or polypeptide having transport activity provides improved production and/or enabled and/or enhanced efflux of said compound comprising a structure of Formula I, II or III.

24. Method according to any one of previous embodiments, wherein said cell is a genetically engineered cell.

25. Method according to embodiment 24, wherein said cell is modified with one or more gene expression modules, characterized in that the expression from any of said expression modules is either constitutive or is created by a natural inducer.

26. Method according to any one of embodiment 24 or 25, wherein said cell comprises multiple copies of the same coding DNA sequence encoding for one protein.

27. Method according to any one of embodiment 24 to 26, wherein said cell comprises a modification for reduced production of acetate.

28. Method according to any one of embodiments 24 to 27, wherein said cell comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose: undecaprenyl-phosphate glucose-1-phosphate transferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IclR, Ion protease, glucose-specific translocating phosphotransferase enzyme IIBC component ptsG, glucose-specific translocating phosphotransferase (PTS) enzyme IIBC component maIX, enzyme IIA^{Glc}, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, pyruvate decarboxylase.

29. Method according to any one of previous embodiments, wherein the cell is capable to produce phosphoenolpyruvate (PEP).

30. Method according to any one of previous embodiments, wherein said cell is modified for enhanced production and/or supply of phosphoenolpyruvate (PEP).

31. Method according to any one of previous embodiments, wherein the cell comprises a catabolic pathway for selected mono-, di- or oligosaccharides which is at least partially inactivated, the mono-, di-, or oligosaccharides being involved in and/or required for said production of said compound comprising a structure of Formula I, II or III.

32. Method according to any one of the previous embodiments, wherein said cell resists the phenomenon of lactose killing when grown in an environment in which lactose is combined with one or more other carbon source(s).

33. Method according to any one of the previous embodiments, wherein said cell produces 90 g/L or more of said compound comprising a structure of Formula I, II or III in the whole broth and/or supernatant and/or wherein said compound comprising a structure of Formula I, II or III in the whole broth and/or supernatant has a purity of at least 80% measured on the total amount of said compound comprising a structure of Formula I, II or III and its precursor(s) in the whole broth and/or supernatant, respectively.

34. Method according to any one of the previous embodiments, wherein said cell is a bacterium, fungus, yeast, a plant cell, an animal cell, or a protozoan cell,
preferably said bacterium is an Escherichia coli strain, more preferably an Escherichia coli strain which is a K-12 strain, even more preferably the Escherichia coli K-12 strain is E. coli MG1655,
preferably said fungus belongs to a genus chosen from the group comprising Rhizopus, Dictyostelium, Penicillium, Mucor or Aspergillus,
preferably said yeast belongs to a genus chosen from the group comprising Saccharomyces, Zygosaccharomyces, Pichia, Komagataella, Hansenula, Yarrowia, Starmerella, Kluyveromyces or Debaromyces,
preferably said plant cell is an algal cell or is derived from tobacco, alfalfa, rice, tomato, cotton, rapeseed, soy, maize, or corn plant,
preferably said animal cell is derived from non-human mammals, birds, fish, invertebrates, reptiles, amphibians or insects or is a genetically modified cell line derived from human cells excluding embryonic stem cells, more preferably said human and non-human mammalian cell is an epithelial cell, an embryonic kidney cell, a fibroblast cell, a COS cell, a Chinese hamster ovary (CHO) cell, a murine myeloma cell, an NIH-3T3 cell, a non-mammary adult stem cell or derivatives thereof, more preferably said insect cell is derived from Spodoptera frugiperda, Bombyx mori, Mamestra brassicae, Trichoplusia ni or Drosophila melanogaster,
preferably said protozoan cell is a Leishmania tarentolae cell.

35. Method according to embodiment 34, wherein said cell is a viable Gram-negative bacterium that comprises a reduced or abolished synthesis of poly-N-acetyl-glucosamine (PNAG), Enterobacterial Common Antigen (ECA), cellulose, colanic acid, core oligosaccharides, Osmoregulated Periplasmic Glucans (OPG), Glucosylglycerol, glycan, and/or trehalose.

36. Method according to any one of previous embodiments, wherein said cell is stably cultured in a medium.

37. Method according to any one of previous embodiments, wherein said conditions comprise:
use of a culture medium comprising at least one precursor and/or acceptor for the production of said compound comprising a structure of Formula I, II or III, and/or
adding to the culture medium at least one precursor and/or acceptor feed for the production of said compound comprising a structure of Formula I, II or III.

38. Method according to any one of previous embodiments, the method comprising at least one of the following steps:
i) Use of a culture medium comprising at least one precursor and/or acceptor;
ii) Adding to the culture medium in a reactor at least one precursor and/or acceptor feed wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than two-fold of the volume of the culture medium before the addition of said precursor and/or acceptor feed;
iii) Adding to the culture medium in a reactor at least one precursor and/or acceptor feed wherein the total reactor volume ranges from 250 ml (millilitre) to 10.000 m³ (cubic meter), preferably in a continuous manner, and preferably so that the final volume of the culture medium is not more than three-fold, preferably not more than two-fold, more preferably less than two-fold of the volume of the culture medium before the addition of said precursor and/or acceptor feed and wherein preferably, the pH of said precursor and/or acceptor feed is set between 3 and 7 and wherein preferably, the temperature of said precursor and/or acceptor feed is kept between 20° C. and 80° C.;
iv) Adding at least one precursor and/or acceptor feed in a continuous manner to the culture medium over the course of 1 day, 2 days, 3 days, 4 days, 5 days by means of a feeding solution;
v) Adding at least one precursor and/or acceptor feed in a continuous manner to the culture medium over the course of 1 day, 2 days, 3 days, 4 days, 5 days by means of a feeding solution and wherein preferably, the pH of said feeding solution is set between 3 and 7 and wherein preferably, the temperature of said feeding solution is kept between 20° C. and 80° C.; said method resulting in said compound comprising a structure of Formula I, II or III with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final cultivation.

39. Method according to any one of embodiments 1 to 37, the method comprising at least one of the following steps:
i) Use of a culture medium comprising at least 50, more preferably at least 75, more preferably at least 100, more preferably at least 120, more preferably at least 150 gram of lactose per litre of initial reactor volume wherein the reactor volume ranges from 250 mL to 10.000 m³ (cubic meter);
ii) Adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 ml to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed;
iii) Adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 ml to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed and wherein preferably the pH of said lactose feed is set between 3 and 7 and wherein preferably the temperature of said lactose feed is kept between 20° C. and 80° C.;
iv) Adding a lactose feed in a continuous manner to the culture medium over the course of 1 day, 2 days, 3 days, 4 days, 5 days by means of a feeding solution;
v) Adding a lactose feed in a continuous manner to the culture medium over the course of 1 day, 2 days, 3 days, 4 days, 5 days by means of a feeding solution and wherein the concentration of said lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said feeding solution is set between 3 and 7 and wherein preferably the temperature of said feeding solution is kept between 20° C. and 80° C.;
said method resulting in said compound comprising a structure of Formula I, II or III with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation.

40. Method according to embodiment 39, wherein the lactose feed is accomplished by adding lactose from the beginning of the cultivation in a concentration of at least 5 mM, preferably in a concentration of 30, 40, 50, 60, 70, 80, 90, 100, 150 mM, more preferably in a concentration >300 mM.

41. Method according to any one of embodiment 39 or 40, wherein said lactose feed is accomplished by adding lactose to the cultivation in a concentration, such, that throughout the production phase of the cultivation a lactose concentration of at least 5 mM, preferably 10 mM or 30 mM is obtained.

42. Method according to any one of previous embodiments, wherein the cells are cultivated for at least about 60, 80, 100, or about 120 hours or in a continuous manner.

43. Method according to any one of previous embodiments, wherein said cell is cultivated in culture medium comprising a carbon source comprising a monosaccharide, disaccharide, oligosaccharide, polysaccharide, polyol, glycerol, a complex medium including molasses, corn steep liquor, peptone, tryptone or yeast extract; preferably, wherein said carbon source is chosen from the list comprising glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate.

44. Method according to any one of previous embodiments, wherein the culture medium contains at least one precursor selected from the group comprising lactose, galactose, fucose, sialic acid, GlcNAc, GalNAc, lacto-N-biose (LNB), N-acetyllactosamine (LacNAc).

45. Method according to any one of previous embodiments, wherein a first phase of exponential cell growth is provided by adding a carbon-based substrate, preferably glucose or sucrose, to the culture medium comprising a precursor, preferably lactose, followed by a second phase wherein only a carbon-based substrate, preferably glucose or sucrose, is added to the culture medium.

46. Method according to any one of embodiments 1 to 44, wherein a first phase of exponential cell growth is provided by adding a carbon-based substrate, preferably glucose or sucrose, to the culture medium comprising a precursor, preferably lactose, followed by a second phase wherein a carbon-based substrate, preferably glucose or sucrose, and a precursor, preferably lactose, are added to the culture medium.

47. Method according to any one of previous embodiments, wherein the cell produces a mixture of charged, preferably sialylated, and/or neutral di- and oligosaccharides comprising at least one compound comprising a structure of Formula I, II or III, wherein R¹, when present, is a monosaccharide, a disaccharide or an oligosaccharide.

48. Method according to any one of previous embodiments, wherein the cell produces a mixture of charged, preferably sialylated, and/or neutral oligosaccharides comprising at least one compound comprising a structure of Formula I, II or III, wherein R¹, when present, is a monosaccharide, a disaccharide or an oligosaccharide.

49. Method according to any one of previous embodiments, wherein said separation comprises at least one of the following steps: clarification, ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, affinity chromatography, ion exchange chromatography, hydrophobic interaction chromatography and/or gel filtration, ligand exchange chromatography.

50. Method according to any one of previous embodiments, further comprising purification of said compound comprising a structure of Formula I, II or III from said cell.

51. Method according to embodiment 50, wherein said purification comprises at least one of the following steps: use of activated charcoal or carbon, use of charcoal, nanofiltration, ultrafiltration, electrophoresis, enzymatic treatment or ion exchange, use of alcohols, use of aqueous alcohol mixtures, crystallization, evaporation, precipitation, drying, spray drying, lyophilization, spray freeze drying, freeze spray drying, band drying, belt drying, vacuum band drying, vacuum belt drying, drum drying, roller drying, vacuum drum drying or vacuum roller drying.

52. A cell genetically engineered for the production of a compound comprising a structure of Formula I, II or III:

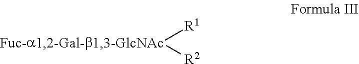

Fuc-α1,2-Gal-β1,3-GlcNAc Formula I

Fuc-α1,2-Gal-β1,3-GlcNAc-R¹ Formula II
wherein:
R¹ is a monosaccharide, disaccharide, oligosaccharide, protein, glycoprotein, peptide, glycopeptide, lipid or glycolipid; R² is a monosaccharide, disaccharide or oligosaccharide;
wherein said cell is capable to express, preferably expresses, an alpha-1,2-fucosyltransferase, characterized in that said alpha-1,2-fucosyltransferase has galactoside alpha-1,2-fucosyltransferase activity on the galactose residue of Gal-b1,3-GlcNAc (LNB, lacto-N-biose) and:
is a polypeptide belonging to the gt11 fucosyltransferase family and comprising the motif X (no M) X (no F) XXXGNX (no N) [ILMV]X (no E,S) X (no E) XXXX (no F, S) X (no Y) XXXXX (no H, S, Y) with SEQ ID NO 38 wherein X can be any amino acid residue, or
is a polypeptide belonging to the gt74 fucosyltransferase family, or
comprises a polypeptide sequence according to any one of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 or 37, preferably any one of SEQ ID NO 01, 02, 03, 04, 05, 06, 07, 08 or 09, more preferably any one of SEQ ID NO 01, 02, 03, 04, 05, 06, 07 or 08, even more preferably any one of SEQ ID NO 05, 06, 07 or 08, most preferably any one of SEQ ID NO 01, 02, 03 or 04, or
is a functional homolog, variant or derivative of SEQ ID NO 03 having at least 15.0% overall sequence identity to the full-length of said polypeptide with SEQ ID NO 03, or
is a functional homolog, variant or derivative of any one of SEQ ID NO 15, 34, 35, 36 or 37 having at least 22.0% overall sequence identity to the full-length of any one of said polypeptide with SEQ ID NO 15, 34, 35, 36 or 37, or
is a functional homolog, variant or derivative of any one of SEQ ID NO 05, 08, 11, 21, 30 or 31, preferably SEQ ID NO 05 or 08, having at least 30.0% overall sequence identity to the full-length of any one of said polypeptide with SEQ ID NO 05, 08, 11, 21, 30 or 31, preferably SEQ ID NO 05 or 08, or
is a functional homolog, variant or derivative of any one of SEQ ID NO 06, 07, 09, 19, 25, 27, 32 or 33, preferably SEQ ID NO 06, 07 or 09, more preferably SEQ ID NO 06 or 07, having at least 35.0% overall sequence identity to the full-length of any one of said polypeptide with SEQ ID NO 06, 07, 09, 19, 25, 27, 32 or 33, preferably SEQ ID NO 06, 07 or 09, more preferably SEQ ID NO 06 or 07, or
is a functional homolog, variant or derivative of any one of SEQ ID NO 02, 04, 14, 16, 17 or 28, preferably SEQ ID NO 02 or 04, having at least 40.0% overall sequence identity to the full-length of any one of said polypeptide with SEQ ID NO 02, 04, 14, 16, 17 or 28, preferably SEQ ID NO 02 or 04, or
is a functional homolog, variant or derivative of any one of SEQ ID NO 01, 10, 12, 13, 18, 20, 22,-24 or 26, preferably SEQ ID NO 01, having at least 45.0% overall sequence identity to the full-length of any one of said polypeptide with SEQ ID NO 01, 10, 12, 13, 18, 20, 22, 24 or 26, preferably SEQ ID NO 01, or
is a functional homolog, variant or derivative of SEQ ID NO 23 having at least 50.0% overall sequence identity to the full-length of said polypeptide with SEQ ID NO 23, or
is a functional homolog, variant or derivative of SEQ ID NO 29 having at least 70.0% overall sequence identity to the full-length of said polypeptide with SEQ ID NO 29, or
is a functional fragment comprising an oligopeptide sequence of at least 10 consecutive amino acid residues from any one of SEQ ID NO 03, 05, 11, 15, 21, 31, 34, 35, 36 or 37, preferably SEQ ID NO 03 or 05, more preferably SEQ ID NO 03, or
is a functional fragment comprising an oligopeptide sequence of at least 13 consecutive amino acid residues from any one of SEQ ID NO 06, 08, 13, 17, 19, 20, 25, 28 or 30, preferably SEQ ID NO 06 or 08, or
is a functional fragment comprising an oligopeptide sequence of at least 15 consecutive amino acid residues from any one of SEQ ID NO 04, 07, 09, 10, 16, 26, 27, 32 or 33, preferably SEQ ID NO 04, 07 or 09, more preferably SEQ ID NO 04 or 07, most preferably SEQ ID NO 04, or
is a functional fragment comprising an oligopeptide sequence of at least 18 consecutive amino acid residues from any one of SEQ ID NO 01, 02, 14, 18, 22 or 24, preferably SEQ ID NO 01 or 02, or
is a functional fragment comprising an oligopeptide sequence of at least 22 consecutive amino acid residues from any one of SEQ ID NO 12, 23 or 29.

53. Cell according to embodiment 52, wherein said alpha-1,2-fucosyltransferase has additional galactoside alpha-1,2-fucosyltransferase activity on the galactose residue at the non-reducing end of Gal-b1,3-GlcNAc-b1,3-Gal-b1,4-Glc (LNT, lacto-N-tetraose) and wherein said alpha-1,2-fucosyltransferase:
is a polypeptide belonging to the gt74 fucosyltransferase family and comprising the motif [DE]CC [FWY]XXX (no D,E) (Xn) [FWY]X [ILMV][DE][DE] with SEQ ID NO 39 wherein X can be any amino acid residue and wherein n is 10 to 40, or
comprises a polypeptide sequence according to any one of SEQ ID NO 01, 02, 03, 04, 05, 06, 07, 08, 09, 10, 11, 12, 13, 14, 15, 16, 17 or 18, preferably any one of SEQ ID NO 01, 02, 03, 04, 05, 06, 07, 08 or 09, more preferably any one of SEQ ID NO 01, 02, 03, 04, 05, 06, 07 or 08, even more preferably any one of SEQ ID NO 05, 06, 07 or 08, most preferably any one of SEQ ID NO 01, 02, 03 or 04, or
is a functional homolog, variant or derivative of any one of SEQ ID NO 03 or 15, preferably SEQ ID NO 03, having at least 20.0% overall sequence identity to the full-length of any one of said polypeptide with SEQ ID NO 03 or 15, preferably SEQ ID NO 03, or
is a functional homolog, variant or derivative of any one of SEQ ID NO 05, 08 or 11, preferably SEQ ID NO 05 or 08, having at least 30.0% overall sequence identity to the full-length of any one of said polypeptide with SEQ ID NO 05, 08 or 11, preferably SEQ ID NO 05 or 08, or
is a functional homolog, variant or derivative of any one of SEQ ID NO 02, 04, 06, 07, 09 or 17, preferably SEQ ID NO 02, 04, 06, 07 or 09, more preferably SEQ ID NO 02, 04, 06 or 07, even more preferably SEQ ID 02 or 04, having at least 37.50% overall sequence identity to the full-length of any one of said polypeptide with SEQ ID NO 02, 04, 06, 07, 09 or 17, preferably SEQ ID NO 02, 04, 06, 07 or 09, more preferably SEQ ID NO 02, 04, 06 or 07, even more preferably SEQ ID 02 or 04, or
is a functional homolog, variant or derivative of any one of SEQ ID NO 01, 10, 12, 13, 14, 16 or 18, preferably SEQ ID NO 01, having at least 45.0% overall sequence identity to the full-length of any one of said polypeptide with SEQ ID NO 01, 10, 12, 13, 14, 16 or 18, preferably SEQ ID NO 01, or
is a functional fragment comprising an oligopeptide sequence of at least 10 consecutive amino acid residues from any one of SEQ ID NO 03, 05, 11 or 15, preferably SEQ ID NO 03 or 05, or
is a functional fragment comprising an oligopeptide sequence of at least 13 consecutive amino acid residues from any one of SEQ ID NO 06, 08, 13 or 17, preferably SEQ ID NO 06 or 08, or
is a functional fragment comprising an oligopeptide sequence of at least 15 consecutive amino acid residues from any one of SEQ ID NO 04, 07, 09, 10, 16, preferably SEQ ID NO 04, 07 or 09, more preferably SEQ ID NO 04 or 07, even more preferably SEQ ID NO 04, or
is a functional fragment comprising an oligopeptide sequence of at least 18 consecutive amino acid residues from any one of SEQ ID NO 01, 02, 14 or 18, preferably SEQ ID NO 01 or 02, or
is a functional fragment comprising an oligopeptide sequence of at least 20 consecutive amino acid residues from SEQ ID NO 12.

54. Cell according to embodiment 53, wherein said alpha-1,2-fucosyltransferase has no additional galactoside alpha-1,2-fucosyltransferase activity on lactose or has additional galactoside alpha-1,2-fucosyltransferase activity on lactose which is lower than its additional galactoside alpha-1,2-fucosyltransferase activity on the galactose residue at the non-reducing end of LNT, and
is a polypeptide belonging to the gt74 fucosyltransferase family and comprising the motif [DE]CC [FWY]XXX (no D,E) (Xn) [FWY]X [ILMV][DE][DE] with SEQ ID NO 39 wherein X can be any amino acid residue and wherein n is 10 to 40, or comprises a polypeptide sequence according to any one of SEQ ID NO 01, 02, 03, 04, 07, 09, 10, 12, 13, 14, 15, 16, 17 or 18, preferably any one of SEQ ID NO 01, 02, 03, 04, 07 or 09, more preferably any one of SEQ ID NO 01, 02, 03, 04 or 07, even more preferably any one of SEQ ID NO 01, 02, 03 or 04, or
is a functional homolog, variant or derivative of any one of SEQ ID NO 03 or 15, preferably SEQ ID NO 03, having at least 20.0% overall sequence identity to the full-length of any one of said polypeptide with SEQ ID NO 03 or 15, preferably SEQ ID NO 03, or
is a functional homolog, variant or derivative of any one of SEQ ID NO 02, 04, 07, 09 or 17, preferably SEQ ID NO 02, 04, 07 or 09, more preferably SEQ ID NO 02, 04 or 07, even more preferably SEQ ID NO 02 or 04, having at least 37.50% overall sequence identity to the full-length of any one of said polypeptide with SEQ ID NO 02, 04, 06, 07, 09 or 17, preferably SEQ ID NO 02, 04, 07 or 09, more preferably SEQ ID NO 02, 04 or 07, even more preferably SEQ ID NO 02 or 04, or
is a functional homolog, variant or derivative of any one of SEQ ID NO 01, 10, 12, 13, 14, 16 or 18, preferably SEQ ID NO 01, having at least 45.0% overall sequence identity to the full-length of any one of said polypeptide with SEQ ID NO 01, 10, 12, 13, 14, 16 or 18, preferably SEQ ID NO 01, or
is a functional fragment comprising an oligopeptide sequence of at least 10 consecutive amino acid residues from any one of SEQ ID NO 03 or 15, preferably SEQ ID NO 03, or
is a functional fragment comprising an oligopeptide sequence of at least 13 consecutive amino acid residues from any one of SEQ ID NO 13 or 17, or
is a functional fragment comprising an oligopeptide sequence of at least 15 consecutive amino acid residues from any one of SEQ ID NO 04, 07, 09, 10 or 16, preferably SEQ ID NO 04, 07 or 09, more preferably SEQ ID NO 04 or 07, even more preferably SEQ ID NO 04, or
is a functional fragment comprising an oligopeptide sequence of at least 18 consecutive amino acid residues from any one of SEQ ID NO 01, 02, 14 or 18, preferably SEQ ID NO 01 or 02, or
is a functional fragment comprising an oligopeptide sequence of at least 20 consecutive amino acid residues from SEQ ID NO 12.

55. Cell according to embodiment 53, wherein said alpha-1,2-fucosyltransferase has additional galactoside alpha-1,2-fucosyltransferase activity on lactose that is higher than its additional galactoside alpha-1,2-fucosyltransferase activity on the galactose residue at the non-reducing end of LNT, and
comprises a polypeptide sequence according to any one of SEQ ID NO 05, 06, 08 or 11, preferably SEQ ID NO 05, 06 or 08, or
is a functional homolog, variant or derivative of any one of SEQ ID NO 05, 06, 08 or 11, preferably SEQ ID NO 05, 06 or 08, having at least 35.0% overall sequence identity to the full-length of any one of said polypeptide with SEQ ID NO 05, 06, 08 or 11, preferably SEQ ID NO 05, 06 or 08, or
is a functional fragment comprising an oligopeptide sequence of at least 13 consecutive amino acid residues from SEQ ID NO 05, 06, 08 or 11, preferably SEQ ID NO 05, 06 or 08.

56. Cell according to embodiment 52, wherein said alpha-1,2-fucosyltransferase has no galactoside alpha-1,2-fucosyltransferase activity on the galactose residue at the non-reducing end of LNT, and
comprises a polypeptide sequence according to any one of SEQ ID NO 19, 20, 21, 22, 23, 24, 25, 26, 27, 28, 29, 30, 31, 32, 33, 34, 35, 36 or 37, or
is a functional homolog, variant or derivative of any one of SEQ ID NO 34, 35, 36 or 37 having at least 22.0% overall sequence identity to the full-length of any one of said polypeptide with SEQ ID NO 34, 35, 36 or 37, or
is a functional homolog, variant or derivative of any one of SEQ ID NO 21, 30 or 31 having at least 30.0% overall sequence identity to the full-length of any one of said polypeptide with SEQ ID NO 21, 30 or 31, or
is a functional homolog, variant or derivative of any one of SEQ ID NO 19, 25, 27, 32 or 33 having at least 35.0% overall sequence identity to the full-length of any one of said polypeptide with SEQ ID NO 19, 25, 27, 32 or 33, or
is a functional homolog, variant or derivative of any one of SEQ ID NO 20, 22, 24, 26 or 28 having at least 45.0% overall sequence identity to the full-length of any one of said polypeptide with SEQ ID NO 20, 22, 24, 26 or 28, or
is a functional homolog, variant or derivative of SEQ ID NO 23 having at least 50.0% overall sequence identity to the full-length of said polypeptide with SEQ ID NO 23, or
is a functional homolog, variant or derivative of SEQ ID NO 29 having at least 70.0% overall sequence identity to the full-length of said polypeptide with SEQ ID NO 29, or
is a functional fragment comprising an oligopeptide sequence of at least 10 consecutive amino acid residues from any one of SEQ ID NO 21, 31, 34, 35, 36 or 37, or
is a functional fragment comprising an oligopeptide sequence of at least 13 consecutive amino acid residues from any one of SEQ ID NO 19, 20, 25, 28 or 30, or
is a functional fragment comprising an oligopeptide sequence of at least 15 consecutive amino acid residues from any one of SEQ ID NO 26, 27, 32 or 33, or
is a functional fragment comprising an oligopeptide sequence of at least 17 consecutive amino acid residues from any one of SEQ ID NO 22 or 24, or
is a functional fragment comprising an oligopeptide sequence of at least 22 consecutive amino acid residues from any one of SEQ ID NO 23 or 29.

57. Cell according to embodiment 56, wherein said alpha-1,2-fucosyltransferase has no galactoside alpha-1,2-fucosyltransferase activity on lactose or has an additional galactoside alpha-1,2-fucosyltransferase activity on lactose which is lower than 3.0% of the galactoside alpha-1,2-fucosyltransferase activity on lactose of the alpha-1,2-fucosyltransferase with SEQ ID NO 06 and
comprises a polypeptide sequence according to any one of SEQ ID NO 19, 20, 21, 22, 23, 24, 25, 26, 27, 30, 33, 34, 35, 36 or 37, or
is a functional homolog, variant or derivative of any one of SEQ ID NO 34, 35, 36 or 37 having at least 22.0% overall sequence identity to the full-length of any one of said polypeptide with SEQ ID NO 34, 35, 36 or 37, or
is a functional homolog, variant or derivative of any one of SEQ ID NO 21 or 30 having at least 30.0% overall sequence identity to the full-length of any one of said polypeptide with SEQ ID NO 21 or 30, or
is a functional homolog, variant or derivative of any one of SEQ ID NO 19, 25, 27 or 33 having at least 35.0% overall sequence identity to the full-length of any one of said polypeptide with SEQ ID NO 19, 25, 27 or 33, or
is a functional homolog, variant or derivative of any one of SEQ ID NO 20, 22, 24 or 26 having at least 45.0% overall sequence identity to the full-length of any one of said polypeptide with SEQ ID NO 20, 22, 24 or 26, or
is a functional homolog, variant or derivative of SEQ ID NO 23 having at least 50.0% overall sequence identity to the full-length of said polypeptide with SEQ ID NO 23, or
is a functional fragment comprising an oligopeptide sequence of at least 10 consecutive amino acid residues from any one of SEQ ID NO 21, 34, 35, 36 or 37, or
is a functional fragment comprising an oligopeptide sequence of at least 13 consecutive amino acid residues from any one of SEQ ID NO 19, 20, 25 or 30, or
is a functional fragment comprising an oligopeptide sequence of at least 15 consecutive amino acid residues from any one of SEQ ID NO 26, 27 or 33, or
is a functional fragment comprising an oligopeptide sequence of at least 17 consecutive amino acid residues from any one of SEQ ID NO 22 or 24, or
is a functional fragment comprising an oligopeptide sequence of at least 20 consecutive amino acid residues from SEQ ID NO 23.

58. Cell according to embodiment 56, wherein said alpha-1,2-fucosyltransferase has additional galactoside alpha-1,2-fucosyltransferase activity on lactose that is between 4.0 and 20.0% of the galactoside alpha-1,2-fucosyltransferase activity on lactose of the alpha-1,2-fucosyltransferase with SEQ ID NO 06 and
comprises a polypeptide sequence according to any one of SEQ ID NO 28, 29, 31 or 32, or
is a functional homolog, variant or derivative of any one of SEQ ID NO 31 or 32 having at least 35.0% overall sequence identity to the full-length of any one of said polypeptide with SEQ ID NO 31 or 32, or
is a functional homolog, variant or derivative of SEQ ID NO 28 having at least 40.0% overall sequence identity to the full-length of said polypeptide with SEQ ID NO 28, or
is a functional homolog, variant or derivative of SEQ ID NO 29 having at least 70.0% overall sequence identity to the full-length of said polypeptide with SEQ ID NO 29, or
is a functional fragment comprising an oligopeptide sequence of at least 10 consecutive amino acid residues from SEQ ID NO 31, or
is a functional fragment comprising an oligopeptide sequence of at least 14 consecutive amino acid residues from any one of SEQ ID NO 28 or 32, or
is a functional fragment comprising an oligopeptide sequence of at least 22 consecutive amino acid residues from SEQ ID NO 29.

59. Cell according to any one of embodiments 52 to 58, wherein said cell is modified with one or more gene expression modules, characterized in that the expression from any of said expression modules is either constitutive or is created by a natural inducer.

60. Cell according to any one of embodiments 52 to 59, wherein said cell is modified in the expression or activity of any one of said alpha-1,2-fucosyltransferases.

61. Cell according to any one of embodiments 52 to 60, wherein said cell is capable to produce one or more nucleotide-activated sugars chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy--L-arabino-4-hexulose, UDP-2-acetamido-2,6-dideoxy--L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7 (8,9) Ac₂, CMP-N-glycolylneuraminic acid (CMP-Neu5Gc), GDP-rhamnose and UDP-xylose.

62. Cell according to any one of embodiments 52 to 61, wherein said cell expresses one or more polypeptides chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, GDP-fucose pyrophosphorylase, fucose-1-phosphate guanylyltransferase, L-glutamine-D-fructose-6-phosphate aminotransferase, glucosamine-6-phosphate deaminase, phosphoglucosamine mutase, N-acetylglucosamine-6-phosphate deacetylase, N-acylglucosamine 2-epimerase, UDP-N-acetylglucosamine 2-epimerase, N-acetylmannosamine-6-phosphate 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridylyltransferase, glucosamine-1-phosphate acetyltransferase, N-acetylneuraminate synthase, N-acetylneuraminate lyase, N-acylneuraminate-9-phosphate phosphatase, N-acylneuraminate cytidylyltransferase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase, preferably wherein said cell is modified in the expression or activity of any one of said polypeptides.

63. Cell according to any one of embodiments 52 to 62, wherein said cell expresses one or more glycosyltransferases chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases, preferably, said fucosyltransferase is chosen from the list comprising alpha-1,2-fucosyltransferase, alpha-1,3-fucosyltransferase, alpha-1,4-fucosyltransferase and alpha-1,6-fucosyltransferase,
preferably, said sialyltransferase is chosen from the list comprising alpha-2,3-sialyltransferase, alpha-2,6-sialyltransferase and alpha-2,8-sialyltransferase,
preferably, said galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase, preferably, said glucosyltransferase is chosen from the list comprising alpha-glucosyltransferase, beta-1,2-glucosyltransferase, beta-1,3-glucosyltransferase and beta-1,4-glucosyltransferase,
preferably, said mannosyltransferase is chosen from the list comprising alpha-1,2-mannosyltransferase, alpha-1,3-mannosyltransferase and alpha-1,6-mannosyltransferase,
preferably, said N-acetylglucosaminyltransferase is chosen from the list comprising galactoside beta-1,3-N-acetylglucosaminyltransferase and beta-1,6-N-acetylglucosaminyltransferase,
preferably, said N-acetylgalactosaminyltransferase is an alpha-1,3-N-acetylgalactosaminyltransferase,
preferably, said cell is modified in the expression or activity of any one of said glycosyltransferases.

64. Cell according to any one of embodiments 52 to 63, wherein said compound comprising a structure of Formula I, II or III is an oligosaccharide, preferably said oligosaccharide is a mammalian milk oligosaccharide (MMO), more preferably a human milk oligosaccharide (HMO).

65. Cell according to any one of embodiments 52 to 64, wherein said compound comprising a structure of Formula I, II or III is a charged, preferably sialylated, compound or a neutral compound, preferably wherein said compound comprising a structure of Formula I, II or III is a charged, preferably sialylated, oligosaccharide or a neutral oligosaccharide.

66. Cell according to any one of embodiments 52 to 65, wherein said compound comprising a structure of Formula I, II or III is Fuc-α1,2-Gal-b1,3-GlcNAc-b1,3-R comprising one R group chosen from the list comprising a monosaccharide, a disaccharide or an oligosaccharide, preferably wherein said compound comprising a structure of Formula I, II or III is Fuc-α1,2-Gal-b1,3-GlcNAc-b1,3-Gal-R comprising one R group chosen from the list comprising a monosaccharide, a disaccharide or an oligosaccharide, more preferably wherein said compound comprising a structure of Formula I, II or III is lacto-N-fucopentaose I (LNFP-I, Fuc-α1,2-Gal-b1,3-GlcNAc-b1,3-Gal-b1,4-Glc).

67. Cell according to any one of embodiments 52 to 66, wherein said cell is using one or more precursor(s) for the production of said compound comprising a structure of Formula I, II or III, said precursor(s) being fed to the cell from the cultivation medium.

68. Cell according to any one of embodiments 52 to 67, wherein said cell is producing one or more precursor(s) for the production of said compound comprising a structure of Formula I, II or III.

69. Cell according to any one of embodiments 67 or 68, wherein said precursor for the production of said compound comprising a structure of Formula I, II or III is completely converted into said compound comprising a structure of Formula I, II or III.

70. Cell according to any one of embodiments 52 to 69, wherein said cell is capable to produce a compound comprising a structure of Formula IV, V or VI:

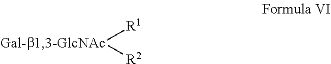

Gal-β1,3-GlcNAc Formula IV

Gal-β1,3-GlcNAc-R¹ Formula V
wherein:

R¹ is a monosaccharide, disaccharide, oligosaccharide, protein, glycoprotein, peptide, glycopeptide, lipid or glycolipid; R² is a monosaccharide, disaccharide or oligosaccharide.

71. Cell according to any one of embodiments 52 to 70, wherein said cell produces said compound comprising a structure of Formula I, II or III intracellularly and wherein a fraction or substantially all of said produced compound comprising a structure of Formula I, II or III remains intracellularly and/or is excreted outside said cell via passive or active transport.

72. Cell according to any one of embodiments 52 to 71, wherein said cell expresses a membrane transporter protein or a polypeptide having transport activity hereby transporting compounds across the outer membrane of the cell wall, preferably, said cell is modified in the expression or activity of said membrane transporter protein or polypeptide having transport activity.

73. Cell according to embodiment 72, wherein said membrane transporter protein or polypeptide having transport activity is chosen from the list comprising porters, P-P-bond-hydrolysis-driven transporters, b-barrel porins, auxiliary transport proteins, putative transport proteins and phosphotransfer-driven group translocators, preferably, said porters comprise MFS transporters, sugar efflux transporters and siderophore exporters, preferably, said P-P-bond-hydrolysis-driven transporters comprise ABC transporters and siderophore exporters.

74. Cell according to any one of embodiments 72 or 73, wherein said membrane transporter protein or polypeptide having transport activity controls the flow over the outer membrane of the cell wall of said compound comprising a structure of Formula I, II or III and/or of one or more precursor(s) and/or acceptor(s) to be used in said production of said compound comprising a structure of Formula I, II or III.

75. Cell according to any one of embodiments 72 to 74, wherein said membrane transporter protein or polypeptide having transport activity provides improved production and/or enabled and/or enhanced efflux of said compound comprising a structure of Formula I, II or III.

76. Cell according to any one of embodiment 52 to 75, wherein said cell comprises multiple copies of the same coding DNA sequence encoding for one protein.

77. Cell according to any one of embodiment 52 to 76, wherein said cell comprises a modification for reduced production of acetate.

78. Cell according to any one of embodiments 52 to 77, wherein said cell comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose: undecaprenyl-phosphate glucose-1-phosphate transferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 6-1, ATP-dependent phosphofructokinase isozyme 2, glucose-6-phosphate isomerase, aerobic respiration control protein, transcriptional repressor IclR, Ion protease, glucose-specific translocating phosphotransferase enzyme IIBC component ptsG, glucose-specific translocating phosphotransferase (PTS) enzyme IIBC component maIX, enzyme IIA^{Glc}, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, pyruvate decarboxylase.

79. Cell according to any one of embodiments 52 to 78, wherein the cell is capable to produce phosphoenolpyruvate (PEP).

80. Cell according to any one of embodiments 52 to 79, wherein said cell is modified for enhanced production and/or supply of phosphoenolpyruvate (PEP).

81. Cell according to any one of embodiments 52 to 80, wherein the cell comprises a catabolic pathway for selected mono-, di- or oligosaccharides which is at least partially inactivated, the mono-, di-, or oligosaccharides being involved in and/or required for said production of said compound comprising a structure of Formula I, II or III.

82. Cell according to any one of embodiments 52 to 81, wherein said cell resists the phenomenon of lactose killing when grown in an environment in which lactose is combined with one or more other carbon source(s).

83. Cell according to any one of embodiments 52 to 82, wherein said cell produces 90 g/L or more of said compound comprising a structure of Formula I, II or III in the whole broth and/or supernatant and/or wherein said compound comprising a structure of Formula I, II or III in the whole broth and/or supernatant has a purity of at least 80% measured on the total amount of said compound comprising a structure of Formula I, II or III and its precursor(s) in the whole broth and/or supernatant, respectively.

84. Cell according to any one of embodiments 52 to 83, wherein said cell is a bacterium, fungus, yeast, a plant cell, an animal cell, or a protozoan cell,
preferably said bacterium is an Escherichia coli strain, more preferably an Escherichia coli strain which is a K-12 strain, even more preferably the Escherichia coli K-12 strain is E. coli MG1655,
preferably said fungus belongs to a genus chosen from the group comprising Rhizopus, Dictyostelium, Penicillium, Mucor or Aspergillus,
preferably said yeast belongs to a genus chosen from the group comprising Saccharomyces, Zygosaccharomyces, Pichia, Komagataella, Hansenula, Yarrowia, Starmerella, Kluyveromyces or Debaromyces,
preferably said plant cell is an algal cell or is derived from tobacco, alfalfa, rice, tomato, cotton, rapeseed, soy, maize, or corn plant,
preferably said animal cell is derived from non-human mammals, birds, fish, invertebrates, reptiles, amphibians or insects or is a genetically modified cell line derived from human cells excluding embryonic stem cells, more preferably said human and non-human mammalian cell is an epithelial cell, an embryonic kidney cell, a fibroblast cell, a COS cell, a Chinese hamster ovary (CHO) cell, a murine myeloma cell, an NIH-3T3 cell, a non-mammary adult stem cell or derivatives thereof, more preferably said insect cell is derived from Spodoptera frugiperda, Bombyx mori, Mamestra brassicae, Trichoplusia ni or Drosophila melanogaster,
preferably said protozoan cell is a Leishmania tarentolae cell.

85. Cell according to embodiment 84, wherein said cell is a viable Gram-negative bacterium that comprises a reduced or abolished synthesis of poly-N-acetyl-glucosamine (PNAG), Enterobacterial Common Antigen (ECA), cellulose, colanic acid, core oligosaccharides, Osmoregulated Periplasmic Glucans (OPG), Glucosylglycerol, glycan, and/or trehalose.

86. Cell according to any one of embodiments 52 to 85, wherein the cell produces a mixture of charged, preferably sialylated, and/or neutral di- and oligosaccharides comprising at least one compound comprising a structure of Formula I, II or III, wherein R¹, when present, is a monosaccharide, a disaccharide or an oligosaccharide.

87. Cell according to any one of embodiments 52 to 86, wherein the cell produces a mixture of charged, preferably sialylated, and/or neutral oligosaccharides comprising at least one compound comprising a structure of Formula I, II or III, wherein R¹, when present, is a monosaccharide, a disaccharide or an oligosaccharide.

88. Use of a cell according to any one of embodiments 52 to 87, or method according to any one of embodiments 1 to 51 for the production of a compound comprising a structure of Formula I, II or III. The invention will be described in more detail in the examples. The following examples will serve as further illustration and clarification of the present invention and are not intended to be limiting.

### Examples

### Example 1. Calculation of Percentage Identity Between Nucleotide or Polypeptide Sequences

Methods for the alignment of sequences for comparison are well known in the art, such methods include GAP, BESTFIT, BLAST, FASTA and TFASTA. GAP uses the algorithm of Needleman and Wunsch (J. Mol. Biol. (1970) 48:443-453) to find the global (i.e. spanning the full-length sequences) alignment of two sequences that maximizes the number of matches and minimizes the number of gaps. The BLAST algorithm (Altschul et al., J. Mol. Biol. (1990) 215:403-10) calculates the global percentage sequence identity (i.e. over the full-length sequence) and performs a statistical analysis of the similarity between the two sequences. The software for performing BLAST analysis is publicly available through the National Centre for Biotechnology Information (NCBI). Homologs may readily be identified using, for example, the ClustalW multiple sequence alignment algorithm (version 1.83), with the default pairwise alignment parameters, and a scoring method in percentage. Global percentages of similarity and identity ((i.e. spanning the full-length sequences) may also be determined using one of the methods available in the MatGAT software package (Campanella et al., BMC Bioinformatics (2003) 4:29). Minor manual editing may be performed to optimize alignment between conserved motifs, as would be apparent to a person skilled in the art. Furthermore, instead of using full-length sequences for the identification of homologs, specific domains may also be used, to determine the so-called local sequence identity. The sequence identity values may be determined over the entire nucleic acid or amino acid sequence (=local sequence identity search over the full-length sequence resulting in a global sequence identity score) or over selected domains or conserved motif(s) (=local sequence identity search over a partial sequence resulting in a local sequence identity score), using the programs mentioned above using the default parameters. For local alignments, the Smith-Waterman algorithm is particularly useful (Smith T F, Waterman M S (1981) J. Mol. Biol 147 (1); 195-7).

### Example 2. Materials and Methods Escherichia coli

### Media

The Luria Broth (LB) medium consisted of 1% tryptone peptone (Difco, Erembodegem, Belgium), 0.5% yeast extract (Difco) and 0.5% sodium chloride (VWR, Leuven, Belgium). The minimal medium used in the cultivation experiments in 96-well plates or in shake flasks contained 2.00 g/L NH4Cl, 5.00 g/L (NH4) 2SO4, 2.993 g/L KH2PO4, 7.315 g/L K2HPO4, 8.372 g/L MOPS, 0.5 g/L NaCl, 0.5 g/L MgSO4.7H₂O, 30 g/L sucrose or 30 g/L glycerol, 1 ml/L vitamin solution, 100 μl/L molybdate solution, and 1 mL/L selenium solution. As specified in the respective examples, 0.30 g/L sialic acid, 0.30 g/L GlcNAc, 20 g/L lactose, 20 g/L LacNAc and/or 20 g/L LNB were additionally added to the medium as precursor(s). The minimal medium was set to a pH of 7 with 1M KOH. Vitamin solution consisted of 3.6 g/L FeCl2.4H₂O, 5 g/L CaCl2).2H2O, 1,3 g/L MnCl2.2H2O, 0.38 g/L CuCl2.2H2O, 0.5 g/L CoCl2.6H2O, 0.94 g/L ZnCl2, 0.0311 g/L H3BO4, 0.4 g/L Na2EDTA.2H2O and 1.01 g/L thiamine.HCl. The molybdate solution contained 0.967 g/L NaMoO4.2H2O. The selenium solution contained 42 g/L Seo2.

The minimal medium for fermentations contained 6.75 g/L NH4Cl, 1,25 g/L (NH4) 2SO4, 2.93 g/L KH2PO4 and 7.31 g/L KH2PO4, 0.5 g/L NaCl, 0.5 g/L MgSO4.7H2O, 30 g/L sucrose or 30 g/L glycerol, 1 mL/L vitamin solution, 100 μL/L molybdate solution, and 1 mL/L selenium solution with the same composition as described above. As specified in the respective examples, 0.30 g/L sialic acid, 0.30 g/L GlcNAc, 20 g/L lactose, 20 g/L LacNAc and/or 20 g/L LNB were additionally added to the medium as precursor(s).

Complex medium was sterilized by autoclaving (121° C., 21 min) and minimal medium by filtration (0.22 μm Sartorius). When necessary, the medium was made selective by adding an antibiotic: e.g. chloramphenicol (20 mg/L), carbenicillin (100 mg/L), spectinomycin (40 mg/L) and/or kanamycin (50 mg/L).

### Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007). Plasmids were maintained in the host E. coli DH5alpha (F⁻, phi80dlacZΔM15, Δ(lacZYA-argF) U169, deoR, recA1, endA1, hsdR17 (rk, mk+), phoA, supE44, lambda, thi-1, gyrA96, relA1) bought from Invitrogen.

### Strains and Mutations

Escherichia coli K12 MG1655 [X], F, rph-1] was obtained from the Coli Genetic Stock Center (US), CGSC Strain #: 7740, in March 2007. Gene disruptions, gene introductions and gene replacements were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain. Transformants carrying a Red helper plasmid pKD46 were grown in 10 mL LB media with ampicillin, (100 mg/L) and L-arabinose (10 mM) at 30° C. to an OD600 nm of 0.6. The cells were made electrocompetent by washing them with 50 mL of ice-cold water, a first time, and with 1 mL ice cold water, a second time. Then, the cells were resuspended in 50 μl of ice-cold water. Electroporation was done with 50 μL of cells and 10-100 ng of linear double-stranded-DNA product by using a Gene Pulser™ (BioRad) (600 Ω, 25 uFD, and 250 volts). After electroporation, cells were added to 1 mL LB media incubated 1 h at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity. The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knock-out, the region of homology was designed 50-nt upstream and 50-nt downstream of the start and stop codon of the gene of interest. For the genomic knock-in, the transcriptional starting point (+1) had to be respected. PCR products were PCR-purified, digested with Dpnl, re-purified from an agarose gel, and suspended in elution buffer (5 mM Tris, pH 8.0). Selected mutants were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knock outs and knock ins are checked with control primers.

In one example for GDP-fucose production, the mutant strain was derived from E. coli K12 MG1655 comprising knock-outs of the E. coli wcaI and thyA genes and genomic knock-ins of constitutive transcriptional units containing a sucrose transporter like e.g. CscB from E. coli W (UniProt ID EOIXR1, sequence version 1), a fructose kinase like e.g. Frk originating from Zymomonas mobilis (UniProt ID Q03417, sequence version 1) and a sucrose phosphorylase like e.g. BaSP originating from Bifidobacterium adolescentis (UniProt ID AOZZH6, sequence version 1). GDP-fucose production can further be optimized in the mutant E. coli strain by genomic knock-outs of any one or more of the E. coli genes comprising glgC, agp, pfkA, pfkB, pgi, arcA, icIR, pgi and Ion as described in WO2016075243 and WO2012007481. GDP-fucose production can additionally be optimized comprising genomic knock-ins of constitutive transcriptional units for a mannose-6-phosphate isomerase like e.g. manA from E. coli (UniProt ID P00946, sequence version 1), a phosphomannomutase like e.g. manB from E. coli (UniProt ID P24175, sequence version 1), a mannose-1-phosphate guanylyltransferase like e.g. manC from E. coli (UniProt ID P24174, sequence version 3), a GDP-mannose 4,6-dehydratase like e.g. gmd from E. coli (UniProt ID POAC88, sequence version 1) and a GDP-L-fucose synthase like e.g. fcl from E. coli (UniProt ID P32055, sequence version 2). GDP-fucose production can also be obtained by genomic knock-outs of the E. coli fucK and fucI genes and genomic knock-ins of constitutive transcriptional units containing a fucose permease like e.g. fucP from E. coli (UniProt ID P11551, sequence version 3) and a bifunctional enzyme with fucose kinase/fucose-1-phosphate guanylyltransferase activity like e.g. fkp from Bacteroides fragilis (UniProt ID SUV40286.1, sequence version 1). All mutant strains can be additionally modified with genomic knock-outs of the E. coli LacZ, LacY and LacA genes and with a genomic knock-in of a constitutive transcriptional unit for a lactose permease like e.g. the E. coli LacY (UniProt ID P02920, sequence version 1).

For production of fucosylated oligosaccharides, the mutant GDP-fucose production strain was additionally modified with expression plasmids comprising constitutive transcriptional units for an alpha-1,2-fucosyltransferase like e.g. any one or more of SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 or 37 and/or an alpha-1,3-fucosyltransferase like e.g. HpFucT from H. pylori (UniProt ID 030511, sequence version 1) and with a constitutive transcriptional unit for the E. coli thyA (UniProt ID POA884, sequence version 1) as selective marker. Additionally, and/or alternatively, the constitutive transcriptional units of the fucosyltransferase genes could be present in the mutant E. coli strain via genomic knock-ins.

Alternatively, and/or additionally, GDP-fucose and/or fucosylated oligosaccharide production can further be optimized in the mutant E. coli strains with genomic knock-ins of constitutive transcriptional units comprising a membrane transporter protein like e.g. MdfA from Cronobacter muytjensii (UniProt ID A0A2T7ANQ9, sequence version 1), MdfA from Citrobacter youngae (UniProt ID D4BC23, sequence version 1), MdfA from E. coli (UniProt ID POAEY8, sequence version 1), MdfA from Yokenella regensburgei (UniProt ID G9Z5F4, sequence version 1), iceT from E. coli (UniProt ID A0A024L207, sequence version 1) or iceT from Citrobacter youngae (UniProt ID D4B8A6, sequence version 1).

In an example for production of lacto-N-biose (LNB, Gal-b1,3-GlcNAc) the strains were modified with genomic knock-ins or expression plasmids comprising constitutive transcriptional units for a glucosamine 6-phosphate N-acetyltransferase like e.g. GNA1 from S. cerevisiae (UniProt ID P43577, sequence version 1) and an N-acetylglucosamine beta-1,3-galactosyltransferase like e.g. WbgO from E. coli 055: H7 (Uniprot ID D3QY14, sequence version 1).

In an example to produce lacto-N-triose (LN3, GlcNAc-b1,3-Gal-b1,4-Glc), the mutant strain was derived from E. coli K12 MG1655 and modified with a knock-out of the E. coli lacZ, lacY, lacA and nagB genes and with genomic knock-ins of constitutive transcriptional units for a lactose permease like e.g. the E. coli LacY (UniProt ID P02920, sequence version 1) and a galactoside beta-1,3-N-acetylglucosaminyltransferase like e.g. IgtA (UniProt ID Q9JXQ6, sequence version 1) from N. meningitidis.

In an example for production of LN3 derived oligosaccharides like lacto-N-tetraose (LNT, Gal-b1,3-GlcNAc-b1,3-Gal-b1,4-Glc), the mutant LN3 producing strain was further modified with a constitutive transcriptional unit delivered to the strain either via genomic knock-in or from an expression plasmid for an N-acetylglucosamine beta-1,3-galactosyltransferase like e.g. wbgO (Uniprot ID D3QY14, sequence version 1) from E. coli 055: H7. LNB, LN3 and/or LNT production can further be optimized in the mutant E. coli strains with genomic knock-outs of the E. coli genes comprising any one or more of galT, ushA, IdhA and agp.

The mutant LNB, LN3 and LNT producing strains can also be optionally modified for enhanced UDP-GlcNAc production with a genomic knock-in of a constitutive transcriptional unit for an L-glutamine-D-fructose-6-phosphate aminotransferase like e.g. the mutant glmS*54 from E. coli (differing from the wild-type E. coli glmS protein, having UniProt ID P17169, sequence version 4, by an A39T, an R250C and an G472S mutation as described by Deng et al. (Biochimie 2006, 88:419-429).

The mutant E. coli strains can also optionally be adapted with a genomic knock-in of a constitutive transcriptional unit for an UDP-glucose-4-epimerase like e.g. galE from E. coli (UniProt ID P09147, sequence version 1), a phosphoglucosamine mutase like e.g. glmM from E. coli (UniProt ID P31120, sequence version 3) and an N-acetylglucosamine-1-phosphate uridylyltransferase/glucosamine-1-phosphate acetyltransferase like e.g. glmU from E. coli (UniProt ID P0ACC7, sequence version 1).

The mutant LNB, LN3 and LNT producing E. coli strains can also optionally be adapted for growth on sucrose via genomic knock-ins of constitutive transcriptional units containing a sucrose transporter like e.g. CscB from E. coli W (UniProt ID EOIXR1, sequence version 1), a fructose kinase like e.g. Frk originating from Zymomonas mobilis (UniProt ID Q03417, sequence version 1) and a sucrose phosphorylase like e.g. BaSP originating from Bifidobacterium adolescentis (UniProt ID AOZZH6, sequence version 1).

Alternatively, and/or additionally, production of LNB, LN3, LNT and oligosaccharides derived thereof can further be optimized in the mutant E. coli strains with genomic knock-ins of constitutive transcriptional units comprising a membrane transporter protein like e.g. MdfA from Cronobacter muytjensii (UniProt ID A0A2T7ANQ9, sequence version 1), MdfA from Citrobacter youngae (UniProt ID D4BC23, sequence version 1), MdfA from E. coli (UniProt ID POAEY8, sequence version 1), MdfA from Yokenella regensburgei (UniProt ID G9Z5F4, sequence version 1), iceT from E. coli (UniProt ID A0A024L207, sequence version 1) or iceT from Citrobacter youngae (UniProt ID D4B8A6, sequence version 1).

In one example for sialic acid production, the mutant strain was derived from E. coli K12 MG1655 comprising genomic knock-ins of constitutive transcriptional units containing one or more copies of a glucosamine 6-phosphate N-acetyltransferase like e.g. GNA1 from Saccharomyces cerevisiae (UniProt ID P43577, sequence version 1), an N-acetylglucosamine 2-epimerase like e.g. AGE from Bacteroides ovatus (UniProt ID A7LVG6, sequence version 1) and an N-acetylneuraminate synthase like e.g. from Neisseria meningitidis (UniProt ID EONCD4, sequence version 1) or Campylobacter jejuni (UniProt ID Q93MP9, sequence version 1).

Alternatively, and/or additionally, sialic acid production can be obtained by genomic knock-ins of constitutive transcriptional units containing an UDP-N-acetylglucosamine 2-epimerase like e.g. NeuC from C. jejuni (UniProt ID Q93MP8, sequence version 1) and an N-acetylneuraminate synthase like e.g. from Neisseria meningitidis (UniProt ID EONCD4, sequence version 1) or Campylobacter jejuni (UniProt ID Q93MP9, sequence version 1).

Alternatively and/or additionally, sialic acid production can be obtained by genomic knock-ins of constitutive transcriptional units containing a phosphoglucosamine mutase like e.g. glmM from E. coli (UniProt ID P31120, 3), sequence version an N-acetylglucosamine-1-phosphate uridyltransferase/glucosamine-1-phosphate acetyltransferase like e.g. glmU from E. coli (UniProt ID P0ACC7, sequence version 1), an UDP-N-acetylglucosamine 2-epimerase like e.g. NeuC from C. jejuni (UniProt ID Q93MP8, sequence version 1) and an N-acetylneuraminate synthase like e.g. from Neisseria meningitidis (UniProt ID EONCD4, sequence version 1) or Campylobacter jejuni (UniProt ID Q93MP9, sequence version 1).

Alternatively, and/or additionally, sialic acid production can be obtained by genomic knock-ins of constitutive transcriptional units containing a bifunctional UDP-GlcNAc 2-epimerase/N-acetylmannosamine kinase like e.g. from Mus musculus (strain C57BL/6J) (UniProt ID Q91WG8, sequence version 1), an N-acylneuraminate-9-phosphate synthetase like e.g. from Pseudomonas sp. UW4 (UniProt ID K9NPH9, sequence version 1) and an N-acylneuraminate-9-phosphatase like e.g. from Candidatus Magnetomorum sp. HK-1 (UniProt ID KPA15328.1, sequence version 1) or from Bacteroides thetaiotaomicron (UniProt ID Q8A712, sequence version 1).

Alternatively, and/or additionally, sialic acid production can be obtained by genomic knock-ins of constitutive transcriptional units containing a phosphoglucosamine mutase like e.g. glmM from E. coli (UniProt ID P31120, sequence version 3), an N-acetylglucosamine-1-phosphate uridyltransferase/glucosamine-1-phosphate acetyltransferase like e.g. glmU from E. coli (UniProt ID P0ACC7, sequence version 1), a bifunctional UDP-GlcNAc 2-epimerase/N-acetylmannosamine kinase like e.g. from M. musculus (strain C57BL/6J) (UniProt ID Q91WG8, sequence version 1), an N-acylneuraminate-9-phosphate synthetase like e.g. from Pseudomonas sp. UW4 (UniProt ID K9NPH9, sequence version 1) and an N-acylneuraminate-9-phosphatase like e.g. from Candidatus Magnetomorum sp. HK-1 (UniProt ID KPA15328.1, sequence version 1) or from Bacteroides thetaiotaomicron (UniProt ID Q8A712, sequence version 1).

Sialic acid production can further be optimized in the mutant E. coli strain with genomic knock-outs of the E. coli genes comprising any one or more of nagA, nagB, nagC, nagD, nagE, nanA, nanE, nanK, manX, manY and manZ as described in WO18122225, and/or genomic knock-outs of the E. coli genes comprising any one or more of nanT, poxB, IdhA, adhE, aldB, pflA, pfIC, ybiY, ackA and/or pta and with genomic knock-ins of constitutive transcriptional units comprising one or more copies of an L-glutamine-D-fructose-6-phosphate aminotransferase like e.g. the mutant glmS*54 from E. coli (differing from the wild-type E. coli glmS, having UniProt ID P17169, sequence version 4, by an A39T, an R250C and an G472S mutation as described by Deng et al. (Biochimie 88, 419-29 (2006)), preferably a phosphatase like any one of e.g. the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonas putida, ScDOG1 from S. cerevisiae or BsAraL from Bacillus subtilis as described in WO18122225 and an acetyl-CoA synthetase like e.g. acs from E. coli (UniProt ID P27550, sequence version 2).

For sialylated oligosaccharide production, said sialic acid production strains were further modified to express an N-acylneuraminate cytidylyltransferase like e.g. the NeuA enzyme from C. jejuni (UniProt ID Q93MP7, sequence version 1), the NeuA enzyme from Haemophilus influenzae (GenBank No. AGV11798.1) or the NeuA enzyme from Pasteurella multocida (GenBank No. AMK07891.1) and to express one or more copies of a beta-galactoside alpha-2,3-sialyltransferase like e.g. PmultST3 from P. multocida (UniProt ID Q9CLP3, sequence version 1) or a PmultST3-like polypeptide consisting of amino acid residues 1 to 268 of UniProt ID Q9CLP3, sequence version 1, having beta-galactoside alpha-2,3-sialyltransferase activity, NmeniST3 from N. meningitidis (GenBank No. ARC07984.1) or PmultST2 from P. multocida subsp. multocida str. Pm70 (GenBank No. AAK02592.1), a beta-galactoside alpha-2,6-sialyltransferase like e.g. PdST6 from Photobacterium damselae (UniProt ID 066375, sequence version 1) or a PdST6-like polypeptide consisting of amino acid residues 108 to 497 of UniProt ID 066375, sequence version 1, having beta-galactoside alpha-2,6-sialyltransferase activity or P-JT-ISH-224-ST6 from Photobacterium sp. JT-ISH-224 (UniProt ID A8QYL1, sequence version 1) or a P-JT-ISH-224-ST6-like polypeptide consisting of amino acid residues 18 to 514 of UniProt ID A8QYL1, sequence version 1, having beta-galactoside alpha-2,6-sialyltransferase activity, and/or an alpha-2,8-sialyltransferase like e.g. from M. musculus (UniProt ID Q64689, sequence version 2). Constitutive transcriptional units of the N-acylneuraminate cytidylyltransferase and the sialyltransferases can be delivered to the mutant strain either via genomic knock-in or via expression plasmids. If the mutant strains producing sialic acid and CMP-sialic acid were intended to make sialylated lactose structures, the strains were additionally modified with genomic knock-outs of the E. coli LacZ, LacY and LacA genes and with a genomic knock-in of a constitutive transcriptional unit for a lactose permease like e.g. E. coli LacY (UniProt ID P02920, sequence version 1).

All mutant strains producing sialic acid, CMP-sialic acid and/or sialylated oligosaccharides could optionally be adapted for growth on sucrose via genomic knock-ins of constitutive transcriptional units containing a sucrose transporter like e.g. CscB from E. coli W (UniProt ID EOIXR1, sequence version 1), a fructose kinase like e.g. Frk originating from Z. mobilis (UniProt ID Q03417, sequence version 1) and a sucrose phosphorylase like e.g. BaSP from B. adolescentis (UniProt ID AOZZH6, sequence version 1).

Alternatively, and/or additionally, sialic acid and/or sialylated oligosaccharide production can further be optimized in the mutant E. coli strains with genomic knock-ins of constitutive transcriptional units comprising a membrane transporter protein like e.g. a sialic acid transporter like e.g. nanT from E. coli K-12 MG1655 (UniProt ID P41036, sequence version 2), nanT from E. coli 06: H1 (UniProt ID Q8FD59, sequence version 2), nanT from E. coli 0157: H7 (UniProt ID Q8X9G8, sequence version 2) or nanT from E. albertii (UniProt ID B1EFH1, sequence version 1) or a porter like e.g. EntS from E. coli (UniProt ID P24077, sequence version 2), EntS from Kluyvera ascorbata (UniProt ID A0A378GQ13, sequence version 1) or EntS from Salmonella enterica subsp. arizonae (UniProt ID AOA6Y2K4E8, sequence version 1), MdfA from Cronobacter muytjensii (UniProt ID A0A2T7ANQ9, sequence version 1), MdfA from Citrobacter youngae (UniProt ID D4BC23, sequence version 1), MdfA from E. coli (UniProt ID POAEY8, sequence version 1), MdfA from Yokenella regensburgei (UniProt ID G9Z5F4, sequence version 1), iceT from E. coli (UniProt ID A0A024L207, sequence version 1), iceT from Citrobacter youngae (UniProt ID D4B8A6, sequence version 1), SetA from E. coli (UniProt ID P31675, sequence version 3), SetB from E. coli (UniProt ID P33026, sequence version 1) or SetC from E. coli (UniProt ID P31436, sequence version 1) or an ABC transporter like e.g. oppF from E. coli (UniProt ID P77737, sequence version 1), ImrA from Lactococcus lactis subsp. lactis bv. diacetylactis (UniProt ID A0A1VONEL4, sequence version 1), or Blon_2475 from Bifidobacterium longum subsp. infantis (UniProt ID B7GPD4, sequence version 1).

Preferably but not necessarily, any one or more of the glycosyltransferases, the proteins involved in nucleotide-activated sugar synthesis and/or the membrane transporter protein were N- and/or C-terminally fused to a solubility enhancer tag like e.g. a SUMO-tag, an MBP-tag, His, FLAG, Strep-II, Halo-tag, NusA, thioredoxin, GST and/or the Fh8-tag to enhance their solubility (Costa et al., Front. Microbiol. 2014, https://doi.org/10.3389/fmicb.2014.00063; Fox et al., Protein Sci. 2001, 10 (3), 622-630; Jia and Jeaon, Open Biol. 2016, 6:160196).

Optionally, the mutant E. coli strains were modified with a genomic knock-ins of a constitutive transcriptional unit encoding a chaperone protein like e.g. Dnak, DnaJ, GrpE or the GroEL/ES chaperonin system (Baneyx F., Palumbo J. L. (2003) Improving Heterologous Protein Folding via Molecular Chaperone and Foldase Co-Expression. In: Vaillancourt P. E. (eds) E. coli Gene Expression Protocols. Methods in Molecular Biology™, vol 205. Humana Press).

Optionally, the mutant E. coli strains are modified to create a glycominimized E. coli strain comprising genomic knock-out of any one or more of non-essential glycosyltransferase genes comprising pgaC, pgaD, rfe, rffT, rffM, bcsA, bcsB, bcsC, wcaA, wcaC, wcaE, wcaI, wcaJ, wcaL, waaH, waaF, waaC, waaU, waaZ, waaJ, waaO, waaB, waaS, waaG, waaQ, wbbI, arnC, arnT, yfdH, wbbK, opgG, opgH, ycjM, glgA, glgB, malQ, otsA and yaiP.

All constitutive promoters, UTRs and terminator sequences originated from the libraries described by Cambray et al. (Nucleic Acids Res. 2013, 41 (9), 5139-5148), Dunn et al. (Nucleic Acids Res. 1980, 8, 2119-2132), Edens et al. (Nucleic Acids Res. 1975, 2, 1811-1820), Kim and Lee (FEBS Letters 1997, 407, 353-356) and Mutalik et al. (Nat. Methods 2013, No. 10, 354-360).

The SEQ ID NOs described in present invention are summarized in Table 1.

All genes were ordered synthetically at Twist Bioscience (twistbioscience.com) or IDT (eu.idtdna.com) and the codon usage was adapted using the tools of the supplier.

All strains were stored in cryovials at −80° C. (overnight LB culture mixed in a 1:1 ratio with 70% glycerol).

TABLE 1
Overview of SEQ ID NOs described in the present invention

| | | | Country of |
|---|---|---|---|
| SEQ | | | origin of digital |
| ID | | | sequence |
| NO | Organism | Origin | information |
| 01 | Polaribacter vadi | Synthetic | South Korea |
| 02 | Dysgonomonas mossii | Synthetic | United States |
| 03 | Desulfovibrio alaskensis G20 | Synthetic | United States |
| 04 | Dechlorosoma suillum PS | Synthetic | United States |
| 05 | Capnocytophaga canis | Synthetic | Unknown |
| 06 | Butyrivibrio proteoclasticus | Synthetic | Unknown |
| | B316 | | |
| 07 | Helicobacter sp. MIT 01-6242 | Synthetic | United States |
| 08 | Butyrivibrio proteoclasticus | Synthetic | Unknown |
| | B316 | | |
| 09 | Akkermansia muciniphila | Synthetic | Netherlands |
| 10 | Thermosynechococcus sp. NK55 | Synthetic | Japan |
| 11 | Porphyromonas catoniae | Synthetic | Unknown |
| 12 | Francisella sp. FSC1006 | Synthetic | Italy |
| 13 | Chitinophaga sp. CF118 | Synthetic | Unknown |
| 14 | Selenomonas sp. CM52 | Synthetic | Unknown |
| 15 | Desulfotomaculum reducens MI-1 | Synthetic | Unknown |
| 16 | Bacteroides fragilis str. 3397 T10 | Synthetic | United States |
| 17 | Pseudovibrio sp. FO-BEG1 | Synthetic | United States |
| 18 | Colwellia psychrerythraea 34H | Synthetic | Unknown |
| 19 | Bacteroides fragilis | Synthetic | United States |
| 20 | Geobacter uraniireducens Rf4 | Synthetic | Unknown |
| 21 | Dyadobacter fermentans | Synthetic | United States |
| 22 | Escherichia albertii KF1 | Synthetic | Japan |
| 23 | Parabacteroides sp. YL27 | Synthetic | Switzerland |
| 24 | Sphingobacteriaceae bacterium | Synthetic | United States |
| 25 | Prevotella sp. oral taxon 299 str. | Synthetic | Unknown |
| | F0039 | | |
| 26 | Pseudomonas asplenii | Synthetic | Unknown |
| 27 | Bacteroides nordii CL02T12C05 | Synthetic | Unknown |
| 28 | Selenomonas ruminantium | Synthetic | Unknown |
| 29 | Bacteroides sp. A51A | Synthetic | Unknown |
| 30 | Selenomonas sputigena | Synthetic | Unknown |
| 31 | Capnocytophaga leadbetteri | Synthetic | Unknown |
| 32 | Helicobacter sp. CLO-3 | Synthetic | United States |
| 33 | Helicobacter sp. MIT 17-337 | Synthetic | United States |
| 34 | Cyanobium gracile PCC 6307 | Synthetic | United States |
| 35 | Desulfococcus oleovorans Hxd3 | Synthetic | Unknown |
| 36 | Elizabethkingia bruuniana G0146 | Synthetic | United Kingdom |
| 37 | Flammeovirgaceae bacterium 311 | Synthetic | Unknown |
| 38 | N.A. | Synthetic | Artificial |
| | | | sequence |
| 39 | N.A. | Synthetic | Artificial |
| | | | sequence |

### Cultivation Conditions

A preculture of 96-well microtiter plate experiments was started from a cryovial, in 150 μL LB and was incubated overnight at 37° C. on an orbital shaker at 800 rpm. This culture was used as inoculum for a 96well square microtiter plate, with 400 μl minimal medium by diluting 400×. These final 96-well culture plates were then incubated at 37° C. on an orbital shaker at 800 rpm for 72h, or shorter, or longer. To measure sugar concentrations at the end of the cultivation experiment whole broth samples were taken from each well by boiling the culture broth for 15 min at 60° C. before spinning down the cells (=average of intra- and extracellular sugar concentrations).

A preculture for the bioreactor was started from an entire 1 mL cryovial of a certain strain, inoculated in 250 ml or 500 mL minimal medium in a 1 L or 2.5 L shake flask and incubated for 24 h at 37° C. on an orbital shaker at 200 rpm. A 5 L bioreactor (having a 5 L working volume) was then inoculated (250 mL inoculum in 2 L batch medium); the process was controlled by MFCS control software (Sartorius Stedim Biotech, Melsungen, Germany). Culturing condition were set to 37° C., and maximal stirring; pressure gas flow rates were dependent on the strain and bioreactor. The pH was controlled at 6.8 using 0.5 M H2SO4 and 20% NH4OH. The exhaust gas was cooled. 10% solution of silicone antifoaming agent was added when foaming raised during the fermentation.

### Optical Density

Cell density of the cultures was frequently monitored by measuring optical density at 600 nm (Implen Nanophotometer NP80, Westburg, Belgium or with a Spark 10M microplate reader, Tecan, Switzerland).

### Analytical Analysis

Standards such as but not limited to sucrose, lactose, lacto-N-biose (LNB, Gal-b1,3-GlcNAc), fucosylated LNB (2′FLNB, 4′FLNB), lacto-N-triose II (LN3), lacto-N-tetraose (LNT), lacto-N-neo-tetraose (LNnT), LNFP-I, LNFP-II, LNFP-III, LNFP-V, LNFP-VI, were purchased from Carbosynth (UK), Elicityl (France) and IsoSep (Sweden). Other compounds were analysed with in-house made standards.

Neutral oligosaccharides were analysed on a Waters Acquity H-class UPLC with Evaporative Light Scattering Detector (ELSD) or a Refractive Index (RI) detection. A volume of 0.7 μL sample was injected on a Waters Acquity UPLC BEH Amide column (2.1×100 mm; 130 Å;1.7 μm) column with an Acquity UPLC BEH Amide VanGuard column, 130 Å, 2.1×5 mm. The column temperature was 50° C. The mobile phase consisted of a ¼ water and ¾ acetonitrile solution to which 0.2% triethylamine was added. The method was isocratic with a flow of 0.130 mL/min. The ELS detector had a drift tube temperature of 50° C. and the N2 gas pressure was 50 psi, the gain 200 and the data rate 10 pps. The temperature of the RI detector was set at 35° C.

Sialylated oligosaccharides were analysed on a Waters Acquity H-class UPLC with Refractive Index (RI) detection. A volume of 0.5 μl sample was injected on a Waters Acquity UPLC BEH Amide column (2.1×100 mm;130 Å;1.7 μm). The column temperature was 50° C. The mobile phase consisted of a mixture of 70% acetonitrile, 26% ammonium acetate buffer (150 mM) and 4% methanol to which 0.05% pyrrolidine was added. The method was isocratic with a flow of 0.150 ml/min. The temperature of the RI detector was set at 35° C.

Both neutral and sialylated sugars were analysed on a Waters Acquity H-class UPLC with Refractive Index (RI) detection. A volume of 0.5 L sample was injected on a Waters Acquity UPLC BEH Amide column (2.1×100 mm;130 Å;1.7 μm). The column temperature was 50° C. The mobile phase consisted of a mixture of 72% acetonitrile and 28% ammonium acetate buffer (100 mM) to which 0.1% triethylamine was added. The method was isocratic with a flow of 0.260 mL/min. The temperature of the RI detector was set at 35° C.

For analysis on a mass spectrometer, a Waters Xevo TQ-MS with Electron Spray Ionisation (ESI) was used with a desolvation temperature of 450° C., a nitrogen desolvation gas flow of 650 L/h and a cone voltage of 20 V. The MS was operated in selected ion monitoring (SIM) in negative mode for all oligosaccharides. Separation was performed on a Waters Acquity UPLC with a Thermo Hypercarb column (2.1×100 mm; 3 μm) on 35° C. A gradient was used wherein eluent A was ultrapure water with 0.1% formic acid and wherein eluent B was acetonitrile with 0.1% formic acid. The oligosaccharides were separated in 55 min using the following gradient: an initial increase from 2 to 12% of eluent B over 21 min, a second increase from 12 to 40% of eluent B over 11 min and a third increase from 40 to 100% of eluent B over 5 min. As a washing step 100% of eluent B was used for 5 min. For column equilibration, the initial condition of 2% of eluent B was restored in 1 min and maintained for 12 min.

Both neutral and sialylated sugars at low concentrations (below 50 mg/L) were analysed on a Dionex HPAEC system with pulsed amperometric detection (PAD). A volume of 5 μL of sample was injected on a Dionex CarboPac PA200 column 4×250 mm with a Dionex CarboPac PA200 guard column 4×50 mm. The column temperature was set to 30° C. A gradient was used wherein eluent A was deionized water, wherein eluent B was 200 mM Sodium hydroxide and wherein eluent C was 500 mM Sodium acetate. The oligosaccharides were separated in 60 min while maintaining a constant ratio of 25% of eluent B using the following gradient: an initial isocratic step maintained for 10 min of 75% of eluent A, an initial increase from 0 to 4% of eluent C over 8 min, a second isocratic step maintained for 6 min of 71% of eluent A and 4% of eluent C, a second increase from 4 to 12% of eluent C over 2.6 min, a third isocratic step maintained for 3.4 min of 63% of eluent A and 12% of eluent C and a third increase from 12 to 48% of eluent C over 5 min. As a washing step 48% of eluent C was used for 3 min. For column equilibration, the initial condition of 75% of eluent A and 0% of eluent C was restored in 1 min and maintained for 11 min. The applied flow was 0.5 mL/min.

### Example 3. Materials and Methods Saccharomyces cerevisiae

### Media

Strains were grown on Synthetic Defined yeast medium with Complete Supplement Mixture (SD CSM) or CSM drop-out (SD CSM-Ura, SD CSM-Trp, SD CSM-His) containing 6.7 g/L Yeast Nitrogen Base without amino acids (YNB w/o AA, Difco), 20 g/L agar (Difco) (solid cultures), 22 g/L glucose monohydrate or 20 g/L lactose and 0.79 g/L CSM or 0.77 g/L CSM-Ura, 0.77 g/L CSM-Trp, or 0.77 g/L CSM-His (MP Biomedicals).

### Strains

S. cerevisiae BY4742 created by Brachmann et al. (Yeast (1998) 14:115-32) was used, available in the Euroscarf culture collection. All mutant strains were created by homologous recombination or plasmid transformation using the method of Gietz (Yeast 11:355-360, 1995).

### Plasmids

In one example to produce GDP-fucose, a yeast expression plasmid like p2a_2μ_Fuc (Chan 2013, Plasmid 70, 2-17) can be used for expression of foreign genes in S. cerevisiae. This plasmid contains an ampicillin resistance gene and a bacterial origin of replication to allow for selection and maintenance in E. coli and the 2μ yeast ori and the Ura3 selection marker for selection and maintenance in yeast. This plasmid is further modified with constitutive transcriptional units for a lactose permease like e.g. LAC12 from K. lactis (UniProt ID P07921, sequence version 1), a GDP-mannose 4,6-dehydratase like e.g. gmd from E. coli (UniProt ID POAC88, sequence version 1) and a GDP-L-fucose synthase like e.g. fcl from E. coli (UniProt ID P32055, sequence version 2). The yeast expression plasmid p2a_2μ_Fuc2 can be used as an alternative expression plasmid of the p2a_2μ_Fuc plasmid comprising next to the ampicillin resistance gene, the bacterial ori, the 2μ yeast ori and the Ura3 selection marker constitutive transcriptional units for a lactose permease like e.g. LAC12 from K. lactis (UniProt ID P07921, sequence version 1), a fucose permease like e.g. fucP from E. coli (UniProt ID P11551, sequence version 3) and a bifunctional enzyme with fucose kinase/fucose-1-phosphate guanylyltransferase activity like e.g. fkp from Bacteroides fragilis (UniProt ID SUV40286.1, sequence version 1). To further produce fucosylated oligosaccharides, the p2a_2μ_Fuc and its variant the p2a_2μ_Fuc2, additionally contained a constitutive transcriptional unit for a fucosyltransferases, like e.g. an alpha-1,2-fucosyltransferase chosen from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 or 37 and/or an alpha-1,3-fucosyltransferase.

In one example to produce UDP-galactose, a yeast expression plasmid can be derived from the pRS420-plasmid series (Christianson et al., 1992, Gene 110:119-122) containing the HIS3 selection marker and a constitutive transcriptional unit for an UDP-glucose-4-epimerase like e.g. galE from E. coli (UniProt ID P09147, sequence version 1). This plasmid can be further modified with constitutive transcriptional units for a lactose permease like e.g. LAC12 from K. lactis (UniProt ID P07921, sequence version 1) and a galactoside beta-1,3-N-acetylglucosaminyltransferase activity like e.g. IgtA from N. meningitidis (UniProt ID Q9JXQ6, sequence version 1) to produce LN3. To further produce LN3-derived oligosaccharides like LNT, the mutant LN3 producing strains were further modified with a constitutive transcriptional unit for an N-acetylglucosamine beta-1,3-galactosyltransferase like e.g. WbgO (Uniprot ID D3QY14, sequence version 1) from E. coli 055: H7.

In an example for production of Lacto-N-biose, a yeast expression plasmid can be derived from the pRS420-plasmid series (Christianson et al., 1992, Gene 110:119-122) containing the TRP1 selection marker and constitutive transcriptional units for one or more copies of an L-glutamine-D-fructose-6-phosphate aminotransferase like e.g. the mutant glmS*54 from E. coli (differing from the wild-type E. coli glmS, having UniProt ID P17169, sequence version 4, by an A39T, an R250C and an G472S mutation as described by Deng et al. (Biochimie 88, 419-29 (2006)), a phosphatase like any one of e.g. the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonas putida, ScDOG1 from S. cerevisiae or BsAraL from Bacillus subtilis as described in WO18122225, a glucosamine 6-phosphate N-acetyltransferase like e.g. GNA1 from S. cerevisiae (UniProt ID P43577, sequence version 1) and an N-acetylglucosamine beta-1,3-galactosyltransferase like e.g. WbgO from E. coli 055: H7 (Uniprot ID D3QY14, sequence version 1).

Preferably but not necessarily, any one or more of the glycosyltransferase and/or the proteins involved in nucleotide-activated sugar synthesis were N- and/or C-terminally fused to a SUMOstar tag (e.g. obtained from pYSUMOstar, Life Sensors, Malvern, PA) to enhance their solubility.

Optionally, the mutant yeast strains were modified with a genomic knock-in of a constitutive transcriptional unit encoding a chaperone protein like e.g. Hsp31, Hsp32, Hsp33, Sno4, Kar2, Ssb1, Sse1, Sse2, Ssa1, Ssa2, Ssa3, Ssa4, Ssb2, Ecm10, Ssc1, Ssq1, Ssz1, Lhs1, Hsp82, Hsc82, Hsp78, Hsp104, Tcp1, Cct4, Cct8, Cct2, Cct3, Cct5, Cct6 or Cct7 (Gong et al., 2009, Mol. Syst. Biol. 5:275). Plasmids were maintained in the host E. coli DH5alpha (F, phi80dlacZdeltaM15, delta (lacZYA-argF) U169, deoR, recA1, endA1, hsdR17 (rk, mk+), phoA, supE44, lambda “, thi-1, gyrA96, relA1) bought from Invitrogen.

### Heterologous and Homologous Expression

Genes that needed to be expressed, be it from a plasmid or from the genome were synthetically synthetized with one of the following companies: DNA2.0, Gen9, IDT or Twist Bioscience. Expression could be further facilitated by optimizing the codon usage to the codon usage of the expression host. Genes were optimized using the tools of the supplier.

### Cultivations Conditions

In general, yeast strains were initially grown on SD CSM plates to obtain single colonies. These plates were grown for 2-3 days at 30° C. Starting from a single colony, a preculture was grown over night in 5 mL at 30° C., shaking at 200 rpm. Subsequent 125 ml shake flask experiments were inoculated with 2% of this preculture, in 25 mL media. These shake flasks were incubated at 30° C. with an orbital shaking of 200 rpm.

### Gene Expression Promoters

Genes were expressed using synthetic constitutive promoters, as described by Blazeck (Biotechnology and Bioengineering, Vol. 109, No. 11, 2012).

### Example 4. Production of 2′FLNB with a Modified E. coli Strain

A mutant E. coli strain modified for production of GDP-fucose and LNB (Gal-b1,3-GlcNAc) as described in Example 2 was transformed with an expression plasmid comprising a constitutive transcriptional unit for one alpha-1,2-fucosyltransferase selected from SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 and 37. The novel strains were evaluated in a growth experiment for production of 2′FLNB (Fuc-a1,2-Gal-b1,3-GlcNAc) according to the culture conditions provided in Example 2, in which the strains were cultivated in minimal medium with 30 g/L sucrose. The strains were grown in four biological replicates in a 96-well plate. After 72h of incubation, the culture broth was harvested, and the sugars were analysed on UPLC. For each strain with a particular a1,2-fucosyltransferase tested, the measured 2′FLNB concentration was averaged over all biological replicates, and then normalized to the averaged 2′FLNB concentration of a reference strain expressing the alpha-1,2-fucosyltransferase with SEQ ID NO 06. As demonstrated in Table 2, all novel strains demonstrated to produce 2′FLNB. The strains expressing SEQ ID NO 31, 32, 33 or 36 produced less 2′FLNB compared to the reference strain, whereas the other tested strains produced more 2′FLNB than the reference strain. The strains expressing SEQ ID NO 05, 09, 12, 16, 13, 19, 21, 22, 24, 25, 26, 27, 29 or 30 produced more than two times more 2′FLNB than the reference strain, whereas the strains expressing SEQ ID NO 01, 02, 07, 10, 14, 17, 18, 20, 23 or 28 produced more than three times more 2′FLNB than the reference strain.

TABLE 2
Relative production of 2′FLNB (Fuc-a1,2-Gal-b1,3-GlcNAc) (%)
in mutant E. coli strains expressing an alpha-1,2-fucosyltransferase
and producing GDP-fucose and LNB, when evaluated in a growth
experiment according to the culture conditions provided in Example
2, in which the culture medium contained 30 g/L sucrose as carbon
source, and compared to a reference strain expressing the alpha-
1,2-fucosyltransferase with SEQ ID NO 06.

| | alpha-1,2-fucosyltransferase | |
|---|---|---|
| Strain | expressed | 2′FLNB (%) |
| 06 | SEQ ID NO 06 | 100 |
| 33 | SEQ ID NO 33 | 11 |
| 32 | SEQ ID NO 32 | 44 |
| 31 | SEQ ID NO 31 | 70 |
| 36 | SEQ ID NO 36 | 77 |
| 34 | SEQ ID NO 34 | 118 |
| 35 | SEQ ID NO 35 | 131 |
| 04 | SEQ ID NO 04 | 131 |
| 37 | SEQ ID NO 37 | 137 |
| 11 | SEQ ID NO 11 | 142 |
| 08 | SEQ ID NO 08 | 155 |
| 03 | SEQ ID NO 03 | 156 |
| 15 | SEQ ID NO 15 | 166 |
| 24 | SEQ ID NO 24 | 226 |
| 21 | SEQ ID NO 21 | 228 |
| 19 | SEQ ID NO 19 | 235 |
| 29 | SEQ ID NO 29 | 235 |
| 22 | SEQ ID NO 22 | 235 |
| 13 | SEQ ID NO 13 | 247 |
| 30 | SEQ ID NO 30 | 248 |
| 12 | SEQ ID NO 12 | 252 |
| 26 | SEQ ID NO 26 | 256 |
| 27 | SEQ ID NO 27 | 261 |
| 05 | SEQ ID NO 05 | 263 |
| 25 | SEQ ID NO 25 | 265 |
| 09 | SEQ ID NO 09 | 286 |
| 16 | SEQ ID NO 16 | 296 |
| 20 | SEQ ID NO 20 | 309 |
| 28 | SEQ ID NO 28 | 322 |
| 01 | SEQ ID NO 01 | 324 |
| 23 | SEQ ID NO 23 | 337 |
| 14 | SEQ ID NO 14 | 342 |
| 02 | SEQ ID NO 02 | 352 |
| 07 | SEQ ID NO 07 | 356 |
| 17 | SEQ ID NO 17 | 357 |
| 18 | SEQ ID NO 18 | 361 |
| 10 | SEQ ID NO 10 | 383 |

### Example 5. Evaluation of Mutant E. coli 2′FLNB Production Strains in Fed-Batch Fermentations

The mutant E. coli strains as described in Example 4 were evaluated in a fed-batch fermentation process. Fed-batch fermentations at bioreactor scale were performed as described in Example 2. Sucrose is used as a carbon source. No lactose is added to the fermentation process. In contrast to the cultivation experiments that are described herein and wherein only end samples were taken at the end of cultivation (i.e. 72 hours as described herein), regular broth samples were taken at several time points during the fermentation process and the production of LNB and 2′FLNB (Fuc-α1,2-Gal-b1,3-GlcNAc) at each of said time points was measured using UPLC as described in Example 2. A fermentation with the mutant E. coli strain 06 expressing the alpha-1,2-fucosyltransferase with SEQ ID NO 06 as described in Example 4 showed to produce equal titres of LNB and 2′FLNB in whole broth samples taken at the end of fermentation.

### Example 6. Production of LNFP-I with a Modified E. coli Strain

A mutant E. coli strain modified for production of GDP-fucose as described in Example 2 was further modified with a genomic knock-in of constitutive transcriptional units for the galactoside beta-1,3-N-acetylglucosaminyltransferase IgtA from N. meningitidis (UniProt ID Q9JXQ6, sequence version 1) and the N-acetylglucosamine beta-1,3-galactosyltransferase wbgO from E. coli 055: H7 (Uniprot ID D3QY14, sequence version 1) and transformed with an expression plasmid comprising a constitutive transcriptional unit for one alpha-1,2-fucosyltransferase selected from SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 and 37. The novel strains were evaluated in a growth experiment for production of LNFP-I (Fuc-a1,2-Gal-b1,3-GlcNAc-b1,3-Gal-b1,4-Glc) and 2′-FL (Fuc-a1,2-Gal-b1,4-Glc) according to the culture conditions provided in Example 2, in which the strains were cultivated in minimal medium with 30 g/L sucrose and 20 g/L lactose. The strains were grown in four biological replicates in a 96-well plate. After 72h of incubation, the culture broth was harvested, and the sugars were analysed on UPLC. For each strain with a particular a1,2-fucosyltransferase tested, the measured LNFP-I and 2′-FL concentration was averaged over all biological replicates, and then normalized to the averaged LNFP-I or 2′-FL concentration, respectively, of a reference strain expressing the alpha-1,2-fucosyltransferase with SEQ ID NO 06. In this experiment, the strain expressing the alpha-1,2-fucosyltransferases with SEQ ID NO 01, 02, 03, 04, 05, 06, 07, 08, 09, 10, 11, 12, 13, 14, 15, 16, 17 or 18 demonstrated to produce LNFP-I (see Table 3). The strains expressing an alpha-1,2-fucosyltransferase with SEQ ID NO 19, 20, 21, 22, 23, 24, 25, 26, 27, 28, 29, 30, 31, 32, 33, 34, 35, 36 or 37 did not produce LNFP-I (Results not shown). The strains expressing the alpha-1,2-fucosyltransferases with SEQ ID NO 01, 02, 04, 05, 06, 08, 11, 17 and 18 showed additional production of 2′-FL in the cell next to production of LNFP-I (Table 3). The strains expressing SEQ ID NO 01, 02, 04, 17 and 18 showed low production of 2′-FL, being less than 50% of the amount of LNFP-I formed in the cell. The strains expressing SEQ ID NO 05, 06, 08 or 11 were the best 2′-FL producing strains, having 4 to 8 times more 2′-FL produced 5 than LNFP-I.

TABLE 3
Relative production of LNFP-I (Fuc-a1,2-Gal-b1,3-GlcNAc-b1,3-Gal-
b1,4-Glc) (%) and 2′-FL (%) in mutant E. coli strains expressing
an alpha-1,2-fucosyltransferase and producing GDP-fucose, when
evaluated in a growth experiment according to the culture conditions
provided in Example 2, in which the culture medium contained
30 g/L sucrose as carbon source and 20 g/L lactose as precursor,
and compared to the production of LNFP-I or 2′-FL, respectively,
in a reference strain expressing the alpha-1,2-fucosyltransferase
with SEQ ID NO 06. The relative production (%) of 2′-FL to
LNFP-I is also given for each strain.

| | alpha-1,2- | | | |
|---|---|---|---|---|
| | fucosyltransferase | | | 2′-FL/LNFP-I |
| Strain | expressed | LNFP-I (%) | 2′-FL (%) | (%) |
| 38 | SEQ ID NO 06 | 100 | 100 | 650 |
| 39 | SEQ ID NO 18 | 9.0 | 0.70 | 50 |
| 40 | SEQ ID NO 17 | 12.0 | 0.70 | 40 |
| 41 | SEQ ID NO 16 | 16.0 | 0 | 0 |
| 42 | SEQ ID NO 15 | 25.0 | 0 | 0 |
| 43 | SEQ ID NO 14 | 30.8 | 0 | 0 |
| 44 | SEQ ID NO 13 | 46.8 | 0 | 0 |
| 45 | SEQ ID NO 12 | 56.7 | 0 | 0 |
| 46 | SEQ ID NO 11 | 67.1 | 84.4 | 803 |
| 47 | SEQ ID NO 10 | 76.8 | 0 | 0 |
| 48 | SEQ ID NO 09 | 94.7 | 0 | 0 |
| 49 | SEQ ID NO 08 | 96.1 | 57.1 | 388 |
| 50 | SEQ ID NO 07 | 99.0 | 0 | 0 |
| 51 | SEQ ID NO 05 | 131 | 90.7 | 454 |
| 52 | SEQ ID NO 04 | 229 | 6.70 | 18.8 |
| 53 | SEQ ID NO 03 | 261 | 0 | 0 |
| 54 | SEQ ID NO 02 | 318 | 10.5 | 21.4 |
| 55 | SEQ ID NO 01 | 355 | 0.1 | 0 |

### Example 7. Production of 2′-FL with a Modified E. coli Strain

A mutant E. coli strain modified for production of GDP-fucose as described in Example 2 was further transformed with an expression plasmid comprising a constitutive transcriptional unit for one alpha-1,2-fucosyltransferase selected from SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 and 37. The novel strains were evaluated in a growth experiment for production of 2′-FL (Fuc-α1,2-Gal-b1,4-Glc) according to the culture conditions provided in Example 2, in which the strains were cultivated in minimal medium with 30 g/L sucrose and 20 g/L lactose. The strains were grown in four biological replicates in a 96-well plate. After 72h of incubation, the culture broth was harvested, and the sugars were analysed on UPLC. For each strain with a particular a1,2-fucosyltransferase tested, the measured 2′-FL concentration was averaged over all biological replicates, and then normalized to the averaged 2′-FL concentration of a reference strain expressing the alpha-1,2-fucosyltransferase with SEQ ID NO 06.

The strains expressing an alpha-1,2-fucosyltransferase with SEQ ID NO 02, 03, 04, 07, 13, 14, 16, 17, 18, 19, 20, 21, 22, 23, 26, 27, 30, 33, 34, 35, 36 or 37 did not produce 2′-FL (Results not shown) and the strains expressing an alpha-1,2-fucosyltransferase with SEQ ID NO 01, 09, 10, 12, 15, 24, 25, 28, 29, 31 or 32 demonstrated to produce low 2′-FL titres, being less than 20% of the 2′FL produced by the reference strain 56 expressing the alpha-1,2-fucosyltransferase with SEQ ID NO 06. The strains expressing an alpha-1,2-fucosyltransferase with SEQ ID NO 05, 08 or 11 produced 80% or more 2′FL compared to the 2′FL titres measured in the reference strain with SEQ ID NO 06 (Table 4).

TABLE 4
Relative production of 2′-FL (Fuc-a1,2-Gal-b1,4-Glc) (%) in mutant
E. coli strains expressing an alpha-1,2-fucosyltransferase and
producing GDP-fucose, when evaluated in a growth experiment according
to the culture conditions provided in Example 2, in which the culture
medium contained 30 g/L sucrose as carbon source and 20 g/L lactose
as precursor, and compared to a reference strain expressing the
alpha-1,2-fucosyltransferase with SEQ ID NO 06.

| | alpha-1,2-fucosyltransferase | |
|---|---|---|
| Strain | expressed | 2′-FL (%) |
| 56 | SEQ ID NO 06 | 100 |
| 57 | SEQ ID NO 01 | 1.47 |
| 58 | SEQ ID NO 24 | 2.19 |
| 59 | SEQ ID NO 15 | 2.7 |
| 60 | SEQ ID NO 09 | 2.84 |
| 61 | SEQ ID NO 25 | 2.92 |
| 62 | SEQ ID NO 31 | 4.77 |
| 63 | SEQ ID NO 12 | 5.24 |
| 64 | SEQ ID NO 28 | 16.19 |
| 65 | SEQ ID NO 32 | 17.21 |
| 66 | SEQ ID NO 29 | 18.95 |
| 67 | SEQ ID NO 10 | 21.31 |
| 68 | SEQ ID NO 05 | 81.15 |
| 69 | SEQ ID NO 08 | 85.43 |
| 70 | SEQ ID NO 11 | 108 |

### Example 8. Evaluation of Mutant E. coli Strains in Fed-Batch Fermentations

The mutant E. coli strains as described in Example 6 were evaluated in a fed-batch fermentation process. Fed-batch fermentations at bioreactor scale were performed as described in Example 2. Sucrose was used as a carbon source and lactose was added in the batch medium as precursor to the fermentation process. In contrast to the cultivation experiments that are described herein and wherein only end samples were taken at the end of cultivation (i.e. 72 hours as described herein), regular broth samples were taken at several time points during the fermentation process and the production of LNFP-I and/or 2′FL at each of said time points was measured using UPLC as described in Example 2. The relative production of 2′-FL or

LNFP-I obtained in the broth samples of each strain was calculated by dividing the production titre of 2′-FL or LNFP by the sum of the production titres of 2′FL and LNFP-I produced by that strain. Fermentations with the mutant E. coli strains 38 and 54, expressing the alpha-1,2-fucosyltransferase with SEQ ID NO 06 or 02, respectively, showed production of both 2′-FL and LNFP-I in different ratios in whole broth samples taken at the end of fermentation. Mutant E. coli strain 38 showed a relative production of 55% 2′-FL and 45% LNFP-I in whole broth samples taken at the end of fermentation when calculated on the total sum of 2′-FL and LNFP-I produced. Mutant E. coli strain 54 showed a relative production of 3.60% 2′-FL and 96.7% LNFP-I in whole broth samples taken at the end of fermentation when calculated on the total sum of 2′-FL and LNFP-I produced. A similar fermentation was performed with mutant E. coli strain 53, expressing the alpha-1,2-fucosyltransferase with SEQ ID NO 03. Here, a high production titer of LNFP-I was obtained and no 2′FL could be detected in the medium.

### Example 9. Production of 2′FLNB, 2′-FL and LNFP-I in a Modified E. coli Strain

The mutant E. coli strains producing GDP-Fucose, expressing an alpha-1,2-fucosyltransferase selected form the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 and 37 and producing LNB and 2′-FLNB as described in Example 4, are further modified with a genomic knock-in of a constitutive transcriptional unit for the galactoside beta-1,3-N-acetylglucosaminyltransferase IgtA from N. meningitidis (UniProt ID Q9JXQ6, sequence version 1). The novel strains are evaluated in a growth experiment for the production of 2′FL, LN3, LNT, LNFP-I and 2′-FLNB according to the culture conditions provided in Example 1, in which the culture medium contains 30 g/L sucrose and 20 g/L lactose. The strains are grown in four biological replicates in a 96-well plate. After 72h of incubation, the culture broth is harvested, and the sugars are analysed on UPLC.

### Example 10. Production of 2′FLNB, 2′-FL and LNFP-I in a Modified E. coli Strain

The mutant E. coli strains producing GDP-fucose, LN3 and LNT and expressing an alpha-1,2-fucosyltransferase selected form the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 and 37 as described in Example 6 were further modified with a genomic knock-in of a constitutive transcriptional unit for the mutant glmS*54 from E. coli (differing from the wild-type E. coli glmS, having UniProt ID P17169, sequence version 4, by an A39T, an R250C and an G472S mutation as described by Deng et al. (Biochimie 88, 419-29 (2006)), the glucosamine 6-phosphate N-acetyltransferase GNA1 from S. cerevisiae (UniProt ID P43577, sequence version 1) and one phosphatase chosen from the list comprising any one or more of the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonas putida, ScDOG1 from S. cerevisiae and BsAraL from Bacillus subtilis as described in WO18122225. The novel strains are evaluated in a growth experiment for the production of 2′FL, LN3, LNT, LNFP-I, LNB and 2′-FLNB according to the culture conditions provided in Example 1, in which the culture medium contains 30 g/L sucrose and 20 g/L lactose. The strains are grown in four biological replicates in a 96-well plate. After 72h of incubation, the culture broth is harvested, and the sugars are analysed on UPLC.

### Example 11. Production of 2′FLNB with a Modified S. cerevisiae Strain

An S. cerevisiae strain is adapted for production of GDP-fucose and LNB and for expression of an a-1,2-fucosyltransferase as described in Example 3 with a first yeast expression plasmid comprising constitutive transcriptional units for the lactose permease LAC12 from K. lactis (UniProt ID P07921, sequence version 1), the GDP-mannose 4,6-dehydratase gmd from E. coli (UniProt ID POAC88, sequence version 1), the GDP-L-fucose synthase fcl from E. coli (UniProt ID P32055, sequence version 2) and one a1,2-fucosyltransferase chosen from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 and 37 and with a second yeast expression plasmid comprising constitutive transcriptional units for the UDP-glucose 4-epimerase galE from E. coli (UniProt ID P09147, sequence version 1) and the N-acetylglucosamine beta-1,3-galactosyltransferase WbgO from E. coli 055: H7 (UniProt ID D3QY14, sequence version 1). The novel strains are evaluated in a growth experiment for the production of 2′-FLNB according to the culture conditions provided in Example 3, in which the SD CSM-Ura-His drop-out medium comprises glucose as carbon source and GlcNAc as precursor. The strains are grown in four biological replicates in a 96-well plate. After 72h of incubation, the culture broth is harvested, and the sugars are analysed on UPLC.

### Example 12. Production of 2′FLNB with a Modified S. cerevisiae Strain

Mutant S. cerevisiae strains adapted for production of GDP-fucose and LNB and for expression of an a-1,2-fucosyltransferase chosen from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 and 37 as described in Example 11 are further modified for production of GlcNAc by genomic knock-ins of constitutive transcriptional units for the mutant glmS*54 from E. coli (differing from the wild-type E. coli glmS, having UniProt ID P17169, sequence version 4, by an A39T, an R250C and an G472S mutation as described by Deng et al. (Biochimie 88, 419-29 (2006)), an additional copy of the glucosamine 6-phosphate N-acetyltransferase GNA1 from S. cerevisiae (UniProt ID P43577, sequence version 1) and one phosphatase chosen from the list comprising any one or more of the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonas putida, ScDOG1 from S. cerevisiae and BsAraL from Bacillus subtilis as described in WO18122225. The novel strains are evaluated in a growth experiment for the production of 2′-FLNB according to the culture conditions provided in Example 3, in which the SD CSM-Ura-His drop-out medium comprises glucose as carbon source and no precursor. The strains are grown in four biological replicates in a 96-well plate. After 72h of incubation, the culture broth is harvested, and the sugars are analysed on UPLC.

### Example 13. Production of LNFP-I with a Modified S. cerevisiae Strain

An S. cerevisiae strain is adapted for production of GDP-fucose and LNT and for expression of an a-1,2-fucosyltransferase as described in Example 3 with a first yeast expression plasmid comprising constitutive transcriptional units for the lactose permease LAC12 from K. lactis (UniProt ID P07921, sequence version 1), the GDP-mannose 4,6-dehydratase gmd from E. coli (UniProt ID POAC88, sequence version 1), the GDP-L-fucose synthase fcl from E. coli (UniProt ID P32055, sequence version 2) and one a1,2-fucosyltransferase chosen from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 and 37 and with a second yeast expression plasmid comprising constitutive transcriptional units for the UDP-glucose 4-epimerase galE from E. coli (UniProt ID P09147, version sequence 1), the galactoside beta-1,3-N-acetylglucosaminyltransferase IgtA from N. meningitidis (UniProt ID Q9JXQ6, sequence version 1) and the N-acetylglucosamine beta-1,3-galactosyltransferase WbgO from E. coli 055: H7 (UniProt ID D3QY14, sequence version 1). The novel strains are evaluated in a growth experiment for the production of LNFP-I according to the culture conditions provided in Example 3, in which the SD CSM-Ura-His drop-out medium comprises glucose as carbon source and lactose as precursor. The strains are grown in four biological replicates in a 96-well plate. After 72h of incubation, the culture broth is harvested, and the sugars are analysed on UPLC. When GlcNAc is added as additional precursor, the mutant strains are also evaluated for LNB and 2′FLNB production.

### Example 14. Material and Methods Bacillus subtilis Media

Two different media are used, namely a rich Luria Broth (LB) and a minimal medium for shake flask (MMsf). The minimal medium uses a trace element mix.

Trace element mix consisted of 0.735 g/L CaCl2).2H2O, 0.1 g/L MnCl2.2H2O, 0.033 g/L CuCl2.2H2O, 0.06 g/L CoCl2.6H2O, 0.17 g/L ZnCl2, 0.0311 g/L H3BO4, 0.4 g/L Na2EDTA.2H2O and 0.06 g/L Na2MoO4. The Fe-citrate solution contained 0.135 g/L FeCl3.6H2O, 1 g/L Na-citrate (Hoch 1973 PMC1212887).

The Luria Broth (LB) medium consisted of 1% tryptone peptone (Difco, Erembodegem, Belgium), 0.5% yeast extract (Difco) and 0.5% sodium chloride (VWR. Leuven, Belgium). Luria Broth agar (LBA) plates consisted of the LB media, with 12 g/L agar (Difco, Erembodegem, Belgium) added.

The minimal medium for the shake flasks (MMsf) experiments contained 2.00 g/L (NH4) 2SO4, 7.5 g/L KH2PO4, 17.5 g/L K2HPO4, 1,25 g/L Na-citrate, 0.25 g/L MgSO4.7H2O, 0.05 g/L tryptophan, from 10 up to 30 g/L glucose or another carbon source including but not limited to fructose, maltose, sucrose, glycerol and maltotriose when specified in the examples, 10 ml/L trace element mix and 10 ml/L Fe-citrate solution. The medium was set to a pH of 7 with 1M KOH. Depending on the experiment lactose, GlcNAc, LNB or LacNAc could be added as a precursor.

Complex medium, e.g. LB, was sterilized by autoclaving (121° C., 21′) and minimal medium by filtration (0.22 μm Sartorius). When necessary, the medium was made selective by adding an antibiotic (e.g. zeocin (20 mg/L)).

### Strains, Plasmids and Mutations

Bacillus subtilis 168, available at Bacillus Genetic Stock Center (Ohio, USA).

Plasmids for gene deletion via Cre/lox are constructed as described by Yan et al. (Appl. & Environm. Microbial., September 2008, p 5556-5562). Gene disruption is done via homologous recombination with linear DNA and transformation via electroporation as described by Xue et al. (J. Microb. Meth. 34 (1999) 183-191). The method of gene knockouts is described by Liu et al. (Metab. Engine. 24 (2014) 61-69). This method uses 1000 bp homologies up- and downstream of the target gene.

Integrative vectors as described by Popp et al. (Sci. Rep., 2017, 7, 15158) are used as expression vector and could be further used for genomic integrations if necessary. A suitable promoter for expression can be derived from the part repository (iGem): sequence id: Bba_K143012, Bba_K823000, Bba_K823002 or Bba_K823003. Cloning can be performed using Gibson Assembly, Golden Gate assembly, Cliva assembly, LCR or restriction ligation.

In an example for the production of LNB, Bacillus subtilis mutant strains are modified with genomic knock-ins comprising constitutive transcriptional units for the mutant glmS*54 from E. coli (differing from the wild-type E. coli glmS, having UniProt ID P17169, sequence version 4, by an A39T, an R250C and an G472S mutation as described by Deng et al. (Biochimie 88, 419-29 (2006)), the glucosamine 6-phosphate N-acetyltransferase GNA1 from S. cerevisiae (UniProt ID P43577, sequence version 1), one phosphatase chosen from the list comprising any one or more of the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonas putida, DOG1 from S. cerevisiae and AraL from Bacillus subtilis as described in WO18122225 and an N-acetylglucosamine beta-1,3-galactosyltransferase WbgO from E. coli 055: H7 (UniProt ID D3QY14, sequence version 1). To further fucosylate said LNB into 2′FLNB, the mutant strain is further modified with a constitutive transcriptional unit for an alpha-1,2-fucosyltransferase selected from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 and 37.

In an example for the production of lactose-based oligosaccharides, Bacillus subtilis mutant strains are created to contain a gene coding for a lactose importer (such as e.g. the E. coli lacY with UniProt ID P02920, sequence version 1).

In an example for the production of lacto-N-triose (LNT-II, LN3, GlcNAc-b1,3-Gal-b1,4-Glc), the B. subtilis strain is modified with a genomic knock-in of constitutive transcriptional units comprising a lactose importer (such as e.g. the E. coli lacY with UniProt ID P02920, sequence version 1) and a galactoside beta-1,3-N-acetylglucosaminyltransferase like e.g. LgtA from N. meningitidis (GenBank: AAM33849.1). For LNT production, the LN3 producing strain is further modified with a constitutive transcriptional unit for an N-acetylglucosamine beta-1,3-galactosyltransferase like e.g. WbgO from E. coli 055: H7 (UniProt ID D3QY14, sequence version 1). The N-acetylglucosamine beta-1,3-galactosyltransferase can be delivered to the strain either via genomic knock-in or from an expression plasmid. For the production of LNFP-I, the LNT producing strain is further modified with a constitutive transcriptional unit for an alpha-1,2-fucosyltransferase like e.g. SEQ ID NO 01, 02, 03, 04, 05, 06, 07, 08, 09, 10, 11, 12,13, 14, 15, 16, 17 or 18. For growth on sucrose, the mutant strains can additionally be modified with genomic knock-ins of constitutive transcriptional units comprising the sucrose transporter (CscB) from E. coli W (UniProt ID EOIXR1, sequence version 1), the fructose kinase (Frk) from Z. mobilis (UniProt ID Q03417, sequence version 1) and the sucrose phosphorylase (BaSP) from B. adolescentis (UniProt ID AOZZH6, sequence version 1).

### Heterologous and Homologous Expression

Genes that needed to be expressed, be it from a plasmid or from the genome were synthetically synthetized with one of the following companies: DNA2.0, Gen9, Twist Biosciences or IDT. Expression could be further facilitated by optimizing the codon usage to the codon usage of the expression host. Genes were optimized using the tools of the supplier.

### Cultivation Conditions

A preculture of 96-well microtiter plate experiments was started from a cryovial or a single colony from an LB plate, in 150 μL LB and was incubated overnight at 37° C. on an orbital shaker at 800 rpm. This culture was used as inoculum for a 96-well square microtiter plate, with 400 μL MMsf medium by diluting 400×. Each strain was grown in multiple wells of the 96-well plate as biological replicates. These final 96-well culture plates were then incubated at 37° C. on an orbital shaker at 800 rpm for 72h, or shorter, or longer.

At the end of the cultivation experiment samples were taken from each well to measure the supernatant concentration (extracellular sugar concentrations, after 5 min. spinning down the cells), or by boiling the culture broth for 15 min at 90° C. or for 60 min at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the oligosaccharide concentrations by the biomass, in relative percentages compared to a reference strain. The biomass is empirically determined to be approximately ⅓rd of the optical density measured at 600 nm.

### Example 15. Production of 2′FLNB with a Modified B. subtilis Strain

A B. subtilis strain is first modified for LNB production and growth on sucrose by genomic knock-out of the nagB, glmS and gamA genes and genomic knock-ins of constitutive transcriptional units comprising genes encoding the native fructose-6-P-aminotransferase (UniProt ID POCl73, sequence version 1), the mutant glmS*54 from E. coli (differing from the wild-type E. coli glmS, having UniProt ID P17169, sequence version 4, by an A39T, an R250C and an G472S mutation as described by Deng et al. (Biochimie 88, 419-29 (2006)), the glucosamine 6-phosphate N-acetyltransferase GNA1 from S. cerevisiae (UniProt ID P43577, sequence version 1), the phosphatase Aral from B. subtilis (UniProt ID P94526, sequence version 1), the N-acetylglucosamine beta-1,3-galactosyltransferase WbgO from E. coli 055: H7 (UniProt ID D3QY14, sequence version 1), the sucrose transporter (CscB) from E. coli W (UniProt ID EOIXR1, sequence version 1), the fructose kinase (Frk) from Z. mobilis (UniProt ID Q03417, sequence version 1) and the sucrose phosphorylase (BaSP) from B. adolescentis (UniProt ID AOZZH6, sequence version 1). In a next step, the LNB producing strain is transformed with an expression plasmid comprising a constitutive transcriptional unit for an alpha-1,2-fucosyltransferase chosen from the list comprising SEQ ID 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 and 37. The novel strains are evaluated for the production 2′FLNB in a growth experiment on MMsf medium lacking a precursor according to the culture conditions provided in Example 14. After 72h of incubation, the culture broth is harvested, and the sugars are analysed on UPLC.

### Example 16. Production of LNFP-I with a Modified B. subtilis Strain

A B. subtilis strain is first modified for LN3 production and growth on sucrose by genomic knock-out of the nagB, glmS and gamA genes and genomic knock-ins of constitutive transcriptional units comprising genes encoding the lactose permease (LacY) from E. coli (UniProt ID P02920, sequence version 1), the native fructose-6-P-aminotransferase (UniProt ID POCl73, sequence version 1), the galactoside beta-1,3-N-acetylglucosaminyltransferase LgtA from N. meningitidis (GenBank: AAM33849.1, sequence version 1), the sucrose transporter (CscB) from E. coli W (UniProt ID EOIXR1, sequence version 1), the fructose kinase (Frk) from Z. mobilis (UniProt ID Q03417, sequence version 1) and the sucrose phosphorylase (BaSP) from B. adolescentis (UniProt ID AOZZH6, sequence version 1). In a next step, the mutant strain is further modified with a genomic knock-in of a constitutive transcriptional unit comprising the N-acetylglucosamine beta-1,3-galactosyltransferase WbgO from E. coli 055: H7 (UniProt ID D3QY14, sequence version 1) to produce LNT. In a subsequent step, the LNT producing strain is transformed with an expression plasmid comprising constitutive transcriptional units for an alpha-1,2-fucosyltransferase chosen from the list comprising SEQ ID 01, 02, 03, 04, 05, 06, 07, 08, 09, 10, 11, 12, 13, 14, 15, 16, 17 and 18. The novel strains are evaluated for the production LNFP-I and 2′-FL in a growth experiment on MMsf medium comprising lactose as precursor according to the culture conditions provided in Example 14. After 72h of incubation, the culture broth is harvested, and the sugars are analysed on UPLC.

### Example 17. Material and Methods Corynebacterium glutamicum Media

Two different media are used, namely a rich tryptone-yeast extract (TY) medium and a minimal medium for shake flask (MMsf). The minimal medium uses a 1000× stock trace element mix.

Trace element mix consisted of 10 g/L CaCl2, 10 g/L FeSO4.7H2O, 10 g/L MnSO4.H2O, 1 g/L ZnSO4.7H2O, 0.2 g/L CuSO4, 0.02 g/L NiCl2.6H2O, 0.2 g/L biotin (pH 7.0) and 0.03 g/L protocatechuic acid.

The minimal medium for the shake flasks (MMsf) experiments contained 20 g/L (NH4) 2SO4, 5 g/L urea, 1 g/L KH2PO4, 1 g/L K2HPO4, 0.25 g/L MgSO4.7H2O, 42 g/L MOPS, from 10 up to 30 g/L glucose or another carbon source including but not limited to fructose, maltose, sucrose, glycerol and maltotriose when specified in the examples and 1 ml/L trace element mix. Depending on the experiment lactose, GlcNAc, LNB or LacNAc could be added as a precursor.

The TY medium consisted of 1.6% tryptone (Difco, Erembodegem, Belgium), 1% yeast extract (Difco) and 0.5% sodium chloride (VWR. Leuven, Belgium). TY agar (TYA) plates consisted of the TY media, with 12 g/L agar (Difco, Erembodegem, Belgium) added.

Complex medium, e.g. TY, was sterilized by autoclaving (121° C., 21′) and minimal medium by filtration (0.22 μm Sartorius). When necessary, the medium was made selective by adding an antibiotic (e.g. kanamycin, ampicillin).

### Strains and Mutations

Corynebacterium glutamicum ATCC 13032, available at the American Type Culture Collection.

Integrative plasmid vectors based on the Cre/loxP technique as described by Suzuki et al. (Appl. Microbiol. Biotechnol., 2005 April, 67 (2): 225-33) and temperature-sensitive shuttle vectors as described by Okibe et al. (Journal of Microbiological Methods 85, 2011, 155-163) are constructed for gene deletions, mutations and insertions. Suitable promoters for (heterologous) gene expression can be derived from Yim et al. (Biotechnol. Bioeng., 2013 November, 110 (11): 2959-69). Cloning can be performed using Gibson Assembly, Golden Gate assembly, Cliva assembly, LCR or restriction ligation.

In an example for the production of LNB, the C. glutamicum strain is modified with a genomic knock-in of constitutive expression units comprising the mutant glmS*54 from E. coli (differing from the wild-type E. coli glmS, having UniProt ID P17169, sequence version 4, by an A39T, an R250C and an G472S mutation as described by Deng et al. (Biochimie 88, 419-29 (2006)), the glucosamine 6-phosphate N-acetyltransferase GNA1 from S. cerevisiae (UniProt ID P43577, sequence version 1), one phosphatase chosen from the list comprising any one or more of the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonas putida, ScDOG1 from S. cerevisiae and BsAraL from Bacillus subtilis as described in WO18122225 and an N-acetylglucosamine beta-1,3-galactosyltransferase WbgO from E. coli 055: H7 (UniProt ID D3QY14, sequence version 1). To further fucosylate said LNB into 2′FLNB, the mutant strain is further modified with a constitutive transcriptional unit for an alpha-1,2-fucosyltransferase selected from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 and 37.

In an example for the production of lactose-based oligosaccharides, C. glutamicum mutant strains are created to contain a gene coding for a lactose importer (such as e.g. the E. coli lacY with UniProt ID P02920, sequence version 1).

In an example for the production of lacto-N-triose (LNT-II, LN3, GlcNAc-b1,3-Gal-b1,4-Glc), the C. glutamicum strain is modified with a genomic knock-in of constitutive expression units comprising a lactose importer (such as e.g. the E. coli lacY with UniProt ID P02920, sequence version 1) and a galactoside beta-1,3-N-acetylglucosaminyltransferase like e.g. LgtA from N. meningitidis (GenBank: AAM33849.1). For LNT production, the LN3 producing strain is further modified with a constitutive transcriptional unit for an N-acetylglucosamine beta-1,3-galactosyltransferase like e.g. WbgO from E. coli 055: H7 (UniProt ID D3QY14, sequence version 1). The N-acetylglucosamine beta-1,3-galactosyltransferase can be delivered to the strain either via genomic knock-in or from an expression plasmid. For the production of LNFP-I, the LNT producing strain can further be modified with an alpha-1,2-fucosyltransferase expression construct chosen from the list comprising SEQ ID NO 01, 02, 03, 04, 05, 06, 07, 08, 09, 10, 11, 12, 13, 14, 15, 16, 17 and 18.

For growth on sucrose, the mutant strains can additionally be modified with genomic knock-ins of constitutive transcriptional units comprising the sucrose transporter (CscB) from E. coli W (UniProt ID EOIXR1, sequence version 1), the fructose kinase (Frk) from Z. mobilis (UniProt ID Q03417, sequence version 1) and the sucrose phosphorylase (BaSP) from B. adolescentis (UniProt ID AOZZH6, sequence version 1).

### Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

### Cultivation Conditions

A preculture of 96-well microtiter plate experiments was started from a cryovial or a single colony from a TY plate, in 150 μL TY and was incubated overnight at 37° C. on an orbital shaker at 800 rpm. This culture was used as inoculum for a 96-well square microtiter plate, with 400 μL MMsf medium by diluting 400×. Each strain was grown in multiple wells of the 96-well plate as biological replicates. These final 96-well culture plates were then incubated at 37° C. on an orbital shaker at 800 rpm for 72h, or shorter, or longer.

At the end of the cultivation experiment samples were taken from each well to measure the supernatant concentration (extracellular sugar concentrations, after 5 min. spinning down the cells), or by boiling the culture broth for 15 min at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the oligosaccharide concentrations, e.g. 2′FLNB concentrations, measured in the whole broth by the biomass, in relative percentages compared to the reference strain. The biomass is empirically determined to be approximately ⅓rd of the optical density measured at 600 nm.

### Example 18. Production of 2′FLNB with a Modified C. glutamicum Strain

A C. glutamicum strain is first modified for LNB production and growth on sucrose by genomic knock-out of the Idh, cgl2645 and nagB genes and genomic knock-ins of constitutive transcriptional units comprising genes encoding the mutant glmS*54 from E. coli (differing from the wild-type E. coli glmS, having UniProt ID P17169, sequence version 4, by an A39T, an R250C and an G472S mutation as described by Deng et al. (Biochimie 88, 419-29 (2006)), the glucosamine 6-phosphate N-acetyltransferase GNA1 from S. cerevisiae (UniProt ID P43577, sequence version 1), the phosphatase Aral from B. subtilis (UniProt ID P94526, sequence version 1), the N-acetylglucosamine beta-1,3-galactosyltransferase WbgO from E. coli 055: H7 (UniProt ID D3QY14, sequence version 1), the sucrose transporter (CscB) from E. coli W (UniProt ID EOIXR1, sequence version 1), the fructose kinase (Frk) from Z. mobilis (UniProt ID Q03417, sequence version 1) and the sucrose phosphorylase (BaSP) from B. adolescentis (UniProt ID AOZZH6). In a next step, the LNB producing strain is transformed with an expression plasmid comprising a constitutive transcriptional unit for an alpha-1,2-fucosyltransferase chosen from the list comprising SEQ ID 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 and 37. The novel strains are evaluated for the production 2′FLNB in a growth experiment on MMsf medium lacking a precursor according to the culture conditions provided in Example 17. After 72h of incubation, the culture broth is harvested, and the sugars are analysed on UPLC.

### Example 19. Production of LNFP-I with a Modified C. glutamicum Strain

A C. glutamicum strain is first modified for LN3 production and growth on sucrose by genomic knock-out of the Idh, cgl2645 and nagB genes and genomic knock-ins of constitutive transcriptional units comprising genes encoding the lactose permease (LacY) from E. coli (UniProt ID P02920, sequence version 1), the native fructose-6-P-aminotransferase (UniProt ID POCl73, sequence version 1), the galactoside beta-1,3-N-acetylglucosaminyltransferase LgtA from N. meningitidis (GenBank: AAM33849.1), the sucrose transporter (CscB) from E. coli W (UniProt ID EOIXR1, sequence version 1), the fructose kinase (Frk) from Z. mobilis (UniProt ID Q03417, sequence version 1) and the sucrose phosphorylase (BaSP) from B. adolescentis (UniProt ID AOZZH6, sequence version 1). In a next step, the mutant strain is further modified with a genomic knock-in of a constitutive transcriptional unit comprising the N-acetylglucosamine beta-1,3-galactosyltransferase WbgO from E. coli 055: H7 (UniProt ID D3QY14, sequence version 1) to produce LNT. In a subsequent step, the LNT producing strain is transformed with an expression plasmid comprising constitutive transcriptional units for an alpha-1,2-fucosyltransferase chosen from the list comprising SEQ ID 01, 02, 03, 04, 05, 06, 07, 08, 09, 10, 11, 12, 13, 14, 15, 16, 17 and 18. The novel strains are evaluated for the production LNFP-I and 2′-FL in a growth experiment on MMsf medium comprising lactose as precursor according to the culture conditions provided in Example 17. After 72h of incubation, the culture broth is harvested, and the sugars are analysed on UPLC.

### Example 20. Materials and Methods Chlamydomonas reinhardtii Media

C. reinhardtii cells were cultured in Tris-acetate-phosphate (TAP) medium (pH 7.0). The TAP medium uses a 1000× stock Hutner's trace element mix. Hutner's trace element mix consisted of 50 g/L Na2EDTA.H2O (Titriplex III), 22 g/L ZnSO4.7H2O, 11.4 g/L H3BO3, 5 g/L MnCl2.4H2O, 5 g/L FeSO4.7H2O, 1.6 g/L CoCl2.6H2O, 1.6 g/L CuSO4.5H2O and 1.1 g/L (NH4) 6MoO3.

The TAP medium contained 2.42 g/L Tris (tris(hydroxymethyl)aminomethane), 25 mg/L salt stock solution, 0.108 g/L K2HPO4, 0.054 g/L KH2PO4 and 1.0 mL/L glacial acetic acid. The salt stock solution consisted of 15 g/L NH4CL, 4 g/L MgSO4.7H2O and 2 g/L CaCl2.2H2O. As precursor for saccharide synthesis, precursors like e.g. galactose, glucose, fructose, fucose, GlcNAc, LNB and/or LacNAc could be added. Medium was sterilized by autoclaving (121° C., 21′). For stock cultures on agar slants TAP medium was used containing 1% agar (of purified high strength, 1000 g/cm2).

### Strains, Plasmids and Mutations

C. reinhardtii wild-type strains 21 gr (CC-1690, wild-type, mt+), 6145C (CC-1691, wild-type, mt−), CC-125 (137c, wild-type, mt+), CC-124 (137c, wild-type, mt−) as available from Chlamydomonas Resource Center (https://www.chlamycollection.org), University of Minnesota, U.S.A. Expression plasmids originated from pSI103, as available from Chlamydomonas Resource Center. Cloning can be performed using Gibson Assembly, Golden Gate assembly, Cliva assembly, LCR or restriction ligation. Suitable promoters for (heterologous) gene expression can be derived from e.g. Scranton et al. (Algal Res. 2016, 15:135-142). Targeted gene modification (like gene knock-out or gene replacement) can be carried using the Crispr-Cas technology as described e.g. by Jiang et al. (Eukaryotic Cell 2014, 13 (11): 1465-1469).

Transformation via electroporation was performed as described by Wang et al. (Biosci. Rep. 2019, 39: BSR2018210). Cells were grown in liquid TAP medium under constant aeration and continuous light with a light intensity of 8000 Lx until the cell density reached 1.0-2.0×107 cells/mL. Then, the cells were inoculated into fresh liquid TAP medium in a concentration of 1.0×106 cells/mL and grown under continuous light for 18-20 h until the cell density reached 4.0×106 cells/mL. Next, cells were collected by centrifugation at 1250 g for 5 min at room temperature, washed and resuspended with pre-chilled liquid TAP medium containing 60 mM sorbitol (Sigma, U.S.A.), and iced for 10 min. Then, 250 μl of cell suspension (corresponding to 5.0×107 cells) were placed into a pre-chilled 0.4 cm electroporation cuvette with 100 ng plasmid DNA (400 ng/ml). Electroporation was performed with 6 pulses of 500 V each having a pulse length of 4 ms and pulse interval time of 100 ms using a BTX ECM830 electroporation apparatus (1575 (2, 50 pFD). After electroporation, the cuvette was immediately placed on ice for 10 min. Finally, the cell suspension was transferred into a 50 ml conical centrifuge tube containing 10 ml of fresh liquid TAP medium with 60 mM sorbitol for overnight recovery at dim light by slowly shaking. After overnight recovery, cells were recollected and plated with starch embedding method onto selective 1.5% (w/v) agar-TAP plates containing ampicillin (100 mg/L) or chloramphenicol (100 mg/L). Plates were then incubated at 23+−0.5° C. under continuous illumination with a light intensity of 8000 Lx. Cells were analysed 5-7 days later.

In an example for production of UDP-galactose, C. reinhardtii cells are modified with transcriptional units comprising the gene encoding the galactokinase from Arabidopsis thaliana (KIN, UniProt ID Q9SEE5, sequence version 2) and the gene encoding the UDP-sugar pyrophosphorylase (USP) from A. thaliana (UniProt ID Q9C511, sequence version 1).

In an example for production of LNB, C. reinhardtii cells modified for UDP-galactose production are further modified with an expression plasmid comprising a transcriptional unit for the N-acetylglucosamine beta-1,3-galactosyltransferase WbgO from E. coli 055: H7 (UniProt ID D3QY14, sequence version 1). Additionally, the mutant C. reinhardtii cells can be modified with an expression plasmid comprising transcriptional units for an alpha-1,2-fucosyltransferase chosen from the list comprising SEQ ID NO ID 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 and 37.

### Heterologous and Homologous Expression

Genes that needed to be expressed, be it from a plasmid or from the genome were synthetically synthetized with one of the following companies: DNA2.0, Gen9, Twist Biosciences or IDT. Expression could be further facilitated by optimizing the codon usage to the codon usage of the expression host. Genes were optimized using the tools of the supplier.

### Cultivation Conditions

Cells of C. reinhardtii were cultured in selective TAP-agar plates at 23+/−0.5° C. under 14/10 h light/dark cycles with a light intensity of 8000 Lx. Cells were analysed after 5 to 7 days of cultivation. For high-density cultures, cells could be cultivated in closed systems like e.g. vertical or horizontal tube photobioreactors, stirred tank photobioreactors or flat panel photobioreactors as described by Chen et al. (Bioresour. Technol. 2011, 102:71-81) and Johnson et al. (Biotechnol. Prog. 2018, 34:811-827).

### Example 21. Production of 2′FLNB in Modified C. reinhardtii Cells

C. reinhardtii cells are engineered as described in Example 20 for production of UDP-Gal with genomic knock-ins of constitutive transcriptional units comprising the galactokinase from A. thaliana (KIN, UniProt ID Q9SEE5, sequence version 2) and the UDP-sugar pyrophosphorylase (USP) from A. thaliana (UniProt ID Q9C511, sequence version 1). In a next step, the mutant cells are transformed with an expression plasmid comprising transcriptional units comprising the N-acetylglucosamine beta-1,3-galactosyltransferase WbgO from E. coli 055: H7 (UniProt ID D3QY14, sequence version 1) and an alpha-1,2-fucosyltransferase chosen from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 and 37. The novel strains are evaluated in a cultivation experiment on TAP-agar plates comprising galactose and GlcNAc as precursors according to the culture conditions provided in Example 20. After 5 days of incubation, the cells are harvested, and the production of 2′FLNB is analysed on UPLC.

### Example 22. Materials and Methods Animal Cells

Isolation of Mesenchymal Stem Cells from Adipose Tissue of Different Mammals

Fresh adipose tissue is obtained from slaughterhouses (e.g. cattle, pigs, sheep, chicken, ducks, catfish, snake, frogs) or liposuction (e.g., in case of humans, after informed consent) and kept in phosphate buffer saline supplemented with antibiotics. Enzymatic digestion of the adipose tissue is performed followed by centrifugation to isolate mesenchymal stem cells. The isolated mesenchymal stem cells are transferred to cell culture flasks and grown under standard growth conditions, e.g., 37° C., 5% CO2. The initial culture medium includes DMEM-F12, RPMI, and Alpha-MEM medium (supplemented with 15% foetal bovine serum), and 1% antibiotics. The culture medium is subsequently replaced with 10% FBS (foetal bovine serum)-supplemented media after the first passage. For example, Ahmad and Shakoori (2013, Stem Cell Regen. Med. 9 (2): 29-36), which is incorporated herein by reference in its entirety for all purposes, describes certain variation(s) of the method(s) described herein in this example.

Isolation of Mesenchymal Stem Cells from Milk

This example illustrates isolation of mesenchymal stem cells from milk collected under aseptic conditions from human or any other mammal(s) such as described herein. An equal volume of phosphate buffer saline is added to diluted milk, followed by centrifugation for 20 min. The cell pellet is washed thrice with phosphate buffer saline and cells are seeded in cell culture flasks in DMEM-F12, RPMI, and Alpha-MEM medium supplemented with 10% foetal bovine serum and 1% antibiotics under standard culture conditions. For example, Hassiotou et al. (2012, Stem Cells. 30 (10): 2164-2174), which is incorporated herein by reference in its entirety for all purposes, describes certain variation(s) of the method(s) described herein in this example.

### Differentiation of Stem Cells Using 2D and 3D Culture Systems

The isolated mesenchymal cells can be differentiated into mammary-like epithelial and luminal cells in 2D and 3D culture systems. See, for example, Huynh et al. 1991. Exp Cell Res. 197 (2): 191-199; Gibson et al. 1991, In Vitro Cell Dev Biol Anim. 27 (7): 585-594; Blatchford et al. 1999; Animal Cell Technology': Basic & Applied Aspects, Springer, Dordrecht. 141-145; Williams et al. 2009, Breast Cancer Res 11 (3): 26-43; and Arevalo et al. 2015, Am J Physiol Cell Physiol. 310 (5): C348-C356; each of which is incorporated herein by reference in their entireties for all purposes.

For 2D culture, the isolated cells were initially seeded in culture plates in growth media supplemented with 10 ng/ml epithelial growth factor and 5 pg/ml insulin. At confluence, cells were fed with growth medium supplemented with 2% fetal bovine serum, 1% penicillin-streptomycin (100 U/ml penicillin, 100 ug/ml streptomycin), and 5 pg/ml insulin for 48h. To induce differentiation, the cells were fed with complete growth medium containing 5 pg/ml insulin, 1 pg/ml hydrocortisone, 0.65 ng/ml triiodothyronine, 100 nM dexamethasone, and 1 pg/ml prolactin. After 24h, serum is removed from the complete induction medium.

For 3D culture, the isolated cells were trypsinized and cultured in Matrigel, hyaluronic acid, or ultra-low attachment surface culture plates for six days and induced to differentiate and lactate by adding growth media supplemented with 10 ng/ml epithelial growth factor and 5 pg/ml insulin. At confluence, cells were fed with growth medium supplemented with 2% foetal bovine serum, 1% penicillin-streptomycin (100 U/ml penicillin, 100 ug/ml streptomycin), and 5 pg/ml insulin for 48h. To induce differentiation, the cells were fed with complete growth medium containing 5 pg/ml insulin, 1 pg/ml hydrocortisone, 0.65 ng/ml triiodothyronine, 100 nM dexamethasone, and 1 pg/ml prolactin. After 24h, serum is removed from the complete induction medium.

### Method of Making Mammary-Like Cells

Mammalian cells are brought to induced pluripotency by reprogramming with viral vectors encoding for Oct4, Sox2, Klf4, and c-Myc. The resultant reprogrammed cells are then cultured in Mammocult media (available from Stem Cell Technologies), or mammary cell enrichment media (DMEM, 3% FBS, estrogen, progesterone, heparin, hydrocortisone, insulin, EGF) to make them mammary-like, from which expression of select milk components can be induced. Alternatively, epigenetic remodelling are performed using remodelling systems such as CRISPR/Cas9, to activate select genes of interest, such as casein, a-lactalbumin to be constitutively on, to allow for the expression of their respective proteins, and/or to down-regulate and/or knock-out select endogenous genes as described e.g. in WO21067641, which is incorporated herein by reference in its entirety for all purposes.

### Cultivation

Completed growth media includes high glucose DMEM/F12, 10% FBS, 1% NEAA, 1% pen/strep, 1% ITS-X, 1% F-Glu, 10 ng/ml EGF, and 5 pg/ml hydrocortisone. Completed lactation media includes high glucose DMEM/F12, 1% NEAA, 1% pen/strep, 1% ITS-X, 1% F-Glu, 10 ng/ml EGF, 5 pg/ml hydrocortisone, and 1 pg/ml prolactin (5 ug/ml in Hyunh 1991). Cells are seeded at a density of 20,000 cells/cm2 onto collagen coated flasks in completed growth media and left to adhere and expand for 48 hours in completed growth media, after which the media is switched out for completed lactation media. Upon exposure to the lactation media, the cells start to differentiate and stop growing. Within about a week, the cells start secreting lactation product(s) such as milk lipids, lactose, casein and whey into the media. A desired concentration of the lactation media can be achieved by concentration or dilution by ultrafiltration. A desired salt balance of the lactation media can be achieved by dialysis, for example, to remove unwanted metabolic products from the media. Hormones and other growth factors used can be selectively extracted by resin purification, for example the use of nickel resins to remove His-tagged growth factors, to further reduce the levels of contaminants in the lactated product.

### Example 23. Evaluation of 2′FL, LNFP-I and 2′FLNB Production in a Non-Mammary Adult Stem Cell

Isolated mesenchymal cells and re-programmed into mammary-like cells as described in Example 22 are modified via CRISPR-CAS to over-express the beta-1,4-galactosyltransferase 1 B4GalT1 from Homo sapiens (UniProt ID P15291, sequence version 5), the GDP-fucose synthase GFUS from Homo sapiens (UniProt ID Q13630, sequence version 1), and a codon-optimized alpha-1,2-fucosyltransferase chosen from the list comprising SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 and 37. Cells are seeded at a density of 20,000 cells/cm2 onto collagen coated flasks in completed growth media and left to adhere and expand for 48 hours in completed growth media, after which the media is switched out for completed lactation media for about 7 days. After cultivation as described in Example 22, cells are subjected to UPLC to analyse for production of 2′FL, LNFP-I and 2′FLNB.

### Example 24. Production of LNDFH I with Modified E. coli Strains

The mutant E. coli strains modified for production of GDP-fucose and LNFP-I, each expressing one alpha-1,2-fucosyltransferase selected from SEQ ID NO 03, 07, 09, 10, 12, 13, 14, 15 and 16 as described in Example 6 is further modified with an expression plasmid comprising a constitutive transcriptional unit for a second fucosyltransferase from Helicobacter pylori with UniProt ID O30511, sequence version 1. The novel strains are evaluated in a growth experiment for the production of LNDFH I (Fuc-α1,2-Gal-b1,3-[Fuc-a1,4]-GlcNAc-b1,3-Gal-b1,4-Glc) according to the culture conditions provided in Example 1, in which the culture medium contains 30 g/L sucrose and 20 g/L lactose. The strains are grown in four biological replicates in a 96-well plate. After 72h of incubation, the culture broth is harvested, and the sugars are analysed on UPLC.

### Example 25. Evaluation of Alpha-1,2-Fucosyltransferase Activity

Another example provides the evaluation of alpha-1,2-fucosyltransferase activity of the enzymes with SEQ ID NO 01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36 or 37 of the present invention. These enzymes can be produced in a cell-free expression system such as but not limited to the PURExpress system (NEB), or in a host organism such as but not limited to Escherichia coli or Saccharomyces cerevisiae, after which the above listed enzymes can be isolated and optionally further purified. Each of the above enzyme extracts or purified enzymes are added to a reaction mixture together with GDP-fucose and a buffering component such as Tris-HCl or HEPES and a substrate like e.g. lactose, lacto-N-biose (LNB) or lacto-N-tetraose (LNT). Said reaction mixture is then incubated at a certain temperature (for example 37° C.) for a certain amount of time (for example 24 hours), during which the lactose, LNB or LNT will be converted by the enzyme using GDP-fucose to 2′-fucosyllactose, 2′FLNB or LNFP-I, respectively. The oligosaccharides are then separated from the reaction mixture by methods known in the art. Further purification of lactose, 2′FLNB or LNFP-I can be performed if preferred. At the end of the reaction or after separation and/or purification, the production of 2-fucosyllactose, 2′FLNB or LNFP-I is measured via analytical methods as described in Example 2 and known by the person skilled in the art.

### Example 26. Production of LNDFH-I Using Mutant E. coli Strains in Fed-Batch Fermentations

In another experiment, the mutant E. coli strain 53 as described in Example 6, expressing the a1,2-fucosyltransferase with SED ID NO 03, was transformed with a second plasmid expressing the fucosyltransferase with uniprot ID A0A0G4K5H1 (sequence version 1 dated 16 Sep. 2015). This strain was evaluated in a fed-batch fermentation process. Fed-batch fermentations at bioreactor scale were performed as described in Example 2. Sucrose was used as a carbon source and lactose was added in the batch medium as precursor to the fermentation process. In contrast to the cultivation experiments that are described herein and wherein only end samples were taken at the end of cultivation (i.e. 72 hours as described herein), regular broth samples were taken at several time points during the fermentation process and the production of LN3, LNT, LNFP-I, LNFP-II, LNDFH-I and/or LNDFH-II at each of said time points was measured using UPLC as described in Example 2. Fermentations with a mutant E. coli strain expressing the fucosyltransferase with uniprot ID A0A0G4K5H1 (sequence version 1 dated 16 Sep. 2015) and the alpha-1,2-fucosyltransferase with SEQ ID 03 demonstrated a relative production of 17.0% LNT, 48.0% LNFP-I, 10.5% LNFP-II, 20.7% LNDFH-I and 3.8% LNDFH-II in whole broth samples taken at the end of fermentation when calculated by dividing the production titers of LNT, LNFP-I, LNFP-II, LNDFH-I or LNDFH-II by the total sum of the production of LNT, LNFP-I, LNFP-II, LNDFH-I and LNDFH-II produced by that strain.

1.-88. (canceled)

89. A method for producing a compound comprising a structure of Formula I, II or III: 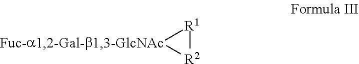
Fuc-α1,2-Gal-β1,3-GlcNAc Formula I
Fuc-α1,2-Gal-β1,3-GlcNAc-R¹ Formula II
wherein:
R¹ is a monosaccharide, disaccharide, oligosaccharide, protein, glycoprotein, peptide, glycopeptide, lipid or glycolipid; R² is a monosaccharide, disaccharide or oligosaccharide;
by a cell, wherein the method comprises the steps of:
i. providing a cell that is genetically engineered for producing the compound, wherein the cell expresses an alpha-1,2-fucosyltransferase, and
ii. cultivating and/or incubating the cell under conditions permissive to express the compound,
wherein the alpha-1,2-fucosyltransferase has galactoside alpha-1,2-fucosyltransferase activity on a galactose residue of Gal-b1,3-GlcNAc (LNB, lacto-N-biose) of the compound, and:
comprises a polypeptide according to any one of SEQ ID NO:1, 3, 5, 6, 8, 11, 12, 13, 15, 19, 21, 22, 23, 24, 26, 28, 29, 30, 31, 32, 33, 34, 35, 36 or 37, or
comprises a polypeptide having at least 80% sequence identity to a full-length sequence of any one of SEQ ID NO: 1, 3, 5, 6, 8, 11, 12, 13, 15, 19, 21, 22, 23, 24, 26, 28, 29, 30, 31, 32, 33, 34, 35, 36 or 37, or
is a functional fragment comprising at least 70.0% of a full-length of any one of SEQ ID NO: 1, 3, 5, 6, 8, 11, 12, 13, 15, 19, 21, 22, 23, 24, 26, 28, 29, 30, 31, 32, 33, 34, 35, 36 or 37.

90. The method according to claim 89, wherein the alpha-1,2-fucosyltransferase:
comprises a polypeptide according to any one of SEQ ID NO:1, 3, 12, 13, 15, 19, 21, 22, 23, 24, 26, 28, 29, 30, 34, 35, 36 or 37, or
comprises a polypeptide having at least 80% sequence identity to the full-length sequence of any one of SEQ ID NO: 1, 3, 12, 13, 15, 19, 21, 22, 23, 24, 26, 28, 29, 30, 34, 35, 36 or 37, or
is a functional fragment comprising at least 70.0% of the full-length of any one of SEQ ID NO: 1, 3, 12, 13, 15, 19, 21, 22, 23, 24, 26, 28, 29, 30, 34, 35, 36 or 37.

91. The method according to claim 89, wherein the alpha-1,2-fucosyltransferase has additional galactoside alpha-1,2-fucosyltransferase activity on the galactose residue at a non-reducing end of Gal-b1,3-GlcNAc-b1,3-Gal-b1,4-Glc (LNT, lacto-N-tetraose) and wherein the alpha-1,2-fucosyltransferase:
comprises a polypeptide according to any one of SEQ ID NO:1, 3, 5, 6, 8, 11, 12, 13 or 15, 16, 17 or 18, or
comprises a polypeptide having at least 80% sequence identity to the full-length sequence of any one of SEQ ID NO: 1, 3, 5, 6, 8, 11, 12, 13, 15, 16, 17 or 18, or
is a functional fragment comprising at least 70.0% of the full-length of any one of SEQ ID NO: 1, 3, 5, 6, 8, 11, 12, 13, 15, 16, 17, or 18.

92. The method according to claim 91, wherein the alpha-1,2-fucosyltransferase has no additional galactoside alpha-1,2-fucosyltransferase activity on lactose or has additional galactoside alpha-1,2-fucosyltransferase activity on lactose, which is lower than its additional galactoside alpha-1,2-fucosyltransferase activity on the galactose residue at the non-reducing end of LNT, and
comprises a polypeptide according to any one of SEQ ID NO:1, 3, 12, 13 or 15, or
comprises a polypeptide having at least 80% sequence identity to the full-length sequence of any one of SEQ ID NO:1,3, 12, 13 or 15, or
is a functional fragment comprising at least 70.0% of the full-length of any one of SEQ ID NO: 1, 3, 12, 13 or 15.

93. The method according to claim 91, wherein the alpha-1,2-fucosyltransferase has additional galactoside alpha-1,2-fucosyltransferase activity on lactose that is higher than its additional galactoside alpha-1,2-fucosyltransferase activity on the galactose residue at the non-reducing end of LNT, and
comprises a polypeptide according to any one of SEQ ID NO:5, 6, 8 or 11, or
comprises a polypeptide having at least 80% sequence identity to the full-length sequence of any one of any one of SEQ ID NO:5, 6, 8 or 11, or
is a functional fragment comprising at least 70.0% of the full-length of SEQ ID NO:5, 6, 8 or 11.

94. The method according to claim 89, wherein the alpha-1,2-fucosyltransferase has no galactoside alpha-1,2-fucosyltransferase activity on the galactose residue at a non-reducing end of LNT, and
comprises a polypeptide according to any one of SEQ ID NO: 19, 21, 22, 23, 24, 26, 28, 29, 30, 31, 32, 33, 34, 35, 36 or 37, or
comprises a polypeptide having at least 80% sequence identity to the full-length sequence of any one of SEQ ID NO: 19, 21, 22, 23, 24, 26, 28, 29, 30, 31, 32, 33, 34, 35, 36 or 37, or
is a functional fragment comprising at least 70.0% of the full-length of any one of SEQ ID NO: 19, 21, 22, 23, 24, 26, 28, 29, 30, 31, 32, 33, 34, 35, 36 or 37.

95. The method according to claim 94, wherein the alpha-1,2-fucosyltransferase has no galactoside alpha-1,2-fucosyltransferase activity on lactose or has an additional galactoside alpha-1,2-fucosyltransferase activity on lactose, which is lower than 3.0% of the galactoside alpha-1,2-fucosyltransferase activity on lactose of the alpha-1,2-fucosyltransferase with SEQ ID NO: 6 and comprises a polypeptide according to any one of SEQ ID NO: 19, 21, 22, 23, 24, 26, 30, 33, 34, 35, 36 or 37, or
comprises a polypeptide having at least 80% sequence identity to the full-length sequence of any one of SEQ ID NO: 19, 21, 22, 23, 24, 26, 30, 33, 34, 35, 36 or 37, or
is a functional fragment comprising at least 70.0% of the full-length of any one of SEQ ID NO: 19, 21, 22, 23, 24, 26, 30, 33, 34, 35, 36 or 37.

96. The method according to claim 94, wherein the alpha-1,2-fucosyltransferase has additional galactoside alpha-1,2-fucosyltransferase activity on lactose that is between 4.0 and 20.0% of the galactoside alpha-1,2-fucosyltransferase activity on lactose of the alpha-1,2-fucosyltransferase with SEQ ID NO:6 and
comprises a polypeptide according to any one of SEQ ID NO:28, 29, 31 or 32, or
comprises a polypeptide having at least 80% sequence identity to the full-length sequence of any one of SEQ ID NO:28, 29, 31 or 32, or
is a functional fragment comprising an oligopeptide sequence of at least 10 consecutive amino acid residues from SEQ ID NO:28, 29, 31 or 32.

97. The method according to claim 89, wherein the compound is an oligosaccharide, a mammalian milk oligosaccharide (MMO), or a human milk oligosaccharide (HMO).

98. The method according to claim 89, wherein the compound is Fuc-α1,2-Gal-b1,3-GlcNAc-b1,3-R wherein R is a monosaccharide, a disaccharide or an oligosaccharide.

99. The method according to claim 89, wherein the compound is lacto-N-fucopentaose I (LNFP-I, Fuc-α1,2-Gal-b1,3-GlcNAc-b1,3-Gal-b1,4-Glc).

100. The method according to claim 89, wherein the cell produces the compound intracellularly.

101. The method according to claim 89, wherein the cell expresses a membrane transporter protein or a polypeptide having transport activity hereby transporting compounds across an outer membrane of a cell wall.

102. The method according to claim 89, wherein the cell produces a mixture of (i) charged disaccharides/oligosaccharides or (ii) neutral disaccharides/oligosaccharides or (iii) charged disaccharides/oligosaccharides and neutral disaccharides/oligosaccharides, wherein the mixture comprises at least one compound comprising a structure of Formula I, II or III, wherein R1, when present, is a monosaccharide, a disaccharide or an oligosaccharide.

103. The method according to claim 89, further comprising:
iii. separating the compound from the cultivation.

104. The method according to claim 89, further comprising the step of purifying the compound from the cell.

105. The method according to claim 89, wherein the cell is a bacterium, fungus, yeast, a plant cell, an animal cell or a protozoan cell.

106. A cell genetically engineered to produce a compound comprising a structure of Formula I, II or III: 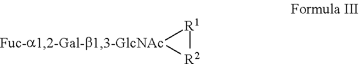
Fuc-α1,2-Gal-β1,3-GlcNAc Formula I
Fuc-α1,2-Gal-β1,3-GlcNAc-R¹ Formula II
wherein:
R¹ is a monosaccharide, disaccharide, oligosaccharide, protein, glycoprotein, peptide, glycopeptide, lipid or glycolipid; R² is a monosaccharide, disaccharide or oligosaccharide;
wherein the cell expresses an alpha-1,2-fucosyltransferase,
wherein the alpha-1,2-fucosyltransferase has galactoside alpha-1,2-fucosyltransferase activity on the galactose residue of Gal-b1,3-GlcNAc (LNB, lacto-N-biose) of the compound and:
comprises a polypeptide according to any one of SEQ ID NO:1, 3, 5, 6, 8, 11, 12, 13, 15, 19, 21, 22, 23, 24, 26, 28, 29, 30, 31, 32, 33, 34, 35, 36, or 37, or
comprises a polypeptide having at least 80% sequence identity to a full-length sequence of any one of SEQ ID NO: 1, 3, 5, 6, 8, 11, 12, 13, 15, 19, 21, 22, 23, 24, 26, 28, 29, 30, 31, 32, 33, 34, 35, 36, or 37, or
is a functional fragment comprising at least 70.0% of a full-length of any one of SEQ ID NO: 1, 3, 5, 6, 8, 11, 12, 13, 15, 19, 21, 22, 23, 24, 26, 28, 29, 30, 31, 32, 33, 34, 35, 36, or 37.

107. The cell of claim 106, wherein the alpha-1,2-fucosyltransferase:
comprises a polypeptide according to any one of SEQ ID NO:1, 3, 12, 13, 15, 19, 21, 22, 23, 24, 26, 28, 29, 30, 34, 35, 36, or 37, or
comprises a polynucleotide having at least 80% sequence identity to the full-length sequence of any one of SEQ ID NO:1, 3, 12, 13, 15, 19, 21, 22, 23, 24, 26, 28, 29, 30, 34, 35, 36, or 37, or
is a functional fragment comprising at least 70.0% of the full-length of any one of SEQ ID NO: 1, 3, 12, 13, 15, 19, 21, 22, 23, 24, 26, 28, 29, 30, 34, 35, 36, or 37.

108. A method of using the cell of claim 106 to produce a compound comprising a structure of Formula I, II or III: 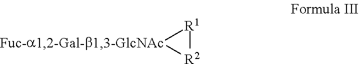
Fuc-α1,2-Gal-β1,3-GlcNAc Formula I
Fuc-α1,2-Gal-β1,3-GlcNAc-R¹ Formula II
wherein:
R¹ is a monosaccharide, disaccharide, oligosaccharide, protein, glycoprotein, peptide, glycopeptide, lipid or glycolipid; R² is a monosaccharide, disaccharide or oligosaccharide,


SEQUENCE LISTING (39 sequences)

SEQ ID NO: 1 - protein, 267 aa, Polaribacter vadi
MIIVRILGGLGNQMFQYAFAKSLQQKGFEVQIDISKFKTYKLHGGYHLDTYNIDLETANSFDTFLALIKLKKNVKEKSLLFDENMLKLSGNEFVKGYFQTEKYFSTIRGILLQQFTIKTELSDSTKKYSKAIHQHKNSCSLHIRRGDYITDKKANSVHGTCDLNYYASAIKLINEKFENTHFFVFSDDIIWTKENLQLENATYIDHKTIPHEDMFLMSLCKHNITANSSFSWWGAWLNQHKNKTVIAPKKWFVSQENEVASKNWIQL

SEQ ID NO: 2 - protein, 292 aa, Dysgonomonas mossii
MKIVKLQGGLGNQMFQYAIARTLETNKKKDIFLDLSFLRMNNVSTDCFTARDFELSIFPHLRAKKLNSLQEKFLLSDRVRYKFIRKIANINFHKINQLENEIVGIPFGIKNVYLDGFFQSESYFKHIRFDLIKDFEFPELDTRNEALKKTIVNNNSVSIHIRRGDYVHLKNANTYHGVLSLEYYLNCIKRIGEETKEQLSFFIFSDDPEYASKSLSFLPNMQIVDWNLGKNSWKDMALMLACKHHIIANSSFSWWGAWLSERNGITYAPVKWFNNESQYNINNIIPSDWVII

SEQ ID NO: 3 - protein, 642 aa, Desulfovibrio alaskensis
  source 1..642: note = G20
MRTQAVVFSKDRALQLRATLESLLLRCTDAAEELRVRVLYKACGEMHRRQYDSLKESFPQVEFVAETDFRIQLLRLLADTPYVLFLVDDNIFYRDFSLGGVLECLEAAPGAVGFSLRLGRNTTYCHTQDAPQRVPGCTVLRDNVLCCRWRGADHDFGYPLEVSSSVYRTAHMLALLHDMPFTNPNLLEAGLSRRTGAVEDSFPQLLFFDRSVTFCNPLNRVQDTFANRVADEPVDAQALAQLYARGQMLDVAAYGDALPDAAHYEMPLRLRSVPAEPEHLSVTDRPFISAVIPVFNGASFLPDAVRSLHRQRYEPMEVIIVNDGSTDDSSAVGAALAAQYPALRIRVADKENGGLASARNAGIGQAGGGWILPLDCDDCFAPEFVGRAAEIISQRPGVNLVFANMQEFGARQGRWNPEPYSLQELMRRNTFPYASLYRKELWEQSGGYDPSMPWGAEDWLFWLSCAPFGLCPHRIEEPLFLYRTHPHGSMYTRMMERWDVVRACLRTLLPALYPAAALLQDHTLVAGMDADTAGVIADIRTRHPRAAMPAFWQGLVHEAAGRTAEAVAGYMHAAAHAPYAQWQPHLRLFMCNLRLGRKKAAHGAAVQAVLRRPELAPLFSGYRELDLQALLPRNMCPETPPR

SEQ ID NO: 4 - protein, 311 aa, Dechlorosoma suillum
  source 1..311: note = PS
MQSPACIAGARAWWVGYGMAEAMQPVVVGLSGGLGNQMFQYAAGRALAHRLGHPLSLDLSWFQGRGDRHFALAPFHIAASLERAWPRLPPAMQAQLSRLSRRWAPRIMGAPVFREPHFHYVPAFAALAAPVFLEGYWQSERYFRELREPLLQDFSLRQPLPASCQPILAAIGNSDAICVHVRRGDYLSNPVAAKVHGVCPVDYYQQGVAELSASLARPHCFVFSDDPEWVRGSLAFPCPMTVVDVNGPAEAHFDLALMAACQHFVIANSSLSWWGAWLGQAAGKRVIAPSRWFLTSDKDARDLLPPSWERR

SEQ ID NO: 5 - protein, 289 aa, Capnocytophaga canis
MMRHYICLQGGLGNQLYILGYAFYLQEQGCKNICLFYEQKQNGDTVDTQKRNIIDDLPKELGFKSLYISSLWLKVLRNLCKLPMISKLVDYYEEPTEEWAVFTPFSPLRKKRVSVHIGYYQSFYYQTPLFIERLKRFFFKNVLLERFSPVENDAAIHIRRGDFLTGVNTMIYSEIGVKYYLEGLEKLNREQGIGKIYVFSDDFQAITNDIEEISKIYTVELMQGNSVLQDIRMLMCFKRYVLGNSTFAWWGAKLSEKQNPIVVVPATPWKINLKKEVTPYPKDWILLEN

SEQ ID NO: 6 - protein, 311 aa, Helicobacter sp.
  source 1..311: note = MIT 01-6242
MFSVRLMGGLGNQMFIYAFAKAIKAQGYPVRLFYYDTDYNVPQTHNIRNLEIVDFGIAMCIETMCYEEPQIKKSFFERALGFIKRKLKIHSPHSSSLISDHCEIALTKDFLDTLNPNAMFNGYFQNVVFFDHLRESLLRDFTLKRPLTPANEALKHQILQTPNSCFLHIRRGDYLQIPIYVKLGSTYYNNAIKALKDKISKPHIFVFSNDIAWCKEFFLDSLDPLVIENVTFSFIENNDEGNAIEEMELMRSCQHAIIANSTFSWWAAYLIDSAQKLCIMPKHFFNDPQQEVAHKLIPPPLHSLSQTIVIG

SEQ ID NO: 7 - protein, 267 aa, Butyrivibrio proteoclasticus
MNYVEVKGGLGNQLFQYTFYKYLEKKSGHKVLLHTDFFKNIDSFEEATKRKLGLDRFDCDFVAVSGFISCEKLVKESDYKDSMLSQDEVFYSGYWQNKRFFLEVMDDIRKDLLLKDENIQDEVKELAKELRAVDSVAIHFRRGDYLSEQNKKIFTSLSVDYYQKAIAQLAERNGADLKGYIFTDEPEYVSGIIDQLGSIDIKLMPVREDYEDLYLMSCARHHIIANSSFSWWGAALGDTESGITIAPAKWYVDGRTPDLYLRNWISI

SEQ ID NO: 8 - protein, 261 aa, Akkermansia muciniphila
MNMERKTGLMNKKYVSPCFLPGMRLGNIMFTLAAACAHARTVGVECRVPWAYNDASLMLRSRLGGWVLPSTPCGTNEPPSWQEPSFAYCPVPSRIRTGGLRGYFQSARYFEGQEAFIRALFAPLTAEKEPGAVGIHIRLGDYRRLRDKHRILDPGFLRRAAGHLSSGKNRLVLFSDEPDEAAEMLARVPAFGRFALEIDRGAPCESLRRMTAMEELVMSCSSFSWWGAWLGNTRKVIVPRDWFVGGVEDYRDIYLPHWVTL

SEQ ID NO: 9 - protein, 290 aa, Butyrivibrio proteoclasticus
  source 1..290: note = B316
MRKRIIALNGGLGNQMFQYAFARMLEDRKHCLIEFDTGFYSTVNDRKLAIQNYNIHKYDFCNHEYYNKIRLLFQKIPFVAWLAGTYKEYSEYQLDPRVFLFNYRFYYGYWQNKQYFENISNDIRNELSYIGNVSEKENALLNMLEAHNAIAIHVRRGDYTQEGYNKIYISLSKEYYKRAVSIACKELGDNNIPLYVFSDDIDWCKANLADIGNVTFVDNTISSSADIDMLMMKKSRCLITANSTFSWWSAWLSDRDDKIVLVPDKWLQDEEKNTKLMKAFICDKWKIVPV

SEQ ID NO: 10 - protein, 293 aa, Thermosynechococcus sp.
  source 1..293: note = NK55
MIIVRLYGGLGNQMFQYAAGLALSLRHAVPLRFDLDWFDGVRLHQGLELHRVFDLDLPRAAPSEMRQVLGSFSHPLVRRLLVRRRLRWLLPQGYALEPHFHYWPGFEALGPKAYLDGYWQSERYFSEYQDAVRAAFRFAQPLDERNRQIVEEMAACESVSLHVRRGDFVQDPVVRRVHGVDLSAYYPRAVALLMERMREPRFYVFSDDPDWVRANLKLPAPMIVIDHNRGEHSFRDMQLMSACRHHILANSSFSWWGAWLNSQPHKLVIAPKRWFNVDDFDTRDLYCSGWTVL

SEQ ID NO: 11 - protein, 297 aa, Porphyromonas catoniae
MKRIYLSIYGGLGNQLYILAYADYLQRMLGTRPYLLNELQRTKADTSSLDRTRRDLFSELIAYLGFQFVDTDSREFRLLKKWERHIKHYQEEPNKHGIYLQNILPPLQENHRWTLPLVCRVSGYFQSCHYVGTDFRMRVSKFLERHATSADLVREYTSMIQPEDVAIHLRRGDFVALQHTGIQLFGAEHYTKGLALQAQQQPIGRVFVFSDDFEAIGEELSLLANNYQLVLVKGLTPLQDLFLLTCFRRYVLANSTFSWWGALCSKYGDAVKVVVPKKPLLISYPEDSYFPPSWEQI

SEQ ID NO: 12 - protein, 280 aa, Francisella sp.
  source 1..280: note = FSC1006
MKVVKIQGGLGNQMFQYAFYMSLKQKYKDCCIDIRDFETYTQHNGFELDRVFENIRQSISLCRCKRKFFRSLFSKFLNRFIKYHKNYFSQDDFGFNKKYYNKDNCYLDGYWQSEKYFKSVEKQIREIFKFQTLDDKNAKILEEYKNRSLVSIHVRRGDYINHPLHGDICNLDYYNNAIDIIKSRVESPHFFVFSDDIEWCKQCLDIEDVTYICTNTGSDSYRDMQIMSICKHNIIANSSFSWWGAWLNQNSEKIIIAPNRWFNDDSINQSDICPESWIKI

SEQ ID NO: 13 - protein, 292 aa, Chitinophaga sp.
  source 1..292: note = CF118
MVVIKITGGLGNQMFQYAFGRRLSLQWNVPLKLDMSELESGRLTTSRNYELHHFNVVQDFVEASELKAVKLDLHGHIMNKLLIGIQEKVWRIKSSHFMENGNVDSLLAGNSRPVYLDGYWQSEKYFKQYGDVIRREFTAKDEWDGHNKQILNSILNADAVSIHIRRGDYVTNAVTNKFHGTCDIDYYQKAIALVEERMEAPFYYIFSDDLPWAKEHLVFSKGTAHFVDNNADAAFMDIMLMSKCKYNIIANSTFSWWGAWLNNNPGKLVIAPKKWFNHKDDSDIIPDTWMKI

SEQ ID NO: 14 - protein, 307 aa, Selenomonas sp.
  source 1..307: note = CM52
MILMRLVGGLGNQMFQYAMASSVARRAGEILKLDLSWIRQMEKKLSADDIYGLGIFSFDEKFSTSNEVQKFLPSGKFSAKIYRAVNRRMPFSWRRVLEEGGMGWHPQIMEIRRSVYFYMGYWQSEKYFSDFIQEIRKDFTFREEVRQSIEERRPIVEKIRKSDAVSLHIRRGDYAQNPALGEIFLSFTMQYYIDAARYISERVKTPVFFIFSDDIPWAKENLPLPYEVCYIDDNIQTNEREIGHKSKGYEDMYLMTQCQHNIIANSSFSWWGAWLNHNPNKIVVAPKKWCNGSFNYADIVPEQWVKL

SEQ ID NO: 15 - protein, 289 aa, Desulfotomaculum reducens
  source 1..289: note = MI-1
MNVMNGSMENHTVGLIFSKDRAMQLDATIRSLYLHCKDVQNIDLKILYKASDAYHLKHYNQLIKEYSSVQFISENNFREQVLSNLFNYAYILFLVDDNIFVHDFSINKIILNLEKQSDALGFSLRLGSNINYCYALNSAQARPVFCELEHETLKYDWTVAEHDFGYPLEVSSSVYRVEDILPLLFQLPFANPNTLEAYMSSNKGCYVDSKPYILCYKQSITFCNPVNKVQNIFNNRAGDEISYSIHELSKMFEQGYRIDVENYSGFIPNACHQEVKLQFLKPEQEYSEK

SEQ ID NO: 16 - protein, 279 aa, Bacteroides fragilis
  source 1..279: note = str. 3397 T10
MKKVIFSGGLGNQMFQYAFYLFLKKKGIKAVIDNSLYSEFKMHNGFELIKVFDIKESIYRTYFLKVHLIFIKLLMKIPPVRKLSCKDDVIPIGDHEFDPPYARFYLGYWQSKKIVNYVIEELRAQFIFRNIPQMTIEKGDFLSSINSVSIHIRRGDYMGIPAYQGICNEIYYERAISFMKEHFLNPRFYVFSNDSIWAKLFLEKFDIDMEIIVTPPIYSYWDMYLMSRCRNHIIANSTFSWWAAVLNMNKDKIVISPTIFKKDECIDIIFDDWVKISNI

SEQ ID NO: 17 - protein, 309 aa, Pseudovibrio sp.
  source 1..309: note = FO-BEG1
MSVASQVRISGAARRRKLKPTLIVRIRGGIGNQLFQYALGRKIALETGMKLRFDRSEYDQYFNRSYCLNLFKTQGLSATESEMSAVLWPAQSFGQTVKLCRKFYPFYQRRYIREDELLQDSETPVLKQSAYLDGYWQTWEIPFSIMEQLRDEITLKKPMVLERLKLLQRIKSGPSAALHVRYGDYSQAHNLQNFGLCSAGYYKGAMDFLTERVPGLTFYVFSDSPERAREVVPQQENVYFSDPMQDGKDHEDLMVMSSCDHIVTANSTFSWWAAFLNGNEDKHVIAPLKWFKNPNLDDSLIVPPHWQRL

SEQ ID NO: 18 - protein, 294 aa, Colwellia psychrerythraea
  source 1..294: note = 34H
MKVVRVCGGFGNQLFQYAFYLAVKHKFNETTKLDIHDMASYELHNGYELERIFNLNENYCSAEEKLAVQSTKNIFTKLLKEIKKYTPFIPRTYIKEKKHLHFSYQEVDLGTKDTSIYYRGSWQNPQYFNSIASEIREKLTFPEFTEPKSLALHQEISEHETVAVHIRRGDYLKHKALGGICDLPYYQNAIKEIEGLVEKPLFVIFSDDITWCRANINVEKVRFVDWNSGEQSFQDMHLMSLCTHNIIANSSFSWWGAWLNANPNKIVISPNKWIHYTDSMGIVPSEWIKVETSI

SEQ ID NO: 19 - protein, 289 aa, Bacteroides fragilis
MIVSSLRGGLGNQMFIYAMVKAMALRNNVPFAFNLTTDFANDEVYKRKLLLSYFALDLPENKKLTFDFSYGNYYRRLSRNLGCHILHPSYRYICEERPPHFESRLISSKITNAFLEGYWQSEKYFLDYKQEIKEDFVIQKKLEYTSYLELEEIKLLDKNAIMIGVRRYQESDVAPGGVLEDDYYKCAMDIMASKVTSPVFFCFSQDLEWVEKHLAGKYPVRLISKKEDDSGTIDDMFLMMHFRNYIISNSSFYWWGAWLSKYDDKLVIAPGNFINKDSVPESWFKLNVR

SEQ ID NO: 20 - protein, 295 aa, Geobacter uraniireducens
  source 1..295: note = Rf4
MIIARLQGGLGNQMFQYAVGLHLALTHNVELKIDITMFSDYKWHTYSLRPFNIRESIATEEEIKALTDVKMDRPYKKIDNFLCRLLRKSQKISATHVKEKHFHYDPDILKLPDNVYLDGYWQSEKYFKEIENIIRQTFIIKNPQLGRDKELACKILSTESVCLHIRRGNYVTDKTTNSVLGPCDLSYYSNCIKSLAGNNKDPHFFVFSNDHEWVSKNLKLDYPTIYVDHNNEDKDYEDLRLMSQCKHHIIANSTFSWWSAWLCSNPDKVIYAPQKWFRVDEYNTKDLLPSNWLIL

SEQ ID NO: 21 - protein, 269 aa, Dyadobacter fermentans
MVGVYFSGRLGNQLFQYYFLLYLKTRNPRLTYFFPNPHHAYLSKYFDLGGYHNLTLGSKLYSGFTRIIPRFISFAPVYVHNFFSPKEFSPKNGQMYYGYYQSDFYIKHLPKGTLPKLKPEFEKAFDEQYGAIFRNEKTVVVHIRRTDYLSYGKRDISLPVEYFQKQLSSIENLDAYKVYFVSDDMDFVKAAFPARDNFHFVSNPEIIDFQLIMNADVAIISNSTFAWWAAYICKKKNHVIAPKNWFGFRIGREHPKGIMTDRFEWRDVL

SEQ ID NO: 22 - protein, 300 aa, Escherichia albertii
  source 1..300: note = KF1
MMMIIRFSGGLGNQLFQFAAAKVLEVKFGGSIVVDDSYYDNQPSKDTFRKLEIFQFNVQYTRKSNCKEKSNTRKKVLILKILTRIPWYSNSSAFRKITRLIHIYNEDAFTYHMNARENDYVIGYFQNYSLLKENINIIRQQFTLTPEVDAEMRRLDSYRTISQHDNTIAVHIRRGDYVTNVNASAFHGICDIEYYKKSIELITSRIKDPKFVFFSDDIAWVKEAFSGVSDAYFVENPSSTSSAVDMYLMSLCKHNIIANSTYSWWGAVLNANPEKIVICPERWTLNDSIGQLYVDGWIKL

SEQ ID NO: 23 - protein, 290 aa, Parabacteroides sp.
  source 1..290: note = YL27
MKIVKFLGGLGNQMFQLALLKSLQNLYPEERILADIFSYNGYGLHNGFEVADIFNLSVEFATLKNVASVAWPFPNYRTWQIGSRILPQRRSMCIDLKNADWDNPKYKYYEGYWQNEKFFKSIRPSLLDVFSFPLDDNIHNNKLLNDLTNTSSISIHIRRGDYIGNKLYQNICTSEYYRNAIEFVTSQLQAVDIACIFSNDMEWCKKNIPVLLPSGVDIRYVDWNTGINSFQDIHLMSKCNHNIIANSSFSWWGAWLNQNPNKIVVTPNKWTNNTIGENPICDSWNRVKIN

SEQ ID NO: 24 - protein, 286 aa, Sphingobacteriaceae bacterium
MVIIKLMGGLGNQMFQYAFALIHMLGNKEVKFDLSYYDSDNRHGGYWLDKAFNINIAKANKRDVFYFIEAIKDEVGNLGYRLKSDKFIIEEVLEEESSYNPDLANLDGAHFSGYWQNVNYFKKIENEVRHQFRFRELEKADLGNLANKKQILDSNAVSIHIRRGDYLQSPIHLNLGLEYYANAIEYIEMKTNEPWFYIFTDDMHWAKQNLGQVNNIQYVETNKKNKCHLDMYLMSLCKHNIIANSTFSWWGAWLNNNPNKIVITPKKWFNHSKNIDGILPKEWIKL

SEQ ID NO: 25 - protein, 292 aa, Prevotella sp.
  source 1..292: note = oral taxon 299 str. F0039
MDSQLLKHIKLSGGFGNQLFQYFFGEYLKEKYNCSISFFSEPALDINQLQIHRFFPTLRISHNTELRRFHYAFTQQLAYRCMRKLLLLFPFLNRKVKIENGSNYQNQSFNDTYCFDGYWQSYRYLSAFTPSLQFEDQLINDISADYINAIEQSEAVFLHIRRGDYLNKENQKVFAECPLNYFENAVNKIKEGNKTYHFFVFSNDIEWVKCHLKLNNNEVTFIQNEGSSCDLKDFYLMTRCKHAIISNSTFSWWAAYLINNNDKKVIAPKRWYNDLSMNNATKDLIPPTWIRL

SEQ ID NO: 26 - protein, 288 aa, Pseudomonas asplenii
MIISNIIGGLGNQMFQYAMARSLSLELKSDLLLDISSYDSYPLHQGYELDRVFKVRSSLAKVEDVKSVLGWQQNLFIHRVLRRPQFSWLRKKSLAIEPFFQYWEGVNFLPKNCYLFGYWQSEKYFNKFSEVIRQDFSFDSNMSEENSFYSERIRKSNSVSVHIRRGDYLNNSVYASCSLEYYRSAIAHVSARSGNPVFFVFSDDIEWVKDNLEFEAESYFVAHNKAGESYNDMRLMSYCKHHVIANSSFSWWGAWLNPSPEKIVIAPKQWFTDGTNTKDLIPSEWMVL

SEQ ID NO: 27 - protein, 288 aa, Bacteroides nordii
  source 1..288: note = CL02T12C05
MMGIEKTNMVIVRLWGGIGNQLFQYSFGEFLREKYQVDVIYDIASFGKSDKLRKLELSVVVPGIPVTTDISFSKYVGTKNRLLRFIYGLKNSFIEEKYFSDEQLFKYLSKRGDVYLQGYWQKTIYAETLRRKGSFFLSQEEPIVLHTIKAKIQEAEGAIALHVRRGDYFSSKHINTFGVCDAHYYEKAVDIMRGRVSNAMIFVFSDDLDWVRRYVNLPTNVIYVPNYDIPQYWYIYLMSLCRHNIISNSSFSWWGAFLNMNTNKIVVSPSKWTLNSDKTIALDEWFKI

SEQ ID NO: 28 - protein, 286 aa, Selenomonas ruminantium
MIIVRFMGGLGNQMFQYAFGLAVEQRGIDVKYDISWMKYHSKKMEIQDVFCLDLSGKIATIDECNELGYTRYDVYGRLRQIFNAKKSFIYQRKAIDSILFDESLMYKDNTYFAGYFQCEQYFLSIKEQVKNVFKFNDSYVGDEVNNRELIKKIVTSESIALHIRRGDYLNNNIYDALDDDYYTRAINKMHRIYSKPMFIIFSNDIPYCKDKYDDINAEFVDWNTGRFSWVDMYLMTLCKGVVIPNSTFSWWGAWLNNNDVKTVIAPRQWFRNIRSGDIIPDSWIRL

SEQ ID NO: 29 - protein, 280 aa, Bacteroides sp.
  source 1..280: note = A51A
MRLIKVTGGLGNQMFIYACYLQMKKRFPRVYIDLTDMMHYHVHYGYEMHRVFGLPCVEICMNQVLKKVIEFLFFKTILERKQHGSMRPYTRSYLWPLIYFKGFYQSEKYFADIKDEVRQAFTFDLKQANGQSLRMREQIDKDEHAVSLHVRRGDYLQPKHWEAIGCICQLSYYRNALEEMGKRVERPRYYVFSDDLAWVKENLDLPDAVYIDWNKGEDSWQDMMLMSRCRHHIICNSTFSWWGAWLNPRNGKIVIAPERWTRDADSREIVPAEWLRVSIK

SEQ ID NO: 30 - protein, 318 aa, Selenomonas sputigena
MVHYRGNYRKRGDKIRMQIIMLNGGLGNQMFQYIFALYLAQKDAHVVLEDSAFFDAHVEPDLFKIPKFFPAGIFPRLSEYFSPDVWEEMLLLKERGKSMIEQLYEGGLPIHYVQEMNSAGPSCAAPTTIIKKDAPAVSVNLSHEQTVYFWGYWITDFYFREIQDTFLSQFHFPSFPTTAQEEMARRICSFQNAAAIHVRRGDMAKLGRSNPLQYFRQAIRKLESLIHVDHFYLFSDDLPYCMNHAKELGLTSIRSRLTVVDGNRGAHDYVDMQLMSLCRFRIIDQSSFSFLAGLLCRLPGNLTIFYDASKLSGCSFSI

SEQ ID NO: 31 - protein, 289 aa, Capnocytophaga leadbetteri
MKANYVLFQGGLGNQLYQLAYTDFLKRNGYSNVKLITPSNKNKGDTKDKNKRPLITQLPEKIGIDCVDFGHKYFYSFLQRLPKFPLYKSFLRRLINVEKEPPYQWAIFHPITREKYRLNIHIGYYQSNLYISDSFKQQVAKVIESLSPNIKFSITNNDVAIHIRRGDFLIGNNASVFNKIELPHYLQGLTILSERMNIQKVYIFSDDFEAIKEDIKTIAENYEVVLVEGQSVLADFALLQKFTNFVIGNSTFAWWGAMLANASNVIVPKKPWKIEMENMSPYPDNWTTI

SEQ ID NO: 32 - protein, 340 aa, Helicobacter sp.
  source 1..340: note = CLO-3
MTIINIRDGLGNQMFQYAFAKVLESKGESVVLDTSWYSEEPDSVSKNIANKKNIRNLEITRFDIKIVPYMEIETMLQTQDKFYHFFSFIHKLIAYAPKECLRVGLKSFLPKTRRFHPYKYHIKEVYDSRDLSALPHDILKNALIFGFFQKLSYFKHIDSEIRADFTLCTPLSPANEAMKKRILSTPNAAFIHIRRGDYLNVWQVIKLGKAYYASAIKEILTHVKNPKFFIFSNDIEWCKNNFINLIDSSVFAGKEYEFVEQNGEGDAIEELELMRSCKHGIIANSTFSWWAAYLMENPKKTVIAPSKFFLIPPPQEPNHIDDILPEGWIKTDPTWGNVES

SEQ ID NO: 33 - protein, 320 aa, Helicobacter sp.
  source 1..320: note = MIT 17-337
MLIVRLDGGLGNQMFQYAFAKKLESKGYEVGIDISWYADKKNTNPTIANFAQNQHATIRNLEISHYNLSLPLLSDFNPYDFFILHDRFYVLRNKINRFLPRPLRMKNYKFFIPDDVKLCKIIQENKPYFPDYAYFSGFYQNLSCIVEPQDISGDFSLKNPLSQANQALKEQIASFQDSVFLHIRRGDYLAFERYIHLTERYYNQALYMMKTKKDSFHVFVFSNDIQWCKEYFIKQLDNTLMQNLTFEFIGNNDEGNAIIDMELMRTCKHGIIANSTFSWWAAYLLKSKDKIIIAPSRFLQDSTIERMKLLYPQEWTLIEV

SEQ ID NO: 34 - protein, 300 aa, Cyanobium gracile
  source 1..300: note = PCC 6307
MRVLTLIFSKDRPLQLQASLASFALHCQEAAQTPITVLHRASSEAFAQGYAQLRQEFQGRLLIDWVEERSFRRDLLASLREPPPSSRWRRLLDRLRLRSWRPRCDQLLFLVDDNIFVRPFSLRSIAEALEQQPSAIGFSLRVGRNTTRCYSMNCAQPLPEFQPMASGLRFRWVGQTGDFGYPIEVSSSVYRLADLIGLLRTLPYTNPNRLEQVLSSSSSLFALGKPDLLCFEQSVAFCAPINKVQTILDNRAGASDDYSSEALLERFLEGQRVDVEALRDFVPRAAHVEIELPLQSASAL

SEQ ID NO: 35 - protein, 281 aa, Desulfococcus oleovorans
  source 1..281: note = Hxd3
MSFTAPAARPRLNIIIFSKNRACQVESLLRSIRDHLVYVPLTVTVLYRATDKRFKAGYEKTIRRHPSPGIQWIEETNFYRDLTSRVLALPPEQLIIFLVDDNIVFKKIDLQFIISQFTTEHLFISLRAGRHYTKDVPVPDFDRRDELLEWQWRIQRKASTTWNYPFSVDGNIYHVGRMQQVMRHIRFVAPNSFESAMHDYRKTRWVRKRNRAIAPVEPVIVNNPLNRVQTEGETWHKQLDPAAINEKYLAGLVLDNAKLYNCDPTDTHCDLGLHWSEKRES

SEQ ID NO: 36 - protein, 313 aa, Elizabethkingia bruuniana
  source 1..313: note = G0146
MKKIIKSKILAFLDKKGFQRKPIGADPKIALYHQMKDIFAPANKEIVSLIFSKDRAMQLDGFLASYFENVDNYSSVKVLFHVSNEEHRNSYKDLERIYAEFPVEFIPETNFRNDLIGILEKANEDRIIFYVDDMLFSQKVDYNWLKEVDPLLDIVSLSRGRDLNYSTALAMKQEIPSFSKISNNLYRFKWNEISEFSDWTYPIGVSGYMFSRQEIMAMIKVTDFKAPNSLEHNLQKFLPYFDTRGGVCLENVATPCVHTNLTQTEGYNNILGYFSLEELLVLWNENKRIDYKEFFGLKVSEAEVKKYNFINRT

SEQ ID NO: 37 - protein, 317 aa, Flammeovirgaceae bacterium
  source 1..317: note = 311
MISLVKGYTRKLIDNFLGKYEYRLLNNKHLAYYEDFAINILKEVERTMDNKEIESIVFSKDRAMQLHAFLASYIERVSNRGRMYILYKCTNQRHQKSYDQLKEGFAGEDFVFIEEKEFRKQLIDICEQSGAGKIMFYVDDMIFTHKIDYAVLGGINTAESILSLSRGKDMDFSIVLQKPLVLPPFTSEENNLERFSWNYLKEFSEWTYPLGVSGYMYGRIEVLAMLKSISFKAPNSLEISMQIYLPYYIRRSGLCTKFAACVCVHANLVQSEIENPTLGTYSIEELLILWEKGKRINIHEFYDTPISETQIKAYTFV

SEQ ID NO: 38-39 - skipped (2 entries)